SEQUENCE LISTING (58 sequences)

SEQ ID NO: 1 - protein, 450 aa, synthetic construct
EVQLVQSGAEVKKPGATVKISCKVSGYTFTNYGMNWVQQAPGKGLEWMGWINTYTGEPTYTEKFQGRVTITADTSTTTAYMELSSLRSEDTAVYYCARGGFGSSYWYFDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 450 aa, synthetic construct
QVQLVQSGAEVKKPGASVKVSCKASGYTFTNYGMNWVRQAPGQRLEWMGWINTYTGEPTYSQEFQGRVTITLDTSASTAYMELSSLRSEDMAVYYCARGGFGSSYWYFDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 3 - protein, 214 aa, synthetic construct
DIQLTQSPSFLSASVGDRVTITCRASQDVSIALAWYQQKPGKAPKLLIYSASYLYSGVPSRFSGSGSGTEFTLTISSLQPEDFATYYCQQHYITPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 214 aa, synthetic construct
DIVMTQSPDSLAVSLGERATINCKSSQDVSIALAWYQQKPGQPPKLLIYSASYRYTGVPDRFSGSGSGTDFTLTISSLQAEDVAVYYCQQHYITPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 450 aa, synthetic construct
  VARIANT 122: note = non-natural amino acid
EVQLVQSGAEVKKPGATVKISCKVSGYTFTNYGMNWVQQAPGKGLEWMGWINTYTGEPTYTEKFQGRVTITADTSTTTAYMELSSLRSEDTAVYYCARGGFGSSYWYFDVWGQGTMVTVSSXSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 6 - protein, 450 aa, synthetic construct
  VARIANT 122: note = non-natural amino acid
QVQLVQSGAEVKKPGASVKVSCKASGYTFTNYGMNWVRQAPGQRLEWMGWINTYTGEPTYSQEFQGRVTITLDTSASTAYMELSSLRSEDMAVYYCARGGFGSSYWYFDVWGQGTMVTVSSXSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 7 - protein, 214 aa, synthetic construct
  VARIANT 121: note = non-natural amino acid
DIQLTQSPSFLSASVGDRVTITCRASQDVSIALAWYQQKPGKAPKLLIYSASYLYSGVPSRFSGSGSGTEFTLTISSLQPEDFATYYCQQHYITPLTFGQGTKLEIKRTVAAPSVFIFPPXDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 214 aa, synthetic construct
  VARIANT 110: note = non-natural amino acid
DIVMTQSPDSLAVSLGERATINCKSSQDVSIALAWYQQKPGQPPKLLIYSASYRYTGVPDRFSGSGSGTDFTLTISSLQAEDVAVYYCQQHYITPLTFGQGTKLEIKRTXAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 214 aa, synthetic construct
  VARIANT 112: note = non-natural amino acid
DIVMTQSPDSLAVSLGERATINCKSSQDVSIALAWYQQKPGQPPKLLIYSASYRYTGVPDRFSGSGSGTDFTLTISSLQAEDVAVYYCQQHYITPLTFGQGTKLEIKRTVAXPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 214 aa, synthetic construct
  VARIANT 114: note = non-natural amino acid
DIVMTQSPDSLAVSLGERATINCKSSQDVSIALAWYQQKPGQPPKLLIYSASYRYTGVPDRFSGSGSGTDFTLTISSLQAEDVAVYYCQQHYITPLTFGQGTKLEIKRTVAAPXVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 214 aa, synthetic construct
  VARIANT 121: note = non-natural amino acid
DIVMTQSPDSLAVSLGERATINCKSSQDVSIALAWYQQKPGQPPKLLIYSASYRYTGVPDRFSGSGSGTDFTLTISSLQAEDVAVYYCQQHYITPLTFGQGTKLEIKRTVAAPSVFIFPPXDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 214 aa, synthetic construct
  VARIANT 127: note = non-natural amino acid
DIVMTQSPDSLAVSLGERATINCKSSQDVSIALAWYQQKPGQPPKLLIYSASYRYTGVPDRFSGSGSGTDFTLTISSLQAEDVAVYYCQQHYITPLTFGQGTKLEIKRTVAAPSVFIFPPSDEQLKXGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 214 aa, synthetic construct
  VARIANT 149: note = non-natural amino acid
DIVMTQSPDSLAVSLGERATINCKSSQDVSIALAWYQQKPGQPPKLLIYSASYRYTGVPDRFSGSGSGTDFTLTISSLQAEDVAVYYCQQHYITPLTFGQGTKLEIKRTVAAPSVFIFPPSDEQLKSGTASVVCLLNNFYPREAKVQWXVDNALQSGNSQESVTEQDSKDSTYSLSSTLTLSKADYEKHKVYACEVTHQGLSSPVTKSFNRGEC

SEQ ID NO: 14 - protein, 214 aa, synthetic construct
  VARIANT 156: note = non-natural amino acid
DIVMTQSPDSLAVSLGERATINCKSSQDVSIALAWYQQKPGQPPKLLIYSASYRYTGVPDRFSGSGSGTDFTLTISSLQAEDVAVYYCQQHYITPLTFGQGTKLEIKRTVAAPSVFIFPPSDEQLKSGTASVVCLLNNFYPREAKVQWKVDNALQXGNSQESVTEQDSKDSTYSLSSTLTLSKADYEKHKVYACEVTHQGLSSPVTKSFNRGEC

SEQ ID NO: 15 - protein, 214 aa, synthetic construct
  VARIANT 168: note = non-natural amino acid
DIVMTQSPDSLAVSLGERATINCKSSQDVSIALAWYQQKPGQPPKLLIYSASYRYTGVPDRFSGSGSGTDFTLTISSLQAEDVAVYYCQQHYITPLTFGQGTKLEIKRTVAAPSVFIFPPSDEQLKSGTASVVCLLNNFYPREAKVQWKVDNALQSGNSQESVTEQDXKDSTYSLSSTLTLSKADYEKHKVYACEVTHQGLSSPVTKSFNRGEC

SEQ ID NO: 16 - protein, 214 aa, synthetic construct
  VARIANT 202: note = non-natural amino acid
DIVMTQSPDSLAVSLGERATINCKSSQDVSIALAWYQQKPGQPPKLLIYSASYRYTGVPDRFSGSGSGTDFTLTISSLQAEDVAVYYCQQHYITPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLXSPVTKSFNRGEC

SEQ ID NO: 17 - protein, 214 aa, synthetic construct
  VARIANT 205: note = non-natural amino acid
DIVMTQSPDSLAVSLGERATINCKSSQDVSIALAWYQQKPGQPPKLLIYSASYRYTGVPDRFSGSGSGTDFTLTISSLQAEDVAVYYCQQHYITPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPXTKSFNRGEC

SEQ ID NO: 18 - protein, 447 aa, synthetic construct
EVQLVQSGAEVKKPGATVKISCKVSGYTFTDYYMNWVQQAPGKGLEWMGIINPYNGGTHYNQKFKGRVTITADTSTDTAYMELSSLRSEDTAVYYCATSGYDLYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 19 - protein, 214 aa, synthetic construct
EIVMTQSPATLSVSPGERATLSCKASQNVGTAVAWYQQKPGQAPRLLIYSAFNRYNGIPARFSGSGPGTDFTLTISSLQSEDFAVYYCQQYSTYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 447 aa, synthetic construct
  VARIANT 119: note = non-natural amino acid
EVQLVQSGAEVKKPGATVKISCKVSGYTFTDYYMNWVQQAPGKGLEWMGIINPYNGGTHYNQKFKGRVTITADTSTDTAYMELSSLRSEDTAVYYCATSGYDLYFDYWGQGTLVTVSSXSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 21 - protein, 214 aa, synthetic construct
  VARIANT 110: note = non-natural amino acid
EIVMTQSPATLSVSPGERATLSCKASQNVGTAVAWYQQKPGQAPRLLIYSAFNRYNGIPARFSGSGPGTDFTLTISSLQSEDFAVYYCQQYSTYPLTFGGGTKVEIKRTXAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 214 aa, synthetic construct
  VARIANT 112: note = non-natural amino acid
EIVMTQSPATLSVSPGERATLSCKASQNVGTAVAWYQQKPGQAPRLLIYSAFNRYNGIPARFSGSGPGTDFTLTISSLQSEDFAVYYCQQYSTYPLTFGGGTKVEIKRTVAXPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 214 aa, synthetic construct
  VARIANT 114: note = non-natural amino acid
EIVMTQSPATLSVSPGERATLSCKASQNVGTAVAWYQQKPGQAPRLLIYSAFNRYNGIPARFSGSGPGTDFTLTISSLQSEDFAVYYCQQYSTYPLTFGGGTKVEIKRTVAAPXVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 214 aa, synthetic construct
  VARIANT 121: note = non-natural amino acid
EIVMTQSPATLSVSPGERATLSCKASQNVGTAVAWYQQKPGQAPRLLIYSAFNRYNGIPARFSGSGPGTDFTLTISSLQSEDFAVYYCQQYSTYPLTFGGGTKVEIKRTVAAPSVFIFPPXDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 447 aa, synthetic construct
  VARIANT 119: note = non-natural amino acid
EVQLVQSGAEVKKPGATVKISCKVSGYTFTDYYMNWVQQAPGKGLEWMGIINPYNGGTHYNQKFKGRVTITADTSTDTAYMELSSLRSEDTAVYYCATSGYDLYFDYWGQGTLVTVSSXSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 26 - protein, 118 aa, synthetic construct
EVQLVQSGAEVKKPGATVKISCKVSGYTFTDYYMNWVQQAPGKGLEWMGIINPYNGGTHYNQKFKGRVTITADTSTDTAYMELSSLRSEDTAVYYCATSGYDLYFDYWGQGTLVTVSS

SEQ ID NO: 27 - protein, 107 aa, synthetic construct
EIVMTQSPATLSVSPGERATLSCKASQNVGTAVAWYQQKPGQAPRLLIYSAFNRYNGIPARFSGSGPGTDFTLTISSLQSEDFAVYYCQQYSTYPLTFGGGTKVEIK

SEQ ID NO: 28 - protein, 449 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 29 - protein, 449 aa, synthetic construct
  VARIANT 121: note = non-natural amino acid
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSXSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 30 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 214 aa, synthetic construct
  VARIANT 121: note = non-natural amino acid
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPXDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - protein, 115 aa, synthetic construct
QVQLVQSGAEVKKPGASVKVSCKASGYTFTEYTIHWVRQAPGQRLEWMGNINPNNGGTTYNQKFEDRVTITRDTSASTAYMELSSLRSEDTAVYYCAAGWNFDYWGQGTTLTVSS

SEQ ID NO: 33 - protein, 110 aa, synthetic construct
DIQLTQSPSFLSASVGDRVTITCKASQDVGTAVDWYQQKPGKAPKLLIYWASTRHTGVPSRFSGSGSGTEFTLTISSLQPEDFATYYCQQYNSYPLTFGGGTKVEIKRTV

SEQ ID NO: 34 - protein, 110 aa, synthetic construct
DIQLTQSPSFLSASVGDRVTITCKASQDVGTAVDWYQQKPGKAPKLLIYWASTRHTGVPDRFSGSGSGTEFTLTISSLQPEDFATYYCQQYNSYPLTFGGGTKVEIKRTV

SEQ ID NO: 35 - protein, 110 aa, synthetic construct
DIQLTQSPSFLSASVGDRVTITCKASQDVGTAVDWYQQKPGKAPKLLIYWASTRHTGVPSRFSGSGSGTEFTLTISSLQSEDFATYYCQQYNSYPLTFGGGTKVEIKRTV

SEQ ID NO: 36 - protein, 110 aa, synthetic construct
DIQLTQSPSFLSASVGDRVTITCKASQDVGTAVDWYQQKPGKAPKLLIYWASTRHTGVPSRFSGSGSGTEFTLTISNLQPEDFATYYCQQYNSYPLTFGGGTKVEIKRTV

SEQ ID NO: 37 - protein, 115 aa, synthetic construct
EVQLQQSGPELVKPGTSVRISCKTSGYTFTEYTIHWVKQSHGKSLEWIGNINPNNGGTTYNQKFEDKATLTVDKSSSTAYMELRSLTSEDSAVYYCAAGWNFDYWGQGTTLTVSS

SEQ ID NO: 38 - protein, 110 aa, synthetic construct
DIVMTQSHKFMSTSVGDRVSIICKASQDVGTAVDWYQQKPGQSPKLLIYWASTRHTGVPDRFTGSGSGTDFTLTITNVQSEDLADYFCQQYNSYPLTFGAGTKVEIKRTV

SEQ ID NO: 39 - protein, 444 aa, synthetic construct
  VARIANT 116: note = non-natural amino acid
QVQLVQSGAEVKKPGASVKVSCKASGYTFTEYTIHWVRQAPGQRLEWMGNINPNNGGTTYNQKFEDRVTITRDTSASTAYMELSSLRSEDTAVYYCAAGWNFDYWGQGTTLTVSSXSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 40 - protein, 214 aa, synthetic construct
DIQLTQSPSFLSASVGDRVTITCKASQDVGTAVDWYQQKPGKAPKLLIYWASTRHTGVPSRFSGSGSGTEFTLTISSLQPEDFATYYCQQYNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 214 aa, synthetic construct
DIQLTQSPSFLSASVGDRVTITCKASQDVGTAVDWYQQKPGKAPKLLIYWASTRHTGVPSRFSGSGSGTEFTLTISSLQSEDFATYYCQQYNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - protein, 214 aa, synthetic construct
DIQLTQSPSFLSASVGDRVTITCKASQDVGTAVDWYQQKPGKAPKLLIYWASTRHTGVPDRFSGSGSGTEFTLTISSLQPEDFATYYCQQYNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 214 aa, synthetic construct
DIQLTQSPSFLSASVGDRVTITCKASQDVGTAVDWYQQKPGKAPKLLIYWASTRHTGVPSRFSGSGSGTEFTLTISNLQPEDFATYYCQQYNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - protein, 444 aa, synthetic construct
  VARIANT 116: note = non-natural amino acid
EVQLQQSGPELVKPGTSVRISCKTSGYTFTEYTIHWVKQSHGKSLEWIGNINPNNGGTTYNQKFEDKATLTVDKSSSTAYMELRSLTSEDSAVYYCAAGWNFDYWGQGTTLTVSSXSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 45 - protein, 190 aa, synthetic construct
DIVMTQSHKFMSTSVGDRVSIICKASQDVGTAVDWYQQKPGQSPKLLIYWASTRHTGVPDRFTGSGSGTDFTLTITNVQSEDLADYFCQQYNSYPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHK

SEQ ID NO: 46 - protein, 446 aa, synthetic construct
QVQLQQWGAGLLKPSETLSLTCAVYGGSFSGYYWSWIRQPPGKGLEWIGEINHSGSTNYNPSLKSRVTISVETSKNQFSLKLSSVTAADTAVYYCARDKWTWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 47 - protein, 220 aa, synthetic construct
DIEMTQSPDSLAVSLGERATINCRSSQSVLYSSSNRNYLAWYQQNPGQPPKLLIYWASTRESGVPDRFSGSGSGTDFTLTISSLQAEDVAVYYCQQYYSTPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - protein, 220 aa, synthetic construct
  VARIANT 116: note = non-natural amino acid
DIEMTQSPDSLAVSLGERATINCRSSQSVLYSSSNRNYLAWYQQNPGQPPKLLIYWASTRESGVPDRFSGSGSGTDFTLTISSLQAEDVAVYYCQQYYSTPRTFGQGTKVEIKRTXAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 220 aa, synthetic construct
  VARIANT 118: note = non-natural amino acid
DIEMTQSPDSLAVSLGERATINCRSSQSVLYSSSNRNYLAWYQQNPGQPPKLLIYWASTRESGVPDRFSGSGSGTDFTLTISSLQAEDVAVYYCQQYYSTPRTFGQGTKVEIKRTVAXPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 50 - protein, 220 aa, synthetic construct
  VARIANT 120: note = non-natural amino acid
DIEMTQSPDSLAVSLGERATINCRSSQSVLYSSSNRNYLAWYQQNPGQPPKLLIYWASTRESGVPDRFSGSGSGTDFTLTISSLQAEDVAVYYCQQYYSTPRTFGQGTKVEIKRTVAAPXVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 220 aa, synthetic construct
  VARIANT 127: note = non-natural amino acid
DIEMTQSPDSLAVSLGERATINCRSSQSVLYSSSNRNYLAWYQQNPGQPPKLLIYWASTRESGVPDRFSGSGSGTDFTLTISSLQAEDVAVYYCQQYYSTPRTFGQGTKVEIKRTVAAPSVFIFPPXDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 220 aa, synthetic construct
  VARIANT 133: note = non-natural amino acid
DIEMTQSPDSLAVSLGERATINCRSSQSVLYSSSNRNYLAWYQQNPGQPPKLLIYWASTRESGVPDRFSGSGSGTDFTLTISSLQAEDVAVYYCQQYYSTPRTFGQGTKVEIKRTVAAPSVFIFPPSDEQLKXGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - protein, 220 aa, synthetic construct
  VARIANT 155: note = non-natural amino acid
DIEMTQSPDSLAVSLGERATINCRSSQSVLYSSSNRNYLAWYQQNPGQPPKLLIYWASTRESGVPDRFSGSGSGTDFTLTISSLQAEDVAVYYCQQYYSTPRTFGQGTKVEIKRTVAAPSVFIFPPSDEQLKSGTASVVCLLNNFYPREAKVQWXVDNALQSGNSQESVTEQDSKDSTYSLSSTLTLSKADYEKHKVYACEVTHQGLSSPVTKSFNRGEC

SEQ ID NO: 54 - protein, 220 aa, synthetic construct
  VARIANT 162: note = non-natural amino acid
DIEMTQSPDSLAVSLGERATINCRSSQSVLYSSSNRNYLAWYQQNPGQPPKLLIYWASTRESGVPDRFSGSGSGTDFTLTISSLQAEDVAVYYCQQYYSTPRTFGQGTKVEIKRTVAAPSVFIFPPSDEQLKSGTASVVCLLNNFYPREAKVQWKVDNALQXGNSQESVTEQDSKDSTYSLSSTLTLSKADYEKHKVYACEVTHQGLSSPVTKSFNRGEC

SEQ ID NO: 55 - protein, 220 aa, synthetic construct
  VARIANT 174: note = non-natural amino acid
DIEMTQSPDSLAVSLGERATINCRSSQSVLYSSSNRNYLAWYQQNPGQPPKLLIYWASTRESGVPDRFSGSGSGTDFTLTISSLQAEDVAVYYCQQYYSTPRTFGQGTKVEIKRTVAAPSVFIFPPSDEQLKSGTASVVCLLNNFYPREAKVQWKVDNALQSGNSQESVTEQDXKDSTYSLSSTLTLSKADYEKHKVYACEVTHQGLSSPVTKSFNRGEC

SEQ ID NO: 56 - protein, 220 aa, synthetic construct
  VARIANT 208: note = non-natural amino acid
DIEMTQSPDSLAVSLGERATINCRSSQSVLYSSSNRNYLAWYQQNPGQPPKLLIYWASTRESGVPDRFSGSGSGTDFTLTISSLQAEDVAVYYCQQYYSTPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLXSPVTKSFNRGEC

SEQ ID NO: 57 - protein, 220 aa, synthetic construct
  VARIANT 211: note = non-natural amino acid
DIEMTQSPDSLAVSLGERATINCRSSQSVLYSSSNRNYLAWYQQNPGQPPKLLIYWASTRESGVPDRFSGSGSGTDFTLTISSLQAEDVAVYYCQQYYSTPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPXTKSFNRGEC

SEQ ID NO: 58 - protein, 446 aa, synthetic construct
  VARIANT 118: note = non-natural amino acid
QVQLQQWGAGLLKPSETLSLTCAVYGGSFSGYYWSWIRQPPGKGLEWIGEINHSGSTNYNPSLKSRVTISVETSKNQFSLKLSSVTAADTAVYYCARDKWTWYFDLWGRGTLVTVSSXSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

(19) United States
(12) Patent Application Publication
(10) Pub. No.: US 2026/0317329 A1
(43) Pub. Date: Oct. 15, 2026

(54) DRUG LINKERS AND ANTIBODY CONJUGATES THEREOF

(71) Applicant: Ambrx, Inc., La Jolla, CA (US)

(72) Inventors: Jianing Wang, San Diego, CA (US); Nickolas Knudsen, Escondido, CA (US); Lillian Skidmore, San Diego, CA (US); Ying Sun, San Diego, CA (US); Ji Young Kim, San Diego, CA (US)

(21) Appl. No.: 19/118,223

(22) PCT Filed: Oct. 6, 2023
(86) PCT No.: PCT/US2023/076296

§ 371 Date: Apr. 3, 2025

Related U.S. Application Data
(60) Provisional application No. 63/378,852, filed on Oct. 7, 2022.

(51) Int. Cl. C07D 487/04; A61K 47/68; C07D 519/00; C07F 9/6561

(52) U.S. Cl. CPC C07D 487/04; A61K 47/6835; C07D 519/00; C07F 9/6561

(57) ABSTRACT

Disclosed herein are antibody drug conjugates (ADCs), drug-linkers for ADCs, and tunable phosphate-based linkers. ADCs of the present disclosure include antibodies such as anti-CD70 antibodies conjugated to duocarmycin analogs via phosphate-based Uniters. Also disclosed are methods and compositions for using ADCs in inhibiting, preventing or treating diseases or conditions such as cancer.

### REFERENCE TO RELATED APPLICATIONS

The present application claims priority to U.S. Provisional Application No. 63/378,852, filed on Oct. 7, 2022, the entire contents of which are hereby incorporated herein in their entirety.

### SEQUENCE LISTING

The instant application contains a Sequence Listing which has been submitted electronically in XML file format and is hereby incorporated by reference in its entirety. Said XML copy, created on May 12, 2026, is named AMB1034USPCT1_SL.xml and is 78,369 bytes in size.

### FIELD OF THE INVENTION

This invention relates to antibody-drug conjugates (ADCs), cytotoxic duocarmycin analog drugs and drug-linkers. In particular, the invention relates to non-natural amino acid-containing antibodies conjugated to drug-linkers containing duocarmycin analogs and PEGylated phosphate-based linkers. The invention also relates to methods of using the ADCs, drugs and drug-linkers, including the treatment of cancer.

### BACKGROUND

Antibody-drug conjugates (ADCs) are a potent class of therapeutic constructs advancing the field of cancer therapeutics by allowing targeted delivery of cytotoxic agents to target cells, such as cancer cells. Currently, only a few ADCs have been approved for therapeutic use including gemtuzumab ozogamicin for AML (subsequently withdrawn from the market), brentuximab vedotin for ALCL and Hodgkin lymphoma, and trastuzumab emtansine for HER2-positive metastatic breast cancer (Verma et al., N Engl J Med 367:1783-91, 2012; Bross et al., Clin Cancer Res 7:1490-96, 2001; Francisco et al., Blood 102:1458-65, 2003); and sacituzumab govitecan for metastatic triple negative breast cancer (TNBC) (Zaman et al., OncoTargets and Therapy 12: 1781-1790, 2019). However, ADCs face challenges due to lack of therapeutic index and toxicity. The linker technology for attachment of the cytotoxic drug to an antibody impacts the stability of ADCs during the systemic circulation. Therefore, there is a need in the art to design improved linkers such as phosphate-based linkers and drug design for antibody conjugation.

The present disclosure provides phosphate-based linkers with tunable stability for intracellular delivery of drug payloads. The phosphate-based linkers have differentiated and tunable stability in blood versus an intracellular environment and can further include a self-immolating linker. Antibody-drug conjugates that comprise these linkers are stable in circulation (plasma/blood) but reactive or cleavable in intracellular compartments, such as lysosomal compartments, making them useful for intracellular delivery, the rate being dependent on the structure of the tuning element.

Cluster of differentiation 70 (CD70) is a member of the tumor necrosis factor superfamily and the ligand for CD27 (Goodwin, R. G. et al., Cell, 73:447-456 (1993); Hintzen, R. Q. et al., Int Immunol, 6:477-480 (1994)). CD70 was first identified in activated T- and B-lymphocytes. The binding of CD70 to CD27 on activated lymphocytes signals the co-stimulation of T cells, B cells and natural killer (NK) cells (Grewal, I. S., Expert Opin Ther Targets, 12(3):341-351 (2008); Borst, J. et al., Curr Opin Immunol., 17(3):275-281 (2005)) and regulates cell differentiation and T-helper 1/2 switching (Wajant, H., Expert Opin Ther Targets, 20(8):959-973 (2016)). The primary amino acid sequence of CD70 predicts a transmembrane type II protein with its carboxyl terminus exposed to the outside of cells and its amino terminus found in the cytosolic side of the plasma membrane. Human CD70 is composed of a 20 amino acid cytoplasmic domain, an 18 amino acid transmembrane domain, and a 155 amino acid extracytoplasmic domain with two potential N-linked glycosylation sites (Bowman et al., J Immunol, 152:1756-1761 (1994); Goodwin et al., Cell, 73:447-456 (1993)).

CD70 expression has been reported in different types of cancers including lymphomas, carcinomas and tumors of neural origin. In malignant B cells, 71% of diffuse large B-cell lymphomas, 33% of follicle center lymphomas, 25% of mantle lymphomas and 50% of B-CLL have been reported to express CD70 (Lens et al., 1999, Br J Haematol, 106:491-503). CD70 has also been detected on brain tumor cells, especially glioma cell lines, solid human gliomas, and meningiomas (Held-Feindt and Mentlein, Int J Cancer, 98:352-56 (2002); Wischlusen et al., Can Res, 62:2592-2599 (2002)). CD70 is frequently expressed in renal cell carcinoma (RCC; 87%) and non-Hodgkin's lymphoma (NHL; 77%) (Tannir, N. M. et al., Invest New Drugs, 32(6):1246-1257 (2014)), but minimally expressed in normal tissues (Nakae, R. et al., Am J Obstet Gynecol., 224(2):197 (2021)).

Anti-CD70 antibodies and antibody-drug conjugates (ADCs), and methods of making and using them to treat diseases such as cancer, are disclosed in WO2013/192360A1, the entire contents of which are hereby incorporated by reference in their entirety.

Multiple clinical trials evaluating anti-CD70 agents (e.g., antibodies with enhanced antibody-dependent cell-mediated cytotoxicity, ADCs, and chimeric antigen receptor (CAR) T-cell therapy) are being examined in malignancies showing high CD70 expression. Previous studies have shown that anti-CD70 monoclonal antibodies (mAb) and anti-CD70 ADCs exhibit anti-tumor effects in xenograft models of CD70 malignant diseases, such as lymphoma, NHL and RCC (Israel, B. F. et al., Mol Cancer Ther., 4(12):2037-2044 (2005); Law, C. L. et al., Cancer Res., 66:2328-2337 (2006); McEarchern, J. A. et al., Blood, 109(3):1185-92 (2007)). Based on the results of preclinical studies, two separate Phase 1 studies of SGN-75 (anti-CD70 mAb conjugated to maleimidocaproyl-monomethyl auristatin F (MMAF)) in patients with CD70-positive relapsed/refractory NHL or metastatic RCC were conducted; however, SGN-75 exhibited modest effects on these diseases with some intolerable side effects (Tannir, N. M. et al., Invest New Drugs, 32(6):1246-1257 (2014)). An additional anti-CD70 ADC, SGN-CD70A (anti-CD70 mAb conjugated to a pyrrolobenzodiazepine dimer), was introduced into Phase 1 clinical trials (Pal, S. K. et al., Cancer, 125(7):1124-1132 (2019)), but the SGN-CD70A Phase 1 study was discontinued in 2018.

Duocarmycin SA is a highly potent cytotoxic natural product that binds to the DNA minor groove and is capable of inducing sequence-selective alkylation of duplex DNA. Duocarmycin-based ADCs include BMS-936561 (MDX-1203), which contains an anti-CD70 antibody conjugated to duocarmycin derivative MED-A via a maleimide-containing citrulline-valine dipeptide linker (Wang H. et al. (2016) Biopharm Drug Disp 37(2):93-106; Owonikoko T. K. et al., Cancer Chemother Pharmacol (2016) 77(1):155-162). Termination of the development of BMS-936561/MDX-1203 exemplifies the challenges faced by duocarmycin-based ADCs (Hang-Ping Y. et al., Drug Discov Today (2021) 26(8):1857-1874).

There remains a need for anti-CD70 ADCs that are constructed in such a manner so as to be capable of exerting a clinically useful cytotoxic, cytostatic, or immunosuppressive effect on CD70-expressing cells, particularly without exerting undesirable effects on non-CD70-expressing cells. Such ADCs would be useful therapeutic agents against cancers that express CD70 or immune disorders that are mediated by CD70-expressing cells. The present invention provides such ADCs for use in immunology and oncology.

### SUMMARY OF THE INVENTION

The present invention provides novel drugs and drug-linkers suitable for antibody conjugation, wherein the drugs are duocarmycin analogs, and the drug-linkers contain phosphate-based linkers. The present invention further provides ADCs comprising the phosphate-based drug-linkers. The drugs, drug-linkers and ADCs are suitable for the treatment of diseases and conditions in human subjects in need thereof, including cancer.

In some general aspects, there is provided a compound of Formula (I):

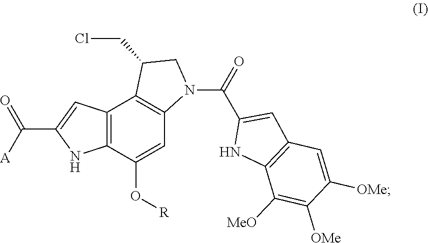

wherein:
R is H or L-W, wherein L is a linker and W is a reactive moiety; and
A is a bicyclic ring system selected from the group consisting of formula (a), (b), (c) and (d), having the following structures:

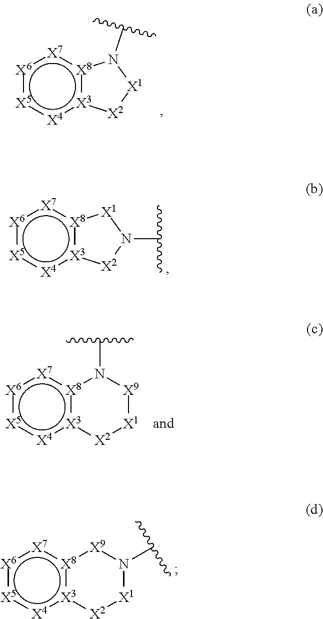

wherein:
   each X¹ is C(R^{1a})(R^{1b}); wherein each R^{1a} and R^{1b} is independently H, halogen, alkyl, alkenyl or alkynyl;
   each X² is C(R^{2a})(R^{2b}); wherein each R^{2a} and R^{2b} is independently H, halogen, alkyl, alkenyl or alkynyl;
   each X³ is C;
   each X⁴ is C(R⁴) or N, wherein R⁴ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
   each X⁵ is C(R⁵) or N, wherein R⁵ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
   each X⁶ is C(R⁶) or N, wherein R⁶ is H, halogen, —OH, —SH, —NO₂, —CN, —N₃, —N(R^{a})(R^{b}) acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s})—;
   each X⁷ is C(R⁷) or N, wherein R⁷ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
   each X⁸ is C; and
   each X⁹, when present, is C(R^{9a})(R^{9b}); wherein each R^{9a} and R^{9b} is independently H, halogen, alkyl, alkenyl or alkynyl;
      wherein:
each R^{a} and R^{b} is independently H, alkyl, alkenyl or alkynyl;
each R^{c} is independently H, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl or heteroarylalkyl;
each R^{s} is independently H, halogen, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl or heteroarylalkyl; and
each m is independently 0, 1, 2 or 3;
   or a salt thereof.

In some embodiments, A-H (the corresponding amine of moiety A) has a C log P value of at least about 1.

In some aspects, A has the structure of formula (a), and the compound is a compound of Formula (Ia) having the following structure:

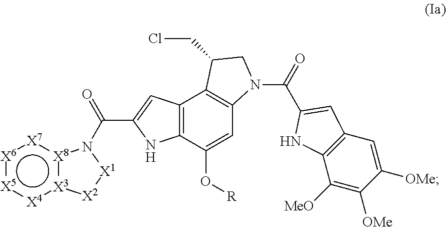

or a salt thereof.

In some aspects, A has the structure of formula (b), and the compound is a compound of Formula (Ib) having the following structure:

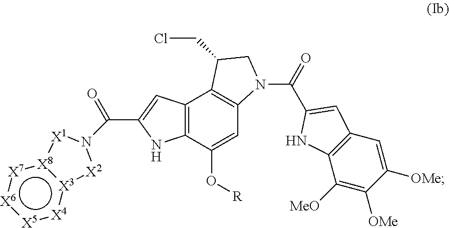

or a salt thereof.

In some aspects, A has the structure of formula (c), and the compound is a compound of Formula (Ic) having the following structure:

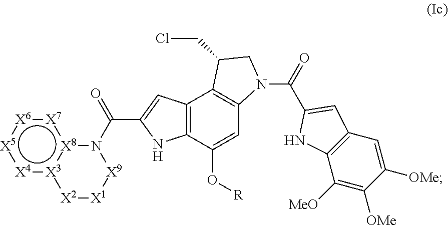

or a salt thereof.

In some aspects, A has the structure of formula (d), the compound is a compound of Formula (Id) having the following structure:

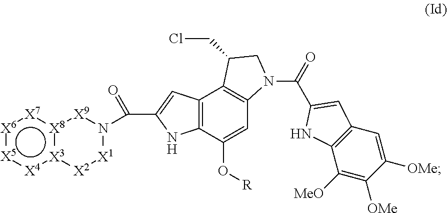

or a salt thereof.

In some aspects, there is provided a compound of Formula (I), or Formula (Ia), or Formula (Ib), or Formula (Ic) or Formula I(d), wherein:
R is H or L-W, wherein L is a linker and W is a reactive moiety;
X¹ is C(R^{1a})(R^{1b}); wherein each R^{1a} and R^{1b} is independently H, halogen, alkyl, alkenyl or alkynyl;
X² is C(R^{2a})(R^{2b}); wherein each R^{2a} and R^{2b} is independently H, halogen, alkyl, alkenyl or alkynyl;
X³ is C;
X⁴ is C(R⁴) or N, wherein R⁴ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{S});
X⁵ is C(R⁵) or N, wherein R⁵ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{S});
X⁶ is C(R⁶) or N, wherein R⁶ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
X⁷ is C(R⁷) or N, wherein R⁷ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{S}); and
X⁸ is C;
   wherein:
each R^{a} and R^{b} is independently H, alkyl, alkenyl or alkynyl;
each R^{c} is independently H, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl or heteroarylalkyl;
each R^{s} is independently H, halogen, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl or heteroarylalkyl; and
each m is independently 0, 1, 2 or 3.

In some further aspects, there is provided a compound of Formula (I), or Formula (Ia), or Formula (Ib), or Formula (Ic) or Formula I(d), wherein:
X¹ is C(R^{1a})(R^{1b}); wherein each R^{1a} and R^{1b} is independently H, halogen or unsubstituted alkyl;
X² is C(R^{2a})(R^{2b}); wherein each R^{2a} and R^{2b} is independently H, halogen or unsubstituted alkyl;
X³ is C;
X⁴ is C(R⁴) or N, wherein R⁴ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl;
X⁵ is C(R⁵) or N, wherein R⁵ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl;
X⁶ is C(R⁶) or N, wherein R⁶ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl;
X⁷ is C(R⁷) or N, wherein R⁷ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl;
X⁸ is C; and
X⁹, when present, is C(R^{9a})(R^{9b}); wherein each R^{9a} and R^{9b} is independently H, halogen or unsubstituted alkyl.

In some aspects, there is provided a compound of Formula (I), or Formula (Ia), or Formula (Ib), or Formula (Ic) or Formula I(d), wherein:
X¹ is C(R^{1a})(R^{1b}); wherein each R^{1a} and R^{1b} is H;
X² is C(R^{2a})(R^{2b}); wherein each R^{2a} and R^{2b} is H;
X³ is C;
X⁴ is C(R⁴) or N, wherein R⁴ is H;
X⁵ is C(R⁵) or N, wherein R⁵ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl;
X⁶ is C(R⁶) or N, wherein R⁶ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl;
X⁷ is C(R⁷) or N, wherein R⁷ is H;
X⁸ is C; and
X⁹, when present, is CH₂.

In some aspects, each said heteroalkyl is alkoxy.

In some aspects, there is provided a compound of Formula (I), or Formula (Ia), or Formula (Ib), or Formula (Ic) or Formula I(d), wherein:
X¹ is C(R^{1a})(R^{1b}); wherein each R^{1a} and R^{1b} is H;
X² is C(R^{2a})(R^{2b}); wherein each R^{2a} and R^{2b} is H;
X³ is C;
X⁴ is C(R⁴) or N, wherein R⁴ is H;
X⁵ is C(R⁵) or N, wherein R⁵ is H, halogen or alkoxy;
X⁶ is C(R⁶) or N, wherein R⁶ is H, halogen or alkoxy;
X⁷ is C(R⁷) or N, wherein R⁷ is H;
X⁸ is C; and
X⁹, when present, is CH₂.

In some aspects, X⁵ is C(R⁵) or N, wherein R⁵ is H or alkoxy; and X⁶ is C(R⁶) or N, wherein R⁶ is H or alkoxy.

In some aspects, X⁴ is N, X⁵ is C(R⁵), X⁶ is C(R⁶) and X⁷ is C(R⁷). In some other aspects, X⁴ is C(R⁴), X⁵ is N, X⁶ is C(R⁶) and X⁷ is C(R⁷). In some other aspects, X⁴ is C(R⁴), X⁵ is C(R⁵), X⁶ is N and X⁷ is C(R⁷). In some other aspects, X⁴ is C(R⁴), X⁵ is C(R⁵), X⁶ is C(R⁶) and X⁷ is C(R⁷). In some other aspects, X⁴ is C(R⁴), X⁵ is C(R⁵), X⁶ is C(R⁶) and X⁷ is N.

In some aspects, at least one of X⁴ and X⁷ is CH. In some aspects, each of X⁴ and X⁷ is CH.

In some aspects, at least one of R⁵ and R⁶ is alkoxy. In some further aspects, each said alkoxy is independently —OR^{k}, wherein each R^{k} is independently alkyl optionally substituted with heterocyclyl or —N(R^{d})(R^{e}); wherein said heterocyclyl contains at least one nitrogen atom, and each R^{d} and R^{c} is independently H, alkyl, alkenyl or alkynyl. In some aspects, each alkoxy is selected from the group consisting of —OCH₃, —OCH₂CH₃, —OCH₂CH₂CH₃, —OCH(CH₃)₂, —OCH₂CH₂N(CH₃)₂,

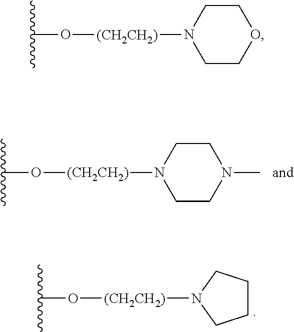

In some aspects, the compound of the present disclosure is a compound of Formula (I), wherein R is H. In some aspects, the compound is a compound of Formula (Ia), wherein R is H. In some aspects, the compound is a compound of Formula (Ib), wherein R is H. In some aspects, the compound is a compound of Formula (Ic), wherein R is H. In some aspects, the compound is a compound of Formula (Id), wherein R is H.

In some aspects, the compound is a compound of Formula (Ia), wherein R is H, and the compound is selected from the group consisting of:

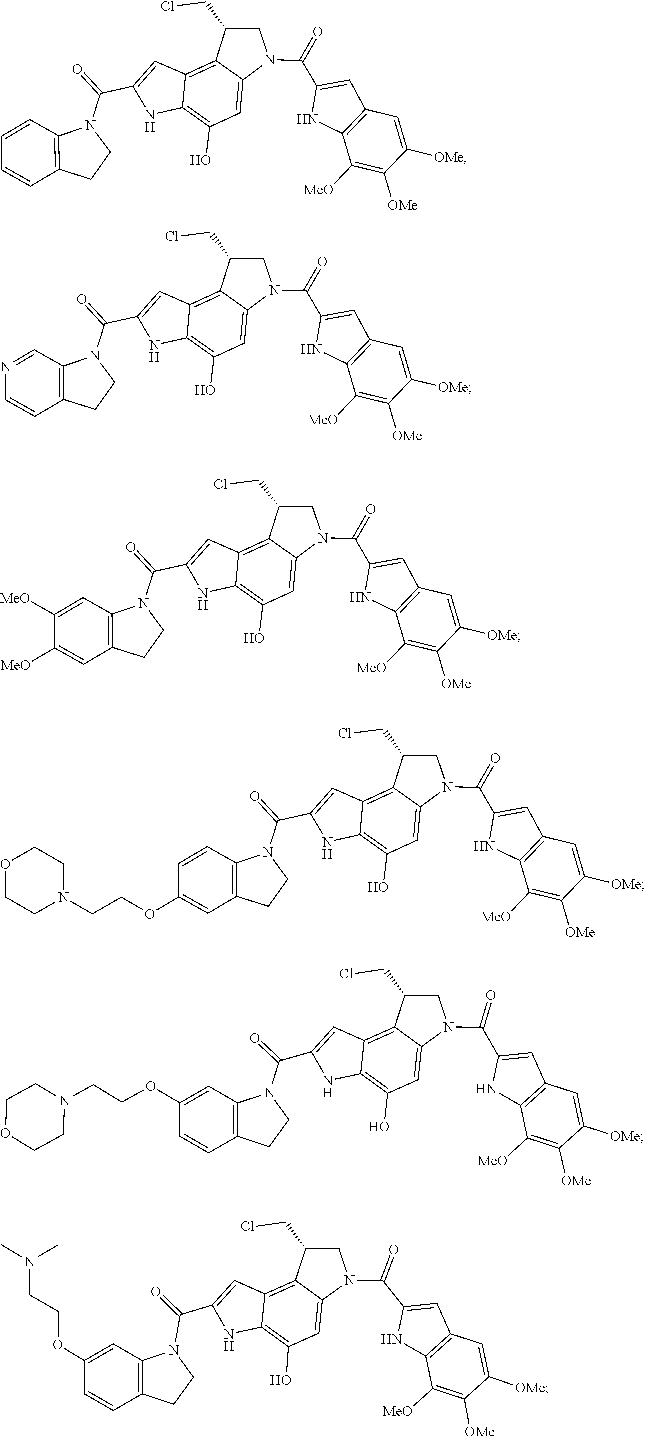

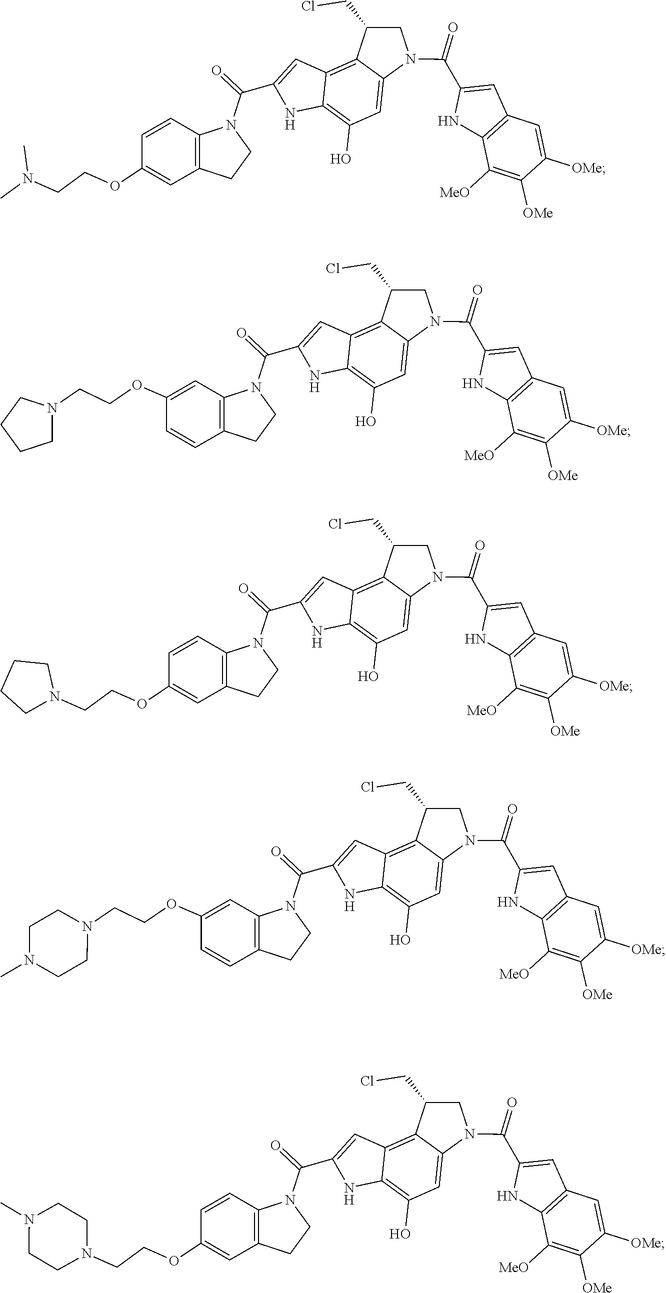

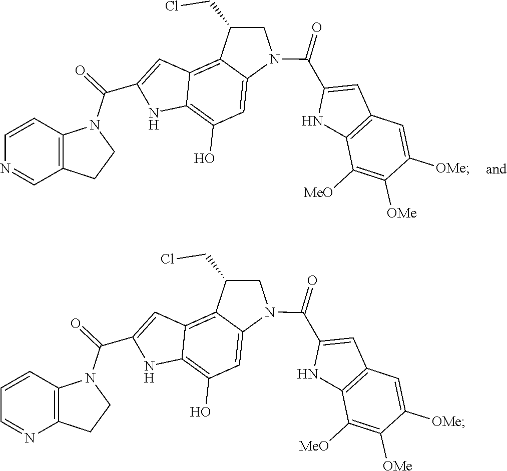 [SMILES: COc1cc2cc(C(=O)N3C[C@@H](CCl)c4c3cc(O)c3[nH]c(C(=O)N5CCc6cnccc65)cc43)[nH]c2c(OC)c1OC ; COc1cc2cc(C(=O)N3C[C@@H](CCl)c4c3cc(O)c3[nH]c(C(=O)N5CCc6ncccc65)cc43)[nH]c2c(OC)c1OC]

and salts thereof.

In some other aspects, the compound is a compound of Formula (I), wherein R is L-W. In some aspects, the compound is a compound of Formula (Ia), wherein R is L-W. In some aspects, the compound is a compound of Formula (Ib), wherein R is L-W. In some aspects, the compound is a compound of Formula (Ic), wherein R is L-W. In some aspects, the compound is a compound of Formula (Id), wherein R is L-W.

In some aspects, L is a phosphate-based linker. In some aspects, the phosphate-based linker comprises a phosphate-based moiety having the following structure:

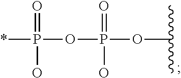 [SMILES: *OP(=O)(O)OP(*)(=O)O]

wherein * denotes the connection to the —O— atom at position R of Formula (I), or Formula (Ia), or Formula (Ib), or Formula (Ic) or Formula I(d); wherein L further comprises at least one additional moiety, and the wavy line of the phosphate-based moiety denotes the connection to one of the at least one additional moiety; wherein the at least one additional moiety is selected from the group consisting of unsubstituted alkylene, substituted alkylene, -(alkylene-O)—, optionally substituted arylene, —O—, —C(O)—, —N(R_{w})—, —S(O)₀₋₂—, a water-soluble polymer and an amino acid; wherein each R_{w} is independently H or C₁-C₈ alkyl; and combinations thereof. In some aspects, each at least one additional moiety is independently selected from the group consisting of unsubstituted alkylene, -(alkylene-O)—, —C(O)—, —N(R_{w})—, a water-soluble polymer and an amino acid; wherein each R_{w} is independently H or C₁-C₈ alkyl; and combinations thereof.

In some aspects, L is selected from the group of linkers of Table 6. In some other aspects, L is selected from the group of linkers of Table 7. In some other aspects, L is selected from the group of linkers of Table 8. In some aspects, L has the following structure:

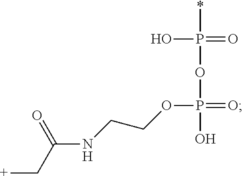

wherein * denotes the connection to the —O— atom at position R of Formula (I), or Formula (Ia), or Formula (Ib), or Formula (Ic) or Formula I(d); and + denotes the connection to W. In some other aspects, L has the following structure:

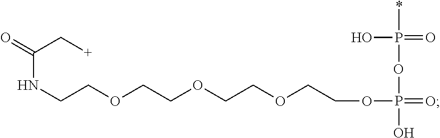

wherein * denotes the connection to the —O— atom at position R of Formula (I), or Formula (Ia), or Formula (Ib), or Formula (Ic) or Formula I(d); and + denotes the connection to W. In some other aspects, L has the following structure:

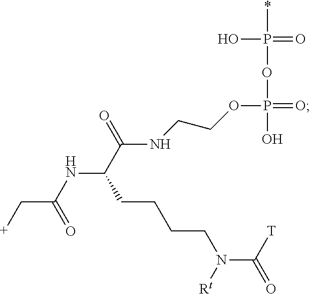

wherein T is a water-soluble polymer; R^{t} is H or methyl; * denotes the connection to the —O— atom at position R of Formula (I), or Formula (Ia), or Formula (Ib), or Formula (Ic) or Formula I(d); and + denotes the connection to W. In some aspect, the water-soluble polymer is a (polyethylene)glycol (PEG) moiety. In some aspects, the PEG moiety has a molecular weight within a range of about 100 Da to about 100,000 Da, about 100 Da to about 10,000 Da, about 100 Da to about 5,000 Da, or about 100 Da to about 1,000 Da. In some aspects, the PEG moiety is —(CH₂CH₂O)ₙCH₃, wherein n is an integer from 1 to 24. In some aspects, the PEG moiety is —(CH₂CH₂O)ₙCH₃, wherein n is 8, 9, 10, 11 or 12.

In some aspects, the reactive moiety W comprises —N₃, —OH, —SH, —NH(R^{j}), —C(O)R^{q}, —C(O)OR^{x}, —C(O)CH₂NH₂, an activated ester, —O—NH₂, a maleimide, a tetrazine, an alkyne, a cyclooctyne or an (E)-cyclooctene; wherein R^{j} is H or unsubstituted alkyl, R^{q} is unsubstituted alkyl, and R^{x} is H, unsubstituted alkyl or a carboxylic acid protecting group. In some further aspects, reactive moiety W is selected from the group consisting of:

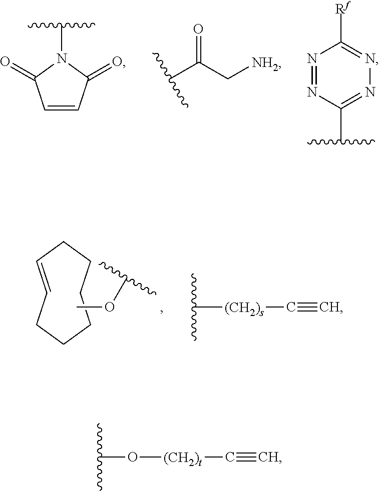

—N₃, —OH, —SH, —NH(R^{j}), —C(O)R^{q}, —C(O)OR^{x}, an activated ester, —O—NH₂ and an optionally substituted monocyclic or polycyclic group comprising the cyclooctyne; wherein: Ri is H or unsubstituted C₁-C₆ alkyl, R^{q} is unsubstituted C₁-C₆ alkyl, R^{x} is H, unsubstituted C₁-C₆ alkyl or a carboxylic acid protecting group, R^{f} is H or unsubstituted C₁-C₆ alkyl, s is 0, 1, 2, 3, 4, 5 or 6, and t is 0, 1, 2, 3, 4, 5 or 6.

In some aspects, W is —ONH₂.

In some aspects, there is provided a compound of Formula (Ia), wherein R is L-W, and the compound is selected from the group consisting of:

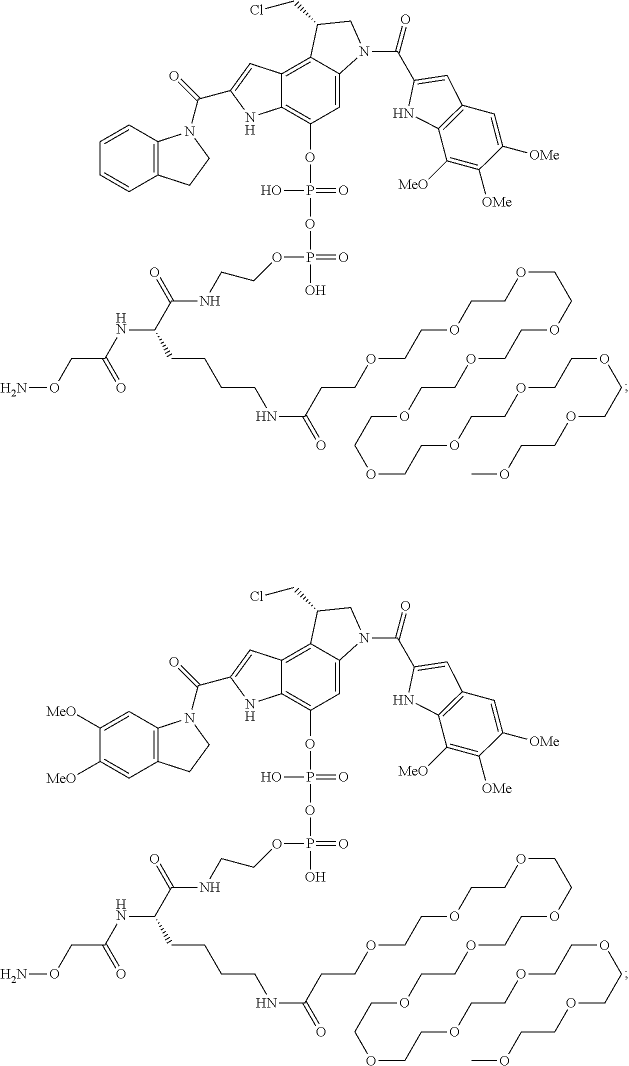 [SMILES: COCCOCCOCCOCCOCCOCCOCCOCCOCCOCCOCCOCCC(=O)NCCCC[C@H](NC(=O)CON)C(=O)NCCOP(=O)(O)OP(=O)(O)Oc1cc2c(c3cc(C(=O)N4CCc5ccccc54)[nH]c13)[C@H](CCl)CN2C(=O)c1cc2cc(OC)c(OC)c(OC)c2[nH]1 ; COCCOCCOCCOCCOCCOCCOCCOCCOCCOCCOCCOCCC(=O)NCCCC[C@H](NC(=O)CON)C(=O)NCCOP(=O)(O)OP(=O)(O)Oc1cc2c(c3cc(C(=O)N4CCc5cc(OC)c(OC)cc54)[nH]c13)[C@H](CCl)CN2C(=O)c1cc2cc(OC)c(OC)c(OC)c2[nH]1]

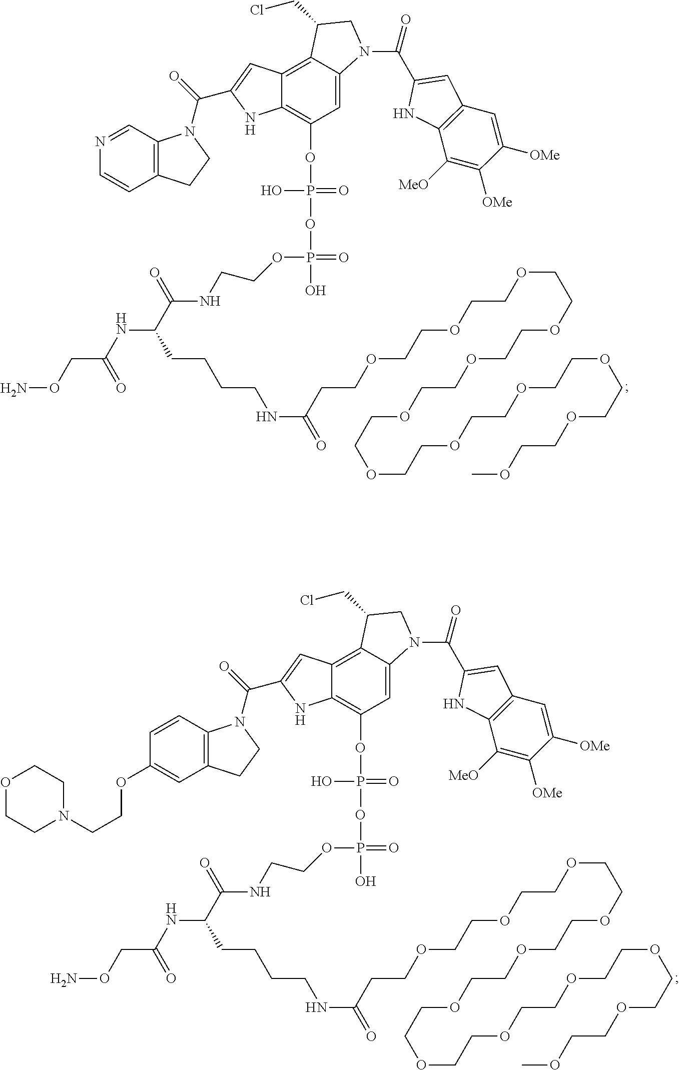 [SMILES: COCCOCCOCCOCCOCCOCCOCCOCCOCCOCCOCCOCCC(=O)NCCCC[C@H](NC(=O)CON)C(=O)NCCOP(=O)(O)OP(=O)(O)Oc1cc2c(c3cc(C(=O)N4CCc5ccncc54)[nH]c13)[C@H](CCl)CN2C(=O)c1cc2cc(OC)c(OC)c(OC)c2[nH]1 ; COCCOCCOCCOCCOCCOCCOCCOCCOCCOCCOCCOCCC(=O)NCCCC[C@H](NC(=O)CON)C(=O)NCCOP(=O)(O)OP(=O)(O)Oc1cc2c(c3cc(C(=O)N4CCc5cc(OCCN6CCOCC6)ccc54)[nH]c13)[C@H](CCl)CN2C(=O)c1cc2cc(OC)c(OC)c(OC)c2[nH]1]

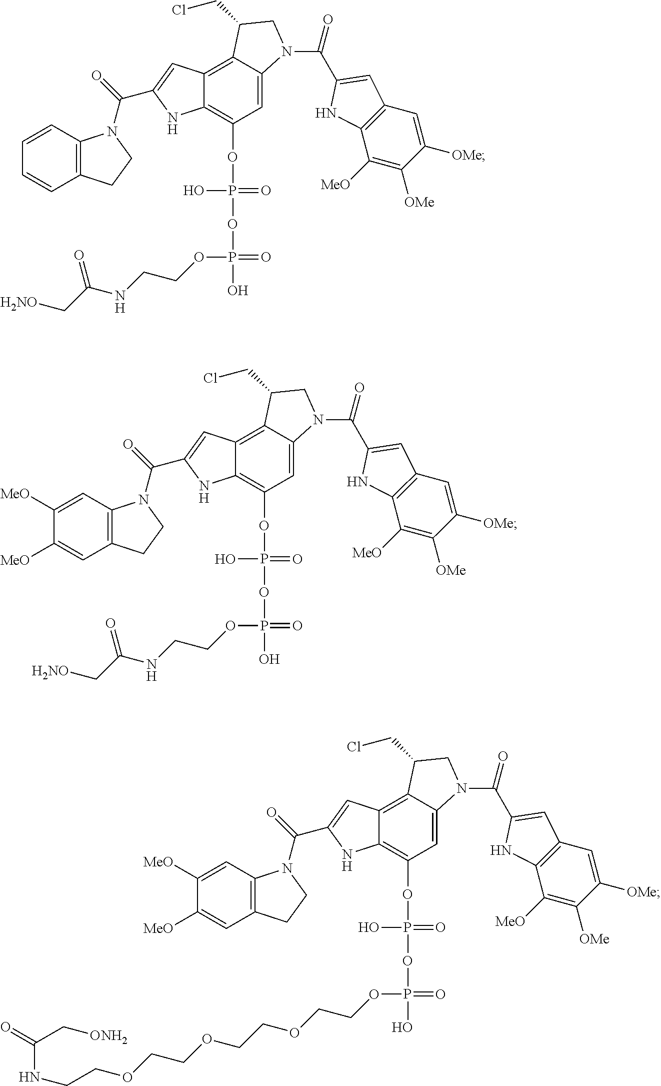

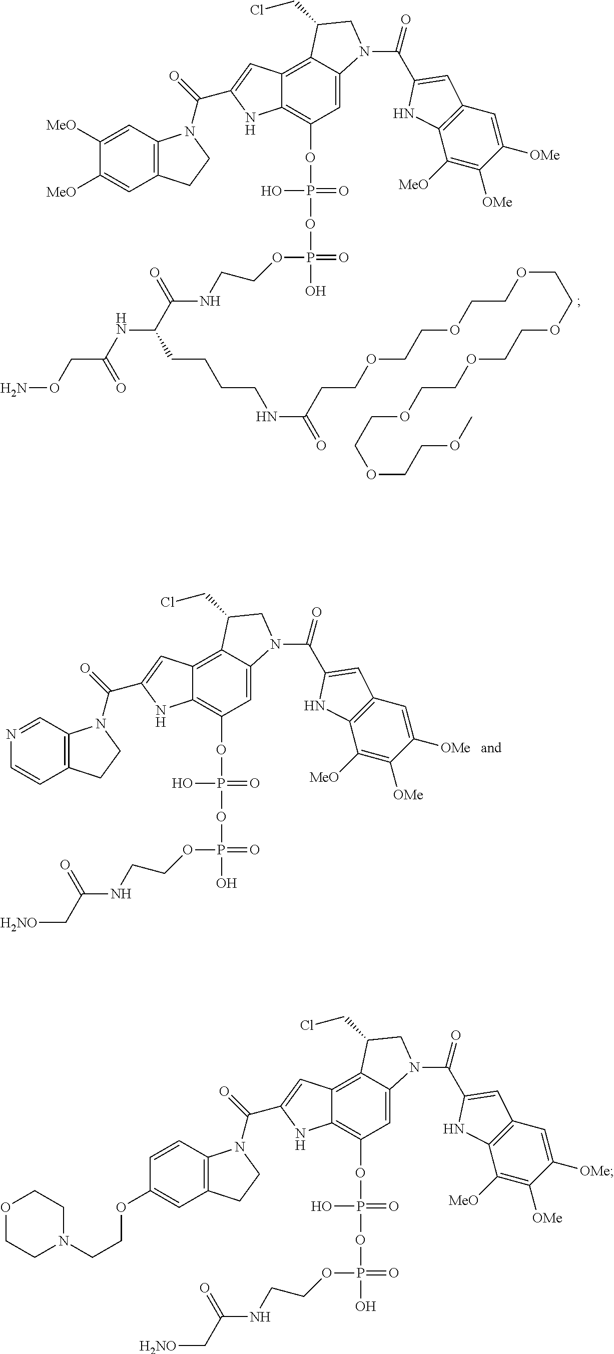

and salts thereof.

In some other general aspects, the present disclosure provides an antibody-drug conjugate (ADC) of Formula (II):

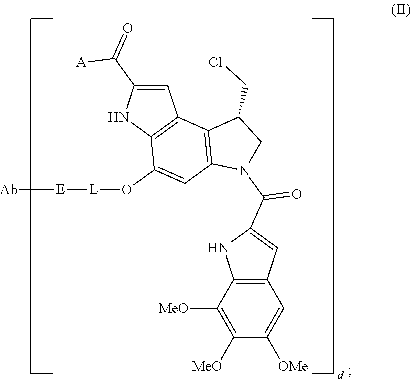

wherein:
Ab is an antibody, wherein Ab comprises one or more non-natural amino acids;
L is a linker;
E is a moiety joining Ab and L;
d is an integer from 1 to 10; and
A is selected from the group consisting of formula (a), (b), (c) and (d), having the following structures:

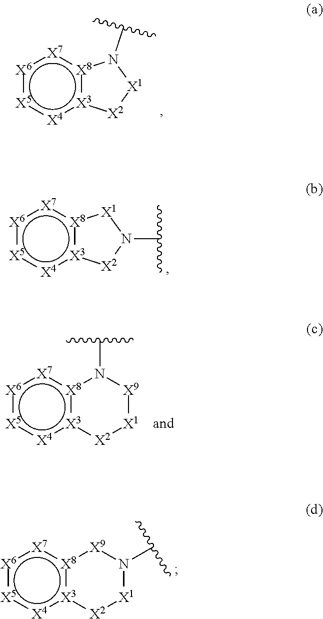

wherein:
   each X¹ is C(R^{1a})(R^{1b}); wherein each R^{1a} and R^{1b} is independently H, halogen, alkyl, alkenyl or alkynyl;
   each X² is C(R^{2a})(R^{2b}); wherein each R^{2a} and R^{2b} is independently H, halogen, alkyl, alkenyl or alkynyl;
   each X³ is C;
   each X⁴ is C(R⁴) or N, wherein R⁴ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
   each X⁵ is C(R⁵) or N, wherein R⁵ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
   each X⁶ is C(R⁶) or N, wherein R⁶ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{S});
   each X⁷ is C(R⁷) or N, wherein R⁷ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
   each X⁸ is C; and
   each X⁹, when present, is C(R^{9a})(R^{9b}); wherein each R^{9a} and R^{9b} is independently H, halogen, alkyl, alkenyl or alkynyl;
      wherein:
each R^{a} and R^{b} is independently H, alkyl, alkenyl or alkynyl;
each R^{c} is independently H, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl or heteroarylalkyl;
each R^{s} is independently H, halogen, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl or heteroarylalkyl; and
each m is independently 0, 1, 2 or 3;
   or a pharmaceutically acceptable salt thereof.

In some embodiments, A-H (the corresponding amine of moiety A) has a C log P value of at least about 1.

In some aspects, there is provided an ADC of Formula (II), wherein A is formula (a). In some aspects, there is provided an ADC of Formula (II), wherein A is formula (b). In some aspects, there is provided an ADC of Formula (II), wherein A is formula (c). In some aspects, there is provided an ADC of Formula (II), wherein A is formula (d).

In some aspects, there is provided an ADC of Formula (II), wherein:
X¹ is C(R^{1a})(R^{1b}); wherein each R^{1a} and R^{1b} is independently H, halogen or unsubstituted alkyl;
X² is C(R^{2a})(R^{2b}); wherein each R^{2a} and R^{2b} is independently H, halogen or unsubstituted alkyl;
X³ is C;
X⁴ is C(R⁴) or N, wherein R⁴ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl;
X⁵ is C(R⁵) or N, wherein R⁵ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl;
X⁶ is C(R⁶) or N, wherein R⁶ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl;
X⁷ is C(R⁷) or N, wherein R⁷ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl;
X⁸ is C; and
X⁹, when present, is C(R^{9a})(R^{9b}); wherein each R^{9a} and R^{9b} is independently H, halogen or unsubstituted alkyl.

In some aspects, there is provided an ADC of Formula (II) wherein:
X¹ is C(R^{1a})(R^{1b}); wherein each R^{1a} and R^{1b} is H;
X² is C(R^{2a})(R^{2b}); wherein each R^{2a} and R^{2b} is H;
X³ is C;
X⁴ is C(R⁴) or N, wherein R⁴ is H;
X⁵ is C(R⁵) or N, wherein R⁵ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl;
X⁶ is C(R⁶) or N, wherein R⁶ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl;
X⁷ is C(R⁷) or N, wherein R⁷ is H;
X⁸ is C; and
X⁹, when present, is CH₂.

In some aspects, each said heteroalkyl is an alkoxy. Thus, in some aspects, there is provided an ADC of Formula (II), wherein:
X¹ is C(R^{1a})(R^{1b}); wherein each R^{1a} and R^{1b} is H;
X² is C(R^{2a})(R^{2b}); wherein each R^{2a} and R^{2b} is H;
X³ is C;
X⁴ is C(R⁴) or N, wherein R⁴ is H;
X⁵ is C(R⁵) or N, wherein R⁵ is H, halogen or alkoxy;
X⁶ is C(R⁶) or N, wherein R⁶ is H, halogen or alkoxy;
X⁷ is C(R⁷) or N, wherein R⁷ is H;
X⁸ is C; and
X⁹, when present, is CH₂.

In some aspects, X⁵ is C(R⁵) or N, wherein R⁵ is H or alkoxy; and X⁶ is C(R⁶) or N, wherein R⁶ is H or alkoxy.

In some aspects, X⁴ is N, X⁵ is C(R⁵), X⁶ is C(R⁶) and X⁷ is C(R⁷). In some other aspects, X⁴ is C(R⁴), X⁵ is N, X⁶ is C(R⁶) and X⁷ is C(R⁷). In some other aspects, X⁴ is C(R⁴), X⁵ is C(R⁵), X⁶ is N and X⁷ is C(R⁷). In some other aspects, X⁴ is C(R⁴), X⁵ is C(R⁵), X⁶ is C(R⁶) and X⁷ is C(R⁷). In some other aspects, X⁴ is C(R⁴), X⁵ is C(R⁵), X⁶ is C(R⁶) and X⁷ is N.

In some aspects, at least one of X⁴ and X⁷ is CH. In some aspects, each of X⁴ and X⁷ is CH.

In some aspects, at least one of R⁵ and R⁶ is alkoxy.

In some aspects, d is 1, 2, 3 or 4. In some aspects, d is 2. In some aspects, d is 4.

In some aspects, there is provided an ADC of Formula (II), wherein L is a phosphate-based linker. In some aspects, the phosphate-based linker comprises a phosphate-based moiety having the following structure:

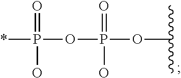 [SMILES: *OP(=O)(O)OP(*)(=O)O]

wherein * denotes the connection to the —O— atom at position L of Formula (II); wherein L further comprises at least one additional moiety, and the wavy line of the phosphate-based moiety denotes the connection to one of the at least one additional moiety. In some aspects, each at least one additional moiety is independently selected from the group consisting of unsubstituted alkylene, -(alkylene-O)—, —C(O)—, —N(R_{w})—, a water-soluble polymer and an amino acid; wherein each R_{w} is independently H or C₁-C₈ alkyl; and combinations thereof. In some aspects, L is selected from the group of linkers of Table 6. I some other aspects, L is selected from the group of linkers of Table 7. In some other aspects, L is selected from the group of linkers of Table 8. In some aspects, L has the following structure:

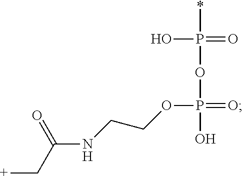

wherein * denotes the connection to the —O— atom at position L of Formula (II); and + denotes the connection to E. In some other aspects, L has the following structure:

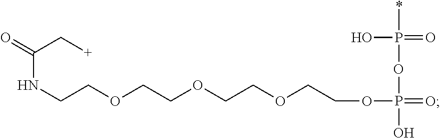

wherein * denotes the connection to the —O— atom at position L of Formula (II); and + denotes the connection to E. In some other aspects, L has the following structure:

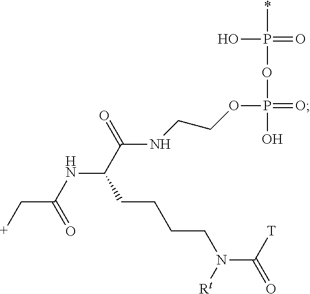

wherein T is a water-soluble polymer; R^{t} is H or methyl; * denotes the connection to the —O— atom at position L of Formula (II); and + denotes the connection to E. In some aspects, the water-soluble polymer is a (polyethylene)glycol (PEG) moiety. In some aspects, the PEG moiety has a molecular weight within a range of about 100 Da to about 100,000 Da, about 100 Da to about 10,000 Da, about 100 Da to about 5,000 Da, or about 100 Da to about 1,000 Da. In some aspects, the PEG moiety is —(CH₂CH₂O)ₙCH₃, wherein n is an integer from 1 to 24. In some aspects, the PEG moiety is —(CH₂CH₂O)ₙCH₃, wherein n is 8, 9, 10, 11 or 12. In some aspects, n is 8. In some aspects, n is 12.

In some aspects, E comprises an amide, an ester, a thioester, a pyrrolidine-2,5-dione, an oxime, a 1,2,3-triazole or a 1,4-dihydropyridazine, wherein the 1,2,3-triazole and the 1,4-dihydropyridazine are each optionally fused to an 8-membered ring. In some aspects, E is selected from the group consisting of:

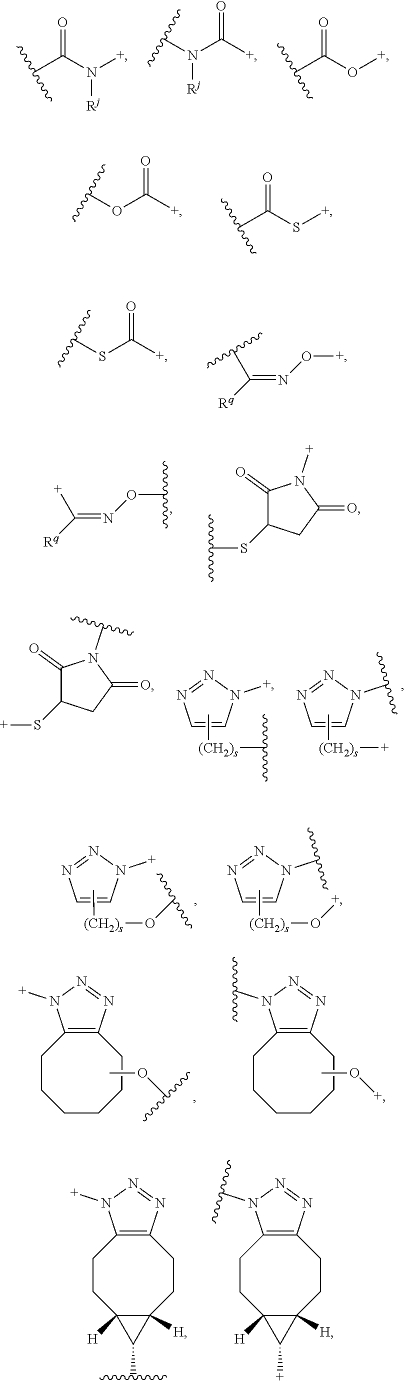

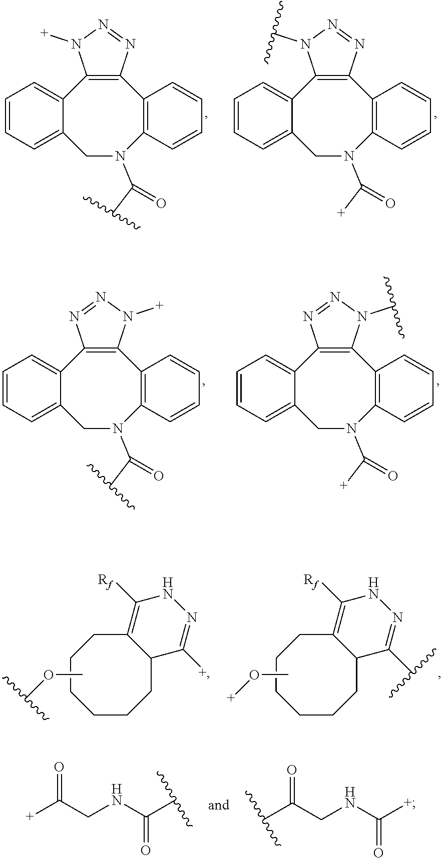

wherein each R^{j} is independently H or unsubstituted C₁-C₆ alkyl; each R^{q} is independently unsubstituted C₁-C₆ alkyl; each R^{f} is independently H or unsubstituted C₁-C₆ alkyl; each s is independently 0, 1, 2, 3, 4, 5 or 6; each t is independently 0, 1, 2, 3, 4, 5 or 6; each + denotes connection to L; and each wavy line denotes connection to Ab. In some aspects, E is:

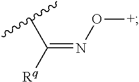

wherein R^{q} is unsubstituted C₁-C₆ alkyl. In some aspects, R^{q} is methyl.

In some aspects, E joins L to a non-natural amino acid of Ab.

In some aspects, R^{q} is a methyl of a non-natural amino acid encoded into Ab. In some embodiments, the non-natural amino acid is para-acetyl-L-phenylalanine (pAF), and R^{q} is the methyl group of the pAF acyl group.

In some aspects, Ab is configured to bind to an antigen. In some aspects, the antigen is selected from the group consisting of PD-1, PD-Li, PSMA, CD70, CD3, HER2, HER3, TROP2, GPC3, VEGFR, EGFR, c-Met (HGFR), CD19, CD22, CD25 (IL-2R alpha), CD30, CD33, CD37, CD46, CD48, CD56 (NCAM-1), CD71 (Transferrin R), CD74, CD79b, CD123 (IL-3R alpha), CD138 (syndecan-1), CD142, CD166 (ALCAM), CD203c (ENPP3), CD205 (LY75), CD221 (IGF-1R), CD262 (TRAIL R2), CD276 (B7-H3), mesothelin, EpCAM, CEACAM5, CEACAM6, DLL3, ROR1, ROR2, GPNMB, GCC, GUCY2c, NaPi2b, Flt-1, Flt-3, folate receptor alpha, Tissue Factor (TF), CA6, MUC1, MUC16 (CA-125), BCMA, SLAMF7 (CSi), TIM1, CanAg, Ckit (CD117), EphA2, Nectin4, SLTRK6, FGFR2, LYPD3 (C4.4a), Cadherin 3, 5T4 (TPBG), STEAP1, PTK7, Ephrin-A4, LIV-1 (SLC39A6 or ZIP6), SLC1A5, TENB2, ETBR, integrin v3, Cripto, AGS-5 (SLC44A4), LY6E, AXL, LAMP1, LRRC15, TNF-alpha and MN/CA IX. In some aspects, the antigen is TROP2, CD70, HER2, PSMA, HER3 or GPC3.

In some aspects, Ab is an anti-CD70 antibody comprising a sequence listed in Table 2. In some aspects, the anti-CD70 antibody comprises a heavy chain variable region having the amino acid sequence of SEQ ID NO: 26. In some aspects, the anti-CD70 antibody comprises a light chain variable region having the amino acid sequence of SEQ ID NO: 27. In some aspects, the anti-CD70 antibody comprises a heavy chain having the amino acid sequence of SEQ ID NO: 25. In some other aspects, the anti-CD70 antibody comprises a heavy chain having the amino acid sequence of SEQ ID NO: 20.

In some aspects, the anti-CD70 antibody comprises a light chain having the amino acid sequence of SEQ ID NO: 19. In some aspects, the anti-CD70 antibody comprises two heavy chains, each having the amino acid sequence of SEQ ID NO: 20, and two light chains, each having the amino acid sequence of SEQ ID NO: 19. In some other aspects, the anti-CD70 antibody comprises two heavy chains, each having the amino acid sequence of SEQ ID NO: 25, and two light chains, each having the amino acid sequence of SEQ ID NO: 19.

In some other aspects, Ab is an anti-TROP2 antibody comprising a sequence listed in Table 1. In some aspects, the anti-TROP2 antibody comprises a heavy chain having the amino acid sequence of SEQ ID NO: 5. In some aspects, the anti-TROP2 antibody comprises a light chain having the amino acid sequence of SEQ ID NO: 4.

In some other aspects, Ab is an anti-HER2 antibody comprising a sequence listed in Table 3. In some aspects, the anti-HER2 antibody comprises a heavy chain having the amino acid sequence of SEQ ID NO: 29. In some aspects, the anti-HER2 antibody comprises a light chain having the amino acid sequence of SEQ ID NO: 30.

In some other aspects, Ab is an anti-PSMA antibody comprising a sequence listed in Table 4. In some aspects, the anti-PSMA antibody comprises a heavy chain having the amino acid sequence of SEQ ID NO: 39. In some aspects, the anti-PSMA antibody comprises a light chain having the amino acid sequence of SEQ ID NO: 40.

In some other aspects, Ab is an anti-HER3 antibody comprising a sequence listed in Table 5. In some aspects, the anti-HER3 antibody comprises a heavy chain having the amino acid sequence of SEQ ID NO: 58. In some aspects, the anti-PSMA antibody comprises a light chain having the amino acid sequence of SEQ ID NO: 47.

In some aspects, antibody (Ab) comprises two heavy chains, and one non-natural amino acid is incorporated into each said heavy chain.

In some aspects, the non-natural amino acid is para-acetyl-L-phenylalanine.

In some other general aspects, the present disclosure provides a pharmaceutical composition comprising a compound of Formula (I), or Formula (Ia), or Formula (Ib), or Formula (Ic) or Formula I(d), or an ADC of Formula (II), and at least one pharmaceutically acceptable adjuvant, binder, buffer, carrier, diluent or excipient.

In some other general aspects, the present disclosure provides a method of treating a disease or condition in a subject, the method comprising administering to the subject a therapeutically effect amount of a compound of Formula (I), or Formula (Ia), or Formula (Ib), or Formula (Ic) or Formula I(d), or an ADC of Formula (II), or a pharmaceutical composition comprising a therapeutically effect amount of a compound of Formula (I), or Formula (Ia), or Formula (Ib), or Formula (Ic) or Formula I(d), or an ADC of Formula (II). In some aspects, the disease or condition is cancer. In some aspects, the cancer is a CD70-expression cancer. In some aspects, the cancer is renal cell carcinoma. In some other aspects, the cancer is a blood cancer. In some aspects, the blood cancer is a leukemia, lymphoma or myeloma.

It is to be understood that the methods and compositions described herein are not limited to the particular methodology, protocols, cell lines, constructs, and reagents described herein and as such may vary. It is also to be understood that the terminology used herein is for the purpose of describing particular embodiments only and is not intended to limit the scope of the methods and compositions described herein.

### INCORPORATION BY REFERENCE

All publications, patents, patent applications and/or other documents mentioned herein are incorporated herein by reference in their entirety for all purposes and to the same extent as if each individual publication, patent, patent application and/or other document was specifically and individually indicated to be incorporated by reference for all purposes, and for the purpose of describing and disclosing, for example, the compositions and other methodologies that are described in the publications, patents, patent applications and/or other documents, which might be used in connection with the presently described inventions. The publications discussed herein are provided solely for their disclosure prior to the filing date of the present application.

### BRIEF DESCRIPTION OF THE DRAWINGS

FIGS. 1A and 1B show evaluation of in vitro cytotoxic activity of duocarmycin analog compounds and anti-CD70 ADCs against CD70 positive cell line 786-O (FIG. 1A); and CD70 negative cell line NCI-H929 (FIG. 1B).

FIGS. 2A and 2B show evaluation of in vitro cytotoxic activity of duocarmycin analog compounds and anti-GPC3 ADCs against GPC3 positive cell line HepG2 (FIG. 2A); and GPC3 negative cell line SUN499 (FIG. 2B).

### DETAILED DESCRIPTION

Before describing the present invention in detail, it is to be understood that this invention is not limited to particular methodologies, or compositions, or biological systems, which can, of course, vary. It is also to be understood that the terminology used herein is for the purpose of describing particular embodiments only and is not intended to be limiting.

While various embodiments have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Definitions

Unless otherwise defined herein or below in the remainder of the specification, all technical and scientific terms used herein have the same meaning as commonly understood by those of ordinary skill in the art to which the invention belongs. As used herein and in the appended claims, the singular forms “a,” “an,” and “the” include plural reference unless the context clearly indicates otherwise.

Unless defined otherwise, all technical and scientific terms used herein have the same meaning as commonly understood to one of ordinary skill in the art to which the inventions described herein belong. Various methods, materials, and the like, similar or equivalent to those described herein can be used in the practice or testing of the inventions described herein.

All publications and patents mentioned herein are incorporated herein by reference in their entirety for the purpose of describing and disclosing, for example, the chemistry, chemical syntheses, compositions and other methodologies that are described in the publications, which might be used in connection with the presently described inventions. The publications discussed herein are provided solely for their disclosure prior to the filing date of the present application.

### Chemical Terms

It is to be understood that the terminology employed herein is for the purpose of describing particular embodiments and is not intended to be limiting.

The term “acyl,” as used herein, represents —C(O)-alkyl, as defined herein, and is exemplified by acetyl (—C(O)CH₃), trifluoroacetyl, propionyl, and butanoyl. Exemplary unsubstituted acyl groups include from 1 to 6, from 1 to 11, or from 1 to 21 carbons.

The term “alkyl,” as used herein, refers to a branched or straight-chain monovalent saturated aliphatic hydrocarbon radical of 1 to 20 carbon atoms. Non-limiting examples of alkyl groups include aliphatic hydrocarbon radicals of 1 to 16 carbon atoms (C₁₋₁₆ alkyl), 1 to 10 carbon atoms (C₁₋₁₀ alkyl), 1 to 6 carbon atoms (C₁₋₆ alkyl), four carbon atoms (e.g., n-butyl, iso-butyl, sec-butyl, t-butyl), three carbon atoms (e.g., isopropyl or n-propyl), two carbon atoms (ethyl) and 1 carbon atom (methyl). An alkylene is a divalent alkyl group.

The term “alkenyl,” as used herein, alone or in combination with other groups, refers to a straight-chain or branched hydrocarbon residue having a carbon-carbon double bond and having 2 to 20 carbon atoms (e.g., 2 to 16 carbon atoms, 2 to 10 carbon atoms, 2 to 6, or 2 carbon atoms).

The term “alkoxy,” as used herein, alone or in combination with other groups, refs to an alkyl group having a single bond to oxygen. Non-limiting examples of alkoxy groups of the present disclosure include methoxy (—OMe) and ethoxy (—OEt). An alkoxy group of the present disclosure is optionally substituted. In some embodiments, an alkoxy group of the present disclosure is optionally substituted with heterocyclyl, or with —N(R^{d})(R^{e}), wherein each R^{d} and R^{c} is independently H, alkyl, alkenyl or alkynyl.

The term “alkynyl,” as used herein, alone or in combination with other groups, refers to a straight-chain or branched hydrocarbon residue having a carbon-carbon triple bond and having 2 to 20 carbon atoms (e.g., 2 to 16 carbon atoms, 2 to 10 carbon atoms, 2 to 6, or 2 carbon atoms).

The term “amino,” as used herein, represents —N(R^{N1})₂, wherein each R^{N1} is, independently, H, OH, NO₂, N(R^{N2})₂, SO₂OR^{N2}, SO₂R^{N2}, SOR^{N2}, an N-protecting group, alkyl, alkoxy, aryl, arylalkyl, cycloalkyl, acyl (e.g., acetyl, trifluoroacetyl, or others described herein), wherein each of these recited R^{N1} groups can be optionally substituted; or two R^{N1} combine to form an alkylene or heteroalkylene, and wherein each R^{N2} is, independently, H, alkyl, or aryl. The amino groups of the invention can be an unsubstituted amino (i.e., —NH₂) or a substituted amino (i.e., —N(R^{N1})₂).

The term “aryl,” as used herein, refers to an aromatic mono- or polycarbocyclic radical of 6 to 12 carbon atoms having at least one aromatic ring. Examples of such groups include, but are not limited to, phenyl, naphthyl, 1,2,3,4-tetrahydronaphthyl, 1,2-dihydronaphthyl, indanyl, and 1H-indenyl.

The term “arylalkyl,” as used herein, represents an alkyl group substituted with an aryl group. Exemplary unsubstituted arylalkyl groups are from 7 to 30 carbons (e.g., from 7 to 16 or from 7 to 20 carbons, such as C₁₋₆ alkyl C₆₋₁₀ aryl, C₁₋₁₀ alkyl C₆₋₁₀ aryl, or C₁₋₂₀ alkyl C₆₋₁₀ aryl), such as, benzyl and phenethyl. In some embodiments, the akyl and the aryl each can be further substituted with 1, 2, 3, or 4 substituent groups as defined herein for the respective groups.

The term “azido,” as used herein, represents a —N₃ group.

The term “bicyclic ring system” as used herein refers to a bicyclic moiety or molecule containing two joined rings, wherein the two rings are joined by the sharing of two or more atoms. In some embodiments, the bicyclic ring system shares two atoms. In some embodiments, the bicyclic ring system contains at least one nitrogen atom.

The term “cyano,” as used herein, represents a —CN group.

The terms “carbocyclyl,” as used herein, refer to a non-aromatic C₃₋₁₂ monocyclic, bicyclic, or tricyclic structure in which the rings are formed by carbon atoms. Carbocyclyl structures include cycloalkyl groups and unsaturated carbocyclyl radicals.

The term “cycloalkyl,” as used herein, refers to a saturated, non-aromatic, monovalent mono- or polycarbocyclic radical of three to ten, preferably three to six carbon atoms. This term is further exemplified by radicals such as cyclopropyl, cyclobutyl, cyclopentyl, cyclohexyl, cycloheptyl, norbornyl, and adamantyl.

The term “halogen,” as used herein, means a fluorine (fluoro), chlorine (chloro), bromine (bromo), or iodine (iodo) radical.

The term “heteroalkyl,” as used herein, refers to an alkyl group, as defined herein, in which one or more of the constituent carbon atoms have been replaced by nitrogen, oxygen, or sulfur. In some embodiments, the heteroalkyl group can be further substituted with 1, 2, 3, or 4 substituent groups as described herein for alkyl groups. Non-limiting examples of heteroalkyl groups include aminoalkyl and “alkoxy.”

A heteroalkylene is a divalent heteroalkyl group.

The term “heteroalkenyl,” as used herein, refers to an alkenyl group, as defined herein, in which one or more of the constituent carbon atoms have been replaced by nitrogen, oxygen, or sulfur. In some embodiments, the heteroalkenyl group can be further substituted with 1, 2, 3, or 4 substituent groups as described herein for alkenyl groups. Examples of heteroalkenyl groups are an “alkenoxy” which, as used herein, refers alkenyl-O—. A heteroalkenylene is a divalent heteroalkenyl group.

The term “heteroalkynyl,” as used herein, refers to an alkynyl group, as defined herein, in which one or more of the constituent carbon atoms have been replaced by nitrogen, oxygen, or sulfur.

In some embodiments, the heteroalkynyl group can be further substituted with 1, 2, 3, or 4 substituent groups as described herein for alkynyl groups. Examples of heteroalkynyl groups are an “alkynoxy” which, as used herein, refers alkynyl-O—. A heteroalkynylene is a divalent heteroalkynyl group.

The term “heteroaryl,” as used herein, refers to an aromatic mono- or polycyclic radical of 5 to 12 atoms having at least one aromatic ring containing one, two, or three ring heteroatoms selected from N, O, and S, with the remaining ring atoms being C. One or two ring carbon atoms of the heteroaryl group may be replaced with a carbonyl group. Examples of heteroaryl groups are pyridyl, pyrazoyl, benzooxazolyl, benzoimidazolyl, benzothiazolyl, imidazolyl, oxaxolyl, and thiazolyl.

The term “heteroarylalkyl,” as used herein, represents an alkyl group substituted with a heteroaryl group. Exemplary unsubstituted heteroarylalkyl groups are from 7 to 30 carbons (e.g., from 7 to 16 or from 7 to 20 carbons, such as C₁₋₆ alkyl C₂₋₉ heteroaryl, C₁₋₁₀ alkyl C₂₋₉ heteroaryl, or C₁₋₂₀ alkyl C₂₋₉ heteroaryl). In some embodiments, the akyl and the heteroaryl each can be further substituted with 1, 2, 3, or 4 substituent groups as defined herein for the respective groups.

The term “heterocyclyl,” as used herein, denotes a mono- or polycyclic radical having 3 to 12 atoms having at least one ring containing one, two, three, or four ring heteroatoms selected from N, O or S, wherein no ring is aromatic. Examples of heterocyclyl groups include, but are not limited to, morpholinyl, thiomorpholinyl, furyl, piperazinyl, piperidinyl, pyranyl, pyrrolidinyl, tetrahydropyranyl, tetrahydrofuranyl, and 1,3-dioxanyl.

The term “heterocyclylalkyl,” as used herein, represents an alkyl group substituted with a heterocyclyl group. Exemplary unsubstituted heterocyclylalkyl groups are from 7 to 30 carbons (e.g., from 7 to 16 or from 7 to 20 carbons, such as C₁₋₆ alkyl C₂₋₉ heterocyclyl, C₁₋₁₀ alkyl C₂₋₉ heterocyclyl, or C₁₋₂₀ alkyl C₂₋₉ heterocyclyl). In some embodiments, the akyl and the heterocyclyl each can be further substituted with 1, 2, 3, or 4 substituent groups as defined herein for the respective groups.

The term “hydroxyl,” as used herein, represents an —OH group.

The term “nitro,” as used herein, represents an —NO₂ group.

The term “thiol,” as used herein, represents an —SH group.

The alkyl, alkenyl, alkynyl, heteroalkyl, heteroalkenyl, heteroalkynyl, carbocyclyl (e.g., cycloalkyl), aryl, heteroaryl, and heterocyclyl groups may be substituted or unsubstituted. When substituted, there will generally be 1 to 4 substituents present, unless otherwise specified. Substituents include, for example: aryl (e.g., substituted and unsubstituted phenyl), carbocyclyl (e.g., substituted and unsubstituted cycloalkyl), halogen (e.g., fluoro), hydroxyl, heteroalkyl (e.g., substituted and unsubstituted methoxy, ethoxy, or thioalkoxy), heteroaryl, heterocyclyl, amino (e.g., NH₂ or mono- or dialkyl amino), azido, cyano, nitro, or thiol. Aryl, carbocyclyl (e.g., cycloalkyl), heteroaryl, and heterocyclyl groups may also be substituted with alkyl (unsubstituted and substituted such as arylalkyl (e.g., substituted and unsubstituted benzyl)).

Compounds of the invention can have one or more asymmetric carbon atoms and can exist in the form of optically pure enantiomers, mixtures of enantiomers such as, for example, racemates, optically pure diastereoisomers, mixtures of diastereoisomers, diastereoisomeric racemates or mixtures of diastereoisomeric racemates. The optically active forms can be obtained for example by resolution of the racemates, by asymmetric synthesis or asymmetric chromatography (chromatography with a chiral adsorbents or eluant). That is, certain of the disclosed compounds may exist in various stereoisomeric forms. Stereoisomers are compounds that differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that are not related as mirror images, most commonly because they contain two or more asymmetrically substituted carbon atoms and represent the configuration of substituents around one or more chiral carbon atoms. Enantiomers of a compound can be prepared, for example, by separating an enantiomer from a racemate using one or more well-known techniques and methods, such as, for example, chiral chromatography and separation methods based thereon. The appropriate technique and/or method for separating an enantiomer of a compound described herein from a racemic mixture can be readily determined by those of skill in the art. “Racemate” or “racemic mixture” means a compound containing two enantiomers, wherein such mixtures exhibit no optical activity; i.e., they do not rotate the plane of polarized light. “Geometric isomer” means isomers that differ in the orientation of substituent atoms in relationship to a carbon-carbon double bond, to a cycloalkyl ring, or to a bridged bicyclic system. Atoms (other than H) on each side of a carbon-carbon double bond may be in an E (substituents are on opposite sides of the carbon-carbon double bond) or Z (substituents are oriented on the same side) configuration. “R,” “S,” “S*,” “R*,” “E,” “Z,” “cis,” and “trans,” indicate configurations relative to the core molecule. Certain of the disclosed compounds may exist in atropisomeric forms. Atropisomers are stereoisomers resulting from hindered rotation about single bonds where the steric strain barrier to rotation is high enough to allow for the isolation of the conformers. The compounds of the invention may be prepared as individual isomers by either isomer-specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods. When the stereochemistry of a disclosed compound is named or depicted by structure, the named or depicted stereoisomer is at least 60%, 70%, 80%, 90%, 99% or 99.9%) by weight relative to the other stereoisomers. When a single enantiomer is named or depicted by structure, the depicted or named enantiomer is at least 60%, 70%, 80%, 90%, 99% or 99.9% by weight optically pure. When a single diastereomer is named or depicted by structure, the depicted or named diastereomer is at least 60%, 70%, 80%, 90%, 99% or 99.9% by weight pure. Percent optical purity is the ratio of the weight of the enantiomer or over the weight of the enantiomer plus the weight of its optical isomer. Diastereomeric purity by weight is the ratio of the weight of one diastereomer or over the weight of all the diastereomers. When the stereochemistry of a disclosed compound is named or depicted by structure, the named or depicted stereoisomer is at least 60%, 70%, 80%, 90%, 99% or 99.9% by mole fraction pure relative to the other stereoisomers. When a single enantiomer is named or depicted by structure, the depicted or named enantiomer is at least 60%, 70%, 80%, 90%, 99% or 99.9% by mole fraction pure. When a single diastereomer is named or depicted by structure, the depicted or named diastereomer is at least 60%, 70%, 80%, 90%, 99% or 99.9% by mole fraction pure. Percent purity by mole fraction is the ratio of the moles of the enantiomer or over the moles of the enantiomer plus the moles of its optical isomer. Similarly, percent purity by moles fraction is the ratio of the moles of the diastereomer or over the moles of the diastereomer plus the moles of its isomer. When a disclosed compound is named or depicted by structure without indicating the stereochemistry, and the compound has at least one chiral center, it is to be understood that the name or structure encompasses either enantiomer of the compound free from the corresponding optical isomer, a racemic mixture of the compound or mixtures enriched in one enantiomer relative to its corresponding optical isomer. When a disclosed compound is named or depicted by structure without indicating the stereochemistry and has two or more chiral centers, it is to be understood that the name or structure encompasses a diastereomer free of other diastereomers, a number of diastereomers free from other diastereomeric pairs, mixtures of diastereomers, mixtures of diastereomeric pairs, mixtures of diastereomers in which one diastereomer is enriched relative to the other diastereomer(s) or mixtures of diastereomers in which one or more diastereomer is enriched relative to the other diastereomers. The invention embraces all of these forms.

In embodiments are provided novel amino acid sequences. The term “amino acid” refers to naturally occurring and non-natural or unnatural amino acids, which may be referred to herein as synthetic amino acids, as well as amino acid analogs and amino acid mimetics that function in a manner similar to the naturally occurring amino acids. Naturally encoded amino acids are the 20 common amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine) and pyrolysine and selenocysteine. Amino acid analogs refer to compounds that have the same basic chemical structure as a naturally occurring amino acid, by way of example only, an α-carbon that is bound to a hydrogen, a carboxyl group, an amino group, and a functional R group. Such analogs may have modified R groups (by way of example, norleucine) or may have modified peptide backbones while still retaining the same basic chemical structure as a naturally occurring amino acid. Non-limiting examples of amino acid analogs include homoserine, norleucine, methionine sulfoxide, methionine methyl sulfonium. Amino acids may be referred to herein by either their name, their commonly known three letter symbols or by the one-letter symbols recommended by the IUPAC-IUB Biochemical Nomenclature Commission. Additionally, nucleotides, may be referred to by their commonly accepted single-letter codes.

An “amino or carboxy terminus modification group” refers to any molecule that can be attached to a terminal amine group or terminal carboxy group respectively. By way of example, such terminal amine groups or terminal carboxy groups may be at the end of polymeric molecules, wherein such polymeric molecules include, but are not limited to, polypeptides, polynucleotides, and polysaccharides. Terminus modification groups include but are not limited to, various water-soluble polymers, peptides or proteins. By way of example only, terminus modification groups include polyethylene glycol or serum albumin. Terminus modification groups may be used to modify therapeutic characteristics of the polymeric molecule, including but not limited to increasing the serum half-life of peptides, polypeptides or proteins.

In some embodiments the disclosure provides novel antibodies and antibody variants. The term “antibody” herein refers to a protein consisting of one or more polypeptides substantially encoded by all or part of the antibody genes. The immunoglobulin genes include, but are not limited to, the kappa, lambda, alpha, gamma (IgG1, IgG2, IgG3, and IgG4), delta, epsilon and mu constant region genes, as well as the myriad immunoglobulin variable region genes. Antibody herein is also meant to include full-length antibodies and antibody fragments, and include antibodies that exist naturally in any organism, antibody variants, engineered antibodies and antibody fragments. Antibody herein is also meant to include intact antibody, monoclonal or polyclonal antibodies. Antibody herein also encompasses, multispecific antibodies and/or bispecific antibodies. Antibodies of the present disclosure include human antibodies. Human antibodies are usually made of two light chains and two heavy chains each comprising variable regions and constant regions. The light chain variable region comprises 3 CDRs, identified herein as CDRL1, CDRL2 and CDRL3 flanked by framework regions. The heavy chain variable region comprises 3 CDRs, identified herein as CDRH1, CDRH2 and CDRH3 flanked by framework regions.

The term “antibody fragment” herein refers to any form of an antibody other than the full-length form. Antibody fragments herein include antibodies that are smaller components that exist within full-length antibodies, and antibodies that have been engineered, such as antibody variants. Antibody fragments include but are not limited to Fv, Fc, Fab, and (Fab′)₂, single chain Fv (scFv), diabodies, triabodies, tetrabodies, bifunctional hybrid antibodies, CDR1, CDR2, CDR3, combinations of CDRs, variable regions, framework regions, constant regions, heavy chains, light chains, and variable regions, and alternative scaffold non-antibody molecules, bispecific antibodies, and the like (Maynard & Georgiou, Annu. Rev. Biomed. Eng. 2:339-76, 2000; Hudson, Curr. Opin. Biotechnol. 9:395-402, 1998). Another functional substructure is a single chain Fv (scFv), comprised of the variable regions of the immunoglobulin heavy and light chain, covalently connected by a peptide linker (Hu et al., Cancer Research, 56, 3055-3061, 1996). These small (Mr 25,000) proteins generally retain specificity and affinity for antigen in a single polypeptide and can provide a convenient building block for larger, antigen-specific molecules. Unless specifically noted otherwise, statements and claims that use the term “antibody” or “antibodies” specifically includes “antibody fragment” and “antibody fragments.”

In embodiments novel antibody drug conjugates (ADCs) are disclosed. The term “antibody-drug conjugate, or “ADC”, as used herein, refers to an antibody molecule, or fragment thereof, that is covalently bonded to one or more biologically active molecule(s). The biologically active molecule may be conjugated to the antibody through a linker, polymer, or other covalent bond. ADCs are a potent class of therapeutic constructs that allow targeted delivery of cytotoxic agents to target cells, such as cancer cells. Because of the targeting function, these compounds show a much higher therapeutic index compared to the same systemically delivered agents. ADCs have been developed as intact antibodies or antibody fragments, such as scFvs. The antibody or fragment is linked to one or more copies of drug via a linker that is stable under physiological conditions, but that may be cleaved once inside the target cell.

The term “antigen-binding fragment”, as used herein, refers to one or more fragments of an antibody that retain the ability to bind to an antigen. It has been shown that the antigen-binding function of an antibody can be performed by fragments of an intact antibody. Examples of binding fragments encompassed within the term “antigen-binding fragment” of an antibody include (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an antibody, (v) a dAb fragment (Ward et al., Nature 341:544-546, 1989), which consists of a V_{H} domain; (vi) an isolated complementarity determining region (CDR), e.g., V_{H} CDR3 comprising or not additional sequence (linker, framework region(s) etc.) and (v) a combination of two to six isolated CDRs comprising or not additional sequence (linker, framework region(s) etc.). Furthermore, although the two domains of the Fv fragment, V_{L} and V_{H}, are coded for by separate genes, they can be joined, using recombinant methods, by a synthetic linker that enables them to be made as a single polypeptide chain in which the V_{L} and V_{H} regions pair to form monovalent molecules (known as single chain Fv (scFv); see e.g., Bird et al., Science 242:423-426, 1988); and (Huston et al., Proc. Natl. Acad. Sci. USA 85:5879-5883, 1988). Such single chain antibodies are also intended to be encompassed within the term “antigen-binding fragment” of an antibody. Furthermore, the antigen-binding fragments include binding-domain immunoglobulin fusion proteins comprising (i) a binding domain polypeptide (such as a heavy chain variable region, a light chain variable region, or a heavy chain variable region fused to a light chain variable region via a linker peptide) that is fused to an immunoglobulin hinge region polypeptide, (ii) an immunoglobulin heavy chain CH2 constant region fused to the hinge region, and (iii) an immunoglobulin heavy chain CH3 constant region fused to the CH2 constant region. The hinge region may be modified by replacing one or more cysteine residues with serine residues to prevent dimerization. Such binding-domain immunoglobulin fusion proteins are further disclosed in US 2003/0118592 and US 2003/0133939. These antibody fragments are obtained using conventional techniques known to those with skill in the art, and the fragments are screened for utility in the same manner as are intact antibodies.

A typical antigen binding site is comprised of the variable regions formed by the pairing of a light chain immunoglobulin and a heavy chain immunoglobulin. The structure of the antibody variable regions is very consistent and exhibits very similar structures. These variable regions are typically comprised of relatively homologous framework regions (FR) interspaced with three hypervariable regions termed Complementarity Determining Regions (CDRs). The overall binding activity of the antigen binding fragment is often dictated by the sequence of the CDRs. The FRs often play a role in the proper positioning and alignment in three dimensions of the CDRs for optimal antigen binding. In fact, because CDR sequences are responsible for most antibody-antigen interactions, it is possible to express recombinant antibodies that shows the properties of specific naturally occurring antibodies by constructing expression vectors that include CDR sequences from the specific naturally occurring antibody grafted onto framework sequences from a different antibody with different properties (see, e.g., Riechmann, L. et al., Nature 332:323-327, 1998; Jones, P. et al., Nature 321:522-525, 1986; and Queen, C. et al., Proc. Natl. Acad. USA 86:10029-10033, 1989). Such framework sequences can be obtained from public DNA databases that include germline antibody gene sequences. These germline sequences will differ from mature antibody gene sequences because they will not include completely assembled variable genes, which are formed by V(D)J joining during B cell maturation. Germline gene sequences will also differ from the sequences of a high affinity secondary repertoire antibody which contains mutations throughout the variable gene but typically clustered in the CDRs. For example, somatic mutations are relatively infrequent in the amino terminal portion of framework region 1 and in the carboxy-terminal portion of framework region 4. Furthermore, many somatic mutations do not significantly alter the binding properties of the antibody. For this reason, it is not necessary to obtain the entire DNA sequence of a particular antibody in order to recreate an intact recombinant antibody having binding properties similar to those of the original antibody. Partial heavy and light chain sequence spanning the CDR regions is typically sufficient for this purpose. The partial sequence is used to determine which germline variable and joining gene segments contributed to the recombined antibody variable genes. The germline sequence is then used to fill in missing portions of the variable regions. Heavy and light chain leader sequences are cleaved during protein maturation and do not contribute to the properties of the final antibody. To add missing sequences, cloned cDNA sequences can be combined with synthetic oligonucleotides by ligation or PCR amplification. Alternatively, the entire variable region can be synthesized to create an entirely synthetic variable region clone. This process has certain advantages such as elimination or inclusion of particular restriction sites, or optimization of particular codons. Of course, the totality or portions of the framework region of the antibody described herein may be used in conjunction with the CDRs in order to optimize the affinity, specificity or any other desired properties of the antibody.

In some embodiments the disclosure concerns polymers such as a bifunctional polymer. A “bifunctional polymer”, also referred to as a “bifunctional linker”, refers to a polymer comprising two functional groups that are capable of reacting specifically with other moieties to form covalent or non-covalent linkages. Such moieties may include, but are not limited to, the side groups on natural or non-natural amino acids or peptides which contain such natural or non-natural amino acids. The other moieties that may be linked to the bifunctional linker or bifunctional polymer may be the same or different moieties. By way of example only, a bifunctional linker may have a functional group reactive with a group on a first peptide, and another functional group which is reactive with a group on a second peptide, whereby forming a conjugate that includes the first peptide, the bifunctional linker and the second peptide. Many procedures and linker molecules for attachment of various compounds to peptides are known. See, for example, European Patent Application No. 0188256; U.S. Pat. Nos. 4,659,839; 4,414,148; 4,699,784; 4,680,338; and 4,569,789 incorporated herein by reference in their entirety. A “multi-functional polymer” also referred to as a “multi-functional linker”, refers to a polymer comprising two or more functional groups that are capable of reacting with other moieties. Such moieties may include, but are not limited to, the side groups on natural or non-natural amino acids or peptides which contain such natural or non-natural amino acids (including but not limited to, amino acid side groups) to form covalent or non-covalent linkages. A bi-functional polymer or multi-functional polymer may be any desired length or molecular weight and may be selected to provide a particular desired spacing or conformation between one or more molecules linked to a compound and molecules it binds to, or to the compound.

The term “bioavailability,” as used herein, refers to the rate and extent to which a substance or its active moiety is delivered from a pharmaceutical dosage form and becomes available at the site of action or in the general circulation. Increases in bioavailability refers to increasing the rate and extent a substance or its active moiety is delivered from a pharmaceutical dosage form and becomes available at the site of action or in the general circulation. By way of example, an increase in bioavailability may be indicated as an increase in concentration of the substance or its active moiety in the blood when compared to other substances or active moieties.

The term “biologically active molecule”, “biologically active moiety” or “biologically active agent” when used herein means any substance which can affect any physical or biochemical properties of a biological system, pathway, molecule, or interaction relating to an organism, including but not limited to, viruses, bacteria, bacteriophage, transposon, prion, insects, fungi, plants, animals, and humans. In particular, as used herein, biologically active molecules include but are not limited to any substance intended for diagnosis, cure, mitigation, treatment, or prevention of disease in humans or other animals, or to otherwise enhance physical or mental well-being of humans or animals. Examples of biologically active molecules include, but are not limited to, peptides, proteins, enzymes, small molecule drugs, hard drugs, soft drugs, prodrugs, carbohydrates, inorganic atoms or molecules, dyes, lipids, nucleosides, radionuclides, oligonucleotides, toxins, cells, viruses, liposomes, microparticles and micelles. Classes of biologically active agents that are suitable for use with the methods and compositions described herein include, but are not limited to, drugs, prodrugs, radionuclides, imaging agents, polymers, antibiotics, fungicides, anti-viral agents, anti-inflammatory agents, anti-tumor agents, cardiovascular agents, anti-anxiety agents, hormones, growth factors, steroidal and nonsteroidal agents, microbially derived toxins, and the like.

By “modulating biological activity” is meant increasing or decreasing the reactivity of a polypeptide, altering the selectivity of the polypeptide, enhancing or decreasing the substrate selectivity of the polypeptide. Analysis of modified biological activity can be performed by comparing the biological activity of the non-natural polypeptide to that of the natural polypeptide.

In some embodiments the disclosure concerns amino acids that have been biosynthetically incorporated in the antibody. The term “biosynthetically,” as used herein, refers to any method utilizing a translation system (cellular or non-cellular), including use of at least one of the following components: a polynucleotide, a codon, a tRNA, and a ribosome. By way of example, non-natural amino acids may be “biosynthetically incorporated” into non-natural amino acid polypeptides using the methods and techniques described herein and as is well known in the art. See for example, WO2010/011735 and WO2005/074650.

The term “conservatively modified variants” applies to both natural and non-natural amino acid and natural and non-natural nucleic acid sequences, and combinations thereof. With respect to particular nucleic acid sequences, “conservatively modified variants” refers to those natural and non-natural nucleic acids which encode identical or essentially identical natural and non-natural amino acid sequences, or where the natural and non-natural nucleic acid does not encode a natural and non-natural amino acid sequence, to essentially identical sequences. By way of example, because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Thus, by way of example every natural or non-natural nucleic acid sequence herein which encodes a natural or non-natural polypeptide also describes every possible silent variation of the natural or non-natural nucleic acid. One of ordinary skill in the art will recognize that each codon in a natural or non-natural nucleic acid (except AUG, which is ordinarily the only codon for methionine, and TGG, which is ordinarily the only codon for tryptophan) can be modified to yield a functionally identical molecule. Accordingly, each silent variation of a natural and non-natural nucleic acid which encodes a natural and non-natural polypeptide is implicit in each described sequence. As to amino acid sequences, individual substitutions, deletions or additions to a nucleic acid, peptide, polypeptide, or protein sequence which alters, adds or deletes a single natural and non-natural amino acid or a small percentage of natural and non-natural amino acids in the encoded sequence is a “conservatively modified variant” where the alteration results in the deletion of an amino acid, addition of an amino acid, or substitution of a natural and non-natural amino acid with a chemically similar amino acid. Conservative substitution tables providing functionally similar natural amino acids are well known in the art. Conservative substitution tables providing functionally similar amino acids are known to those of ordinary skill in the art. The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins: Structures and Molecular Properties (W H Freeman & Co.; 2nd edition, 1993). Such conservatively modified variants are in addition to and do not exclude polymorphic variants, interspecies homologs, and alleles of the compositions described herein.

The term “drug,” as used herein, refers to any substance used in the prevention, diagnosis, alleviation, treatment, or cure of a disease or condition such as cancer, including but not limited to oral, colorectal, gastric, esophageal, hepatocellular, non-small-cell-lung (NSCL), small-cell lung (SCL), ovarian, breast including triple-negative breast, prostate, pancreatic, head and neck, squamous, renal, bladder, cervical, endometrial, thyroid, glioblastoma cancer, or a blood cancer, including a leukemia, a lymphoma or myeloma.

The term “drug-to-antibody ratio” (“DAR”) as used herein refers to the average (mean) number of drugs that are conjugated to an antibody in an antibody-drug conjugate (ADC) composition. The DAR value reflects the homogeneity of the ADC population in the composition, and also indicates the amount of “payload” (e.g, drug or drug-linker) that is loaded onto an antibody and can be delivered to a target (e.g., cell or diseased tissue). DAR can be determined by methods known to a person of ordinary skill in the art, for example, LC-MS (e.g., see Tang, Y. et al., Real-Time Analysis on Drug-Antibody Ratio of Antibody-Drug Conjugates for Synthesis, Process Optimization and Quality Control, Sci Rep 7, 7763 (2017). doi: 10.1038/s41598-017-08151-2; and Chen, Y. Drug-to-antibody ratio (DAR) by UV/Vis spectroscopy, Methods Mol. Biol., 2013; 1045:267-73. doi: 10.1007/978-1-62703-541-5_16). In a non-limiting example, an ADC can have a population distribution of 20% of drug-loaded antibody, wherein the drug load is two (2) drugs per antibody; 25% of drug-loaded antibody, wherein the drug load is three (3) drugs per antibody; and 55% of drug-loaded antibody, wherein the drug load is four (4) drugs per antibody; thus, in this example, DAR is [(0.2×2)+(0.25×3)+(0.55×4)]=3.35.

The term “effective amount,” as used herein, refers to a sufficient amount of an agent, compound or composition being administered which will relieve to some extent one or more of the symptoms of the disease or condition being treated. The result can be reduction and/or alleviation of the signs, symptoms, or causes of a disease, or any other desired alteration of a biological system. By way of example, an agent, compound or composition being administered includes, but is not limited to, a natural amino acid polypeptide, non-natural amino acid polypeptide, modified natural amino acid polypeptide, modified non-amino acid polypeptide, or an antibody or variant thereof. Compositions containing such natural amino acid polypeptides, non-natural amino acid polypeptides, modified natural amino acid polypeptides, modified non-natural amino acid polypeptides, or an antibody or variant thereof can be administered for prophylactic, enhancing, and/or therapeutic treatments. An appropriate “effective” amount in any individual case may be determined using techniques, such as a dose escalation study.

The terms “enhance” or “enhancing” means to increase or prolong either in potency or duration a desired effect. By way of example, “enhancing” the effect of therapeutic agents refers to the ability to increase or prolong, either in potency or duration, the effect of therapeutic agents on during treatment of a disease, disorder or condition. An “enhancing-effective amount,” as used herein, refers to an amount adequate to enhance the effect of a therapeutic agent in the treatment of a disease, disorder or condition. When used in a patient, amounts effective for this use will depend on the severity and course of the disease, disorder or condition, previous therapy, the patient's health status and response to the drugs, and the judgment of the treating physician.

The term “humanized or chimeric antibody” refer to a molecule, generally prepared using recombinant techniques, having an antigen binding site derived from an immunoglobulin from a non-human species, (e.g., murine), and the remaining immunoglobulin structure of the molecule based upon the structure and/or sequence of a human immunoglobulin. In general, the humanized antibody will comprise substantially all of at least one, and typically two, variable domains, in which all or substantially all of the hypervariable loops correspond to those of a non-human immunoglobulin and all or substantially all of the framework residues/regions (FR) are those of a human immunoglobulin sequence. The humanized antibody optionally also will comprise at least a portion of an immunoglobulin constant region (Fc), typically that of a human immunoglobulin. The humanized forms of rodent antibodies will essentially comprise the same CDR sequences of the parental rodent antibodies, although certain amino acid substitutions may be included to increase affinity, increase stability of the humanized antibody, or for other reasons. However, as CDR loop exchanges do not uniformly result in an antibody with the same binding properties as the antibody of origin, changes in framework residues (FR), residues involved in CDR loop support, might also be introduced in humanized antibodies to preserve antigen binding affinity. The antigen-binding site may comprise either complete variable domains fused onto constant domains or only the complementarity determining regions (CDRs) grafted onto appropriate framework regions in the variable domains. Antigen binding sites may be wild type or modified by one or more amino acid substitutions. This eliminates the constant region as an immunogen in human individuals, but the possibility of an immune response to the foreign variable region remains (LoBuglio, A. F. et al., “Mouse/Human Chimeric Monoclonal Antibody in Man: Kinetics and Immune Response,” Proc. Natl. Acad. Sci. (USA) 86:4220-4224, 1989). Another approach focuses not only on providing human-derived constant regions but modifying the variable regions as well so as to reshape them as closely as possible to human form. It is known that the variable regions of both heavy and light chains contain three complementarity-determining regions (CDRs) which vary in response to the antigens in question and determine binding capability, flanked by four framework regions (FRs) which are relatively conserved in a given species and which putatively provide a scaffolding for the CDRs. When nonhuman antibodies are prepared with respect to a particular antigen, the variable regions can be “humanized” by grafting CDRs derived from nonhuman antibody on the FRs present in the human antibody to be modified. Application of this approach to various antibodies has been reported by Kettleborough, C. A. et al., “Humanization Of A Mouse Monoclonal Antibody By CDR-Grafting: The Importance Of Framework Residues On Loop Conformation,” Protein Engineering 4:773-3783, 1991; Co, M. S. et al., “Humanized Antibodies For Antiviral Therapy,” Proc. Natl. Acad. Sci. (USA) 88:2869-2873, 1991; Carter, P. et al., “Humanization Of An Anti-p185her2 Antibody For Human Cancer Therapy,” Proc. Natl. Acad. Sci. (USA) 89:4285-4289, 1992; and Co, M. S. et al., “Chimeric And Humanized Antibodies With Specificity For The CD33 Antigen,” J. Immunol. 148:1149-1154, 1992. In some embodiments, humanized antibodies preserve all CDR sequences (for example, a humanized mouse antibody which contains all six CDRs from the mouse antibodies). In other embodiments, humanized antibodies have one or more CDRs (one, two, three, four, five, six) which are altered with respect to the original antibody, which are also termed one or more CDRs “derived from” one or more CDRs from the original antibody.

The term “identical,” as used herein, refers to two or more sequences or subsequences which are the same. In addition, the term “substantially identical,” as used herein, refers to two or more sequences which have a percentage of sequential units which are the same when compared and aligned for maximum correspondence over a comparison window, or designated region as measured using comparison algorithms or by manual alignment and visual inspection. By way of example only, two or more sequences may be “substantially identical” if the sequential units are about 60% identical, about 65% identical, about 70% identical, about 75% identical, about 80% identical, about 85% identical, about 90% identical, or about 95% identical over a specified region. Such percentages describe the “percent identity” of two or more sequences. The identity of a sequence can exist over a region that is at least about 75-100 sequential units in length, over a region that is about 50 sequential units in length, or, where not specified, across the entire sequence. This definition also refers to the complement of a test sequence. By way of example only, two or more polypeptide sequences are identical when the amino acid residues are the same, while two or more polypeptide sequences are “substantially identical” if the amino acid residues are about 60% identical, about 65% identical, about 70% identical, about 75% identical, about 80% identical, about 85% identical, about 90% identical, or about 95% identical over a specified region. The identity can exist over a region that is at least about 75 to about 100 amino acids in length, over a region that is about 50 amino acids in length, or, where not specified, across the entire sequence of a polypeptide sequence. In addition, by way of example only, two or more polynucleotide sequences are identical when the nucleic acid residues are the same, while two or more polynucleotide sequences are “substantially identical” if the nucleic acid residues are about 60% identical, about 65% identical, about 70% identical, about 75% identical, about 80% identical, about 85% identical, about 90% identical, or about 95% identical over a specified region. The identity can exist over a region that is at least about 75 to about 100 nucleic acids in length, over a region that is about 50 nucleic acids in length, or, where not specified, across the entire sequence of a polynucleotide sequence.

The term “immunogenicity,” as used herein, refers to an antibody response to administration of a therapeutic drug. The immunogenicity toward therapeutic non-natural amino acid polypeptides can be obtained using quantitative and qualitative assays for detection of anti-non-natural amino acid polypeptides antibodies in biological fluids. Such assays include, but are not limited to, Radioimmunoassay (RIA), Enzyme-linked immunosorbent assay (ELISA), luminescent immunoassay (LIA), and fluorescent immunoassay (FIA). Analysis of immunogenicity toward therapeutic non-natural amino acid polypeptides involves comparing the antibody response upon administration of therapeutic non-natural amino acid polypeptides to the antibody response upon administration of therapeutic natural amino acid polypeptides.

The term “isolated,” as used herein, refers to separating and removing a component of interest from components not of interest. Isolated substances can be in either a dry or semi-dry state, or in solution, including but not limited to an aqueous solution. The isolated component can be in a homogeneous state or the isolated component can be a part of a pharmaceutical composition that comprises additional pharmaceutically acceptable carriers and/or excipients. Purity and homogeneity may be determined using analytical chemistry techniques including, but not limited to, polyacrylamide gel electrophoresis or high-performance liquid chromatography. In addition, when a component of interest is isolated and is the predominant species present in a preparation, the component is described herein as substantially purified. The term “purified,” as used herein, may refer to a component of interest which is at least 85% pure, at least 90% pure, at least 95% pure, at least 99% or greater pure. By way of example only, nucleic acids or proteins are “isolated” when such nucleic acids or proteins are free of at least some of the cellular components with which it is associated in the natural state, or that the nucleic acid or protein has been concentrated to a level greater than the concentration of its in vivo or in vitro production. Also, by way of example, a gene is isolated when separated from open reading frames which flank the gene and encode a protein other than the gene of interest.

The term “linkage” or “adduct moiety” as used herein refers to a bond or chemical moiety formed from a chemical reaction between the functional group of one group, such as a linker of the present disclosure, and another molecule. Such bonds may include, but are not limited to, covalent linkages and non-covalent bonds, while such chemical moieties may include, but are not limited to, esters, carbonates, imines, phosphate esters, hydrazones, acetals, orthoesters, peptide linkages, oximes and oligonucleotide linkages. Hydrolytically stable linkages mean that the linkages are substantially stable in water and do not react with water at useful pH values, including but not limited to, under physiological conditions for an extended period of time, perhaps even indefinitely. Hydrolytically unstable or degradable linkages mean that the linkages are degradable in water or in aqueous solutions, including for example, blood. Enzymatically unstable or degradable linkages mean that the linkage can be degraded by one or more enzymes. By way of example only, PEG and related polymers may include degradable linkages in the polymer backbone or in the linker group between the polymer backbone and one or more of the terminal functional groups of the polymer molecule. Such degradable linkages include but are not limited to ester linkages formed by the reaction of PEG carboxylic acids or activated PEG carboxylic acids with alcohol groups on a biologically active agent, wherein such ester groups generally hydrolyze under physiological conditions to release the biologically active agent. Other hydrolytically degradable linkages include but are not limited to carbonate linkages; imine linkages resulted from reaction of an amine and an aldehyde; phosphate ester linkages formed by reacting an alcohol with a phosphate group; hydrazone linkages which are reaction product of a hydrazide and an aldehyde; acetal linkages that are the reaction product of an aldehyde and an alcohol; orthoester linkages that are the reaction product of a formate and an alcohol; peptide linkages formed by an amine group, including but not limited to, at an end of a polymer such as PEG, and a carboxyl group of a peptide; and oligonucleotide linkages formed by a phosphoramidite group, including but not limited to, at the end of a polymer, and a 5′ hydroxyl group of an oligonucleotide.

The term “linker,” as used herein, refers to any multivalent group that connects, or is capable of connecting, a first group to at least one other group. Typically, a linker is a bivalent or a trivalent organic moiety that connects a drug (first group) to a biologically active agent (second group), e.g., via a linkage or adduct moiety, or that connects a drug (first group) to a reactive moiety (second group), wherein the reactive moiety is capable of reacting with a biologically active agent. Linkers can be susceptible to cleavage (cleavable linkers), such as, acid-induced cleavage, photo-induced cleavage, peptidase-induced cleavage, esterase-induced cleavage, and disulfide bond cleavage, and so on, at conditions under which the drug and the at least one other group remains active. Alternatively, linkers can be substantially resistant to cleavage (e.g., stable linker or non-cleavable linker).

In some embodiments, the linker L is a bivalent or trivalent group comprising, or consisting of, at least one moiety, wherein each at least one moiety is independently selected from the group consisting of a bond, unsubstituted alkylene, substituted alkylene, -(alkylene-O)ₙ—, optionally substituted arylene, —O—, —C(O)—, —C(S)—, —N(R_{w})—, —S(O)₀₋₂—, methine (—CH)—, an amino acid, a peptide, a disulfide (—S—S—), a water soluble polymer, and a phosphate-based moiety; and combinations thereof; wherein: each R_{w} is independently H, C₁-C₈alkyl or a bond; and each phosphate-based moiety is independently selected from the group consisting of a phosphate ester, a pyrophosphate ester, a triphosphate ester, a tetraphosphate ester, a phosphonate, a diphosphonate, a phosporamidate, a pyrophosporamidate, a triphosphoramidate, a tetraphosphoramidate, a phosphorthioate and a diphosphorthioate. In some embodiments, the phosphate-based moiety is phosphonate, diphosphonate, tetraphosphate ester or diphosphorthioate. In some embodiments, the phosphate-based moiety is diphosphonate. In some embodiments, the disphosphonate moiety is conjugated to an oxygen atom of a drug (e.g., a duarcomycin of the present disclosure) to provide a drug-linker comprising a pyrophosphate ester. In some embodiments, the water-soluble polymer is a (polyethylene) glycol (PEG) or modified PEG. In some embodiments, the water-soluble polymer is a polysaccharide. Unless expressly indicated otherwise, no orientation of the linker is implied by the direction in which the formula of the linker group is written. By way of example, the formula —C(O)CH₂CH₂— represents both —C(O)CH₂CH₂— and —CH₂CH₂C(O)—. In another example, the formula —C(O)CH₂CH₂-represents both *—C(O)CH₂CH₂— and —C(O)CH₂CH₂—*, wherein * denotes a point of connection, for example, connection to a drug. In some embodiments, when a selected moiety occurs two or more times in the same linker, the two or more occurrences are not adjacent. In some embodiments, a linker is not a bond.

In some embodiments, a linker is a bivalent moiety that connects a first group and a second group. In some other embodiments, the linker is a trivalent moiety that connects a first group, a second group and a third group. In a non-limiting example, a trivalent moiety is C(H) (i.e., methine) or N. In some other embodiments, a linker is a tetravalent moiety that connects a first group, a second group and a third group.

In some embodiments, a linker connects at least a first group and a second group, wherein the first group is a drug, and the second group is a biologically active polypeptide or protein. In some embodiments, the biologically active polypeptide or protein contains at least one non-natural amino acid. In some embodiments, the linker connects the drug to a non-natural amino acid of the biologically active polypeptide or protein. In some embodiments, the biologically active polypeptide or protein is an antibody. Thus, the antibody connected to a drug via a linker can be an antibody-drug conjugate (ADC), such as an ADC of the present disclosure.

In some other embodiments, a linker connects at least a first group and a second group, wherein the first group is a drug, and the second group is a reactive moiety. In some embodiments, the second group is a reactive moiety that is capable of reacting with a biologically active polypeptide or protein. In some embodiments, the biologically active polypeptide or protein contains at least one non-natural amino acid. Thus, in some embodiments, the reactive moiety is capable of reacting with a non-natural amino acid of the biologically active polypeptide or protein. In some embodiments, the biologically active polypeptide or protein is an antibody.

In some embodiments, a first linker is connected to a second linker, and the combined linkers (a composite linker) connects at least a first group and a second group. A composite linker of the present disclosure can contain 2, 3, 4, 5, 6, 7, 8, 9, 10 or more linker groups. In a non-limiting example, a first, second and third linker group are joined together to provide a composite linker that can connect a first group (e.g., a drug) to at least one other group, such as a reactive moiety and/or a biologically active polypeptide or protein (e.g., an antibody. In some embodiments, the biologically active polypeptide or protein (e.g., antibody) contains a non-natural amino acid.

In some embodiments, a linker is linear. In other embodiments, a linker is branched.

In some embodiments, a linker is a phosphate-based linker.

The term “phosphate-based linker” as used herein refers to a linker comprising a phosphate-based moiety, wherein the phosphate-based moiety is a phosphate ester, a pyrophosphate ester, a triphosphate ester, a tetraphosphate ester, a phosphonate, a diphosphonate, a phosporamidate, a pyrophosporamidate, a triphosphoramidate, a tetraphosphoramidate, a phosphorthioate and/or a diphosphorthioate.

The term “metabolite,” as used herein, refers to a derivative of a compound, by way of example natural amino acid polypeptide, a non-natural amino acid polypeptide, a modified natural amino acid polypeptide, or a modified non-natural amino acid polypeptide, that is formed when the compound, by way of example natural amino acid polypeptide, non-natural amino acid polypeptide, modified natural amino acid polypeptide, or modified non-natural amino acid polypeptide, is metabolized. The term “pharmaceutically active metabolite” or “active metabolite” refers to a biologically active derivative of a compound, by way of example natural amino acid polypeptide, a non-natural amino acid polypeptide, a modified natural amino acid polypeptide, or a modified non-natural amino acid polypeptide, that is formed when such a compound, by way of example a natural amino acid polypeptide, non-natural amino acid polypeptide, modified natural amino acid polypeptide, or modified non-natural amino acid polypeptide, is metabolized. The term “pharmaceutically active metabolite” or “active metabolite” also refers to biologically active derivatives of a compound, by way of example metabolizing phosphate linkages including monophosphate, diphosphate, pyrophosphate and triphosphate but not limited to such.

The term “metabolized,” as used herein, refers to the sum of the processes by which a particular substance is changed by an organism. Such processes include, but are not limited to, hydrolysis reactions and reactions catalyzed by enzymes. Further information on metabolism may be obtained from The Pharmacological Basis of Therapeutics, 9th Edition, McGraw-Hill (1996). By way of example only, metabolites of natural amino acid polypeptides, non-natural amino acid polypeptides, modified natural amino acid polypeptides, or modified non-natural amino acid polypeptides may be identified either by administration of the natural amino acid polypeptides, non-natural amino acid polypeptides, modified natural amino acid polypeptides, or modified non-natural amino acid polypeptides to a host and analysis of tissue samples from the host, or by incubation of natural amino acid polypeptides, non-natural amino acid polypeptides, modified natural amino acid polypeptides, or modified non-natural amino acid polypeptides with hepatic cells in vitro and analysis of the resulting compounds.

The term “modified,” as used herein refers to the presence of a change to a natural amino acid, a non-natural amino acid, a natural amino acid polypeptide or a non-natural amino acid polypeptide. Such changes, or modifications, may be obtained by post synthesis modifications of natural amino acids, non-natural amino acids, natural amino acid polypeptides or non-natural amino acid polypeptides, or by co-translational, or by post-translational modification of natural amino acids, non-natural amino acids, natural amino acid polypeptides or non-natural amino acid polypeptides.

A “non-natural amino acid” refers to an amino acid that is not one of the 20 common amino acids or pyrolysine or selenocysteine. Other terms that may be used synonymously with the term “non-natural amino acid” is “non-naturally encoded amino acid,” “unnatural amino acid,” “non-naturally-occurring amino acid,” “synthetic amino acid” and variously hyphenated and non-hyphenated versions thereof. The term “non-natural amino acid” includes, but is not limited to, amino acids which occur naturally by modification of a naturally encoded amino acid (including but not limited to, the 20 common amino acids or pyrrolysine and selenocysteine) but are not themselves incorporated into a growing polypeptide chain by the translation complex. Examples of naturally-occurring amino acids that are not naturally-encoded include, but are not limited to, N-acetylglucosaminyl-L-serine, N-acetylglucosaminyl-L-threonine, and O-phosphotyrosine. Additionally, the term “non-natural amino acid” includes, but is not limited to, amino acids which do not occur naturally and may be obtained synthetically or may be obtained by modification of non-natural amino acids.

The term “nucleic acid,” as used herein, refers to deoxyribonucleotides, deoxyribonucleosides, ribonucleosides or ribonucleotides and polymers thereof in either single- or double-stranded form. By way of example only, such nucleic acids and nucleic acid polymers include, but are not limited to, (i) analogues of natural nucleotides which have similar binding properties as a reference nucleic acid and are metabolized in a manner similar to naturally occurring nucleotides; (ii) oligonucleotide analogs including, but are not limited to, PNA (peptidonucleic acid), analogs of DNA used in antisense technology (phosphorothioates, phosphoroamidates, and the like); (iii) conservatively modified variants thereof (including but not limited to, degenerate codon substitutions) and complementary sequences and sequence explicitly indicated. By way of example, degenerate codon substitutions may be achieved by generating sequences in which the third position of one or more selected (or all) codons is substituted with mixed-base and/or deoxyinosine residues (Batzer et al., Nucleic Acid Res. 19:5081, 1991; Ohtsuka et al., J. Biol. Chem. 260:2605-2608, 1985; and Rossolini et al., Mol. Cell. Probes 8:91-98, 1994).

The term “pharmaceutically acceptable”, as used herein, refers to a material, including but not limited, to a salt, binder, adjuvant, excipient, carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

In some embodiments the disclosure concerns polymers. The term “polymer,” as used herein, refers to a molecule composed of repeated subunits. Such molecules include, but are not limited to, polypeptides, polynucleotides, or polysaccharides or polyalkylene glycols. Polymers of the disclosure can be linear or branched polymeric polyether polyols including, but are not limited to, polyethylene glycol, polypropylene glycol, polybutylene glycol, and derivatives thereof. Polymers can be activated polymers (e.g., activated PEGs) that facilitate conjugation to another group, such as a polypeptide, linker or drug-linker molecule. Polymers can also terminate in a moiety, such as a non-reactive moiety, e.g., alkyl (such as methyl) or alkoxy (such as methoxy). Other exemplary embodiments are listed, for example, in commercial supplier catalogs, such as Shearwater Corporation's catalog “Polyethylene Glycol and Derivatives for Biomedical Applications” (2001). By way of example only, such polymers have average molecular weights between about 0.1 kDa to about 100 kDa. Such polymers include, but are not limited to, between about 100 Da and about 100,000 Da or more. The molecular weight of the polymer may be between about 100 Da and about 100,000 Da, including but not limited to, about 100,000 Da, about 95,000 Da, about 90,000 Da, about 85,000 Da, about 80,000 Da, about 75,000 Da, about 70,000 Da, about 65,000 Da, about 60,000 Da, about 55,000 Da, about 50,000 Da, about 45,000 Da, about 40,000 Da, about 35,000 Da, about 30,000 Da, about 25,000 Da, about 20,000 Da, about 15,000 Da, about 10,000 Da, about 9,000 Da, about 8,000 Da, about 7,000 Da, about 6,000 Da, about 5,000 Da, about 4,000 Da, about 3,000 Da, about 2,000 Da, about 1,000 Da, about 900 Da, about 800 Da, about 700 Da, about 600 Da, about 500 Da, 400 Da, about 300 Da, about 200 Da, and about 100 Da. In some embodiments molecular weight of the polymer is between about 100 Da and about 50,000 Da. In some embodiments, the molecular weight of the polymer is between about 100 Da and about 40,000 Da. In some embodiments, the molecular weight of the polymer is between about 1,000 Da and about 40,000 Da. In some embodiments, the molecular weight of the polymer is between about 2,000 to about 50,000 Da. In some embodiments, the molecular weight of the polymer is between about 5,000 Da and about 40,000 Da. In some embodiments, the molecular weight of the polymer is between about 10,000 Da and about 40,000 Da. In some embodiments, the molecular weight of the polymer is within a range of about 100 Da to about 10,000 Da. In some embodiments, the molecular weight of the polymer is within a range of about 100 Da to about 5,000 Da. In some embodiments, the molecular weight of the polymer is within a range of about 100 Da to about 1,000 Da. In some embodiments, the polymer is a polyethylene glycol (PEG). In some embodiments, the PEG is a linear PEG. In some embodiments, the PEG is a branched PEG. The molecular weight of the linear or branched chain PEG may be between about 1,000 Da and about 100,000 Da, including but not limited to, about 100,000 Da, about 95,000 Da, about 90,000 Da, about 85,000 Da, about 80,000 Da, about 75,000 Da, about 70,000 Da, about 65,000 Da, about 60,000 Da, about 55,000 Da, about 50,000 Da, about 45,000 Da, about 40,000 Da, about 35,000 Da, about 30,000 Da, about 25,000 Da, about 20,000 Da, about 15,000 Da, about 10,000 Da, about 9,000 Da, about 8,000 Da, about 7,000 Da, about 6,000 Da, about 5,000 Da, about 4,000 Da, about 3,000 Da, about 2,000 Da, and about 1,000 Da. In some embodiments, the molecular weight of the linear or branched chain PEG is between about 1,000 Da and about 50,000 Da. In some embodiments, the molecular weight of the linear or branched chain PEG is between about 1,000 Da and about 40,000 Da. In some embodiments, the molecular weight of the linear or branched chain PEG is between about 5,000 Da and about 40,000 Da. In some embodiments, the molecular weight of the linear or branched chain PEG is between about 5,000 Da and about 20,000 Da. In other embodiments, the molecular weight of the linear or branched chain PEG is between about 2,000 to about 50,000 Da. In some embodiments, the molecular weight of the linear or branched chain PEG is within a range of about 100 Da to about 10,000 Da. In some embodiments, the molecular weight of the linear or branched chain PEG is within a range of about 100 Da to about 5,000 Da. In some embodiments, the molecular weight of the linear or branched chain PEG is within a range of about 100 Da to about 1,000 Da. In some embodiments, the PEG is a linear PEG. In some embodiments, the PEG comprises a defined number of repeating (-alkylene-O—) units, such as 2, 4, 6, 8, 10, 12, 14 or more units (e.g., PEG-2, PEG-4, PEG-6, PEG-8, PEG-10, PEG-12, PEG-14). In some embodiments, the PEG is a branched PEG. The term “PEGylating” or “PEGylated” is meant to refer to the covalent bonding of a specified moiety to a polyethylene glycol (PEG) molecule. In some embodiments, the moiety can be present in a drug, a drug-linker, a linker, or a polypeptide or protein. In some embodiments, the moiety is a hydroxyl group, a carboxylic acid, acyl or an amino group, such as a hydroxyl group, carboxylic acid, acyl or amino group present in a drug, drug-linker, linker or polypeptide. In some embodiments, the hydroxyl group, carboxylic acid, acyl or amino group is present in an amino acid. In some embodiments, the amino acid bearing the hydroxyl group, carboxylic acid, acyl or amino group is a natural or non-natural amino acid that is present in a polypeptide (e.g., an antibody), linker or drug-linker. The method can comprise contacting an isolated polypeptide comprising a natural or synthetic amino acid, or contacting a drug-linker comprising a natural or synthetic amino acid, with a water-soluble polymer (e.g., a PEG) comprising a moiety that reacts with the natural or synthetic amino acid. In a non-limiting example, the method can comprise contacting an isolated anti-TROP2 polypeptide, an isolated anti-HER2 polypeptide, an isolated anti-CD70 polypeptide, an isolated anti-PSMA polypeptide, an isolated anti-HER3 polypeptide or an isolated anti-GPC3 polypeptide, each comprising a natural or synthetic amino acid, with a water-soluble polymer comprising a moiety that reacts with the natural or synthetic amino acid.

The terms “polypeptide,” “peptide” and “protein” are used interchangeably herein to refer to a polymer of amino acid residues. That is, a description directed to a polypeptide applies equally to a description of a peptide and a description of a protein, and vice versa. The terms apply to naturally occurring amino acid polymers as well as amino acid polymers in which one or more amino acid residues is a non-natural amino acid. Additionally, such “polypeptides,” “peptides” and “proteins” include amino acid chains of any length, including full length proteins, including but not limited to antibodies, wherein the amino acid residues are linked by covalent peptide bonds.

The term “post-translationally modified” refers to any modification of a natural or non-natural amino acid which occurs after such an amino acid has been translationally incorporated into a polypeptide chain. Such modifications include, but are not limited to, co-translational in vivo modifications, co-translational in vitro modifications (such as in a cell-free translation system), post-translational in vivo modifications, and post-translational in vitro modifications.

The terms “prodrug” or “pharmaceutically acceptable prodrug,” as used herein, refers to an agent that is converted into the parent drug in vivo or in vitro, which does not abrogate the biological activity or properties of the drug, and is relatively nontoxic, i.e., the material may be administered to an individual without causing undesirable biological effects or interacting in a deleterious manner with any of the components of the composition in which it is contained. Prodrugs are generally drug precursors that, following administration to a subject and subsequent absorption, are converted to an active, or a more active species via some process, such as conversion by a metabolic pathway. Some prodrugs have a chemical group present on the prodrug that renders it less active and/or confers solubility or some other property to the drug. Once the chemical group has been cleaved and/or modified from the prodrug the active drug is generated. Prodrugs are converted into active drug within the body through enzymatic or non-enzymatic reactions. Prodrugs may provide improved physiochemical properties such as better solubility, enhanced delivery characteristics, such as specifically targeting a particular cell, tissue, organ or ligand, and improved therapeutic value of the drug. The benefits of such prodrugs include, but are not limited to, (i) ease of administration compared with the parent drug; (ii) the prodrug may be bioavailable by oral administration whereas the parent is not; and (iii) the prodrug may also have improved solubility in pharmaceutical compositions compared with the parent drug. A prodrug includes a pharmacologically inactive, or reduced activity, derivative of an active drug. Prodrugs may be designed to modulate the amount of a drug or biologically active molecule that reaches a desired site of action through the manipulation of the properties of a drug, such as physiochemical, biopharmaceutical, or pharmacokinetic properties. An example, without limitation, of a prodrug would be a non-natural amino acid polypeptide which is administered as an ester (the “prodrug”) to facilitate transmittal across a cell membrane where water solubility is detrimental to mobility and that is then metabolically hydrolyzed to the carboxylic acid, the active entity, once inside the cell where water solubility is beneficial. Prodrugs may be designed as reversible drug derivatives, for use as modifiers to enhance drug transport to site-specific tissues.

The term “prophylactically effective amount,” as used herein, refers to an amount of a composition containing at least one non-natural amino acid polypeptide or at least one modified non-natural amino acid polypeptide prophylactically applied to a patient which will relieve to some extent one or more of the symptoms of a disease, condition or disorder being treated. In such prophylactic applications, such amounts may depend on the patient's state of health, weight, and the like. It is considered well within the skill of the art for one to determine such prophylactically effective amounts by routine experimentation, including, but not limited to, a dose escalation clinical trial.

The term “recombinant host cell,” also referred to as “host cell,” refers to a cell which includes an exogenous polynucleotide, wherein the methods used to insert the exogenous polynucleotide into a cell include, but are not limited to, direct uptake, transduction, f-mating, or other methods known in the art to create recombinant host cells. By way of example only, such exogenous polynucleotide may be a nonintegrated vector, including but not limited to a plasmid, or may be integrated into the host genome.

The term “spacer” or “spacer element” as used herein refers to an atom or functional group that connects a first group to a second group. In some non-limiting embodiments, a spacer is a carbonyl (—C(O)—), —C(O)O—, —C(O)N(R)—, —O—, —S—, —N(R)— wherein each R is H or alkyl. In some embodiments the spacer is a bivalent spacer.

The term “subject” as used herein, refers to an animal which is the object of treatment, observation or experiment. By way of example only, a subject may be, but is not limited to, a mammal including, but not limited to, a human.

The term “substantially purified,” as used herein, refers to a component of interest that may be substantially or essentially free of other components which normally accompany or interact with the component of interest prior to purification. By way of example only, a component of interest may be “substantially purified” when the preparation of the component of interest contains less than about 30%, less than about 25%, less than about 20%, less than about 15%, less than about 10%, less than about 5%, less than about 4%, less than about 3%, less than about 2%, or less than about 1% (by dry weight) of contaminating components. Thus, a “substantially purified” component of interest may have a purity level of about 70%, about 75%, about 80%, about 85%, about 90%, about 95%, about 96%, about 97%, about 98%, about 99% or greater. By way of example only, a natural amino acid polypeptide or a non-natural amino acid polypeptide may be purified from a native cell, or host cell in the case of recombinantly produced natural amino acid polypeptides or non-natural amino acid polypeptides. By way of example a preparation of a natural amino acid polypeptide or a non-natural amino acid polypeptide may be “substantially purified” when the preparation contains less than about 30%, less than about 25%, less than about 20%, less than about 15%, less than about 10%, less than about 5%, less than about 4%, less than about 3%, less than about 2%, or less than about 1% (by dry weight) of contaminating material. By way of example when a natural amino acid polypeptide or a non-natural amino acid polypeptide is recombinantly produced by host cells, the natural amino acid polypeptide or non-natural amino acid polypeptide may be present at about 30%, about 25%, about 20%, about 15%, about 10%, about 5%, about 4%, about 3%, about 2%, or about 1% or less of the dry weight of the cells. By way of example when a natural amino acid polypeptide or a non-natural amino acid polypeptide is recombinantly produced by host cells, the natural amino acid polypeptide or non-natural amino acid polypeptide may be present in the culture medium at about 5 g/L, about 4 g/L, about 3 g/L, about 2 g/L, about 1 g/L, about 750 mg/L, about 500 mg/L, about 250 mg/L, about 100 mg/L, about 50 mg/L, about 10 mg/L, or about 1 mg/L or less of the dry weight of the cells. By way of example, “substantially purified” natural amino acid polypeptides or non-natural amino acid polypeptides may have a purity level of about 30%, about 35%, about 40%, about 45%, about 50%, about 55%, about 60%, about 65%, about 70%, about 75%, about 80%, about 85%, about 90%, about 95%, about 99% or greater as determined by appropriate methods, including, but not limited to, SDS/PAGE analysis, RP-HPLC, SEC, and capillary electrophoresis.

The term “therapeutically effective amount,” as used herein, refers to the amount of a composition containing at least one non-natural amino acid polypeptide and/or at least one modified non-natural amino acid polypeptide administered to a patient already suffering from a disease, condition or disorder, sufficient to cure or at least partially arrest, or relieve to some extent one or more of the symptoms of the disease, disorder or condition being treated. The effectiveness of such compositions depends on conditions including, but not limited to, the severity and course of the disease, disorder or condition, previous therapy, the patient's health status and response to the drugs, and the judgment of the treating physician. By way of example only, therapeutically effective amounts may be determined by routine experimentation, including but not limited to a dose escalation clinical trial.

The term “toxic”, or “toxic moiety” or “toxic group” or “cytotoxic” or “cytotoxic payload” or “payload” or “cytotoxic drug” or “drug” as used herein, refers to a cytotoxic compound which can cause harm, disturbances, or death. Toxic moieties include, but are not limited to, a drug comprising or consisting of a duocarmycin, or an analog or derivative thereof.

The terms “treat,” “treated,” “treating” or “treatment”, as used herein, include alleviating, preventing, abating or ameliorating a disease or condition symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. The terms “treat,” “treated,” “treating” or “treatment”, include, but are not limited to, prophylactic and/or therapeutic treatments. The term “treat”, “treated”, “treating” or “treatment” can refer to the decrease, reduction or amelioration of one or more symptoms or conditions or diseases associated with an antigen related or associated cancer. The term “treat”, “treated”, “treating” or “treatment” can refers to the administration of an ADC of the present disclosure to a subject in need thereof to decrease, reduce, improve, alter, relieve, affect or ameliorate an antigen related or associated cancer or disease or symptom or condition, or the predisposition toward a condition. The term “capable of specific binding” refers to protein or peptide (e.g., antibody) binding to a predetermined target substance (e.g., an antigen and/or groups of antigens), e.g. a target substance that is expressed on the surface of a cell; thus the term “binding to a target cell” or “binding to a cancer cell” is to be understand as referring to protein or peptide (e.g., antibody) binding to a predetermined target substance (e.g. antigen or antigens) that is expressed on such a cell. Typically, the protein or peptide (e.g., antibody) binds with an affinity of at least about 1×10⁷ M1, and/or binds to the predetermined target substance (e.g., antigen, antigens or cell) with an affinity that is at least two-fold greater than its affinity for binding to a non-specific control substance (e.g., BSA, casein, non-cancer cells) other than the predetermined target substance or a closely-related target substance.

As used herein, the term “water-soluble polymer” refers to any polymer that is soluble in aqueous solvents. Such water-soluble polymers include, but are not limited to, polyethylene glycol, polyethylene glycol propionaldehyde, mono C₁-C₁₀ alkoxy or aryloxy derivatives thereof (described in U.S. Pat. No. 5,252,714 which is incorporated by reference herein), monomethoxy-polyethylene glycol, polyvinyl pyrrolidone, polyvinyl alcohol, polyamino acids, divinylether maleic anhydride, N-(2-Hydroxypropyl)-methacrylamide, dextran, dextran derivatives including dextran sulfate, polypropylene glycol, polypropylene oxide/ethylene oxide copolymer, polyoxyethylated polyol, heparin, heparin fragments, polysaccharides, oligosaccharides, glycans, cellulose and cellulose derivatives, including but not limited to methylcellulose and carboxymethyl cellulose, serum albumin, starch and starch derivatives, polypeptides, polyalkylene glycol and derivatives thereof, copolymers of polyalkylene glycols and derivatives thereof, polyvinyl ethyl ethers, and alpha-beta-poly[(2-hydroxyethyl)-DL-aspartamide, and the like, or mixtures thereof. By way of example only, coupling of such water-soluble polymers to natural amino acid polypeptides or non-natural polypeptides may result in changes including, but not limited to, increased water solubility, increased or modulated serum half-life, increased or modulated therapeutic half-life relative to the unmodified form, increased bioavailability, modulated biological activity, extended circulation time, modulated immunogenicity, modulated physical association characteristics including, but not limited to, aggregation and multimer formation, altered receptor binding, altered binding to one or more binding partners, and altered receptor dimerization or multimerization. In addition, such water-soluble polymers may or may not have their own biological activity.

As used herein, the term “modulated serum half-life” refers to positive or negative changes in the circulating half-life of a modified biologically active molecule relative to its non-modified form. By way of example, the modified biologically active molecules include, but are not limited to, natural amino acid, non-natural amino acid, natural amino acid polypeptide or non-natural amino acid polypeptide. By way of example, serum half-life is measured by taking blood samples at various time points after administration of the biologically active molecule or modified biologically active molecule and determining the concentration of that molecule in each sample. Correlation of the serum concentration with time allows calculation of the serum half-life. By way of example, modulated serum half-life may be an increased in serum half-life, which may enable an improved dosing regimen or avoid toxic effects. Such increases in serum may be at least about two-fold, at least about three-fold, at least about five-fold, or at least about ten-fold. Methods for evaluating serum half-life are known in the art and may be used for evaluating the serum half-life of antibodies and antibody drug conjugates of the present disclosure.

The term “modulated therapeutic half-life,” as used herein, refers to positive or negative change in the half-life of the therapeutically effective amount of a modified biologically active molecule, relative to its non-modified form. By way of example, the modified biologically active molecules include, but are not limited to, natural amino acid, non-natural amino acid, natural amino acid polypeptide or non-natural amino acid polypeptide. By way of example, therapeutic half-life is measured by measuring pharmacokinetic and/or pharmacodynamic properties of the molecule at various time points after administration. Increased therapeutic half-life may enable a particular beneficial dosing regimen, a particular beneficial total dose, or avoids an undesired effect. By way of example, the increased therapeutic half-life may result from increased potency, increased or decreased binding of the modified molecule to its target, an increase or decrease in another parameter or mechanism of action of the non-modified molecule, or an increased or decreased breakdown of the molecules by enzymes such as, by way of example only, proteases. Methods for evaluating therapeutic half-life are known in the art and may be used for evaluating the therapeutic half-life of antibodies and antibody drug conjugates of the present disclosure.

### Introduction

Antibody-based therapeutics have emerged as important components of therapies for an increasing number of human malignancies in such fields as oncology, immunology, inflammatory and infectious diseases. In most cases, the basis of the therapeutic function is the high degree of specificity and affinity the antibody-based drug has for its target antigen. Arming monoclonal antibodies with drugs, toxins, or radionuclides is yet another strategy by which monoclonal antibodies may induce therapeutic effect. By combining the exquisite targeting specificity of antibody with the tumor killing power of toxic effector molecules, immunoconjugates permit sensitive discrimination between target and normal tissue thereby resulting in fewer side effects than most conventional chemotherapeutic drugs. The toxins utilized can specifically, stably and irreversibly conjugate to unique sites in the antibody. This unique process of conjugation allows for the precise control of the location of the toxin on the antibody, and also the number of toxins conjugated to each antibody. Both of these features are critical for controlling biophysical characteristics and toxicities associated with ADCs. (See for example Jackson D. et al. (2014) PLoS ONE 9(11):e83865; Tian F. et al. (2014) Proc. Natl. Acad. Sci. U.S.A. 111(15):1766-1771).

Currently ADCs are advancing the field of cancer therapeutics and a number of ADCs targeting various agents have been approved or are in clinical trials. However, ADCs face challenges due to lack of therapeutic index and toxicity. The linker technology for attachment of the cytotoxic drug to an antibody impacts the stability of ADCs during the systemic circulation. The release of free drug in the circulation instead of the release inside the antigen expressing cancer cells can cause ADC potency loss, insufficient immunogenic cancer cell death, and increased toxicity. Therefore, there is a need to design a stable linker such as phosphate-based linkers for drug design and antibody conjugation and for selective release inside the cancer cells.

### ADC Antibodies and Antibody Sequences

The present invention provides novel ADCs comprising antibodies, antibody fragments or variants thereof engineered to have one, or more non-natural amino acids incorporated at any desired position in the heavy and/or light chain amino acid sequence. Further, the present invention provides ADCs comprising one or more antibodies, antibody fragments or variants thereof engineered to have one, or more non-natural amino acids site specifically incorporated in the heavy and/or light chain amino acid sequence conjugated to drug via a phosphate-based linker. In some embodiments, the antibody, antibody fragment or variant thereof binds to a tumor-associated antigen (TAA) selected from the group consisting of PD-1, PD-Li, PSMA, CD70, CD3, HER2, HER3, TROP2, VEGFR, GPC3, EGFR, c-Met (HGFR), CD33, CD19, CD22, CD25 (IL-2R alpha), CD30, CD37, CD46, CD48, CD56 (NCAM-1), CD71 (Transferrin R), CD74, CD79b, C-D123 (IL-3R alpha), CD138 (syndecan-1), CD142, CD166 (ALCAM), CD203c (ENPP3), CD205 (LY75), CD221 (IGF-1R), CD262 (TRAIL R2), CD276 (B7-H3), mesothelin, EpCAM, CEACAM5, CEACAM6, DLL3, ROR1, ROR2, GPNMB, GCC, GUCY2c, NaPi2b, Flt-1, Flt-3, folate receptor alpha, Tissue Factor (TF), CA6, MUC1, MUC16 (CA-125), BCMA, SLAMF7 (CS1), TIM1, CanAg, Ckit (CD117), EphA2, Nectin4, SLTRK6, FGFR2, LYPD3 (C4.4a), Cadherin 3, 5T4 (TPBG), STEAP1, PTK7, Ephrin-A4, LIV-1 (SLC39A6 or ZIP6), SLC1A5, TENB2, ETBR, integrin v3, Cripto, AGS-5 (SLC44A4), LY6E, AXL, LAMP1, LRRC15, TNF-alpha, and MN/CA IX antibody, antibody fragment or variant. In some embodiments, the antibody, antibody fragment or variant thereof is TROP2 antibody, antibody fragment or variant. In some embodiments, the antibody, antibody fragment or variant thereof is HER2 antibody, antibody fragment or variant. In some embodiments, the antibody, antibody fragment or variant thereof is CD3 antibody, antibody fragment or variant. In some embodiments, the antibody, antibody fragment or variant thereof is PSMA antibody, antibody fragment or variant. In some embodiments, the antibody, antibody fragment or variant thereof is CD70 antibody, antibody fragment or variant. In other embodiments the invention provides anti-TROP2 ADCs comprising antibodies, antibody fragments or variants thereof engineered to have one, or more non-natural amino acids incorporated at any desired position in the heavy and/or light chain amino acid sequence. In some embodiments, the present invention provides anti-TROP2 ADCs comprising one or more antibodies, antibody fragments or variants thereof engineered to have one, or more non-natural amino acids site specifically incorporated in the heavy and/or light chain amino acid sequence conjugated to drug via a phosphate-based linker. In other embodiments the invention provides anti-HER2 ADCs comprising antibodies, antibody fragments or variants thereof engineered to have one, or more non-natural amino acids incorporated at any desired position in the heavy and/or light chain amino acid sequence. In some embodiments, the present invention provides anti-HER2 ADCs comprising one or more antibodies, antibody fragments or variants thereof engineered to have one or more non-natural amino acids site specifically incorporated in the heavy and/or light chain amino acid sequence conjugated to drug via a phosphate-based linker. In other embodiments the invention provides anti-CD3 ADCs comprising antibodies, antibody fragments or variants thereof engineered to have one or more non-natural amino acids incorporated at any desired position in the heavy and/or light chain amino acid sequence. In some embodiments, the present invention provides anti-CD3 ADCs comprising one or more antibodies, antibody fragments or variants thereof engineered to have one or more non-natural amino acids site specifically incorporated in the heavy and/or light chain amino acid sequence conjugated to drug via a phosphate-based linker. In other embodiments the invention provides anti-PSMA ADCs comprising antibodies, antibody fragments or variants thereof engineered to have one or more non-natural amino acids incorporated at any desired position in the heavy and/or light chain amino acid sequence. In some embodiments, the present invention provides anti-PSMA ADCs comprising one or more antibodies, antibody fragments or variants thereof engineered to have one or more non-natural amino acids site specifically incorporated in the heavy and/or light chain amino acid sequence conjugated to drug via a phosphate-based linker. In other embodiments the invention provides anti-CD70 ADCs comprising antibodies, antibody fragments or variants thereof engineered to have one or more non-natural amino acids incorporated at any desired position in the heavy and/or light chain amino acid sequence. In some embodiments, the present invention provides anti-CD70 ADCs comprising one or more antibodies, antibody fragments or variants thereof engineered to have one, or more non-natural amino acids site specifically incorporated in the heavy and/or light chain amino acid sequence conjugated to drug via a phosphate-based linker. In other embodiments the invention provides anti-HER3 ADCs comprising antibodies, antibody fragments or variants thereof engineered to have one or more non-natural amino acids incorporated at any desired position in the heavy and/or light chain amino acid sequence. In some embodiments, the present invention provides anti-HER3 ADCs comprising one or more antibodies, antibody fragments or variants thereof engineered to have one or more non-natural amino acids site specifically incorporated in the heavy and/or light chain amino acid sequence conjugated to drug via a phosphate-based linker. In other embodiments the invention provides anti-GPC3 ADCs comprising antibodies, antibody fragments or variants thereof engineered to have one or more non-natural amino acids incorporated at any desired position in the heavy and/or light chain amino acid sequence. In some embodiments, the present invention provides anti-GPC3 ADCs comprising one or more antibodies, antibody fragments or variants thereof engineered to have one or more non-natural amino acids site specifically incorporated in the heavy and/or light chain amino acid sequence conjugated to drug via a phosphate-based linker.

Antibody or antibody fragments or variants of the disclosure may be human, humanized, engineered, non-human, and/or chimeric antibody or antibody fragments. An antibody or antibody fragment or variant provided herein may comprise two or more amino acid sequences. A first amino acid sequence may comprise a first antibody chain and a second amino acid sequence may comprise a second antibody chain. A first antibody chain may comprise a first amino acid sequence, and a second antibody chain may comprise a second amino acid sequence. A chain of an antibody may refer to an antibody heavy chain, an antibody light chain, or a combination of a region or all of an antibody heavy chain and a region or all of an antibody light chain. As a non-limiting example, an antibody provided herein comprises a heavy chain or fragment or variant thereof, and a light chain or fragment or variant thereof. Two amino acid sequences of an antibody, including two antibody chains, may be connected, attached, or linked by one or more disulfide bonds, a chemical linker, a peptide linker, or a combination thereof. A chemical linker includes a linker via a non-natural amino acid. A chemical linker includes a linker via one or more non-natural amino acids. A chemical linker can include a chemical conjugate. A peptide linker includes any amino acid sequence joining the two amino acid sequences. The peptide linker may comprise 1 or more, 5 or more, 10 or more, 15 or more, 20 or more, 25 or more, 30 or more, 35 or more, 40 or more, 45 or more, 50 or more, 55 or more, 60 or more, 65 or more, 70 or more, 75 or more, 80 or more, 85 or more, 90 or more, 95 or more, 100 or more amino acids. The peptide linker may be a portion of any antibody, including a domain of an antibody, such as a variable domain, CDR1, CDR2, CDR3, and/or a combination of CDRs (complementarity determining regions). In some embodiments a heavy and a light chain are connected, attached, or linked, for example, via a peptide linker. In some cases, a heavy chain and a light chain are connected, for example, by one or more disulfide bonds.

Antibodies, antibody fragments and antibody variants of the disclosure may interact or engage with an antigen on an effector cell. The effector cell can include, but is not limited to, an immune cell, a genetically modified cell having increase or decrease cytotoxic activity, a cell involved in the host defense mechanism, an anti-inflammatory cell, a leukocyte, a lymphocyte, a macrophage, an erythrocyte, a thrombocyte, a neutrophil, a monocyte, an eosinophil, a basophil, a mast cell, a NK cell, a B-cell, or a T-cell. In some embodiments the immune cell may be a T cell such as a cytotoxic T cell or natural killer T cell. The antibody or antibody fragment may interact with a receptor on a T-cell such as, but not limited to a T-cell receptor (TCR). The TCR may comprise TCR alpha, TCR beta, TCR gamma, and/or TCR delta or TCR zeta. Antibody or antibody fragments of the disclosure may bind to a receptor on a lymphocyte, dendritic cell, B-cell, macrophage, monocytes, neutrophils and/or NK cells. Antibody or antibody fragments of the disclosure may bind to a cell surface receptor. Antibody or antibody fragments of the disclosure may bind to an antigen receptor, such as for example, a TROP2 antigen receptor, or a HER2 antigen receptor, or a CD70 antigen receptor. Antibody or antibody fragments of the disclosure can be conjugated to a T-cell surface antigen.

Some cell surface antigens have a high overexpression pattern in a large number of tumors, making them excellent targets in the development of ADCs. Thus, the present disclosure provides novel anti-TROP2 antibodies, anti-HER2 antibodies, anti-CD3 antibodies, anti-PSMA antibodies, anti-CD70 antibodies, anti-HER3 antibodies, anti-GPC3 antibodies, or the corresponding antibody fragments, and antibody-drug conjugates thereof for use as therapeutic agents. Disclosed herein are novel anti-TROP2 antibodies, antibody fragments or variants thereof; anti-HER2 antibodies, antibody fragments or variants thereof; anti-CD3 antibodies, antibody fragments or variants thereof; anti-PSMA antibodies, antibody fragments or variants thereof; anti-CD70 antibodies, antibody fragments or variants thereof; anti-HER3 antibodies, antibody fragments or variants thereof; each with at least one non-natural amino acid or unnaturally encoded amino acid. The present invention provides anti-TROP2 antibodies, antibody fragments or variants thereof; anti-HER2 antibodies, antibody fragments or variants thereof; anti-CD3 antibodies, antibody fragments or variants thereof; anti-PSMA antibodies, antibody fragments or variants thereof; anti-CD70 antibodies, antibody fragments or variants thereof; anti-HER3 antibodies, antibody fragments or variants thereof; and anti-GPC3 antibodies, antibody fragments or variants thereof; each having a non-natural amino acid that facilitate antibody conjugation to a drug or drug-linker.

Antibodies, antibody fragments or variants provided in the present disclosure may be human, humanized, engineered, non-human, and/or chimeric antibody or antibody fragments that bind to the extracellular domain of the target antigen, which can be overexpressed in a number of cancers. Thus, novel antibodies, compositions and antibody drug conjugates for the treatment and/or diagnosis of antigen-expressing cancers are beneficial, including but not limited to TROP2-expressing cancers, HER2-expressing cancers, CD3-expressing cancers, PSMA-expressing cancers, CD70-expressing cancers, anti-HER3 expressing cancers and GPC3-expressing cancers.

Antibodies or antibody fragments or variants disclosed herein include, but are not limited to, analogs, isoforms, mimetics, fragments, or hybrids of anti-TROP2, anti-HER2, anti-CD3, anti-PSMA, anti-CD70, anti-HER3 and anti-GPC3. Antibodies or antibody fragments or variants of anti-TROP2, anti-HER2, anti-CD3, anti-PSMA, anti-CD70, anti-HER3 and anti-GPC3 of the present disclosure include but are not limited to Fv, Fc, Fab, and (Fab′)₂, single chain Fv (scFv), diabodies, triabodies, tetrabodies, bifunctional hybrid antibodies, CDR1, CDR2, CDR3, combinations of CDRs, variable regions, framework regions, constant regions, heavy chains, light chains, alternative scaffold non-antibody molecules, bispecific antibodies and the like.

Antibodies comprising non-natural amino acids are also disclosed herein. In certain embodiments, the antibody or antibody fragments or variants include but are not limited to Fv, Fc, Fab, and (Fab′)₂, single chain Fv (scFv), diabodies, triabodies, tetrabodies, bifunctional hybrid antibodies, CDR1, CDR2, CDR3, combinations of CDRs, variable regions, framework regions, constant regions, heavy chains, light chains, alternative scaffold non-antibody molecules, bispecific antibodies and the like. In some embodiments, the anti-TROP2, anti-HER2, anti-CD3, anti-PSMA, anti-CD70 or anti-GPC3 antibody or antibody fragments or variants comprises one or more non-natural amino acids.

Non-limiting examples of antibodies or antibody fragments or variants of the present disclosure comprise the sequences listed in Tables 1 to 5.

In certain embodiments antibody or antibody fragments disclosed herein are anti-TROP2 antibodies or antibody fragments or variants thereof. In certain embodiments, the anti-TROP2 antibodies or antibody fragments or variants disclosed herein can be humanized. Anti-TROP2 antibodies or antibody fragments or variants disclosed herein include, but are not limited to, anti-TROP2 analogs, isoforms, mimetics, fragments, or hybrids. Anti-TROP2 antibodies or antibody fragments or variants of the present disclosure include but are not limited to Fv, Fc, Fab, and (Fab′)₂, single chain Fv (scFv), diabodies, triabodies, tetrabodies, bifunctional hybrid antibodies, CDR1, CDR2, CDR3, combinations of CDRs, variable regions, framework regions, constant regions, heavy chains, light chains, alternative scaffold non-antibody molecules, bispecific antibodies and the like. The anti-TROP2 antibodies or antibody fragments or variants of the present disclosure comprise a sequence of SEQ ID NOs: 1 to 17 (Table 1). The antibodies, fragments or variants of the present disclosure can be an anti-TROP2 antibody, fragment or variant. In certain embodiments, the anti-TROP2 antibody comprises a heavy chain and light chain amino acid sequence selected from a sequence of SEQ ID NOs: 1 to 17. In certain embodiments, the anti-TROP2 antibody consists of a heavy chain and light chain amino acid sequence selected from a sequence of SEQ ID NOs: 1 to 17.

The anti-TROP2 antibody may comprise a heavy chain and/or light chain amino acid sequence selected from a sequence of SEQ ID NOs: 1 to 17. In some embodiments, the anti-TROP2 antibody consists of a heavy chain and/or light chain amino acid sequence selected from a sequence of SEQ ID NOs: 1 to 17. In certain embodiments, the anti-TROP2 antibody comprises a heavy chain amino acid sequence of any one of SEQ ID NOs: 1, 2, 5, and 6; and a light chain amino acid sequence of any one of SEQ ID NOs: 3, 4, 7, 8, 9, 10, 11, 12, 13, 14, 15, 16, and 17.

In some embodiments, the anti-TROP2 antibody comprises a heavy chain amino acid sequence of SEQ ID NO: 1 and a light chain amino acid sequence of SEQ ID NO: 3. In some embodiments, the anti-TROP2 antibody comprises a heavy chain amino acid sequence of SEQ ID NO: 1 and a light chain amino acid sequence of SEQ ID NO: 4. In some embodiments, the anti-TROP2 antibody comprises a heavy chain amino acid sequence of SEQ ID NO: 1 and a light chain amino acid sequence of SEQ ID NO: 7. In some embodiments, the anti-TROP2 antibody comprises a heavy chain amino acid sequence of SEQ ID NO: 1 and a light chain amino acid sequence of SEQ ID NO: 8. In some embodiments, the anti-TROP2 antibody comprises a heavy chain amino acid sequence of SEQ ID NO: 1 and a light chain amino acid sequence of SEQ ID NO: 9. In some embodiments, the anti-TROP2 antibody comprises a heavy chain amino acid sequence of SEQ ID NO: 1 and a light chain amino acid sequence of SEQ ID NO: 10. In some embodiments, the anti-TROP2 antibody comprises a heavy chain amino acid sequence of SEQ ID NO: 1 and a light chain amino acid sequence of SEQ ID NO: 11. In some embodiments, the anti-TROP2 antibody comprises a heavy chain amino acid sequence of SEQ ID NO: 1 and a light chain amino acid sequence of SEQ ID NO: 12. In some embodiments, the anti-TROP2 antibody comprises a heavy chain amino acid sequence of SEQ ID NO: 1 and a light chain amino acid sequence of SEQ ID NO: 13. In some embodiments, the anti-TROP2 antibody comprises a heavy chain amino acid sequence of SEQ ID NO: 1 and a light chain amino acid sequence of SEQ ID NO: 14. In some embodiments, the anti-TROP2 antibody comprises a heavy chain amino acid sequence of SEQ ID NO: 1 and a light chain amino acid sequence of SEQ ID NO: 15. In some embodiments, the anti-TROP2 antibody comprises a heavy chain amino acid sequence of SEQ ID NO: 1 and a light chain amino acid sequence of SEQ ID NO: 16. In some embodiments, the anti-TROP2 antibody comprises a heavy chain amino acid sequence of SEQ ID NO: 1 and a light chain amino acid sequence of SEQ ID NO: 17.

In some embodiments, the anti-TROP2 antibody comprises two heavy chain amino acid sequences of SEQ ID NO: 1 and two light chain amino acid sequences of SEQ ID NO: 3. In some embodiments, the anti-TROP2 antibody comprises two heavy chain amino acid sequences of SEQ ID NO: 1 and two light chain amino acid sequences of SEQ ID NO: 4. In some embodiments, the anti-TROP2 antibody comprises two heavy chain amino acid sequences of SEQ ID NO: 1 and two light chain amino acid sequences of SEQ ID NO: 7. In some embodiments, the anti-TROP2 antibody comprises two heavy chain amino acid sequences of SEQ ID NO: 1 and two light chain amino acid sequences of SEQ ID NO: 8. In some embodiments, the anti-TROP2 antibody comprises two heavy chain amino acid sequences of SEQ ID NO: 1 and two light chain amino acid sequences of SEQ ID NO: 9. In some embodiments, the anti-TROP2 antibody comprises two heavy chain amino acid sequences of SEQ ID NO: 1 and two light chain amino acid sequences of SEQ ID NO: 10. In some embodiments, the anti-TROP2 antibody comprises two heavy chain amino acid sequences of SEQ ID NO: 1 and two light chain amino acid sequences of SEQ ID NO: 11. In some embodiments, the anti-TROP2 antibody comprises two heavy chain amino acid sequences of SEQ ID NO: 1 and two light chain amino acid sequences of SEQ ID NO: 12. In some embodiments, the anti-TROP2 antibody comprises two heavy chain amino acid sequences of SEQ ID NO: 1 and two light chain amino acid sequences of SEQ ID NO: 13. In some embodiments, the anti-TROP2 antibody comprises two heavy chain amino acid sequences of SEQ ID NO: 1 and two light chain amino acid sequences of SEQ ID NO: 14. In some embodiments, the anti-TROP2 antibody comprises two heavy chain amino acid sequences of SEQ ID NO: 1 and two light chain amino acid sequences of SEQ ID NO: 15. In some embodiments, the anti-TROP2 antibody comprises two heavy chain amino acid sequences of SEQ ID NO: 1 and two light chain amino acid sequences of SEQ ID NO: 16. In some embodiments, the anti-TROP2 antibody comprises two heavy chain amino acid sequences of SEQ ID NO: 1 and two light chain amino acid sequences of SEQ ID NO: 17.

In some embodiments, the anti-TROP2 antibody comprises a heavy chain amino acid sequence of SEQ ID NO: 2 and a light chain amino acid sequence of SEQ ID NO: 3. In some embodiments, the anti-TROP2 antibody comprises a heavy chain amino acid sequence of SEQ ID NO: 2 and a light chain amino acid sequence of SEQ ID NO: 4. In some embodiments, the anti-TROP2 antibody comprises a heavy chain amino acid sequence of SEQ ID NO: 2 and a light chain amino acid sequence of SEQ ID NO: 7. In some embodiments, the anti-TROP2 antibody comprises a heavy chain amino acid sequence of SEQ ID NO: 2 and a light chain amino acid sequence of SEQ ID NO: 8. In some embodiments, the anti-TROP2 antibody comprises a heavy chain amino acid sequence of SEQ ID NO: 2 and a light chain amino acid sequence of SEQ ID NO: 9. In some embodiments, the anti-TROP2 antibody comprises a heavy chain amino acid sequence of SEQ ID NO: 2 and a light chain amino acid sequence of SEQ ID NO: 10. In some embodiments, the anti-TROP2 antibody comprises a heavy chain amino acid sequence of SEQ ID NO: 2 and a light chain amino acid sequence of SEQ ID NO: 11. In some embodiments, the anti-TROP2 antibody comprises a heavy chain amino acid sequence of SEQ ID NO: 2 and a light chain amino acid sequence of SEQ ID NO: 12. In some embodiments, the anti-TROP2 antibody comprises a heavy chain amino acid sequence of SEQ ID NO: 2 and a light chain amino acid sequence of SEQ ID NO: 13. In some embodiments, the anti-TROP2 antibody comprises a heavy chain amino acid sequence of SEQ ID NO: 2 and a light chain amino acid sequence of SEQ ID NO: 14. In some embodiments, the anti-TROP2 antibody comprises a heavy chain amino acid sequence of SEQ ID NO: 2 and a light chain amino acid sequence of SEQ ID NO: 15. In some embodiments, the anti-TROP2 antibody comprises a heavy chain amino acid sequence of SEQ ID NO: 2 and a light chain amino acid sequence of SEQ ID NO: 16. In some embodiments, the anti-TROP2 antibody comprises a heavy chain amino acid sequence of SEQ ID NO: 2 and a light chain amino acid sequence of SEQ ID NO: 17.

In some embodiments, the anti-TROP2 antibody comprises two heavy chain amino acid sequences of SEQ ID NO: 2 and two light chain amino acid sequences of SEQ ID NO: 3. In some embodiments, the anti-TROP2 antibody comprises two heavy chain amino acid sequences of SEQ ID NO: 2 and two light chain amino acid sequences of SEQ ID NO: 4. In some embodiments, the anti-TROP2 antibody comprises two heavy chain amino acid sequences of SEQ ID NO: 2 and two light chain amino acid sequences of SEQ ID NO: 7. In some embodiments, the anti-TROP2 antibody comprises two heavy chain amino acid sequences of SEQ ID NO: 2 and two light chain amino acid sequences of SEQ ID NO: 8. In some embodiments, the anti-TROP2 antibody comprises two heavy chain amino acid sequences of SEQ ID NO: 2 and two light chain amino acid sequences of SEQ ID NO: 9. In some embodiments, the anti-TROP2 antibody comprises two heavy chain amino acid sequences of SEQ ID NO: 2 and two light chain amino acid sequences of SEQ ID NO: 10. In some embodiments, the anti-TROP2 antibody comprises two heavy chain amino acid sequences of SEQ ID NO: 2 and two light chain amino acid sequences of SEQ ID NO: 11. In some embodiments, the anti-TROP2 antibody comprises two heavy chain amino acid sequences of SEQ ID NO: 2 and two light chain amino acid sequences of SEQ ID NO: 12. In some embodiments, the anti-TROP2 antibody comprises two heavy chain amino acid sequences of SEQ ID NO: 2 and two light chain amino acid sequences of SEQ ID NO: 13. In some embodiments, the anti-TROP2 antibody comprises two heavy chain amino acid sequences of SEQ ID NO: 2 and two light chain amino acid sequences of SEQ ID NO: 14. In some embodiments, the anti-TROP2 antibody comprises two heavy chain amino acid sequences of SEQ ID NO: 2 and two light chain amino acid sequences of SEQ ID NO: 15. In some embodiments, the anti-TROP2 antibody comprises two heavy chain amino acid sequences of SEQ ID NO: 2 and two light chain amino acid sequences of SEQ ID NO: 16. In some embodiments, the anti-TROP2 antibody comprises two heavy chain amino acid sequences of SEQ ID NO: 2 and two light chain amino acid sequences of SEQ ID NO: 17.

In some embodiments, the anti-TROP2 antibody comprises a heavy chain amino acid sequence of SEQ ID NO: 5 and a light chain amino acid sequence of SEQ ID NO: 3. In some embodiments, the anti-TROP2 antibody comprises a heavy chain amino acid sequence of SEQ ID NO: 5 and a light chain amino acid sequence of SEQ ID NO: 4. In some embodiments, the anti-TROP2 antibody comprises a heavy chain amino acid sequence of SEQ ID NO: 5 and a light chain amino acid sequence of SEQ ID NO: 7. In some embodiments, the anti-TROP2 antibody comprises a heavy chain amino acid sequence of SEQ ID NO: 5 and a light chain amino acid sequence of SEQ ID NO: 8. In some embodiments, the anti-TROP2 antibody comprises a heavy chain amino acid sequence of SEQ ID NO: 5 and a light chain amino acid sequence of SEQ ID NO: 9. In some embodiments, the anti-TROP2 antibody comprises a heavy chain amino acid sequence of SEQ ID NO: 5 and a light chain amino acid sequence of SEQ ID NO: 10. In some embodiments, the anti-TROP2 antibody comprises a heavy chain amino acid sequence of SEQ ID NO: 5 and a light chain amino acid sequence of SEQ ID NO: 11. In some embodiments, the anti-TROP2 antibody comprises a heavy chain amino acid sequence of SEQ ID NO: 5 and a light chain amino acid sequence of SEQ ID NO: 12. In some embodiments, the anti-TROP2 antibody comprises a heavy chain amino acid sequence of SEQ ID NO: 5 and a light chain amino acid sequence of SEQ ID NO: 13. In some embodiments, the anti-TROP2 antibody comprises a heavy chain amino acid sequence of SEQ ID NO: 5 and a light chain amino acid sequence of SEQ ID NO: 14. In some embodiments, the anti-TROP2 antibody comprises a heavy chain amino acid sequence of SEQ ID NO: 5 and a light chain amino acid sequence of SEQ ID NO: 15. In some embodiments, the anti-TROP2 antibody comprises a heavy chain amino acid sequence of SEQ ID NO: 5 and a light chain amino acid sequence of SEQ ID NO: 16. In some embodiments, the anti-TROP2 antibody comprises a heavy chain amino acid sequence of SEQ ID NO: 5 and a light chain amino acid sequence of SEQ ID NO: 17.

In some embodiments, the anti-TROP2 antibody comprises two heavy chain amino acid sequences of SEQ ID NO: 5 and two light chain amino acid sequences of SEQ ID NO: 3. In some embodiments, the anti-TROP2 antibody comprises two heavy chain amino acid sequences of SEQ ID NO: 5 and two light chain amino acid sequences of SEQ ID NO: 4. In some embodiments, the anti-TROP2 antibody comprises two heavy chain amino acid sequences of SEQ ID NO: 5 and two light chain amino acid sequences of SEQ ID NO: 7. In some embodiments, the anti-TROP2 antibody comprises two heavy chain amino acid sequences of SEQ ID NO: 5 and two light chain amino acid sequences of SEQ ID NO: 8. In some embodiments, the anti-TROP2 antibody comprises two heavy chain amino acid sequences of SEQ ID NO: 5 and two light chain amino acid sequences of SEQ ID NO: 9. In some embodiments, the anti-TROP2 antibody comprises two heavy chain amino acid sequences of SEQ ID NO: 5 and two light chain amino acid sequences of SEQ ID NO: 10. In some embodiments, the anti-TROP2 antibody comprises two heavy chain amino acid sequences of SEQ ID NO: 5 and two light chain amino acid sequences of SEQ ID NO: 11. In some embodiments, the anti-TROP2 antibody comprises two heavy chain amino acid sequences of SEQ ID NO: 5 and two light chain amino acid sequences of SEQ ID NO: 12. In some embodiments, the anti-TROP2 antibody comprises two heavy chain amino acid sequences of SEQ ID NO: 5 and two light chain amino acid sequences of SEQ ID NO: 13. In some embodiments, the anti-TROP2 antibody comprises two heavy chain amino acid sequences of SEQ ID NO: 5 and two light chain amino acid sequences of SEQ ID NO: 14. In some embodiments, the anti-TROP2 antibody comprises two heavy chain amino acid sequences of SEQ ID NO: 5 and two light chain amino acid sequences of SEQ ID NO: 15. In some embodiments, the anti-TROP2 antibody comprises two heavy chain amino acid sequences of SEQ ID NO: 5 and two light chain amino acid sequences of SEQ ID NO: 16. In some embodiments, the anti-TROP2 antibody comprises two heavy chain amino acid sequences of SEQ ID NO: 5 and two light chain amino acid sequences of SEQ ID NO: 17.

In some embodiments, the anti-TROP2 antibody comprises a heavy chain amino acid sequence of SEQ ID NO: 6 and a light chain amino acid sequence of SEQ ID NO: 3. In some embodiments, the anti-TROP2 antibody comprises a heavy chain amino acid sequence of SEQ ID NO: 6 and a light chain amino acid sequence of SEQ ID NO: 4. In some embodiments, the anti-TROP2 antibody comprises a heavy chain amino acid sequence of SEQ ID NO: 6 and a light chain amino acid sequence of SEQ ID NO: 7. In some embodiments, the anti-TROP2 antibody comprises a heavy chain amino acid sequence of SEQ ID NO: 6 and a light chain amino acid sequence of SEQ ID NO: 8. In some embodiments, the anti-TROP2 antibody comprises a heavy chain amino acid sequence of SEQ ID NO: 6 and a light chain amino acid sequence of SEQ ID NO: 9. In some embodiments, the anti-TROP2 antibody comprises a heavy chain amino acid sequence of SEQ ID NO: 6 and a light chain amino acid sequence of SEQ ID NO: 10. In some embodiments, the anti-TROP2 antibody comprises a heavy chain amino acid sequence of SEQ ID NO: 6 and a light chain amino acid sequence of SEQ ID NO: 11. In some embodiments, the anti-TROP2 antibody comprises a heavy chain amino acid sequence of SEQ ID NO: 6 and a light chain amino acid sequence of SEQ ID NO: 12. In some embodiments, the anti-TROP2 antibody comprises a heavy chain amino acid sequence of SEQ ID NO: 6 and a light chain amino acid sequence of SEQ ID NO: 13. In some embodiments, the anti-TROP2 antibody comprises a heavy chain amino acid sequence of SEQ ID NO: 6 and a light chain amino acid sequence of SEQ ID NO: 14. In some embodiments, the anti-TROP2 antibody comprises a heavy chain amino acid sequence of SEQ ID NO: 6 and a light chain amino acid sequence of SEQ ID NO: 15. In some embodiments, the anti-TROP2 antibody comprises a heavy chain amino acid sequence of SEQ ID NO: 6 and a light chain amino acid sequence of SEQ ID NO: 16. In some embodiments, the anti-TROP2 antibody comprises a heavy chain amino acid sequence of SEQ ID NO: 6 and a light chain amino acid sequence of SEQ ID NO: 17.

In some embodiments, the anti-TROP2 antibody comprises two heavy chain amino acid sequences of SEQ ID NO: 6 and two light chain amino acid sequences of SEQ ID NO: 3. In some embodiments, the anti-TROP2 antibody comprises two heavy chain amino acid sequences of SEQ ID NO: 6 and two light chain amino acid sequences of SEQ ID NO: 4. In some embodiments, the anti-TROP2 antibody comprises two heavy chain amino acid sequences of SEQ ID NO: 6 and two light chain amino acid sequences of SEQ ID NO: 7. In some embodiments, the anti-TROP2 antibody comprises two heavy chain amino acid sequences of SEQ ID NO: 6 and two light chain amino acid sequences of SEQ ID NO: 8. In some embodiments, the anti-TROP2 antibody comprises two heavy chain amino acid sequences of SEQ ID NO: 6 and two light chain amino acid sequences of SEQ ID NO: 9. In some embodiments, the anti-TROP2 antibody comprises two heavy chain amino acid sequences of SEQ ID NO: 6 and two light chain amino acid sequences of SEQ ID NO: 10. In some embodiments, the anti-TROP2 antibody comprises two heavy chain amino acid sequences of SEQ ID NO: 6 and two light chain amino acid sequences of SEQ ID NO: 11. In some embodiments, the anti-TROP2 antibody comprises two heavy chain amino acid sequences of SEQ ID NO: 6 and two light chain amino acid sequences of SEQ ID NO: 12. In some embodiments, the anti-TROP2 antibody comprises two heavy chain amino acid sequences of SEQ ID NO: 6 and two light chain amino acid sequences of SEQ ID NO: 13. In some embodiments, the anti-TROP2 antibody comprises two heavy chain amino acid sequences of SEQ ID NO: 6 and two light chain amino acid sequences of SEQ ID NO: 14. In some embodiments, the anti-TROP2 antibody comprises two heavy chain amino acid sequences of SEQ ID NO: 6 and two light chain amino acid sequences of SEQ ID NO: 15. In some embodiments, the anti-TROP2 antibody comprises two heavy chain amino acid sequences of SEQ ID NO: 6 and two light chain amino acid sequences of SEQ ID NO: 16. In some embodiments, the anti-TROP2 antibody comprises two heavy chain amino acid sequences of SEQ ID NO: 6 and two light chain amino acid sequences of SEQ ID NO: 17.

In certain embodiments antibody or antibody fragments disclosed herein are anti-CD70 antibodies or antibody fragments or variants thereof. In certain embodiments, the anti-CD70 antibodies or antibody fragments or variants disclosed herein can be humanized. Anti-CD70 antibodies or antibody fragments or variants disclosed herein include, but are not limited to, anti-CD70 analogs, isoforms, mimetics, fragments, or hybrids. Anti-CD70 antibodies or antibody fragments or variants of the present disclosure include but are not limited to Fv, Fc, Fab, and (Fab′)₂, single chain Fv (scFv), diabodies, triabodies, tetrabodies, bifunctional hybrid antibodies, CDR1, CDR2, CDR3, combinations of CDRs, variable regions, framework regions, constant regions, heavy chains, light chains, alternative scaffold non-antibody molecules, bispecific antibodies and the like.

The anti-CD70 antibodies or antibody fragments or variants of the present disclosure comprise one or more sequence of SEQ ID NOs: 18 to 27 (Table 2). The antibodies, fragments or variants of the present disclosure can be an anti-CD70 antibody, fragment or variant. In certain embodiments, the anti-CD70 antibody comprises a heavy chain and light chain amino acid sequence selected from a sequence of SEQ ID NOs: 18 to 27. In certain embodiments, the anti-CD70 antibody consists of a heavy chain and light chain amino acid sequence selected from a sequence of SEQ ID NOs: 18 to 27.

In some embodiments, the anti-CD70 antibody comprises a heavy chain, wherein the heavy chain contains a variable region having the amino acid sequence of SEQ ID NO: 26, and a light chain, wherein the light chain contains a variable region having the amino acid sequence of SEQ ID NO: 27. In some embodiments, the anti-CD70 antibody comprises two heavy chains, wherein each heavy chain contains a variable region having the amino acid sequence of SEQ ID NO: 26, and two light chains, wherein each light chain contains a variable region having the amino acid sequence of SEQ ID NO: 27.

In some embodiments, the anti-CD70 antibody comprises a heavy chain and/or light chain amino acid sequence selected from a sequence of SEQ ID NOs: 18 to 27. In some embodiments, the anti-CD70 antibody consists of a heavy chain and/or light chain amino acid sequence selected from a sequence of SEQ ID NOs: 18 to 27. In certain embodiments, the anti-CD70 antibody comprises a heavy chain amino acid sequence of SEQ ID NO: 18 or 20; and a light chain amino acid sequence of any one of SEQ ID NOs: 19, 21, 22, 23 and 24. In certain embodiments, the anti-CD70 antibody comprises two heavy chain amino acid sequences of SEQ ID NO: 18, 20 or 25; and two light chain amino acid sequences of any one of SEQ ID NOs: 19, 21, 22, 23 and 24.

In some embodiments, the anti-CD70 antibody comprises a heavy chain amino acid sequence of SEQ ID NO: 18 and a light chain amino acid sequence of SEQ ID NO: 19. In some embodiments, the anti-CD70 antibody comprises a heavy chain amino acid sequence of SEQ ID NO: 18 and a light chain amino acid sequence of SEQ ID NO: 21. In some embodiments, the anti-CD70 antibody comprises a heavy chain amino acid sequence of SEQ ID NO: 18 and a light chain amino acid sequence of SEQ ID NO: 22. In some embodiments, the anti-CD70 antibody comprises a heavy chain amino acid sequence of SEQ ID NO: 18 and a light chain amino acid sequence of SEQ ID NO: 23. In some embodiments, the anti-CD70 antibody comprises a heavy chain amino acid sequence of SEQ ID NO: 18 and a light chain amino acid sequence of SEQ ID NO: 24.

In some embodiments, the anti-CD70 antibody comprises two heavy chain amino acid sequences of SEQ ID NO: 18 and two light chain amino acid sequences of SEQ ID NO: 19. In some embodiments, the anti-CD70 antibody comprises two heavy chain amino acid sequences of SEQ ID NO: 18 and two light chain amino acid sequences of SEQ ID NO: 21. In some embodiments, the anti-CD70 antibody comprises two heavy chain amino acid sequences of SEQ ID NO: 18 and two light chain amino acid sequences of SEQ ID NO: 22. In some embodiments, the anti-CD70 antibody comprises two heavy chain amino acid sequences of SEQ ID NO: 18 and two light chain amino acid sequences of SEQ ID NO: 23. In some embodiments, the anti-CD70 antibody comprises two heavy chain amino acid sequences of SEQ ID NO: 18 and two light chain amino acid sequences of SEQ ID NO: 24.

In some embodiments, the anti-CD70 antibody comprises a heavy chain amino acid sequence of SEQ ID NO: 20 and a light chain amino acid sequence of SEQ ID NO: 19. In some embodiments, the anti-CD70 antibody comprises a heavy chain amino acid sequence of SEQ ID NO: 20 and a light chain amino acid sequence of SEQ ID NO: 21. In some embodiments, the anti-CD70 antibody comprises a heavy chain amino acid sequence of SEQ ID NO: 20 and a light chain amino acid sequence of SEQ ID NO: 22. In some embodiments, the anti-CD70 antibody comprises a heavy chain amino acid sequence of SEQ ID NO: 20 and a light chain amino acid sequence of SEQ ID NO: 23. In some embodiments, the anti-CD70 antibody comprises a heavy chain amino acid sequence of SEQ ID NO: 20 and a light chain amino acid sequence of SEQ ID NO: 24.

In some embodiments, the anti-CD70 antibody comprises two heavy chain amino acid sequences of SEQ ID NO: 20 and two light chain amino acid sequences of SEQ ID NO: 19. In some embodiments, the anti-CD70 antibody comprises two heavy chain amino acid sequences of SEQ ID NO: 20 and two light chain amino acid sequences of SEQ ID NO: 21. In some embodiments, the anti-CD70 antibody comprises two heavy chain amino acid sequences of SEQ ID NO: 20 and two light chain amino acid sequences of SEQ ID NO: 22. In some embodiments, the anti-CD70 antibody comprises two heavy chain amino acid sequences of SEQ ID NO: 20 and two light chain amino acid sequences of SEQ ID NO: 23. In some embodiments, the anti-CD70 antibody comprises two heavy chain amino acid sequences of SEQ ID NO: 20 and two light chain amino acid sequences of SEQ ID NO: 24.

In some embodiments, the anti-CD70 antibody comprises a heavy chain amino acid sequence of SEQ ID NO: 25 and a light chain amino acid sequence of SEQ ID NO: 19. In some embodiments, the anti-CD70 antibody comprises a heavy chain amino acid sequence of SEQ ID NO: 25 and a light chain amino acid sequence of SEQ ID NO: 21. In some embodiments, the anti-CD70 antibody comprises a heavy chain amino acid sequence of SEQ ID NO: 25 and a light chain amino acid sequence of SEQ ID NO: 22. In some embodiments, the anti-CD70 antibody comprises a heavy chain amino acid sequence of SEQ ID NO: 25 and a light chain amino acid sequence of SEQ ID NO: 23. In some embodiments, the anti-CD70 antibody comprises a heavy chain amino acid sequence of SEQ ID NO: 25 and a light chain amino acid sequence of SEQ ID NO: 24.

In some embodiments, the anti-CD70 antibody comprises two heavy chain amino acid sequences of SEQ ID NO: 25 and two light chain amino acid sequences of SEQ ID NO: 19. In some embodiments, the anti-CD70 antibody comprises two heavy chain amino acid sequences of SEQ ID NO: 25 and two light chain amino acid sequences of SEQ ID NO: 21. In some embodiments, the anti-CD70 antibody comprises two heavy chain amino acid sequences of SEQ ID NO: 25 and two light chain amino acid sequences of SEQ ID NO: 22. In some embodiments, the anti-CD70 antibody comprises two heavy chain amino acid sequences of SEQ ID NO: 25 and two light chain amino acid sequences of SEQ ID NO: 23. In some embodiments, the anti-CD70 antibody comprises two heavy chain amino acid sequences of SEQ ID NO: 25 and two light chain amino acid sequences of SEQ ID NO: 24.

In certain embodiments antibody or antibody fragments disclosed herein are anti-HER2 antibodies or antibody fragments or variants thereof. In certain embodiments, the anti-HER2 antibodies or antibody fragments or variants disclosed herein can be humanized. Anti-HER2 antibodies or antibody fragments or variants disclosed herein include, but are not limited to, anti-HER2 analogs, isoforms, mimetics, fragments, or hybrids. Anti-HER2 antibodies or antibody fragments or variants of the present disclosure include but are not limited to Fv, Fc, Fab, and (Fab′)₂, single chain Fv (scFv), diabodies, triabodies, tetrabodies, bifunctional hybrid antibodies, CDR1, CDR2, CDR3, combinations of CDRs, variable regions, framework regions, constant regions, heavy chains, light chains, alternative scaffold non-antibody molecules, bispecific antibodies and the like.

The anti-HER2 antibodies or antibody fragments or variants of the present disclosure comprise one or more sequence of SEQ ID NOs: 28 to 31 (Table 3). The antibodies, fragments or variants of the present disclosure can be an anti-HER2 antibody, fragment or variant. In certain embodiments, the anti-HER2 antibody comprises a heavy chain and light chain amino acid sequence selected from a sequence of SEQ ID NOs: 28 to 31. In certain embodiments, the anti-HER2 antibody consists of a heavy chain and light chain amino acid sequence selected from a sequence of SEQ ID NOs: 28 to 31.

The anti-HER2 antibody may comprise a heavy chain and/or light chain amino acid sequence selected from a sequence of SEQ ID NOs: 28 to 31. In some embodiments, the anti-HER2 antibody consists of a heavy chain and/or light chain amino acid sequence selected from a sequence of SEQ ID NOs: 28 to 31. In certain embodiments, the anti-HER2 antibody comprises a heavy chain amino acid sequence of SEQ ID NO: 28 or 29; and a light chain amino acid sequence of SEQ ID NO:30 or 31.

In some embodiments, the anti-HER2 antibody comprises a heavy chain amino acid sequence of SEQ ID NO: 28 and a light chain amino acid sequence of SEQ ID NO: 30. In some embodiments, the anti-HER2 antibody comprises a heavy chain amino acid sequence of SEQ ID NO: 28 and a light chain amino acid sequence of SEQ ID NO: 31.

In some embodiments, the anti-HER2 antibody comprises two heavy chain amino acid sequences of SEQ ID NO: 28 and two light chain amino acid sequences of SEQ ID NO: 30. In some embodiments, the anti-HER2 antibody comprises two heavy chain amino acid sequences of SEQ ID NO: 28 and two light chain amino acid sequences of SEQ ID NO: 31.

In some embodiments, the anti-HER2 antibody comprises a heavy chain amino acid sequence of SEQ ID NO: 29 and a light chain amino acid sequence of SEQ ID NO: 30. In some embodiments, the anti-HER2 antibody comprises a heavy chain amino acid sequence of SEQ ID NO: 29 and a light chain amino acid sequence of SEQ ID NO: 31.

In some embodiments, the anti-HER2 antibody comprises two heavy chain amino acid sequences of SEQ ID NO: 29 and two light chain amino acid sequences of SEQ ID NO: 30. In some embodiments, the anti-HER2 antibody comprises two heavy chain amino acid sequence of SEQ ID NO: 29 and two light chain amino acid sequences of SEQ ID NO: 31.

In certain embodiments antibody or antibody fragments disclosed herein are anti-PSMA antibodies or antibody fragments or variants thereof. In certain embodiments, the anti-PSMA antibodies or antibody fragments or variants disclosed herein can be humanized. Anti-PSMA antibodies or antibody fragments or variants disclosed herein include, but are not limited to, anti-PSMA analogs, isoforms, mimetics, fragments, or hybrids. Anti-PSMA antibodies or antibody fragments or variants of the present disclosure include but are not limited to Fv, Fc, Fab, and (Fab′)₂, single chain Fv (scFv), diabodies, triabodies, tetrabodies, bifunctional hybrid antibodies, CDR1, CDR2, CDR3, combinations of CDRs, variable regions, framework regions, constant regions, heavy chains, light chains, alternative scaffold non-antibody molecules, bispecific antibodies and the like.

The anti-PSMA antibodies or antibody fragments or variants of the present disclosure comprise one or more sequence of SEQ ID NOs: 32 to 45 (Table 4). The antibodies, fragments or variants of the present disclosure can be an anti-PSMA antibody, fragment or variant. In certain embodiments, the anti-PSMA antibody comprises a heavy chain and light chain amino acid sequence selected from a sequence of SEQ ID NOs: 39 to 45. In certain embodiments, the anti-PSMA antibody consists of a heavy chain and light chain amino acid sequence selected from a sequence of SEQ ID NOs: 39 to 45.

The anti-PSMA antibody may comprise a heavy chain and/or light chain amino acid sequence selected from a sequence of SEQ ID NOs: 39 to 45. In some embodiments, the anti-PSMA antibody consists of a heavy chain and/or light chain amino acid sequence selected from a sequence of SEQ ID NOs: 39 to 45. In certain embodiments, the anti-PSMA antibody comprises a heavy chain amino acid sequence of SEQ ID NO: 39; and a light chain amino acid sequence of any one of SEQ ID NOs: 40, 41, 42 or 43. In certain embodiments, the anti-PSMA antibody comprises two heavy chain amino acid sequences of SEQ ID NO: 39; and two light chain amino acid sequences of any one of SEQ ID NOs: 40, 41, 42 or 43.

In some embodiments, the anti-PSMA antibody comprises a heavy chain amino acid sequence of SEQ ID NO: 39 and a light chain amino acid sequence of SEQ ID NO: 40. In some embodiments, the anti-PSMA antibody comprises a heavy chain amino acid sequence of SEQ ID NO: 39 and a light chain amino acid sequence of SEQ ID NO: 41. In some embodiments, the anti-PSMA antibody comprises a heavy chain amino acid sequence of SEQ ID NO: 39 and a light chain amino acid sequence of SEQ ID NO: 43. In some embodiments, the anti-PSMA antibody comprises a heavy chain amino acid sequence of SEQ ID NO: 39 and a light chain amino acid sequence of SEQ ID NO: 43.

In some embodiments, the anti-PSMA antibody comprises two heavy chain amino acid sequences of SEQ ID NO: 39 and two light chain amino acid sequences of SEQ ID NO: 40. In some embodiments, the anti-PSMA antibody comprises two heavy chain amino acid sequences of SEQ ID NO: 39 and two light chain amino acid sequences of SEQ ID NO: 41. In some embodiments, the anti-PSMA antibody comprises two heavy chain amino acid sequences of SEQ ID NO: 39 and two light chain amino acid sequences of SEQ ID NO: 42. In some embodiments, the anti-PSMA antibody comprises two heavy chain amino acid sequences of SEQ ID NO: 39 and two light chain amino acid sequences of SEQ ID NO: 43.

In certain embodiments antibody or antibody fragments disclosed herein are anti-HER3 antibodies or antibody fragments or variants thereof. In certain embodiments, the anti-HER3 antibodies or antibody fragments or variants disclosed herein can be humanized. Anti-HER3 antibodies or antibody fragments or variants disclosed herein include, but are not limited to, anti-HER3 analogs, isoforms, mimetics, fragments, or hybrids. Anti-HER3 antibodies or antibody fragments or variants of the present disclosure include but are not limited to Fv, Fc, Fab, and (Fab′)₂, single chain Fv (scFv), diabodies, triabodies, tetrabodies, bifunctional hybrid antibodies, CDR1, CDR2, CDR3, combinations of CDRs, variable regions, framework regions, constant regions, heavy chains, light chains, alternative scaffold non-antibody molecules, bispecific antibodies and the like. The anti-HER3 antibodies or antibody fragments or variants of the present disclosure comprise a sequence of SEQ ID NOs: 46 to 58 (Table 5). The antibodies, fragments or variants of the present disclosure can be an anti-HER3 antibody, fragment or variant. In certain embodiments, the anti-HER3 antibody comprises a heavy chain and light chain amino acid sequence selected from a sequence of SEQ ID NOs: 46 to 58. In certain embodiments, the anti-HER3 antibody consists of a heavy chain and light chain amino acid sequence selected from a sequence of SEQ ID NOs: 46 to 58.

The anti-HER3 antibody may comprise a heavy chain and/or light chain amino acid sequence selected from a sequence of SEQ ID NOs: 46 to 58. In some embodiments, the anti-HER3 antibody consists of a heavy chain and/or light chain amino acid sequence selected from a sequence of SEQ ID NOs: 46 to 58. In certain embodiments, the anti-HER3 antibody comprises a heavy chain amino acid sequence of SEQ ID NO: 46 or 58; and a light chain amino acid sequence of any one of SEQ ID NOs: 47 to 57.

In some embodiments, the anti-HER3 antibody comprises a heavy chain amino acid sequence of SEQ ID NO: 46 and a light chain amino acid sequence of SEQ ID NO: 47. In some embodiments, the anti-HER3 antibody comprises a heavy chain amino acid sequence of SEQ ID NO: 46 and a light chain amino acid sequence of SEQ ID NO: 48. In some embodiments, the anti-HER3 antibody comprises a heavy chain amino acid sequence of SEQ ID NO: 46 and a light chain amino acid sequence of SEQ ID NO: 49. In some embodiments, the anti-HER3 antibody comprises a heavy chain amino acid sequence of SEQ ID NO: 46 and a light chain amino acid sequence of SEQ ID NO: 50. In some embodiments, the anti-HER3 antibody comprises a heavy chain amino acid sequence of SEQ ID NO: 46 and a light chain amino acid sequence of SEQ ID NO: 51. In some embodiments, the anti-HER3 antibody comprises a heavy chain amino acid sequence of SEQ ID NO: 46 and a light chain amino acid sequence of SEQ ID NO: 52. In some embodiments, the anti-HER3 antibody comprises a heavy chain amino acid sequence of SEQ ID NO: 46 and a light chain amino acid sequence of SEQ ID NO: 53. In some embodiments, the anti-HER3 antibody comprises a heavy chain amino acid sequence of SEQ ID NO: 46 and a light chain amino acid sequence of SEQ ID NO: 54. In some embodiments, the anti-HER3 antibody comprises a heavy chain amino acid sequence of SEQ ID NO: 46 and a light chain amino acid sequence of SEQ ID NO: 55. In some embodiments, the anti-HER3 antibody comprises a heavy chain amino acid sequence of SEQ ID NO: 46 and a light chain amino acid sequence of SEQ ID NO: 56. In some embodiments, the anti-HER3 antibody comprises a heavy chain amino acid sequence of SEQ ID NO: 46 and a light chain amino acid sequence of SEQ ID NO: 57.

In some embodiments, the anti-HER3 antibody comprises two heavy chain amino acid sequences of SEQ ID NO: 46 and two light chain amino acid sequences of SEQ ID NO: 47. In some embodiments, the anti-HER3 antibody comprises two heavy chain amino acid sequences of SEQ ID NO: 46 and two light chain amino acid sequences of SEQ ID NO: 48. In some embodiments, the anti-HER3 antibody comprises two heavy chain amino acid sequences of SEQ ID NO: 46 and two light chain amino acid sequences of SEQ ID NO: 49. In some embodiments, the anti-HER3 antibody comprises two heavy chain amino acid sequences of SEQ ID NO: 46 and two light chain amino acid sequences of SEQ ID NO: 50. In some embodiments, the anti-HER3 antibody comprises two heavy chain amino acid sequences of SEQ ID NO: 46 and two light chain amino acid sequences of SEQ ID NO: 51. In some embodiments, the anti-HER3 antibody comprises two heavy chain amino acid sequences of SEQ ID NO: 46 and two light chain amino acid sequences of SEQ ID NO: 52. In some embodiments, the anti-HER3 antibody comprises two heavy chain amino acid sequences of SEQ ID NO: 46 and two light chain amino acid sequences of SEQ ID NO: 53. In some embodiments, the anti-HER3 antibody comprises two heavy chain amino acid sequences of SEQ ID NO: 46 and two light chain amino acid sequences of SEQ ID NO: 54. In some embodiments, the anti-HER3 antibody comprises two heavy chain amino acid sequences of SEQ ID NO: 46 and two light chain amino acid sequences of SEQ ID NO: 55. In some embodiments, the anti-HER3 antibody comprises two heavy chain amino acid sequences of SEQ ID NO: 46 and two light chain amino acid sequences of SEQ ID NO: 56. In some embodiments, the anti-HER3 antibody comprises two heavy chain amino acid sequences of SEQ ID NO: 46 and two light chain amino acid sequences of SEQ ID NO: 57.

In some embodiments, the anti-HER3 antibody comprises a heavy chain amino acid sequence of SEQ ID NO: 58 and a light chain amino acid sequence of SEQ ID NO: 47. In some embodiments, the anti-HER3 antibody comprises a heavy chain amino acid sequence of SEQ ID NO: 58 and a light chain amino acid sequence of SEQ ID NO: 48. In some embodiments, the anti-HER3 antibody comprises a heavy chain amino acid sequence of SEQ ID NO: 58 and a light chain amino acid sequence of SEQ ID NO: 49. In some embodiments, the anti-HER3 antibody comprises a heavy chain amino acid sequence of SEQ ID NO: 58 and a light chain amino acid sequence of SEQ ID NO: 50. In some embodiments, the anti-HER3 antibody comprises a heavy chain amino acid sequence of SEQ ID NO: 58 and a light chain amino acid sequence of SEQ ID NO: 51. In some embodiments, the anti-HER3 antibody comprises a heavy chain amino acid sequence of SEQ ID NO: 58 and a light chain amino acid sequence of SEQ ID NO: 52. In some embodiments, the anti-HER3 antibody comprises a heavy chain amino acid sequence of SEQ ID NO: 58 and a light chain amino acid sequence of SEQ ID NO: 53. In some embodiments, the anti-HER3 antibody comprises a heavy chain amino acid sequence of SEQ ID NO: 58 and a light chain amino acid sequence of SEQ ID NO: 54. In some embodiments, the anti-HER3 antibody comprises a heavy chain amino acid sequence of SEQ ID NO: 58 and a light chain amino acid sequence of SEQ ID NO: 55. In some embodiments, the anti-HER3 antibody comprises a heavy chain amino acid sequence of SEQ ID NO: 58 and a light chain amino acid sequence of SEQ ID NO: 56. In some embodiments, the anti-HER3 antibody comprises a heavy chain amino acid sequence of SEQ ID NO: 58 and a light chain amino acid sequence of SEQ ID NO: 57.

In some embodiments, the anti-HER3 antibody comprises two heavy chain amino acid sequences of SEQ ID NO: 58 and two light chain amino acid sequences of SEQ ID NO: 47. In some embodiments, the anti-HER3 antibody comprises two heavy chain amino acid sequences of SEQ ID NO: 58 and two light chain amino acid sequences of SEQ ID NO: 48. In some embodiments, the anti-HER3 antibody comprises two heavy chain amino acid sequences of SEQ ID NO: 58 and two light chain amino acid sequences of SEQ ID NO: 49. In some embodiments, the anti-HER3 antibody comprises two heavy chain amino acid sequences of SEQ ID NO: 58 and two light chain amino acid sequences of SEQ ID NO: 50. In some embodiments, the anti-HER3 antibody comprises two heavy chain amino acid sequences of SEQ ID NO: 58 and two light chain amino acid sequences of SEQ ID NO: 51. In some embodiments, the anti-HER3 antibody comprises two heavy chain amino acid sequences of SEQ ID NO: 58 and two light chain amino acid sequences of SEQ ID NO: 52. In some embodiments, the anti-HER3 antibody comprises two heavy chain amino acid sequences of SEQ ID NO: 58 and two light chain amino acid sequences of SEQ ID NO: 53. In some embodiments, the anti-HER3 antibody comprises two heavy chain amino acid sequences of SEQ ID NO: 58 and two light chain amino acid sequences of SEQ ID NO: 54. In some embodiments, the anti-HER3 antibody comprises two heavy chain amino acid sequences of SEQ ID NO: 58 and two light chain amino acid sequences of SEQ ID NO: 55. In some embodiments, the anti-HER3 antibody comprises two heavy chain amino acid sequences of SEQ ID NO: 58 and two light chain amino acid sequences of SEQ ID NO: 56. In some embodiments, the anti-HER3 antibody comprises two heavy chain amino acid sequences of SEQ ID NO: 58 and two light chain amino acid sequences of SEQ ID NO: 57.

In certain embodiments antibody or antibody fragments disclosed herein are anti-GPC3 antibodies or antibody fragments or variants thereof. In certain embodiments, the anti-GPC3 antibodies or antibody fragments or variants disclosed herein can be humanized. Anti-GPC3 antibodies or antibody fragments or variants disclosed herein include, but are not limited to, anti-GPC3 analogs, isoforms, mimetics, fragments, or hybrids. Anti-GPC3 antibodies or antibody fragments or variants of the present disclosure include but are not limited to Fv, Fc, Fab, and (Fab′)₂, single chain Fv (scFv), diabodies, triabodies, tetrabodies, bifunctional hybrid antibodies, CDR1, CDR2, CDR3, combinations of CDRs, variable regions, framework regions, constant regions, heavy chains, light chains, alternative scaffold non-antibody molecules, bispecific antibodies and the like.

In certain embodiments antibody or antibody fragments disclosed herein are anti-CD3 antibodies or antibody fragments or variants thereof. In certain embodiments, the anti-CD3 antibodies or antibody fragments or variants disclosed herein can be humanized. Anti-CD3 antibodies or antibody fragments or variants disclosed herein include, but are not limited to, ant-CD3 analogs, isoforms, mimetics, fragments, or hybrids. Anti-CD3 antibodies or antibody fragments or variants of the present disclosure include but are not limited to Fv, Fc, Fab, and (Fab′)₂, single chain Fv (scFv), diabodies, triabodies, tetrabodies, bifunctional hybrid antibodies, CDR1, CDR2, CDR3, combinations of CDRs, variable regions, framework regions, constant regions, heavy chains, light chains, alternative scaffold non-antibody molecules, bispecific antibodies and the like. The anti-CD3 antibodies or antibody fragments or variants of the present disclosure comprise one or more sequence, as disclosed, for example, in WO2020/047176, the contents of which are hereby incorporated by reference in their entirety.

TABLE 1
Anti-TROP2 heavy chain (HC) and light chain (LC) amino acid sequences including
sequences with Amber sites for non-natural amino acid incorporation. Also disclosed
are: all of the sequences in Table 1, wherein X is replaced by any non-natural amino
acid; all of the sequences in Table 1, wherein any amino acid is replaced by any
non-natural amino acid; all of the sequences in Table 1, wherein X is pAF; all of
the heavy chain sequences in Table 1, wherein a non-natural amino acid is site
specifically incorporated at position 114, according to Kabat numbering, as well
known to the skilled artisan); and all of the heavy chain sequences in Table 1,
wherein EEM is replaced with DEL. WT: Wild Type; HC: Heavy Chain; LC: Light Chain;
X denotes non-natural amino acid.

| SEQ ID NO | Description | Amino Acid Sequence |
|---|---|---|
| 1 | Anti- | EVQLVQSGAEVKKPGATVKISCKVSGYTFTNYGMNW |
| | TROP2 | VQQAPGKGLEWMGWINTYTGEPTYTEKFQGRVTITAD |
| | Heavy | TSTTTAYMELSSLRSEDTAVYYCARGGFGSSYWYFDV |
| | Chain 1 | WGQGTMVTVSSASTKGPSVFPLAPSSKSTSGGTAALG |
| | (HC1) | CLVKDYFPEPVTVSWNSGALTSGVHTFPAVLQSSGLYS |
| | (EEM | LSSVVTVPSSSLGTQTYICNVNHKPSNTKVDKKVEPKS |
| | allotype in | CDKTHTCPPCPAPELLGGPSVFLFPPKPKDTLMISRTPE |
| | Fc constant | VTCVVVDVSHEDPEVKFNWYVDGVEVHNAKTKPREE |
| | region) | QYNSTYRVVSVLTVLHQDWLNGKEYKCKVSNKALPA |
| | | PIEKTISKAKGQPREPQVYTLPPSREEMTKNQVSLTCLV |
| | | KGFYPSDIAVEWESNGQPENNYKTTPPVLDSDGSFFLY |
| | | SKLTVDKSRWQQGNVFSCSVMHEALHNHYTQKSLSLS |
| | | PG |
| 2 | Anti- | QVQLVQSGAEVKKPGASVKVSCKASGYTFTNYGMNW |
| | TROP2 | VRQAPGQRLEWMGWINTYTGEPTYSQEFQGRVTITLD |
| | Heavy | TSASTAYMELSSLRSEDMAVYYCARGGFGSSYWYFDV |
| | Chain 3 | WGQGTMVTVSSASTKGPSVFPLAPSSKSTSGGTAALG |
| | (HC3) | CLVKDYFPEPVTVSWNSGALTSGVHTFPAVLQSSGLYS |
| | (EEM | LSSVVTVPSSSLGTQTYICNVNHKPSNTKVDKKVEPKS |
| | allotype in | CDKTHTCPPCPAPELLGGPSVFLFPPKPKDTLMISRTPE |
| | Fc constant | VTCVVVDVSHEDPEVKFNWYVDGVEVHNAKTKPREE |
| | region) | QYNSTYRVVSVLTVLHQDWLNGKEYKCKVSNKALPA |
| | | PIEKTISKAKGQPREPQVYTLPPSREEMTKNQVSLTCLV |
| | | KGFYPSDIAVEWESNGQPENNYKTTPPVLDSDGSFFLY |
| | | SKLTVDKSRWQQGNVFSCSVMHEALHNHYTQKSLSLS |
| | | PG |
| 3 | Anti- | DIQLTQSPSFLSASVGDRVTITCRASQDVSIALAWYQQ |
| | TROP2 | KPGKAPKLLIYSASYLYSGVPSRFSGSGSGTEFTLTISSL |
| | Light Chain | QPEDFATYYCQQHYITPLTFGQGTKLEIKRTVAAPSVFI |
| | 2 (LC2) WT | FPPSDEQLKSGTASVVCLLNNFYPREAKVQWKVDNAL |
| | | QSGNSQESVTEQDSKDSTYSLSSTLTLSKADYEKHKVY |
| | | ACEVTHQGLSSPVTKSFNRGEC |
| 4 | Anti- | DIVMTQSPDSLAVSLGERATINCKSSQDVSIALAWYQQ |
| | TROP2 | KPGQPPKLLIYSASYRYTGVPDRFSGSGSGTDFTLTISSL |
| | Light Chain | QAEDVAVYYCQQHYITPLTFGQGTKLEIKRTVAAPSVF |
| | 3 (LC3) WT | IFPPSDEQLKSGTASVVCLLNNFYPREAKVQWKVDNA |
| | | LQSGNSQESVTEQDSKDSTYSLSSTLTLSKADYEKHKV |
| | | YACEVTHQGLSSPVTKSFNRGEC |
| 5 | Anti- | EVQLVQSGAEVKKPGATVKISCKVSGYTFTNYGMNW |
| | TROP2 | VQQAPGKGLEWMGWINTYTGEPTYTEKFQGRVTITAD |
| | Heavy | TSTTTAYMELSSLRSEDTAVYYCARGGFGSSYWYFDV |
| | Chain 1 | WGQGTMVTVSSXSTKGPSVFPLAPSSKSTSGGTAALG |
| | (HC1) | CLVKDYFPEPVTVSWNSGALTSGVHTFPAVLQSSGLYS |
| | Mutation | LSSVVTVPSSSLGTQTYICNVNHKPSNTKVDKKVEPKS |
| | A114 (EEM | CDKTHTCPPCPAPELLGGPSVFLFPPKPKDTLMISRTPE |
| | allotype in | VTCVVVDVSHEDPEVKFNWYVDGVEVHNAKTKPREE |
| | Fc constant | QYNSTYRVVSVLTVLHQDWLNGKEYKCKVSNKALPA |
| | region) | PIEKTISKAKGQPREPQVYTLPPSREEMTKNQVSLTCLV |
| | | KGFYPSDIAVEWESNGQPENNYKTTPPVLDSDGSFFLY |
| | | SKLTVDKSRWQQGNVFSCSVMHEALHNHYTQKSLSLS |
| | | PG |
| 6 | Anti- | QVQLVQSGAEVKKPGASVKVSCKASGYTFTNYGMNW |
| | TROP2 | VRQAPGQRLEWMGWINTYTGEPTYSQEFQGRVTITLD |
| | Heavy | TSASTAYMELSSLRSEDMAVYYCARGGFGSSYWYFDV |
| | Chain 3 | WGQGTMVTVSSXSTKGPSVFPLAPSSKSTSGGTAALG |
| | (HC3) | CLVKDYFPEPVTVSWNSGALTSGVHTFPAVLQSSGLYS |
| | Mutation | LSSVVTVPSSSLGTQTYICNVNHKPSNTKVDKKVEPKS |
| | A114 (EEM | CDKTHTCPPCPAPELLGGPSVFLFPPKPKDTLMISRTPE |
| | allotype in | VTCVVVDVSHEDPEVKFNWYVDGVEVHNAKTKPREE |
| | Fc constant | QYNSTYRVVSVLTVLHQDWLNGKEYKCKVSNKALPA |
| | region) | PIEKTISKAKGQPREPQVYTLPPSREEMTKNQVSLTCLV |
| | | KGFYPSDIAVEWESNGQPENNYKTTPPVLDSDGSFFLY |
| | | SKLTVDKSRWQQGNVFSCSVMHEALHNHYTQKSLSLS |
| | | PG |
| 7 | Anti- | DIQLTQSPSFLSASVGDRVTITCRASQDVSIALAWYQQ |
| | TROP2 | KPGKAPKLLIYSASYLYSGVPSRFSGSGSGTEFTLTISSL |
| | Light Chain | QPEDFATYYCQQHYITPLTFGQGTKLEIKRTVAAPSVFI |
| | 2 (LC2) | FPPXDEQLKSGTASVVCLLNNFYPREAKVQWKVDNAL |
| | Mutation | QSGNSQESVTEQDSKDSTYSLSSTLTLSKADYEKHKVY |
| | S121 | ACEVTHQGLSSPVTKSFNRGEC |
| 8 | Anti- | DIVMTQSPDSLAVSLGERATINCKSSQDVSIALAWYQQ |
| | TROP2 | KPGQPPKLLIYSASYRYTGVPDRFSGSGSGTDFTLTISSL |
| | Light Chain | QAEDVAVYYCQQHYITPLTFGQGTKLEIKRTXAAPSVF |
| | 3 (LC3) | IFPPSDEQLKSGTASVVCLLNNFYPREAKVQWKVDNA |
| | Mutation | LQSGNSQESVTEQDSKDSTYSLSSTLTLSKADYEKHKV |
| | V110 | YACEVTHQGLSSPVTKSFNRGEC |
| 9 | Anti- | DIVMTQSPDSLAVSLGERATINCKSSQDVSIALAWYQQ |
| | TROP2 | KPGQPPKLLIYSASYRYTGVPDRFSGSGSGTDFTLTISSL |
| | Light Chain | QAEDVAVYYCQQHYITPLTFGQGTKLEIKRTVAXPSVF |
| | 3 (LC3) | IFPPSDEQLKSGTASVVCLLNNFYPREAKVQWKVDNA |
| | Mutation | LQSGNSQESVTEQDSKDSTYSLSSTLTLSKADYEKHKV |
| | A112 | YACEVTHQGLSSPVTKSFNRGEC |
| 10 | Anti- | DIVMTQSPDSLAVSLGERATINCKSSQDVSIALAWYQQ |
| | TROP2 | KPGQPPKLLIYSASYRYTGVPDRFSGSGSGTDFTLTISSL |
| | Light Chain | QAEDVAVYYCQQHYITPLTFGQGTKLEIKRTVAAPXV |
| | 3 (LC3) | FIFPPSDEQLKSGTASVVCLLNNFYPREAKVQWKVDN |
| | Mutation | ALQSGNSQESVTEQDSKDSTYSLSSTLTLSKADYEKHK |
| | S114 | VYACEVTHQGLSSPVTKSFNRGEC |
| 11 | Anti- | DIVMTQSPDSLAVSLGERATINCKSSQDVSIALAWYQQ |
| | TROP2 | KPGQPPKLLIYSASYRYTGVPDRFSGSGSGTDFTLTISSL |
| | Light Chain | QAEDVAVYYCQQHYITPLTFGQGTKLEIKRTVAAPSVF |
| | 3 (LC3) | IFPPXDEQLKSGTASVVCLLNNFYPREAKVQWKVDNA |
| | Mutation | LQSGNSQESVTEQDSKDSTYSLSSTLTLSKADYEKHKV |
| | S121 | YACEVTHQGLSSPVTKSFNRGEC |
| 12 | Anti- | DIVMTQSPDSLAVSLGERATINCKSSQDVSIALAWYQQ |
| | TROP2 | KPGQPPKLLIYSASYRYTGVPDRFSGSGSGTDFTLTISSL |
| | Light Chain | QAEDVAVYYCQQHYITPLTFGQGTKLEIKRTVAAPSVF |
| | 3 (LC3) | IFPPSDEQLKXGTASVVCLLNNFYPREAKVQWKVDNA |
| | Mutation | LQSGNSQESVTEQDSKDSTYSLSSTLTLSKADYEKHKV |
| | S127 | YACEVTHQGLSSPVTKSFNRGEC |
| 13 | Anti- | DIVMTQSPDSLAVSLGERATINCKSSQDVSIALAWYQQ |
| | TROP2 | KPGQPPKLLIYSASYRYTGVPDRFSGSGSGTDFTLTISSL |
| | Light Chain | QAEDVAVYYCQQHYITPLTFGQGTKLEIKRTVAAPSVF |
| | 3 (LC3) | IFPPSDEQLKSGTASVVCLLNNFYPREAKVQWXVDNA |
| | Mutation | LQSGNSQESVTEQDSKDSTYSLSSTLTLSKADYEKHKV |
| | K149 | YACEVTHQGLSSPVTKSFNRGEC |
| 14 | Anti- | DIVMTQSPDSLAVSLGERATINCKSSQDVSIALAWYQQ |
| | TROP2 | KPGQPPKLLIYSASYRYTGVPDRFSGSGSGTDFTLTISSL |
| | Light Chain | QAEDVAVYYCQQHYITPLTFGQGTKLEIKRTVAAPSVF |
| | 3 (LC3) | IFPPSDEQLKSGTASVVCLLNNFYPREAKVQWKVDNA |
| | Mutation | LQXGNSQESVTEQDSKDSTYSLSSTLTLSKADYEKHKV |
| | S156 | YACEVTHQGLSSPVTKSFNRGEC |
| 15 | Anti- | DIVMTQSPDSLAVSLGERATINCKSSQDVSIALAWYQQ |
| | TROP2 | KPGQPPKLLIYSASYRYTGVPDRFSGSGSGTDFTLTISSL |
| | Light Chain | QAEDVAVYYCQQHYITPLTFGQGTKLEIKRTVAAPSVF |
| | 3 (LC3) | IFPPSDEQLKSGTASVVCLLNNFYPREAKVQWKVDNA |
| | Mutation | LQSGNSQESVTEQDXKDSTYSLSSTLTLSKADYEKHKV |
| | S168 | YACEVTHQGLSSPVTKSFNRGEC |
| 16 | Anti- | DIVMTQSPDSLAVSLGERATINCKSSQDVSIALAWYQQ |
| | TROP2 | KPGQPPKLLIYSASYRYTGVPDRFSGSGSGTDFTLTISSL |
| | Light Chain | QAEDVAVYYCQQHYITPLTFGQGTKLEIKRTVAAPSVF |
| | 3 (LC3) | IFPPSDEQLKSGTASVVCLLNNFYPREAKVQWKVDNA |
| | Mutation | LQSGNSQESVTEQDSKDSTYSLSSTLTLSKADYEKHKV |
| | S202 | YACEVTHQGLXSPVTKSFNRGEC |
| 17 | Anti- | DIVMTQSPDSLAVSLGERATINCKSSQDVSIALAWYQQ |
| | TROP2 | KPGQPPKLLIYSASYRYTGVPDRFSGSGSGTDFTLTISSL |
| | Light Chain | QAEDVAVYYCQQHYITPLTFGQGTKLEIKRTVAAPSVF |
| | 3 (LC3) | IFPPSDEQLKSGTASVVCLLNNFYPREAKVQWKVDNA |
| | Mutation | LQSGNSQESVTEQDSKDSTYSLSSTLTLSKADYEKHKV |
| | V205 | YACEVTHQGLSSPXTKSFNRGEC |

TABLE 2
Anti-CD70 heavy chain (HC) and light chain (LC) amino acid sequences, including
sequences with Amber sites for non-natural amino acid incorporation. Also disclosed
are: all of the sequences in Table 2, wherein X is replaced by any non-natural amino
acid; all of the sequences in Table 2, wherein any amino acid is replaced by any
non-natural amino acid; all of the sequences in Table 2, wherein X is pAF; all of
the heavy chain sequences in Table 2, wherein a non-natural amino acid is site
specifically incorporated at position 114, according to Kabat numbering, as well
known to the skilled artisan; and all of the heavy chain sequences in Table 2,
wherein EEM is replaced with DEL. WT: Wild Type; HC: Heavy Chain; LC: Light Chain;
X denotes non-natural amino acid.

| SEQ ID NO | Description | Amino Acid Sequence |
|---|---|---|
| 18 | Anti-CD70 | EQLVQSGAEVKKPGATVKISCKVSGYTFTDYY |
| | Heavy chain | MNWVQQAPGKGLEWMGIINPYNGGTHYNQKFK |
| | (EEM allotype | GRVTITADTSTDTAYMELSSLRSEDTAVYYCATS |
| | in Fc constant | GYDLYFDYWGQGTLVTVSSASTKGPSVFPLAPSS |
| | region) | KSTSGGTAALGCLVKDYFPEPVTVSWNSGALTSG |
| | | VHTFPAVLQSSGLYSLSSVVTVPSSSLGTQTYICN |
| | | VNHKPSNTKVDKKVEPKSCDKTHTCPPCPAPELL |
| | | GGPSVFLFPPKPKDTLMISRTPEVTCVVVDVSHED |
| | | PEVKFNWYVDGVEVHNAKTKPREEQYNSTYRVV |
| | | SVLTVLHQDWLNGKEYKCKVSNKALPAPIEKTIS |
| | | KAKGQPREPQVYTLPPSREEMTKNQVSLTCLVKG |
| | | FYPSDIAVEWESNGQPENNYKTTPPVLDSDGSFFL |
| | | YSKLTVDKSRWQQGNVFSCSVMHEALHNHYTQK |
| | | SLSLSPG |
| 19 | Anti-CD70 | EIVMTQSPATLSVSPGERATLSCKASQNVGTAVA |
| | Light chain | WYQQKPGQAPRLLIYSAFNRYNGIPARFSGSGPG |
| | WT | TDFTLTISSLQSEDFAVYYCQQYSTYPLTFGGGTK |
| | | VEIKRTVAAPSVFIFPPSDEQLKSGTASVVCLLNNF |
| | | YPREAKVQWKVDNALQSGNSQESVTEQDSKDST |
| | | YSLSSTLTLSKADYEKHKVYACEVTHQGLSSPVT |
| | | KSFNRGEC |
| 20 | Anti-CD70 | EVQLVQSGAEVKKPGATVKISCKVSGYTFTDYY |
| | Heavy Chain | MNWVQQAPGKGLEWMGIINPYNGGTHYNQKFK |
| | Mutation A114 | GRVTITADTSTDTAYMELSSLRSEDTAVYYCATS |
| | (EEM allotype | GYDLYFDYWGQGTLVTVSSXSTKGPSVFPLAPSS |
| | in Fc constant | KSTSGGTAALGCLVKDYFPEPVTVSWNSGALTSG |
| | region) | VHTFPAVLQSSGLYSLSSVVTVPSSSLGTQTYICN |
| | | VNHKPSNTKVDKKVEPKSCDKTHTCPPCPAPELL |
| | | GGPSVFLFPPKPKDTLMISRTPEVTCVVVDVSHED |
| | | PEVKFNWYVDGVEVHNAKTKPREEQYNSTYRVV |
| | | SVLTVLHQDWLNGKEYKCKVSNKALPAPIEKTIS |
| | | KAKGQPREPQVYTLPPSREEMTKNQVSLTCLVKG |
| | | FYPSDIAVEWESNGQPENNYKTTPPVLDSDGSFFL |
| | | YSKLTVDKSRWQQGNVFSCSVMHEALHNHYTQK |
| | | SLSLSPG |
| 21 | Anti-CD70 | EIVMTQSPATLSVSPGERATLSCKASQNVGTAVA |
| | Light chain | WYQQKPGQAPRLLIYSAFNRYNGIPARFSGSGPG |
| | Mutation V110 | TDFTLTISSLQSEDFAVYYCQQYSTYPLTFGGGTK |
| | | VEIKRTXAAPSVFIFPPSDEQLKSGTASVVCLLNNF |
| | | YPREAKVQWKVDNALQSGNSQESVTEQDSKDST |
| | | YSLSSTLTLSKADYEKHKVYACEVTHQGLSSPVT |
| | | KSFNRGEC |
| 22 | Anti-CD70 | EIVMTQSPATLSVSPGERATLSCKASQNVGTAVA |
| | Light chain | WYQQKPGQAPRLLIYSAFNRYNGIPARFSGSGPG |
| | Mutation A112 | TDFTLTISSLQSEDFAVYYCQQYSTYPLTFGGGTK |
| | | VEIKRTVAXPSVFIFPPSDEQLKSGTASVVCLLNNF |
| | | YPREAKVQWKVDNALQSGNSQESVTEQDSKDST |
| | | YSLSSTLTLSKADYEKHKVYACEVTHQGLSSPVT |
| | | KSFNRGEC |
| 23 | Anti-CD70 | EIVMTQSPATLSVSPGERATLSCKASQNVGTAVA |
| | Light chain | WYQQKPGQAPRLLIYSAFNRYNGIPARFSGSGPG |
| | Mutation S114 | TDFTLTISSLQSEDFAVYYCQQYSTYPLTFGGGTK |
| | | VEIKRTVAAPXVFIFPPSDEQLKSGTASVVCLLNN |
| | | FYPREAKVQWKVDNALQSGNSQESVTEQDSKDS |
| | | TYSLSSTLTLSKADYEKHKVYACEVTHQGLSSPV |
| | | TKSFNRGEC |
| 24 | Anti-CD70 | EIVMTQSPATLSVSPGERATLSCKASQNVGTAVA |
| | Light chain | WYQQKPGQAPRLLIYSAFNRYNGIPARFSGSGPG |
| | Mutation S121 | TDFTLTISSLQSEDFAVYYCQQYSTYPLTFGGGTK |
| | | VEIKRTVAAPSVFIFPPXDEQLKSGTASVVCLLNN |
| | | FYPREAKVQWKVDNALQSGNSQESVTEQDSKDS |
| | | TYSLSSTLTLSKADYEKHKVYACEVTHQGLSSPV |
| | | TKSFNRGEC |
| 25 | Anti-CD70 | EVQLVQSGAEVKKPGATVKISCKVSGYTFTDYY |
| | Heavy Chain | MNWVQQAPGKGLEWMGIINPYNGGTHYNQKFK |
| | Mutation A114 | GRVTITADTSTDTAYMELSSLRSEDTAVYYCATS |
| | (DEL allotype | GYDLYFDYWGQGTLVTVSSXSTKGPSVFPLAPSS |
| | in Fc constant | KSTSGGTAALGCLVKDYFPEPVTVSWNSGALTSG |
| | region) | VHTFPAVLQSSGLYSLSSVVTVPSSSLGTQTYICN |
| | | VNHKPSNTKVDKKVEPKSCDKTHTCPPCPAPELL |
| | | GGPSVFLFPPKPKDTLMISRTPEVTCVVVDVSHED |
| | | PEVKFNWYVDGVEVHNAKTKPREEQYNSTYRVV |
| | | SVLTVLHQDWLNGKEYKCKVSNKALPAPIEKTIS |
| | | KAKGQPREPQVYTLPPSRDELTKNQVSLTCLVKG |
| | | FYPSDIAVEWESNGQPENNYKTTPPVLDSDGSFFL |
| | | YSKLTVDKSRWQQGNVFSCSVMHEALHNHYTQK |
| | | SLSLSPG |
| 26 | Anti-CD70 | EVQLVQSGAEVKKPGATVKISCKVSGYTFTDYY |
| | Heavy Chain | MNWVQQAPGKGLEWMGIINPYNGGTHYNQKFK |
| | Variable | GRVTITADTSTDTAYMELSSLRSEDTAVYYCATS |
| | Region | GYDLYFDYWGQGTLVTVSS |
| 27 | Anti-CD70 | EIVMTQSPATLSVSPGERATLSCKASQNVGTAVA |
| | Light Chain | WYQQKPGQAPRLLIYSAFNRYNGIPARFSGSGPG |
| | Variable | TDFTLTISSLQSEDFAVYYCQQYSTYPLTFGGGTK |
| | Region | VEIK |

TABLE 3
Anti-HER2 heavy chain (HC) and light chain (LC) amino acid sequences, including
sequences with amber sites for non-natural amino acid incorporation. Also disclosed
are: all of the sequences in Table 3, wherein X is replaced by any non-natural amino
acid; all of the sequences in Table 3, wherein any amino acid is replaced by any
non-natural amino acid; all of the sequences in Table 3, wherein X is pAF; all of
the heavy chain sequences in Table 3, wherein a non-natural amino acid is site
specifically incorporated at position 114, according to Kabat numbering, as well
known to the skilled artisan; and all of the heavy chain sequences in Table 3,
wherein DEL is replaced with EEM. WT: Wild Type; HC: Heavy Chain; LC: Light Chain;
X denotes non-natural amino acid.

| SEQ ID NO | Description | Amino Acid Sequence |
|---|---|---|
| 28 | Anti-HER2 | EVQLVESGGGLVQPGGSLRLSCAASGFNIKDTYIHWVRQAP |
| | Heavy chain | GKGLEWVARIYPTNGYTRYADSVKGRFTISADTSKNTAYLQ |
| | WT | MNSLRAEDTAVYYCSRWGGDGFYAMDYWGQGTLVTVSSA |
| | | STKGPSVFPLAPSSKSTSGGTAALGCLVKDYFPEPVTVSWNS |
| | | GALTSGVHTFPAVLQSSGLYSLSSVVTVPSSSLGTQTYICNVN |
| | | HKPSNTKVDKKVEPKSCDKTHTCPPCPAPELLGGPSVFLFPP |
| | | KPKDTLMISRTPEVTCVVVDVSHEDPEVKFNWYVDGVEVH |
| | | NAKTKPREEQYNSTYRVVSVLTVLHQDWLNGKEYKCKVSN |
| | | KALPAPIEKTISKAKGQPREPQVYTLPPSRDELTKNQVSL |
| | | TCLVKGFYPSDIAVEWESNGQPENNYKTTPPVLDSDGSFFLY |
| | | SKLTVDKSRWQQGNVFSCSVMHEALHNHYTQKSLSLSPG |
| 29 | Anti-HER2 | EVQLVESGGGLVQPGGSLRLSCAASGFNIKDTYIHWVRQAP |
| | Heavy chain | GKGLEWVARIYPTNGYTRYADSVKGRFTISADTSKNTAYLQ |
| | Mutation | MNSLRAEDTAVYYCSRWGGDGFYAMDYWGQGTLVTVSSX |
| | A114 | STKGPSVFPLAPSSKSTSGGTAALGCLVKDYFPEPVTVSWNS |
| | | GALTSGVHTFPAVLQSSGLYSLSSVVTVPSSSLGTQTYICNVN |
| | | HKPSNTKVDKKVEPKSCDKTHTCPPCPAPELLGGPSVFLFPP |
| | | KPKDTLMISRTPEVTCVVVDVSHEDPEVKFNWYVDGVEVH |
| | | NAKTKPREEQYNSTYRVVSVLTVLHQDWLNGKEYKCKVSN |
| | | KALPAPIEKTISKAKGQPREPQVYTLPPSRDELTKNQVSLTCL |
| | | VKGFYPSDIAVEWESNGQPENNYKTTPPVLDSDGSFFLYSKL |
| | | TVDKSRWQQGNVFSCSVMHEALHNHYTQKSLSLSPG |
| 30 | Anti-HER2 | DIQMTQSPSSLSASVGDRVTITCRASQDVNTAVAWYQQKPG |
| | Light chain | KAPKLLIYSASFLYSGVPSRFSGSRSGTDFTLTISSLQPEDFATY |
| | WT | YCQQHYTTPPTFGQGTKVEIKRTVAAPSVFIFPPSDEQLKSGT |
| | | ASVVCLLNNFYPREAKVQWKVDNALQSGNSQESVTEQDSK |
| | | DSTYSLSSTLTLSKADYEKHKVYACEVTHQGLSSPVTKSFNR |
| | | GEC |
| 31 | Anti-HER2 | DIQMTQSPSSLSASVGDRVTITCRASQDVNTAVAWYQQKPG |
| | Light chain | KAPKLLIYSASFLYSGVPSRFSGSRSGTDFTLTISSLQPEDFATY |
| | Mutation | YCQQHYTTPPTFGQGTKVEIKRTVAAPSVFIFPPXDEQLKSGT |
| | S121 | ASVVCLLNNFYPREAKVQWKVDNALQSGNSQESVTEQDSK |
| | | DSTYSLSSTLTLSKADYEKHKVYACEVTHQGLSSPVTKSFNR |
| | | GEC |

TABLE 4
Anti-PSMA heavy chain (HC) and light chain (LC) amino acid sequences, including
sequences with amber sites for non-natural amino acid incorporation. Also disclosed
are: all of the sequences in Table 4, wherein X is replaced by any non-natural amino
acid; all of the sequences in Table 4, wherein any amino acid is replaced by any
non-natural amino acid; all of the sequences in Table 4, wherein X is pAF; all of
the heavy chain sequences in Table 4, wherein a non-natural amino acid is site
specifically incorporated at position 114, according to Kabat numbering, as well
known to the skilled artisan; and all of the heavy chain sequences in Table 4,
wherein DEL is replaced with EEM. WT: Wild Type; HC: Heavy Chain; LC: Light Chain;
X denotes non-natural amino acid.

| SEQ ID NO | Description | Amino Acid Sequence |
|---|---|---|
| 32 | Anti-PSMA | QVQLVQSGAEVKKPGASVKVSCKASGYTFTEYTIHW |
| | Humanized | VRQAPGQRLEWMGNINPNNGGTTYNQKFEDRVTITR |
| | Heavy Chain | DTSASTAYMELSSLRSEDTAVYYCAAGWNFDYWGQ |
| | Variable Region | GTTLTVSS |
| | Sequence | |
| 33 | Anti-PSMA | DIQLTQSPSFLSASVGDRVTITCKASQDVGTAVDWYQ |
| | Humanized | QKPGKAPKLLIYWASTRHTGVPSRFSGSGSGTEFTLTI |
| | Light Chain | SSLQPEDFATYYCQQYNSYPLTFGGGTKVEIKRTV |
| | Variable Region | |
| | Sequence | |
| 34 | Anti-PSMA | DIQLTQSPSFLSASVGDRVTITCKASQDVGTAVDWYQ |
| | Humanized | QKPGKAPKLLIYWASTRHTGVPDRFSGSGSGTEFTLTI |
| | Light Chain | SSLQPEDFATYYCQQYNSYPLTFGGGTKVEIKRTV |
| | Variable Region | |
| | Sequence | |
| 35 | Anti-PSMA | DIQLTQSPSFLSASVGDRVTITCKASQDVGTAVDWYQ |
| | Humanized | QKPGKAPKLLIYWASTRHTGVPSRFSGSGSGTEFTLTI |
| | Light Chain | SSLQSEDFATYYCQQYNSYPLTFGGGTKVEIKRTV |
| | Variable Region | |
| | Sequence | |
| 36 | Anti-PSMA | DIQLTQSPSFLSASVGDRVTITCKASQDVGTAVDWYQ |
| | Humanized | QKPGKAPKLLIYWASTRHTGVPSRFSGSGSGTEFTLTI |
| | Light Chain | SNLQPEDFATYYCQQYNSYPLTFGGGTKVEIKRTV |
| | Variable Region | |
| | Sequence | |
| 37 | Anti-PSMA | EVQLQQSGPELVKPGTSVRISCKTSGYTFTEYTIHWVK |
| | Chimeric Heavy | QSHGKSLEWIGNINPNNGGTTYNQKFEDKATLTVDKS |
| | Chain Variable | SSTAYMELRSLTSEDSAVYYCAAGWNFDYWGQGTTL |
| | Region Sequence | TVSS |
| 38 | Anti-PSMA | DIVMTQSHKFMSTSVGDRVSIICKASQDVGTAVDWY |
| | Chimeric Light | QQKPGQSPKLLIYWASTRHTGVPDRFTGSGSGTDFTL |
| | Chain Variable | TITNVQSEDLADYFCQQYNSYPLTFGAGTKVEIKRTV |
| | Region Sequence | |
| 39 | Anti-PSMA | QVQLVQSGAEVKKPGASVKVSCKASGYTFTEYTIHW |
| | Humanized | VRQAPGQRLEWMGNINPNNGGTTYNQKFEDRVTITR |
| | Heavy Chain | DTSASTAYMELSSLRSEDTAVYYCAAGWNFDYWGQ |
| | Mutation | GTTLTVSSXSTKGPSVFPLAPSSKSTSGGTAALGCLVK |
| | A114 | DYFPEPVTVSWNSGALTSGVHTFPAVLQSSGLYSLSS |
| | | VVTVPSSSLGTQTYICNVNHKPSNTKVDKKVEPKSCD |
| | | KTHTCPPCPAPELLGGPSVFLFPPKPKDTLMISRTPEVT |
| | | CVVVDVSHEDPEVKFNWYVDGVEVHNAKTKPREEQ |
| | | YNSTYRVVSVLTVLHQDWLNGKEYKCKVSNKALPAP |
| | | IEKTISKAKGQPREPQVYTLPPSRDELTKNQVSLTCLV |
| | | KGFYPSDIAVEWESNGQPENNYKTTPPVLDSDGSFFL |
| | | YSKLTVDKSRWQQGNVFSCSVMHEALHNHYTQKSLS |
| | | LSPG |
| 40 | Anti-PSMA | DIQLTQSPSFLSASVGDRVTITCKASQDVGTAVDWYQ |
| | Humanized | QKPGKAPKLLIYWASTRHTGVPSRFSGSGSGTEFTLTI |
| | Variant 1 Light | SSLQPEDFATYYCQQYNSYPLTFGGGTKVEIKRTVAA |
| | Chain | PSVFIFPPSDEQLKSGTASVVCLLNNFYPREAKVQWK |
| | | VDNALQSGNSQESVTEQDSKDSTYSLSSTLTLSKADY |
| | | EKHKVYACEVTHQGLSSPVTKSFNRGEC |
| 41 | Anti-PSMA | DIQLTQSPSFLSASVGDRVTITCKASQDVGTAVDWYQ |
| | Humanized | QKPGKAPKLLIYWASTRHTGVPSRFSGSGSGTEFTLTI |
| | Variant 2 Light | SSLQSEDFATYYCQQYNSYPLTFGGGTKVEIKRTVAA |
| | Chain | PSVFIFPPSDEQLKSGTASVVCLLNNFYPREAKVQWK |
| | | VDNALQSGNSQESVTEQDSKDSTYSLSSTLTLSKADY |
| | | EKHKVYACEVTHQGLSSPVTKSFNRGEC |
| 42 | Anti-PSMA | DIQLTQSPSFLSASVGDRVTITCKASQDVGTAVDWYQ |
| | Humanized | QKPGKAPKLLIYWASTRHTGVPDRFSGSGSGTEFTLTI |
| | Variant 3 Light | SSLQPEDFATYYCQQYNSYPLTFGGGTKVEIKRTVAA |
| | Chain | PSVFIFPPSDEQLKSGTASVVCLLNNFYPREAKVQWK |
| | | VDNALQSGNSQESVTEQDSKDSTYSLSSTLTLSKADY |
| | | EKHKVYACEVTHQGLSSPVTKSFNRGEC |
| 43 | Anti-PSMA | DIQLTQSPSFLSASVGDRVTITCKASQDVGTAVDWYQ |
| | Humanized | QKPGKAPKLLIYWASTRHTGVPSRFSGSGSGTEFTLTI |
| | Variant 4 Light | SNLQPEDFATYYCQQYNSYPLTFGGGTKVEIKRTVAA |
| | Chain | PSVFIFPPSDEQLKSGTASVVCLLNNFYPREAKVQWK |
| | | VDNALQSGNSQESVTEQDSKDSTYSLSSTLTLSKADY |
| | | EKHKVYACEVTHQGLSSPVTKSFNRGEC |
| 44 | Anti-PSMA | EVQLQQSGPELVKPGTSVRISCKTSGYTFTEYTIHWVKQSHGK |
| | Chimeric | SLEWIGNINPNNGGTTYNQKFEDKATLTVDKSSSTAYMELRSLT |
| | Antibody | SEDSAVYYCAAGWNFDYWGQGTTLTVSSXSTKGPSVFPLAPS |
| | Variant Heavy | SKSTSGGTAALGCLVKDYFPEPVTVSWNSGALTSGVHTFPAVL |
| | Chain | QSSGLYSLSSVVTVPSSSLGTQTYICNVNHKPSNTKVDKKVEP |
| | | KSCDKTHTCPPCPAPELLGGPSVFLFPPKPKDTLMISRTPEVTCV |
| | | VVDVSHEDPEVKFNWYVDGVEVHNAKTKPREEQYNSTYRV |
| | | VSVLTVLHQDWLNGKEYKCKVSNKALPAPIEKTISKAKGQPR |
| | | EPQVYTLPPSRDELTKNQVSLTCLVKGFYPSDIAVEWESNGQPE |
| | | NNYKTTPPVLDSDGSFFLYSKLTVDKSRWQQGNVFSCSVMHE |
| | | ALHNHYTQKSLSLSPG |
| 45 | Anti-PSMA | DIVMTQSHKFMSTSVGDRVSIICKASQDVGTAVDWYQQKPGQ |
| | Chimeric | SPKLLIYWASTRHTGVPDRFTGSGSGTDFTLTITNVQSEDLADY |
| | Antibody | FCQQYNSYPLTFGAGTKVEIKRTVAAPSVFIFPPSDEQLKSGTAS |
| | Variant Light | VVCLLNNFYPREAKVQWKVDNALQSGNSQESVTEQDSKDST |
| | Chain | YSLSSTLTLSKADYEKHK |

TABLE 5
Anti-HER3 heavy chain (HC) and light chain (LC) amino acid sequences, including
sequences with amber sites for non-natural amino acid incorporation. Also disclosed
are: all of the sequences in Table 5, wherein X is replaced by any non-natural amino
acid; all of the sequences in Table 5, wherein any amino acid is replaced by any
non-natural amino acid; all of the sequences in Table 5, wherein X is pAF; all of
the heavy chain sequences in Table 5, wherein a non-natural amino acid is site
specifically incorporated at position 114, according to Kabat numbering, as well
known to the skilled artisan; and all of the heavy chain sequences of Table 5,
wherein EEM is replaced with DEL. WT: Wild Type; HC: Heavy Chain; LC: Light Chain;
X denotes non-natural amino acid.

| SEQ ID NO | Description | Amino Acid Sequence |
|---|---|---|
| 46 | Anti-HER3 | QVQLQQWGAGLLKPSETLSLTCAVYGGSFSGYYW |
| | Humanized | SWIRQPPGKGLEWIGEINHSGSTNYNPSLKSRVTIS |
| | Heavy Chain | VETSKNQFSLKLSSVTAADTAVYYCARDKWTWY |
| | WT | FDLWGRGTLVTVSSASTKGPSVFPLAPSSKSTSGG |
| | | TAALGCLVKDYFPEPVTVSWNSGALTSGVHTFPA |
| | | VLQSSGLYSLSSVVTVPSSSLGTQTYICNVNHKPSN |
| | | TKVDKKVEPKSCDKTHTCPPCPAPELLGGPSVFLF |
| | | PPKPKDTLMISRTPEVTCVVVDVSHEDPEVKFNWY |
| | | VDGVEVHNAKTKPREEQYNSTYRVVSVLTVLHQD |
| | | WLNGKEYKCKVSNKALPAPIEKTISKAKGQPREPQ |
| | | VYTLPPSREEMTKNQVSLTCLVKGFYPSDIAVEWE |
| | | SNGQPENNYKTTPPVLDSDGSFFLYSKLTVDKSRW |
| | | QQGNVFSCSVMHEALHNHYTQKSLSLSPG |
| 47 | Anti-HER3 | DIEMTQSPDSLAVSLGERATINCRSSQSVLYSSSNR |
| | Humanized | NYLAWYQQNPGQPPKLLIYWASTRESGVPDRFSG |
| | Light Chain WT | SGSGTDFTLTISSLQAEDVAVYYCQQYYSTPRTFG |
| | | QGTKVEIKRTVAAPSVFIFPPSDEQLKSGTASVVCL |
| | | LNNFYPREAKVQWKVDNALQSGNSQESVTEQDSK |
| | | DSTYSLSSTLTLSKADYEKHKVYACEVTHQGLSSP |
| | | VTKSFNRGEC |
| 48 | Anti-HER3 | DIEMTQSPDSLAVSLGERATINCRSSQSVLYSSSNR |
| | Humanized | NYLAWYQQNPGQPPKLLIYWASTRESGVPDRFSG |
| | Light Chain | SGSGTDFTLTISSLQAEDVAVYYCQQYYSTPRTFG |
| | Mutation V110 | QGTKVEIKRTXAAPSVFIFPPSDEQLKSGTASVVCL |
| | | LNNFYPREAKVQWKVDNALQSGNSQESVTEQDSK |
| | | DSTYSLSSTLTLSKADYEKHKVYACEVTHQGLSSP |
| | | VTKSFNRGEC |
| 49 | Anti-HER3 | DIEMTQSPDSLAVSLGERATINCRSSQSVLYSSSNR |
| | Humanized | NYLAWYQQNPGQPPKLLIYWASTRESGVPDRFSG |
| | Light Chain | SGSGTDFTLTISSLQAEDVAVYYCQQYYSTPRTFG |
| | Mutation A112 | QGTKVEIKRTVAXPSVFIFPPSDEQLKSGTASVVCL |
| | | LNNFYPREAKVQWKVDNALQSGNSQESVTEQDSK |
| | | DSTYSLSSTLTLSKADYEKHKVYACEVTHQGLSSP |
| | | VTKSFNRGEC |
| 50 | Anti-HER3 | DIEMTQSPDSLAVSLGERATINCRSSQSVLYSSSNR |
| | Humanized | NYLAWYQQNPGQPPKLLIYWASTRESGVPDRFSG |
| | Light Chain | SGSGTDFTLTISSLQAEDVAVYYCQQYYSTPRTFG |
| | Mutation S114 | QGTKVEIKRTVAAPXVFIFPPSDEQLKSGTASVVCL |
| | | LNNFYPREAKVQWKVDNALQSGNSQESVTEQDSK |
| | | DSTYSLSSTLTLSKADYEKHKVYACEVTHQGLSSP |
| | | VTKSFNRGEC |
| 51 | Anti-HER3 | DIEMTQSPDSLAVSLGERATINCRSSQSVLYSSSNR |
| | Humanized | NYLAWYQQNPGQPPKLLIYWASTRESGVPDRFSG |
| | Light Chain | SGSGTDFTLTISSLQAEDVAVYYCQQYYSTPRTFG |
| | Mutation S121 | QGTKVEIKRTVAAPSVFIFPPXDEQLKSGTASVVCL |
| | | LNNFYPREAKVQWKVDNALQSGNSQESVTEQDSK |
| | | DSTYSLSSTLTLSKADYEKHKVYACEVTHQGLSSP |
| | | VTKSFNRGEC |
| 52 | Anti-HER3 | DIEMTQSPDSLAVSLGERATINCRSSQSVLYSSSNR |
| | Humanized | NYLAWYQQNPGQPPKLLIYWASTRESGVPDRFSG |
| | Light Chain | SGSGTDFTLTISSLQAEDVAVYYCQQYYSTPRTFG |
| | Mutation S127 | QGTKVEIKRTVAAPSVFIFPPSDEQLKXGTASVVCL |
| | | LNNFYPREAKVQWKVDNALQSGNSQESVTEQDSK |
| | | DSTYSLSSTLTLSKADYEKHKVYACEVTHQGLSSP |
| | | VTKSFNRGEC |
| 53 | Anti-HER3 | DIEMTQSPDSLAVSLGERATINCRSSQSVLYSSSNR |
| | Humanized | NYLAWYQQNPGQPPKLLIYWASTRESGVPDRFSG |
| | Light Chain | SGSGTDFTLTISSLQAEDVAVYYCQQYYSTPRTFG |
| | Mutation K149 | QGTKVEIKRTVAAPSVFIFPPSDEQLKSGTASVVCL |
| | | LNNFYPREAKVQWXVDNALQSGNSQESVTEQDSK |
| | | DSTYSLSSTLTLSKADYEKHKVYACEVTHQGLSSP |
| | | VTKSFNRGEC |
| 54 | Anti-HER3 | DIEMTQSPDSLAVSLGERATINCRSSQSVLYSSSNR |
| | Humanized | NYLAWYQQNPGQPPKLLIYWASTRESGVPDRFSG |
| | Light Chain | SGSGTDFTLTISSLQAEDVAVYYCQQYYSTPRTFG |
| | Mutation S156 | QGTKVEIKRTVAAPSVFIFPPSDEQLKSGTASVVCL |
| | | LNNFYPREAKVQWKVDNALQXGNSQESVTEQDS |
| | | KDSTYSLSSTLTLSKADYEKHKVYACEVTHQGLSS |
| | | PVTKSFNRGEC |
| 55 | Anti-HER3 | DIEMTQSPDSLAVSLGERATINCRSSQSVLYSSSNR |
| | Humanized | NYLAWYQQNPGQPPKLLIYWASTRESGVPDRFSG |
| | Light Chain | SGSGTDFTLTISSLQAEDVAVYYCQQYYSTPRTFG |
| | Mutation S168 | QGTKVEIKRTVAAPSVFIFPPSDEQLKSGTASVVCL |
| | | LNNFYPREAKVQWKVDNALQSGNSQESVTEQDX |
| | | KDSTYSLSSTLTLSKADYEKHKVYACEVTHQGLSS |
| | | PVTKSFNRGEC |
| 56 | Anti-HER3 | DIEMTQSPDSLAVSLGERATINCRSSQSVLYSSSNR |
| | Humanized | NYLAWYQQNPGQPPKLLIYWASTRESGVPDRFSG |
| | Light Chain | SGSGTDFTLTISSLQAEDVAVYYCQQYYSTPRTFG |
| | Mutation S202 | QGTKVEIKRTVAAPSVFIFPPSDEQLKSGTASVVCL |
| | | LNNFYPREAKVQWKVDNALQSGNSQESVTEQDSK |
| | | DSTYSLSSTLTLSKADYEKHKVYACEVTHQGLXSP |
| | | VTKSFNRGEC |
| 57 | Anti-HER3 | DIEMTQSPDSLAVSLGERATINCRSSQSVLYSSSNR |
| | Humanized | NYLAWYQQNPGQPPKLLIYWASTRESGVPDRFSG |
| | Light Chain | SGSGTDFTLTISSLQAEDVAVYYCQQYYSTPRTFG |
| | Mutation V205 | QGTKVEIKRTVAAPSVFIFPPSDEQLKSGTASVVCL |
| | | LNNFYPREAKVQWKVDNALQSGNSQESVTEQDSK |
| | | DSTYSLSSTLTLSKADYEKHKVYACEVTHQGLSSP |
| | | XTKSFNRGEC |
| 58 | Anti-HER3 | QVQLQQWGAGLLKPSETLSLTCAVYGGSFSGYYW |
| | Humanized | SWIRQPPGKGLEWIGEINHSGSTNYNPSLKSRVTIS |
| | Heavy Chain | VETSKNQFSLKLSSVTAADTAVYYCARDKWTWY |
| | Mutation A114 | FDLWGRGTLVTVSSXSTKGPSVFPLAPSSKSTSGG |
| | | TAALGCLVKDYFPEPVTVSWNSGALTSGVHTFPA |
| | | VLQSSGLYSLSSVVTVPSSSLGTQTYICNVNHKPSN |
| | | TKVDKKVEPKSCDKTHTCPPCPAPELLGGPSVFLF |
| | | PPKPKDTLMISRTPEVTCVVVDVSHEDPEVKFNWY |
| | | VDGVEVHNAKTKPREEQYNSTYRVVSVLTVLHQD |
| | | WLNGKEYKCKVSNKALPAPIEKTISKAKGQPREPQ |
| | | VYTLPPSREEMTKNQVSLTCLVKGFYPSDIAVEWE |
| | | SNGQPENNYKTTPPVLDSDGSFFLYSKLTVDKSRW |
| | | QQGNVFSCSVMHEALHNHYTQKSLSLSPG |

### Non-Natural Amino Acids

The present disclosure provides antibodies, antibody fragments or variants comprising at least one non-natural amino acid. Introduction of at least one non-natural amino acid into an antibody can allow for the application of conjugation chemistries that involve specific chemical reactions with one or more non-natural amino acids while not reacting with the commonly occurring 20 amino acids.

Non-natural amino acid site selection was based on surface exposure/site accessibility within the antibody and hydrophobic or neutral amino acid sites were selected to maintain the charge on the antibody. Methods for introducing non-natural amino acids inserted into sites in a protein are described for example in WO2010/011735 and in WO2005/074650. The present disclosure employs such methodologies and techniques. The non-natural amino acids used in the methods and compositions described herein have at least one of the following four properties: (1) at least one functional group on the sidechain of the non-natural amino acid has at least one characteristics and/or activity and/or reactivity orthogonal to the chemical reactivity of the 20 common, genetically-encoded amino acids (i.e., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine), or at least orthogonal to the chemical reactivity of the naturally occurring amino acids present in the polypeptide that includes the non-natural amino acid; (2) the introduced non-natural amino acids are substantially chemically inert toward the 20 common, genetically-encoded amino acids; (3) the non-natural amino acid can be stably incorporated into a polypeptide, preferably with the stability commensurate with the naturally-occurring amino acids or under typical physiological conditions, and further preferably such incorporation can occur via an in vivo system; and (4) the non-natural amino acid includes an oxime functional group or a functional group that can be transformed into an oxime group by reacting with a reagent, preferably under conditions that do not destroy the biological properties of the polypeptide that includes the non-natural amino acid (unless of course such a destruction of biological properties is the purpose of the modification/transformation), or where the transformation can occur under aqueous conditions at a pH between about 4 and about 8, or where the reactive site on the non-natural amino acid is an electrophilic site. Any number of non-natural amino acids can be introduced into the polypeptide. Non-natural amino acids may also include protected or masked oximes or protected or masked groups that can be transformed into an oxime group after deprotection of the protected group or unmasking of the masked group. Non-natural amino acids may also include protected or masked carbonyl or dicarbonyl groups, which can be transformed into a carbonyl or dicarbonyl group after deprotection of the protected group or unmasking of the masked group and thereby are available to react with hydroxylamines or oximes to form oxime groups. Oxime-based non-natural amino acids may be synthesized by methods well known in the art, (see for example WO2013/185117 and WO2005/074650), including: (a) reaction of a hydroxylamine-containing non-natural amino acid with a carbonyl- or dicarbonyl-containing reagent; (b) reaction of a carbonyl- or dicarbonyl-containing non-natural amino acid with a hydroxylamine-containing reagent; or (c) reaction of an oxime-containing non-natural amino acid with certain carbonyl- or dicarbonyl-containing reagents.

In some embodiments, non-naturally encoded amino acid site selection is based on surface exposure. Example, one possible site is an amino acid having a solvent accessible surface area ratio of 30% or more, 40% or more, 50% or more, 60% or more, 70% or more, 80% or more, 90% or more, or 95% or more. In some embodiments, one possible site is an amino acid having a solvent accessible surface area ratio of about 30%, about 40%, about 50%, about 60%, about 70%, about 80%, about 90%, or about 95%, or more. The solvent accessible surface area can be calculated based on the DSSP program [Biopolymers, 22, 2577-2637 (1983)], using a crystalline structure analyzing data file of antibodies or antibody fragments registered in Protein data bank (PDB).

The ratio of the solvent accessible surface area of the amino acid residues of interest can be calculated by dividing the antibody structural solvent accessible surface area calculated in the above by the solvent accessible surface area of alanine-X-alanine (X represents the amino acid residues of interest). In this connection, there is a case in which two or more PDB files are present on one species of protein, and any one of them can be used in the present invention.

Alternatively, the solvent accessibility of an amino acid can be determined by a solvent accessibility test in which a functional group on the amino acid (a thiol, amino, or carbonyl group) is functionalized when treated with an electrophilic reagent or a nucleophilic reagent, or the like. Based on the test results, the functional group (i.e., the thiol, amino, or carbonyl group) can be called, for example, at least 50% solvent accessible when at least 50% of the functional group is functionalized in the test. In some embodiments, the non-natural amino acid site is at least 30%, at least 40%, at least 50%, at least 60%, at least 70%, at least 75%, at least 80%, at least 85%, at least 90%, or at least 95% solvent accessible. Examples of solvent accessibility test include, but are not limited to, propargylation of a surface thiol group, or α-bromopyruvate reacting with a surface thiol group, etc.

Non-natural amino acids that may be used in the methods and compositions described herein include, but are not limited to, amino acids comprising amino acids with novel functional groups, amino acids that covalently or noncovalently interact with other molecules, glycosylated amino acids such as a sugar substituted serine, other carbohydrate modified amino acids, keto-containing amino acids, aldehyde-containing amino acids, amino acids comprising polyethylene glycol or other polyethers, heavy atom substituted amino acids, chemically cleavable and/or photocleavable amino acids, amino acids with an elongated side chains as compared to natural amino acids, including but not limited to, polyethers or long chain hydrocarbons, including but not limited to, greater than about 5 or greater than about 10 carbons, carbon-linked sugar-containing amino acids, redox-active amino acids, amino thioacid containing amino acids, and amino acids comprising one or more toxic moiety.

In some embodiments disclosed herein are antibodies comprising one or more non-natural amino acids. The one or more non-natural amino acids may be encoded by a codon that does not code for one of the twenty natural amino acids. The one or more non-natural amino acids may be encoded by a nonsense codon (stop codon). The stop codon may be an amber codon. The amber codon may comprise a UAG sequence. The stop codon may be an ochre codon. The ochre codon may comprise a UAA sequence. The stop codon may be an opal or umber codon. The opal or umber codon may comprise a UGA sequence. The one or more non-natural amino acids may be encoded by a four-base codon.

Non-natural amino acids of the present disclosure include, but are not limited to, 1) substituted phenylalanine and tyrosine analogues, such as 4-amino-L-phenylalanine, 4-acetyl-L-phenylalanine, 4-azido-L-phenylalanine, 4-nitro-L-phenylalanine, 3-methoxy-L-phenylalanine, 4-isopropyl-L-phenylalanine, 3-nitro-L-tyrosine, O-methyl-L-tyrosine and O-phosphotyrosine; 2) amino acids that can be photo-cross-linked, e.g., amino acids with aryl azide or benzophenone groups, such as 4-azidophenylalanine or 4-benzoylphenylalanine; 3) amino acids that have unique chemical reactivity, such as 4-acetyl-L-phenylalanine, 3-acetyl-L-phenylalanine, O-allyl-L-tyrosine, O-2-propyn-1-yl-L-tyrosine, N-(ethylthio)thiocarbonyl-L-phenylalanine and p-(3-oxobutanoyl)-L-phenylalanine; 4) heavy-atom-containing amino acids, e.g., for phasing in X-ray crystallography, such as 4-iodo-L-phenylalanine or 4-bromo-L-phenylalanine; 5) a redox-active amino acid, such as 3,4-dihydroxy-L-phenylalanine; 6) a fluorinated amino acid, such as a 2-fluorophenylalanine (e.g., 2-fluoro-L-phenylalanine), a 3-fluorophenylalanine (e.g., 3-fluoro-L-phenylalanine) or a 4-fluorophenylalanine (e.g., 4-fluoro-L-phenylalanine; 7) a fluorescent amino acid, such as an amino acid containing a naphthyl, dansyl or 7-aminocoumarin side chain; 8) a photocleavable or photoisomerizable amino acid, such as an amino acid comprising an azobenzyl or nitrobenzyl, e.g., cysteine, serine or tyrosine comprising azobenzyl or nitrobenzyl; 9) a β-amino acid (e.g., a β² or β³ amino acid); 10) a homo-amino acid, such as homoglutamine (e.g., beta-homoglutamine) or homophenylalanine (e.g., beta-homophenylalanine); 11) a proline or pyruvic acid derivative; 12) a 3-substituted alanine derivative; 14) a glycine derivative; 15) a linear core amino acid; 16) a diamino acid; 17) a D-amino acid; 18) an N-methyl amino acid; 19) a phosphotyrosine mimetic, such as a carboxymethylphenylalanine (pCmF) (e.g., 4-carboxymethyl-L-phenylalanine); 20) 2-aminooctanoic acid; and 21) an amino acid comprising a saccharide moiety, such as N-acetyl-L-glucosaminyl-L-serine, beta-N-acetylglucosamine-O-serine, N-acetyl-L-galactosaminyl-L-serine, alpha-N-acetylgalactosamine-O-serine, O-(3-O-D-galactosyl-N-acetyl-beta-D-galactosaminyl)-L-serine, N-acetyl-L-glucosaminyl-L-threonine, alpha-N-acetylgalactosamine-O-threonine, 3-O—(N-acetyl-beta-D-glucosaminyl)-L-threonine, N-acetyl-L-glucosaminyl-L-asparagine, N4-(β-N-Acetyl-D-glucosaminyl)-L-asparagine and O-(mannosyl)-L-serine; an amino acid wherein the naturally-occurring N- or O-linkage between the amino acid and the saccharide is replaced by a covalent linkage not commonly found in nature, including but not limited to, an alkene, an oxime, a thioether, an amide and the like; or an amino acid containing saccharides that are not commonly found in naturally-occurring polypeptides, such as 2-deoxy-glucose, 2-deoxy-galactose and the like. Specific examples of non-natural amino acids include, but are not limited to, a p-acetylphenylalanine (4-acetyl phenylalanine) (including 4-acetyl-L-phenylalanine, also referred to herein as p-acetyl-L-phenylalanine (pAF)), a 4-boronophenylalanine (pBoF) (e.g., 4-borono-L-phenylalanine, a 4-propargyloxyphenylalanine (pPrF) (e.g., 4-propargyloxy-L-phenylalanine), an O-methyltyrosine (e.g., O-methyl-L-tyrosine), a 3-(2-naphthyl)alanine (NapA) (e.g., 3-(2-naphthyl)-L-alanine), a 3-methylphenylalanine (e.g., 3-methyl-L-phenylalanine), an O-allyltyrosine (e.g., O-allyl-L-tyrosine), an O-isopropyltyrosine (e.g., O-isopropyl-L-tyrosine), a dopamine (e.g., L-Dopa), a 4-isopropylphenylalanine (e.g., 4-isopropyl-L-phenylalanine), a 4-azidophenylalanine (pAz) (e.g., 4-azido-L-phenylalanine), a 4-benzoylphenylalanine (pBpF) (e.g., 4-benzoyl-L-phenylalanine), an O-phosphoserine (e.g., O-phospho-L-serine), an O-phosphotyrosine (e.g., O-phospho-L-tyrosine), a 4-iodophenylalanine (pIF) (e.g., 4-iodo-L-phenylalanine, a 4-bromophenylalanine (e.g., 4-bromo-L-phenylalanine), a 4-aminophenylalanine (e.g., 4-amino-L-phenylalanine), a 4-cyanophenylalanine (pCNF) (e.g., 4-cyano-L-phenylalanine, a (8-hydroxyquinolin-3-yl)alanine (HQA) (e.g., (8-hydroxyquinolin-3-yl)-L-alanine), a (2,2-bipyridin-5-yl)alanine (BipyA) (e.g., (2,2-bipyridin-5-yl)-L-alanine), and the like. Additional non-natural amino acids are disclosed in Liu et al. (2010) Annu Rev Biochem, 79:413-44; Wang et al. (2005) Angew Chem Int Ed, 44:34-66; and Published International Application Nos.: WO 2012/166560, WO 2012/166559, WO 2011/028195, WO 2010/037062, WO 2008/083346, WO 2008/077079, WO 2007/094916, WO 2007/079130, WO 2007/070659 and WO 2007/059312, the entire contents of each of which are hereby incorporated by reference herein in their entirety. In some embodiments, the one or more non-natural amino acids can be p-acetylphenylalanine. In some more particular embodiments, the one or more non-natural amino acids can be p-acetyl-L-phenylalanine (pAF).

In some embodiments, one or more non-natural amino acids is selected from the group consisting of 4-acetyl phenylalanine, 3-O—(N-acetyl-beta-D-glucosaminyl)threonine, N4-(β-N-Acetyl-D-glucosaminyl)asparagine, O-allyltyrosine, alpha-N-acetylgalactosamine-O-serine, alpha-N-acetylgalactosamine-O-threonine, 2-aminooctanoic acid, 2-aminophenylalanine, 3-aminophenylalanine, 4-aminophenylalanine, 2-aminotyrosine, 3-aminotyrosine, 4-azidophenylalanine, 4-benzoylphenylalanine, (2,2-bipyridin-5yl)alanine, 3-boronophenylalanine, 4-boronophenylalanine, 4-bromophenylalanine, p-carboxymethylphenylalanine, 4-carboxyphenylalanine, p-cyanophenylalanine, 3,4-dihydroxyphenylalanine, 4-ethynylphenylalanine, 2-fluorophenylalanine, 3-fluorophenylalanine, 4-fluorophenylalanine, O-(3-O-D-galactosyl-N-acetyl-beta-D-galactosaminyl)serine, homoglutamine, (8-hydroxyquinolin-3-yl)alanine, 4-iodophenylalanine, 4-isopropylphenylalanine, O-i-propyltyrosine, 3-isopropyltyrosine, O-mannopyranosylserine, 2-methoxyphenylalanine, 3-methoxyphenylalanine, 4-methoxyphenylalanine, 3-methylphenylalanine, O-methyltyrosine, 3-(2-naphthyl)alanine, 5-nitrohistidine, 4-nitrohistidine, 4-nitroleucine, 2-nitrophenylalanine, 3-nitrophenylalanine, 4-nitrophenylalanine, 4-nitrotryptophan, 5-nitrotryptophan, 6-nitrotryptophan, 7-nitrotryptophan, 2-nitrotyrosine, 3-nitrotyrosine, O-phosphoserine, O-phosphotyrosine, 4-propargyloxyphenylalanine, O-2-propyn-1-yltyrosine, 4-sulfophenylalanine and O-sulfotyrosine.

In some further embodiments, one or more non-natural amino acids is selected from the group consisting of 4-acetyl-L-phenylalanine (para-acetyl-L-phenylalanine (pAF)), 3-O—(N-acetyl-beta-D-glucosaminyl)-L-threonine, N4-(β-N-Acetyl-D-glucosaminyl)-L-asparagine, O-allyl-L-tyrosine, alpha-N-acetylgalactosamine-O-L-serine, alpha-N-acetylgalactosamine-O-L-threonine, 2-aminooctanoic acid, 2-amino-L-phenylalanine, 3-amino-L-phenylalanine, 4-amino-L-phenylalanine, 2-amino-L-tyrosine, 3-amino-L-tyrosine, 4-azido-L-phenylalanine, 4-benzoyl-L-phenylalanine, (2,2-bipyridin-5yl)-L-alanine, 3-borono-L-phenylalanine, 4-borono-L-phenylalanine, 4-bromo-L-phenylalanine, p-carboxymethyl-L-phenylalanine, 4-carboxy-L-phenylalanine, p-cyano-L-phenylalanine, 3,4-dihydroxy-L-phenylalanine (L-DOPA), 4-ethynyl-L-phenylalanine, 2-fluoro-L-phenylalanine, 3-fluoro-L-phenylalanine, 4-fluoro-L-phenylalanine, O-(3-O-D-galactosyl-N-acetyl-beta-D-galactosaminyl)-L-serine, L-homoglutamine, (8-hydroxyquinolin-3-yl)-L-alanine, 4-iodo-L-phenylalanine, 4-isopropyl-L-phenylalanine, O-i-propyl-L-tyrosine, 3-isopropyl-L-tyrosine, O-mannopyranosyl-L-serine, 2-methoxy-L-phenylalanine, 3-methoxy-L-phenylalanine, 4-methoxy-L-phenylalanine, 3-methyl-L-phenylalanine, O-methyl-L-tyrosine, 3-(2-naphthyl)-L-alanine, 5-nitro-L-histidine, 4-nitro-L-histidine, 4-nitro-L-leucine, 2-nitro-L-phenylalanine, 3-nitro-L-phenylalanine, 4-nitro-L-phenylalanine, 4-nitro-L-tryptophan, 5-nitro-L-tryptophan, 6-nitro-L-tryptophan, 7-nitro-L-tryptophan, 2-nitro-L-tyrosine, 3-nitro-L-tyrosine, O-phospho-L-serine, O-phospho-L-tyrosine, 4-propargyloxy-L-phenylalanine, O-2-propyn-1-yl-L-tyrosine, 4-sulfo-L-phenylalanine and O-sulfo-L-tyrosine. In some embodiments, the one or more non-natural amino acids can be p-acetyl-L-phenylalanine (pAF). Thus, in some embodiments, each and every one of the one or more non-natural amino acids is pAF.

In certain embodiments of the disclosure, an antibody with at least one non-natural amino acid includes at least one post-translational modification. In one embodiment, the at least one post-translational modification comprises attachment of a molecule including but not limited to, a water-soluble polymer, a derivative of polyethylene glycol, a drug, a second protein or polypeptide or polypeptide analog, an antibody or antibody fragment, a biologically active agent, a small molecule, or any combination of the above or any other desirable compound or substance, comprising a second reactive group to at least one non-natural amino acid comprising a first reactive group utilizing chemistry methodology that is known to one of ordinary skill in the art to be suitable for the particular reactive groups. For example, the first reactive group is an alkynyl moiety (including but not limited to, the non-natural amino acid p-propargyloxyphenylalanine, where the propargyl group is also sometimes referred to as an acetylene moiety) and the second reactive group is an azido moiety, and [3+2]cycloaddition chemistry methodologies are utilized. In another example, the first reactive group is the azido moiety (including but not limited to, the non-natural amino acid p-azido-L-phenylalanine) and the second reactive group is the alkynyl moiety. In certain embodiments of the modified antibody polypeptide of the present disclosure at least one non-natural amino acid, (including but not limited to, non-natural amino acid containing a keto functional group), comprising at least one post-translational modification is used where the at least one post-translational modification comprises a saccharide moiety. In certain embodiments, the post-translational modification is made in vivo in a eukaryotic cell or in a non-eukaryotic cell. In other embodiments the post-translational modification is made in vitro. In another embodiment, the post-translational modification is made in vitro and in vivo.

In some embodiments, the non-natural amino acid may be modified to incorporate a chemical group. In some embodiments the non-natural amino acid may be modified to incorporate a ketone group. The one or more non-natural amino acids may comprise at least one oxime, carbonyl, dicarbonyl, hydroxylamine group or a combination thereof. The one or more non-natural amino acids may comprise at least one carbonyl, dicarbonyl, alkoxy-amine, hydrazine, acyclic alkene, acyclic alkyne, cyclooctyne, aryl/alkyl azide, norbornene, cyclopropene, trans-cyclooctene, or tetrazine functional group or a combination thereof.

In some embodiments disclosed herein the non-natural amino acid is site-specifically incorporated into the antibody, antibody fragment or variant. In some embodiments the non-natural amino acid is site-specifically incorporated into an antibody, antibody fragment or variant. Methods for incorporating a non-natural amino acid into a molecule, for example, proteins, polypeptides or peptides, are disclosed in U.S. Pat. Nos. 7,332,571; 7,928,163; 7,696,312; 8,008,456; 8,048,988; 8,809,511; 8,859,802; 8,791,231; 8,476,411; or 9,637,411, (each of which is incorporated herein by reference in its entirety), and in the Examples herein. The one or more non-natural amino acids may be incorporated by methods known in the art. For example, cell-based or cell-free systems may be used, and auxotrophic strains may also be used in place of engineered tRNA and synthetase. In certain embodiments, orthogonal tRNA synthetase are used as disclosed in for example, WO2002085923A2; WO2002086075A2; WO2004035743A2; WO2007021297A1; WO2006068802A2; and WO2006069246A2; the entire contents of each of which are hereby incorporated herein by reference in their entirety. Incorporating one or more non-natural amino acids into the antibody or antibody fragment or variant may comprise modifying one or more amino acid residues in the antibody or antibody fragment or variant. Modifying the one or more amino acid residues in the antibody or antibody fragment or variant may comprise mutating one or more nucleotides in the nucleotide sequence encoding the antibody or antibody fragment or variant. Mutating the one or more nucleotides in the nucleotide sequence encoding the antibody or antibody fragment or variant may comprise altering a codon encoding an amino acid to a nonsense codon. Incorporating one or more non-natural amino acids into the antibody or antibody fragment or variant may comprise modifying one or more amino acid residues in the antibody or antibody fragment or variant to produce one or more amber codons in the antibody or antibody fragment or variant. The one or more non-natural amino acids may be incorporated into the antibody or antibody fragment or variant in response to an amber codon. The one or more non-natural amino acids may be site-specifically incorporated into the antibody or antibody fragment or variant. Incorporating one or more non-natural amino acids into the antibody or antibody fragment or variant may comprise one or more genetically encoded non-natural amino acids with orthogonal chemical reactivity relative to the canonical twenty amino acids to site-specifically modify the biologically active molecule or targeting agent. Incorporating the one or more non-natural amino acids may comprise use of a tRNA/aminoacyl-tRNA synthetase pair to site-specifically incorporate one or more non-natural amino acids at defined sites in the biologically active molecule or targeting agent in response to one or more amber nonsense codon. Additional methods for incorporating non-natural amino acids include, but are not limited to, methods disclosed in Chatterjee et al., A Versatile Platform for Single- and Multiple-Unnatural Amino Acid Mutagenesis in Escherichia coli, Biochemistry, 2013; Kazane et al., J Am Chen Soc, 135(1):340-6, 2013; Kim et al., J Am Chem Soc, 134(24):9918-21, 2012; Johnson et al., Nat Chem Biol, 7(11):779-86, 2011; and Hutchins et al., J Mol Biol, 406(4):595-603, 2011. The one or more non-natural amino acids may be produced through selective reaction of one or more natural amino acids. The selective reaction may be mediated by one or more enzymes. In non-limiting examples, the selective reaction of one or more cysteines with formylglycine generating enzyme (FGE) may produce one or more formylglycines as described in Rabuka et al., Nature Protocols 7: 1052-1067, 2012. The one or more non-natural amino acids may involve a chemical reaction to form a linker. The chemical reaction to form the linker may include a bioorthogonal reaction. The chemical reaction to form the linker may include click chemistry. See for example WO2006/050262 incorporated herein by reference in its entirety.

Any position of the antibody or antibody fragment is suitable for selection to incorporate a non-natural amino acid, and selection may be based on rational design or by random selection for any or no particular desired purpose. Selection of desired sites may be based on producing a non-natural amino acid polypeptide (which may be further modified or remain unmodified) having any desired property or activity, including but not limited to a receptor binding modulators, receptor activity modulators, modulators of binding to binder partners, binding partner activity modulators, binding partner conformation modulators, dimer or multimer formation, no change to activity or property compared to the native molecule, or manipulating any physical or chemical property of the polypeptide such as solubility, aggregation, or stability. Alternatively, the sites identified as critical to biological activity may also be good candidates for substitution with a non-natural amino acid, again depending on the desired activity sought for the polypeptide. Another alternative would be to simply make serial substitutions in each position on the polypeptide chain with a non-natural amino acid and observe the effect on the activities of the polypeptide. Any means, technique, or method for selecting a position for substitution with a non-natural amino acid into any polypeptide is suitable for use in the methods, techniques and compositions described herein.

The structure and activity of naturally-occurring mutants of a polypeptide that contain deletions can also be examined to determine regions of the protein that are likely to be tolerant of substitution with a non-natural amino acid. Once residues that are likely to be intolerant to substitution with non-natural amino acids have been eliminated, the impact of proposed substitutions at each of the remaining positions can be examined using methods including, but not limited to, the three-dimensional structure of the relevant polypeptide, and any associated ligands or binding proteins. X-ray crystallographic and NMR structures of many polypeptides are available in the Protein Data Bank (PDB, see world wide web for rcsb.org), a centralized database containing three-dimensional structural data of large molecules of proteins and nucleic acids, and can be used to identify amino acid positions that can be substituted with non-natural amino acids. In addition, models may be made investigating the secondary and tertiary structure of polypeptides, if three-dimensional structural data is not available. Thus, the identity of amino acid positions that can be substituted with non-natural amino acids can be determined by the skilled person.

Exemplary sites of incorporation of a non-natural amino acid include, but are not limited to, those that are excluded from potential receptor binding regions, or regions for binding to binding proteins or ligands may be fully or partially solvent exposed, have minimal or no hydrogen-bonding interactions with nearby residues, may be minimally exposed to nearby reactive residues, and/or may be in regions that are highly flexible as predicted by the three-dimensional crystal structure of a particular polypeptide with its associated receptor, ligand or binding proteins

A wide variety of non-natural amino acids can be substituted for, or incorporated into, a given position in a polypeptide. By way of example, a particular non-natural amino acid may be selected for incorporation based on an examination of the three-dimensional crystal structure of a polypeptide with its associated ligand, receptor and/or binding proteins, a preference for conservative substitutions.

### Linkers

In some aspects, the present disclosure relates to linkers for intracellular delivery of drug conjugates. Many procedures and linker molecules for attachment of various compounds to peptides are known. See, for example, European Patent Application No. 0188256; U.S. Pat. Nos. 4,671,958, 4,659,839, 4,414,148, 4,699,784, 4,680,338, 4,569,789 and 10,550,190; PCT Application Publication Nos. WO 2012/166559 A1, WO 2012/166560 A1, WO 2013/185117 A1, WO 2013/192360 A1 and WO 2022/040596 A1; and US Patent Application Publication No. US 2017/0182181 A1; the contents of each of which are hereby incorporated by reference in their entirety.

In some embodiments, the present disclosure relates to phosphate-based linkers for intracellular delivery of drug conjugates (see, e.g., U.S. Pat. No. 10,550,190). The phosphate-based linkers of the present disclosure include a monophosphate, diphosphate, triphosphate, or tetraphosphate group (phosphate group) and a linker arm and optionally a spacer. A drug-linker can be covalently linked to a reactive functional group that can be covalently linked to a cell-specific targeting ligand such as an antibody or antibody fragment. Phosphate-based linkers have a differentiated and tunable stability in blood compared to the intracellular environment (e.g., lysosomal compartment). Thus, ADCs comprising these phosphate-based linkers are stable in circulation (plasma/blood) but reactive or cleavable in intracellular compartments (lysosome) making them useful for intracellular delivery of drug conjugates. The phosphate-based linker is capable of being conjugated to a drug and the reactive functional group is capable of being conjugated to a cell-specific targeting ligand such as an anti-TROP2 antibody, an anti-HER2 antibody, an anti-CD70 antibody, an anti-CD3 antibody or an anti-PSMA antibody. The phosphate-based linkers of the present disclosure are designed to engineer ADCs such that the likelihood of the conjugate to form aggregates is reduced compared to conjugates in which the same drug is conjugated to the antibody or targeting ligand using a linker that is not a phosphate-based linker. Further, the phosphate-based linker design, stability, pH, redox sensitivities and protease susceptibility influence circulatory stability and release of the drug.

Methods for selecting and designing linkers are well known in the art. Linkers may be designed de novo, including by way of example only, as part of high-throughput screening process (in which case numerous polypeptides may be designed, synthesized, characterized and/or tested) or based on the interests of the researcher. The linker may also be designed based on the structure of a known or partially characterized polypeptide. The principles for selecting which amino acid(s) to substitute and/or modify and the choice of which modification to employ are described in WO2013/185117, for example. Linkers may be designed to meet the needs of the experimenter or end user. Such needs may include, but are not limited to, manipulating the therapeutic effectiveness of the polypeptide, improving the safety profile of the polypeptide, adjusting the pharmacokinetics, pharmacologics and/or pharmacodynamics of the polypeptide, such as, by way of example only, increasing water solubility, bioavailability, increasing serum half-life, increasing therapeutic half-life, modulating immunogenicity, modulating biological activity, or extending the circulation time. In addition, such modifications include, by way of example only, providing additional functionality to the polypeptide, incorporating an antibody, and any combination of the aforementioned modifications.

Generally, a linker of the present disclosure can be a unit that is combinable with one or more additional units, such that the combined linker units can bond to one or more drugs. Each linker unit can be comprised of one or more moieties, each of which may occur one or more times.

In some embodiments, a linker of the present disclosure comprises at least one phosphate-based moiety, as disclosed herein. In some embodiments, the linker comprises the phosphate-based moiety and further comprises at least one moiety or unit that is not phosphate-based. In some embodiments, the linker is a bivalent linker. In some embodiments, the linker is a trivalent linker. In some embodiments, the linker is a tetravalent linker.

Thus, in some aspects, the present disclosure provides for phosphate-based linkers. A phosphate-based linker of the present disclosure, or a drug-linker of the present disclosure, can comprise a phosphate-based moiety, wherein the phosphate-based moiety is a phosphate ester, a pyrophosphate ester, a triphosphate ester, a tetraphosphate ester, a phosphonate, a diphosphonate, a phosporamidate, a pyrophosporamidate, a triphosphoramidate, a tetraphosphoramidate, a phosphorthioate and/or a diphosphorthioate. Thus, a phosphate-based linker, or a drug-linker, of the present disclosure can comprise:
a phosphate ester having the structure

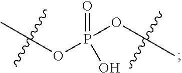 [SMILES: *OP(=O)(O)O*]

a phosphonate having the structure

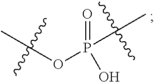 [SMILES: *OP(*)(=O)O]

a pyrophosphate ester having the structure

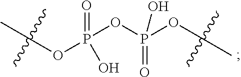 [SMILES: *OP(=O)(O)OP(=O)(O)O*]

a diphosphonate having the structure

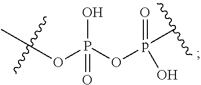

a triphosphate ester having the structure

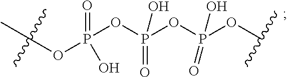 [SMILES: *OP(=O)(O)OP(=O)(O)OP(=O)(O)O*]

a tetraphosphate ester having the structure

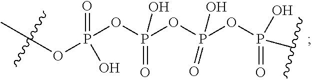 [SMILES: *OP(=O)(O)OP(=O)(O)OP(=O)(O)OP(*)(=O)O]

a phosphorthioate having the structure

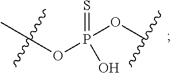 [SMILES: *OP(O)(=S)O*]

a diphosphorthioate having the structure

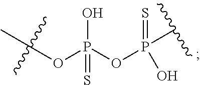 [SMILES: *OP(O)(=S)OP(*)(O)=S]

a phosphoramidate having the structure

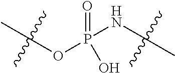 [SMILES: *NP(=O)(O)O*]

a pyrophosphoramidate having the structure

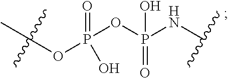 [SMILES: *NP(=O)(O)OP(=O)(O)O*]

a triphosphoramidate having the structure

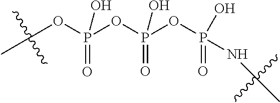 [SMILES: *NP(=O)(O)OP(=O)(O)OP(=O)(O)O*]

and/or
a tetraphosphoramidate having the structure

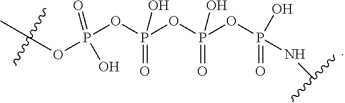 [SMILES: *NP(=O)(O)OP(=O)(O)OP(=O)(O)OP(=O)(O)O*]

In some embodiments, a phosphate-based linker of the present disclosure comprises a phosphate-based moiety selected from the group consisting of a pyrophosphate ester and a diphosphonate. In some embodiments, a drug-linker of the present disclosure comprises a phosphate-based moiety selected from the group consisting of a pyrophosphate ester and a diphosphonate.

In some embodiments, a phosphate-based linker, or a drug-linker, of the present disclosure comprises a pyrophosphate ester. For example, in some embodiments, a drug (e.g., a duocarmycin compound of the present disclosure) comprises an oxygen atom (—O—) that is joined to a phosphorus atom of a diphosphonate moiety, thereby providing a drug-linker comprising a pyrophosphate ester.

In some other embodiments, a phosphate-based linker, or a drug-linker, of the present disclosure comprises a diphosphonate.

In some embodiments, the phosphate-based linker is a bivalent linker.

In some embodiments, the phosphate-based moiety is covalently bound to an —O— atom of a drug, e.g., an —O— atom of a compound of Formula (X), or Formula (I), or Formula (Ia), or Formula (Ib), or Formula (Ic), or Formula (Id), or Formula (IL), or Formula (ILa), or Formula (ILb), or Formula (ILc), or Formula (ILd), or an ADC of Formula (II), as disclosed herein, via a phosphorous atom of the phosphate-based moiety.

In some embodiments, the phosphate-based linker further comprises at least one additional moiety. In some embodiments, each at least one additional moiety is independently selected from the group consisting of unsubstituted alkylene, substituted alkylene, -(alkylene-O)—, optionally substituted arylene, —O—, —C(O)—, —N(R_{w})—, —S(O)₀₋₂—, a water-soluble polymer and an amino acid; wherein each R_{w} is independently H or alkyl, alkenyl or alkynyl; and combinations thereof. In some embodiments, each R_{w} is independently H or unsubstituted C₁-C₈ alkyl, C₁-C₈ alkenyl or C₁-C₈ alkyl alkynyl. In some further embodiments, each R_{w} is independently H or unsubstituted C₁-C₈alkyl. In yet some further embodiments, each R_{w} in independently H or methyl.

In some embodiments, each at least one additional moiety is independently selected from the group consisting of unsubstituted alkylene, -(alkylene-O)—, —C(O)—, —N(R_{w})—, a water-soluble polymer and an amino acid; wherein each R_{w} is independently H or alkyl, alkenyl or alkynyl; and combinations thereof. In some embodiments, each R_{w} is independently H or unsubstituted C₁-C₈ alkyl, C₁-C₈ alkenyl or C₁-C₈ alkyl alkynyl. In some further embodiments, each R_{w} is independently H or unsubstituted C₁-C₈ alkyl. In yet some further embodiments, each R_{w} in independently H or methyl.

It is to be understood that each at least one additional moiety that can be present in a phosphate-based linker of the present disclosure can occur one or more times within said linker. In a non-limiting example, a phosphate-based linker of the present disclosure can comprise one or more unsubstituted alkylene group, wherein, each said unsubstituted alkylene group can be the same or different. In another non-limiting example, a phosphate-based linker of the present disclosure can comprise one or more amino acids, wherein each amino acid is the same or different.

In some embodiments, the linker comprises at least one alkylene group.

In some embodiments, the linker comprises an amino acid. In some embodiments, the amino acid is selected from the group consisting of serine, threonine, cysteine, tyrosine, aspartic acid, glutamic acid, lysine and N_{ϵ}-methyl-lysine. In some embodiments, the amino acid is lysine or N_{ϵ}-methyl-lysine. In some embodiments, the amino acid is lysine. In some embodiments, the amino acid is N_{ϵ}-methyl-lysine.

In some embodiments, the linker comprises a water-soluble polymer.

In some embodiments, the linker comprises a water-soluble polymer and an amino acid, wherein the water-soluble polymer is conjugated to the amino acid. In some embodiments, the water-soluble polymer is conjugated to a side chain of the amino acid. In some embodiments, the water-soluble polymer is conjugated to the amino acid via a spacer element.

In some embodiments, a phosphate-based linker of the present disclosure comprises a water-soluble polymer and an amino acid which is serine, threonine or tyrosine, wherein the water-soluble polymer is conjugated to the side chain —OH group of the serine, threonine or tyrosine. In some embodiments, the water-soluble polymer is conjugated to the amino acid via a spacer element.

In some embodiments, a phosphate-based linker of the present disclosure comprises a water-soluble polymer and an amino acid which is cysteine, wherein the water-soluble polymer is conjugated to the side chain —SH group of the cysteine. In some embodiments, the water-soluble polymer is conjugated to the amino acid via a spacer element.

In some embodiments, a phosphate-based linker of the present disclosure comprises a water-soluble polymer and an amino acid which is aspartic acid or glutamic acid, wherein the water-soluble polymer is conjugated to the side chain carboxylate group of the aspartic acid or glutamic acid. In some embodiments, the water-soluble polymer is conjugated to the amino acid via a spacer element.

In some embodiments, a phosphate-based linker of the present disclosure comprises a water-soluble polymer and an amino acid which is lysine and N_{ϵ}-methyl-lysine, wherein the water-soluble polymer is conjugated to the side chain —N_{ϵ}H(R) group of the lysine or N_{ϵ}-methyl-lysine, wherein R is H or methyl, respectively. In some embodiments, the water-soluble polymer is conjugated to the amino acid via a spacer element.

In some embodiments, the water-soluble polymer is a polysaccharide.

In some embodiments, the water-soluble polymer is a (polyethylene)glycol (PEG) moiety. In some embodiments, the PEG moiety has a molecular weight within a range of about 100 Da to about 100,000 Da. In some embodiments, the PEG moiety has a molecular weight within a range of about 100 Da to about 10,000 Da. In some embodiments, the PEG moiety has a molecular weight within a range of about 100 Da to about 5,000 Da. In some embodiments, the PEG moiety has a molecular weight within a range of about 100 Da to about 1,000 Da.

In some embodiments, the PEG moiety is —(CH₂CH₂O)ₙCH₃, wherein n is an integer from 1 to 100. In some embodiments, the PEG moiety is —(CH₂CH₂O)ₙCH₃, wherein n is an integer from 1 to 24. In some embodiments, the PEG moiety is —(CH₂CH₂O)ₙCH₃, wherein n is an integer from 6 to 12. In some embodiments, the PEG moiety is —(CH₂CH₂O)ₙCH₃, wherein n is an integer from 8 to 12. In some embodiments, the PEG moiety is —(CH₂CH₂O)ₙCH₃, wherein n is 8. In some embodiments, the PEG moiety is —(CH₂CH₂O)ₙCH₃, wherein n is 12.

In some embodiments, the PEG is linear. In some embodiments, the PEG is branched, multi-armed or dendritic.

In some embodiments, a phosphate-based linker of the present disclosure is a linker selected from the group of linkers listed in Table 6.

TABLE 6
Non-limiting examples of linkers of the present disclosure.
Linker

*—P(═O)(OH)—O—P(═O)(OH)—(O)ᵢ—,
*—P(═O)(OH)—O—P(═O)(OH)—(O)ᵢ-alkylene-,
*—P(═O)(OH)—O—P(═O)(OH)—(O)ᵢ-alkylene-C(O)—,
*—P(═O)(OH)—O—P(═O)(OH)—(O)ᵢ-alkylene-N(H)—,
*—P(═O)(OH)—O—P(═O)(OH)—(O)ᵢ-alkylene-J-,
*—P(═O)(OH)—O—P(═O)(OH)—(O)ᵢ-(alkylene-O)ₙ—,
*—P(═O)(OH)—O—P(═O)(OH)—(O)ᵢ-(alkylene-O)ₙ-alkylene-,
*—P(═O)(OH)—O—P(═O)(OH)—(O)ᵢ-alkylene-(O-alkylene)ₙ-,
*—P(═O)(OH)—O—P(═O)(OH)—(O)ᵢ-(alkylene-O)ₙ-alkylene-C(O)—,
*—P(═O)(OH)—O—P(═O)(OH)—(O)ᵢ-(alkylene-O)ₙ-alkylene-N(H)—,
*—P(═O)(OH)—O—P(═O)(OH)—(O)ᵢ-alkylene-J-alkylene-,
*—P(═O)(OH)—O—P(═O)(OH)—(O)ᵢ-(alkylene-O)ₙ-J-alkylene-,
*—P(═O)(OH)—O—P(═O)(OH)—(O)ᵢ-alkylene-(O-alkylene)ₙ-J-alkylene-,
*—P(═O)(OH)—O—P(═O)(OH)—(O)ᵢ-alkylene-J-(alkylene-O)ₙ-alkylene-,
*—P(═O)(OH)—O—P(═O)(OH)—(O)ᵢ-alkylene-U-alkylene-,
*—P(═O)(OH)—O—P(═O)(OH)—(O)ᵢ-alkylene-(O-alkylene)ₙ-U-alkylene-,
*—P(═O)(OH)—O—P(═O)(OH)—(O)ᵢ-alkylene-(O-alkylene)ₙ-U-alkylene-,
*—P(═O)(OH)—O—P(═O)(OH)—(O)ᵢ-alkylene-U-(alkylene-O)ₙ-alkylene-;
—C(O)—O-alkylene-O—P(═O)(OH)—O—P(═O)(OH)—(O)-alkylene-,
—C(O)—O-alkylene-O—P(═O)(OH)—O—P(═O)(OH)—(O-alkylene)ₙ-,
—C(O)—O-alkylene-O—P(═O)(OH)—O—P(═O)(OH)—(O)ᵢ-alkylene-J-alkylene-,
—C(O)—O-alkylene-O—P(═O)(OH)—O—P(═O)(OH)—(O)ᵢ-alkylene-(O-alkylene)ₙ-,
-alkylene-P(═O)(OH)—O—P(═O)(OH)—(O)ᵢ—,
-alkylene-O—P(═O)(OH)—O—P(═O)(OH)—(O)ᵢ— ,
-alkylene-P(═O)(OH)—O—P(═O)(OH)—(O)ᵢ-alkylene-,
-alkylene-O—P(═O)(OH)—O—P(═O)(OH)—(O)ᵢ-alkylene-,
-alkylene-O—P(═O)(OH)—O—P(═O)(OH)—(O-alkylene)ₙ-,
-alkylene-P(=O)(OH)—O—P(═O)(OH)—(O)ᵢ-alkylene-J-alkylene-,
-alkylene-O—P(═O)(OH)—O—P(=O)(OH)—(O)ᵢ-alkylene-J-alkylene-,
-alkylene-P(═O)(OH)—O—P(═O)(OH)—(O)ᵢ₋(alkylene-O)ₙ—,
-alkylene-O—P(═O)(OH)—O—P(═O)(OH)—(O)ᵢ-(alkylene-O)ₙ—,
-alkylene-P(═O)(OH)—O—P(═O)(OH)—(O)ᵢ-(alkylene-O)ₙ-alkylene-,
-alkylene-O—P(═O)(OH)—O—P(═O)(OH)—(O)ᵢ-(alkylene-O)ₙ-alkylene-,
-alkylene-P(═O)(OH)—O—P(═O)(OH)—(O)ᵢ-alkylene-(O-alkylene)ₙ-,
-alkylene-O—P(=O)(OH)—O—P(=O)(OH)—(O)ᵢ-alkylene-(O-alkylene)ₙ-,
-(alkylene-O)ₙ—P(═O)(OH)—O—P(═O)(OH)—(O)ᵢ— ,
-(alkylene-O)ₙ—P(═O)(OH)—O—P(═O)(OH)—(O)ᵢ-alkylene-,
-(alkylene-O)ₙ—P(═O)(OH)—O—P(═O)(OH)—(O)ᵢ-(alkylene-O)ₙ—,
-(alkylene-O)ₙ—P(═O)(OH)—O—P(═O)(OH)—(O)ᵢ-(alkylene-O)ₙ-alkylene-,
-(alkylene-O)ₙ-alkylene-P(═O)(OH)—O—P(═O)(OH)—(O)ᵢ— ,
-(alkylene-O)ₙ-alkylene-O—P(═O)(OH)—O—P(═O)(OH)—(O)ᵢ— ,
-(alkylene-O)ₙ-alkylene-P(═O)(OH)—O—P(═O)(OH)—(O)ᵢ-alkylene-,
-(alkylene-O)ₙ-alkylene-O—P(═O)(OH)—O—P(═O)(OH)—(O)ᵢ-alkylene-,
-(alkylene-O)ₙ-alkylene-P(═O)(OH)—O—P(═O)(OH)—(O)ᵢ-(alkylene-O)ₙ—,
-(alkylene-O)ₙ-alkylene-O—P(═O)(OH)—O—P(═O)(OH)—(O)ᵢ-(alkylene-O)ₙ—,
-(alkylene-O)ₙ-alkylene-P(═O)(OH)—O—P(═O)(OH)—(O)ᵢ-(alkylene-O)ₙ-alkylene-,
-(alkylene-O)ₙ-alkylene-O—P(═O)(OH)—O—P(═O)(OH)—(O)ᵢ—(alkylene-O)ₙ-alkylene-;
wherein:
each U is an amino acid, wherein the alpha-amino group of the amino acid is
optionally joined to a carbonyl, and wherein the alpha-carbonyl of the amino acid is
optionally joined to NH; optionally, each U is independently:

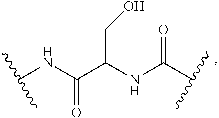 [SMILES: *NC(=O)C(CO)NC(*)=O]

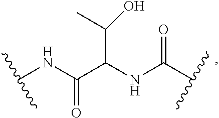 [SMILES: *NC(=O)C(NC(*)=O)C(C)O]

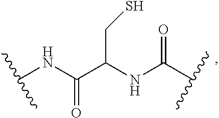 [SMILES: *NC(=O)C(CS)NC(*)=O]

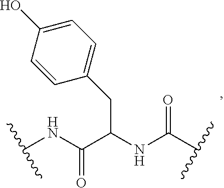 [SMILES: *NC(=O)C(Cc1ccc(O)cc1)NC(*)=O]

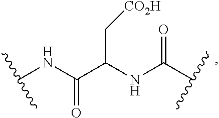 [SMILES: *NC(=O)C(CC(=O)O)NC(*)=O]

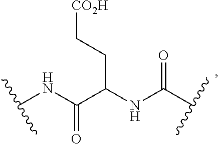 [SMILES: *NC(=O)C(CCC(=O)O)NC(*)=O]

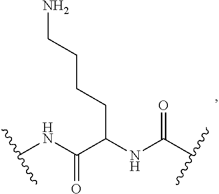 [SMILES: *NC(=O)C(CCCCN)NC(*)=O]

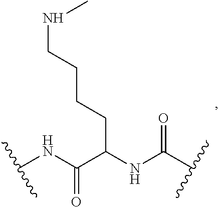 [SMILES: *NC(=O)C(CCCCNC)NC(*)=O]

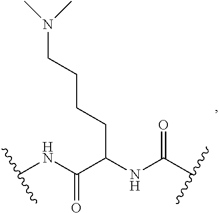 [SMILES: *NC(=O)C(CCCCN(C)C)NC(*)=O]

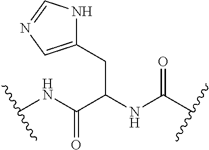 [SMILES: *NC(=O)C(Cc1cnc[nH]1)NC(*)=O]

or

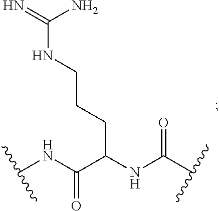 [SMILES: *NC(=O)C(CCCNC(=N)N)NC(*)=O]

each J is independently:

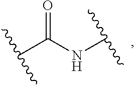 [SMILES: *NC(*)=O]

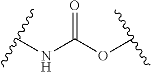 [SMILES: *NC(=O)O*]

or

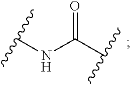 [SMILES: *NC(*)=O]

each alkylene is independently selected from the group consisting of:
—(CH₂)—, —(CH₂)₂—, —(CH₂)₃—, —(CH₂)₄—, —(CH₂)₅—, —(CH₂)₆—, —(CH₂)₇—,
—(CH₂)₈—, —(CH₂)₉—, —(CH₂)₁₀—, —(CH₂)₁₁— and —(CH₂)₁₂—; optionally, each
alkylene is independently —(CH₂)—, —(CH₂)₂— or —(CH₂)₃—;
each n is independently an integer from 1 to 100; optionally, each n is
independently an integer from 1 to 10;
each i is independently 0 or 1; and
*, when present, denotes the connection to drug; and
wherein each linker is optionally substituted with at least one water-soluble polymer.

In some embodiments, each i of Table 6 is 1. In some other embodiments, each i of Table 6 is 0.

In some embodiments, each U is independently optionally substituted with a water-soluble polymer.

In some embodiments, each n of Table 6 is independently an integer from 1 to 10. In some embodiments, each n of Table 6 is independently 1, 2 or 3.

In some embodiments, each alkylene of Table 6 is independently —(CH₂)—, —(CH2)₂— or —(CH2)₃—.

In some embodiments, each linker of Table 6 is substituted with one or more water-soluble polymer. In some embodiments, each U of Table 6 is substituted with one or more water-soluble polymers. In some embodiments, each U of Table 6 is substituted with one water-soluble polymer.

In some embodiments, a water-soluble polymer is conjugated to the amino acid side chain of group U. In some embodiments, a water-soluble polymer is conjugated to the amino acid side chain of group U via a spacer element.

In some embodiments, the water-soluble polymer is a polysaccharide.

In some embodiments, the water-soluble polymer is a (polyethylene)glycol (PEG) moiety. In some embodiments, the PEG moiety has a molecular weight within a range of about 100 Da to about 100,000 Da. In some embodiments, the PEG moiety has a molecular weight within a range of about 100 Da to about 10,000 Da. In some embodiments, the PEG moiety has a molecular weight within a range of about 100 Da to about 5,000 Da. In some embodiments, the PEG moiety has a molecular weight within a range of about 100 Da to about 1,000 Da.

In some embodiments, the PEG moiety is —(CH₂CH₂O)ₙCH₃, wherein n is an integer from 1 to 100. In some embodiments, the PEG moiety is —(CH₂CH₂O)ₙCH₃, wherein n is an integer from 1 to 24. In some embodiments, the PEG moiety is —(CH₂CH₂O)ₙCH₃, wherein n is an integer from 6 to 12. In some embodiments, the PEG moiety is —(CH₂CH₂O)ₙCH₃, wherein n is an integer from 8 to 12. In some embodiments, the PEG moiety is —(CH₂CH₂O)ₙCH₃, wherein n is 8. In some embodiments, the PEG moiety is —(CH₂CH₂O)ₙCH₃, wherein n is 12.

In some embodiments, the PEG is linear. In some embodiments, the PEG is branched, multi-armed or dendritic.

It is understood that, unless expressly indicated otherwise, no orientation of a linker is implied by the direction in which the formula of the linker group is written. By way of example only, the formula -alkylene-O—P(═O)(OH)—O—P(═O)(OH)—(O)ᵢ— represents both -alkylene-O—P(═O)(OH)—O—P(═O)(OH)—(O)ᵢ— and —(O)ᵢ—P(═O)(OH)—O—P(═O)(OH)—O-alkylene-. In another example, the formula -alkylene-O—P(═O)(OH)—O—P(═O)(OH)—(O)ᵢ— represents both *-alkylene-O—P(═O)(OH)—O—P(═O)(OH)—(O)ᵢ— and -alkylene-O—P(═O)(OH)—O—P(═O)(OH)—(O)ᵢ—*, wherein * denotes a point of connection, for example, connection to a drug.

It is also understood that, when each alkylene (or other variable) of a linker is independently selected from a group of variables, the independent selection can be made within a given linker. By way of example only, the formula *-alkylene-O—P(═O)(OH)—O—P(═O)(OH)—(O)ᵢ-alkylene-(O-alkylene)ₙ-, wherein each alkylene is independently —(CH₂)—, —(CH₂)₂— or —(CH₂)₃—, includes but is not limited to the following species:

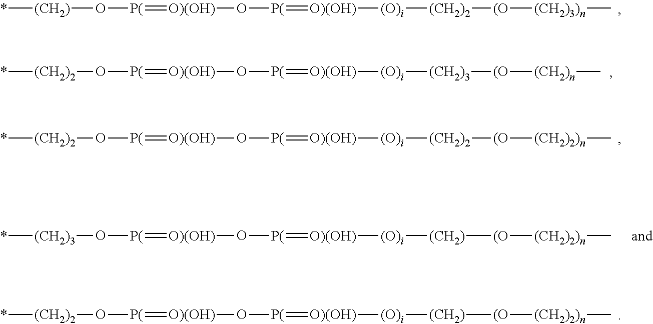

Furthermore, by way of example only, the group *-alkylene-O—P(═O)(OH)—O—P(═O)(OH)—(O)ᵢ-alkylene-(O-alkylene)ₙ-, wherein each alkylene is independently —(CH₂)—, —(CH₂)₂— or —(CH₂)₃—, can be rewritten: *-alkylene-O—P(═O)(OH)—O—P(═O)(OH)—(O)ᵢ-alkylene′-(O-alkylene″)ₙ-, wherein each alkylene, alkylene′ and alkylene″ is independently —(CH₂)—, —(CH₂)₂— or —(CH₂)₃—. Similarly, *-(alkylene-O)ₙ—P(═O)(OH)—O—P(═O)(OH)—(O)ᵢ-(alkylene-O)ₙ-J-alkylene-(alkylene-O)ₙ—, wherein each alkylene is independently —(CH₂)—, —(CH2)₂— or —(CH2)₃—, and each n is independently 1, 2 or 3, can be rewritten: *-(alkylene-O)ₙ—P(═O)(OH)—O—P(═O)(OH)—(O)-(alkylene′-O)_{n′-}J-alkylene″-(alkylene″′—O)_{n″}—, wherein each alkylene, alkylene′, alkylene″′ and alkylene″″ is independently —(CH₂)—, —(CH₂)₂— or —(CH₂)₃—; and each n, n′ and n″ is independently 1, 2 or 3.

In some embodiments, a phosphate-based linker of the present disclosure is a linker selected from the group of linkers listed in Table 7.

TABLE 7
Non-limiting examples of linkers of the present disclosure.
Linker

*—P(═O)(OH)—O—P(═O)(OH)—(O)ᵢ—,
*—P(═O)(OH)—O—P(═O)(OH)—(O)ᵢ-alkylene-,
*—P(═O)(OH)—O—P(═O)(OH)—(O)ᵢ-alkylene-C(O)—,
*—P(═O)(OH)—O—P(═O)(OH)—(O)ᵢ-alkylene-N(H)—,
*—P(═O)(OH)—O—P(═O)(OH)—(O)ᵢ-alkylene-J—,
*—P(═O)(OH)—O—P(═O)(OH)—(O)ᵢ-(alkylene-O)ₙ—,
*—P(═O)(OH)—O—P(═O)(OH)—(O)ᵢ-(alkylene-O)ₙ-alkylene-,
*—P(═O)(OH)—O—P(═O)(OH)—(O)ᵢ-alkylene-(O-alkylene)ₙ—,
*—P(═O)(OH)—O—P(═O)(OH)—(O)ᵢ-(alkylene-O)ₙ-alkylene-C(O)—,
*—P(═O)(OH)—O—P(═O)(OH)—(O)ᵢ-(alkylene-O)ₙ-alkylene-N(H)—,
*—P(═O)(OH)—O—P(═O)(OH)—(O)ᵢ-alkylene-J-alkylene-,
*—P(═O)(OH)—O—P(═O)(OH)—(O)ᵢ-(alkylene-O)ₙ—J-alkylene-,
*—P(═O)(OH)—O—P(═O)(OH)—(O)ᵢ-alkylene-(O-alkylene)ₙ—J-alkylene-,
*—P(═O)(OH)—O—P(═O)(OH)—(O)ᵢ-alkylene-J-(alkylene-O)ₙ-alkylene-,
*—P(═O)(OH)—O—P(═O)(OH)—(O)ᵢ-alkylene-U-alkylene-,
*—P(═O)(OH)—O—P(═O)(OH)—(O)ᵢ-alkylene-(O-alkylene)ₙ-U-alkylene-,
*—P(═O)(OH)—O—P(═O)(OH)—(O)ᵢ-alkylene-(O-alkylene)ₙ-U-alkylene-,
*—P(═O)(OH)—O—P(═O)(OH)—(O)ᵢ-alkylene-U-(alkylene-O)ₙ-alkylene-;
wherein:
each U is an amino acid, wherein the alpha-amino group of the amino acid is
optionally joined to a carbonyl, and wherein the alpha-carbonyl of the amino acid is
optionally joined to NH; optionally, each U is independently:

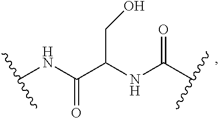 [SMILES: *NC(=O)C(CO)NC(*)=O]

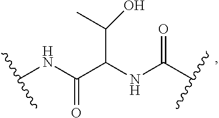 [SMILES: *NC(=O)C(NC(*)=O)C(C)O]

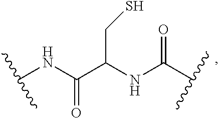 [SMILES: *NC(=O)C(CS)NC(*)=O]

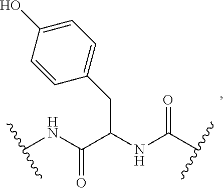 [SMILES: *NC(=O)C(Cc1ccc(O)cc1)NC(*)=O]

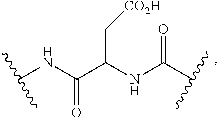 [SMILES: *NC(=O)C(CC(=O)O)NC(*)=O]

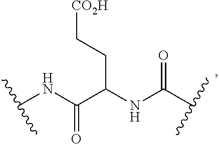 [SMILES: *NC(=O)C(CCC(=O)O)NC(*)=O]

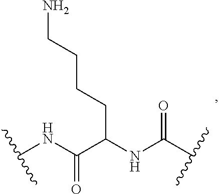 [SMILES: *NC(=O)C(CCCCN)NC(*)=O]

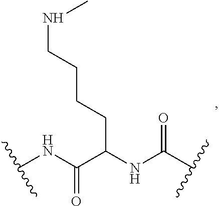 [SMILES: *NC(=O)C(CCCCNC)NC(*)=O]

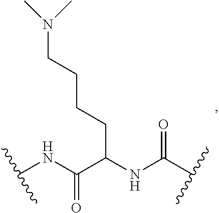 [SMILES: *NC(=O)C(CCCCN(C)C)NC(*)=O]

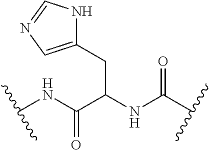 [SMILES: *NC(=O)C(Cc1cnc[nH]1)NC(*)=O]

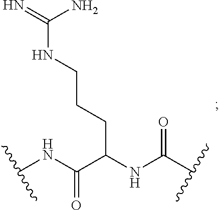 [SMILES: *NC(=O)C(CCCNC(=N)N)NC(*)=O]

each J is independently:

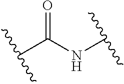 [SMILES: *NC(*)=O]

or

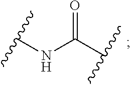 [SMILES: *NC(*)=O]

each alkylene is independently selected from the group consisting of:
—(CH₂)—, —(CH₂)₂—, —(CH₂)₃—, —(CH₂)₄—, —(CH₂)₅—, —(CH₂)₆—, —(CH₂)₇—,
—(CH₂)₈—, —(CH₂)₉—, —(CH₂)₁₀—, —(CH₂)₁₁— and —(CH₂)₁₂—;
each n is independently an integer from 1 to 100; optionally, each n is
independently an integer from 1 to 10;
each i is independently 0 or 1; and
* denotes the connection to drug; and
wherein each linker is optionally substituted with at least one water-soluble polymer.

In some embodiments, each i of Table 7 is 1. In some other embodiments, each i of Table 7 is 0.

In some embodiments, each U is independently optionally substituted with a water-soluble polymer.

In some embodiments, each n of Table 7 is independently an integer from 1 to 10. In some embodiments, each n of Table 7 is independently 1, 2 or 3.

In some embodiments, each alkylene of Table 7 is independently —(CH₂)—, —(CH2)₂— or —(CH2)₃—.

In some embodiments, each linker of Table 7 is substituted with one or more water-soluble polymer. In some embodiments, each U of Table 7 is substituted with one or more water-soluble polymers. In some embodiments, each U of Table 7 is substituted with one water-soluble polymer.

In some embodiments, a water-soluble polymer is conjugated to the amino acid side chain of group U. In some embodiments, a water-soluble polymer is conjugated to the amino acid side chain of group U via a spacer element.

In some embodiments, the water-soluble polymer is a polysaccharide.

In some embodiments, the water-soluble polymer is a (polyethylene)glycol (PEG) moiety. In some embodiments, the PEG moiety has a molecular weight within a range of about 100 Da to about 100,000 Da. In some embodiments, the PEG moiety has a molecular weight within a range of about 100 Da to about 10,000 Da. In some embodiments, the PEG moiety has a molecular weight within a range of about 100 Da to about 5,000 Da. In some embodiments, the PEG moiety has a molecular weight within a range of about 100 Da to about 1,000 Da.

In some embodiments, the PEG moiety is —(CH₂CH₂O)ₙCH₃, wherein n is an integer from 1 to 100. In some embodiments, the PEG moiety is —(CH₂CH₂O)ₙCH₃, wherein n is an integer from 1 to 24. In some embodiments, the PEG moiety is —(CH₂CH₂O)ₙCH₃, wherein n is an integer from 6 to 12. In some embodiments, the PEG moiety is —(CH₂CH₂O)ₙCH₃, wherein n is an integer from 8 to 12. In some embodiments, the PEG moiety is —(CH₂CH₂O)ₙCH₃, wherein n is 8. In some embodiments, the PEG moiety is —(CH₂CH₂O)ₙCH₃, wherein n is 12.

In some embodiments, the PEG is linear. In some embodiments, the PEG is branched, multi-armed or dendritic.

In some further embodiments, a phosphate-based linker of the present disclosure is a linker selected from the group of linkers listed in Table 8.

TABLE 8
Non-limiting examples of linkers of the present disclosure.
Linker

*—P(═O)(OH)—O—P(═O)(OH)—(O)-alkylene-J-alkylene-,
*—P(═O)(OH)—O—P(═O)(OH)—(O)-(alkylene-O)ₙ-J-alkylene-,
*—P(═O)(OH)—O—P(═O)(OH)—(O)-alkylene-(O-alkylene)ₙ-J-alkylene-,
*—P(═O)(OH)—O—P(═O)(OH)—(O)-alkylene-J-(alkylene-O)ₙ-alkylene-,
*—P(═O)(OH)—O—P(═O)(OH)—(O)-alkylene-U-alkylene-,
*—P(═O)(OH)—O—P(═O)(OH)—(O)-alkylene-(O-alkylene)ₙ-U-alkylene-,
*—P(═O)(OH)—O—P(═O)(OH)—(O)-alkylene-(O-alkylene)ₙ-U-alkylene-,
*—P(═O)(OH)—O—P(═O)(OH)—(O)-alkylene-U-(alkylene-O)ₙ-alkylene-;
wherein:
each U is independently:

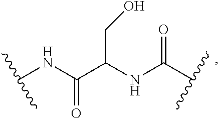 [SMILES: *NC(=O)C(CO)NC(*)=O]

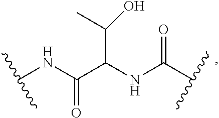 [SMILES: *NC(=O)C(NC(*)=O)C(C)O]

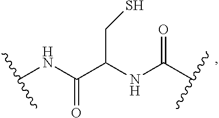 [SMILES: *NC(=O)C(CS)NC(*)=O]

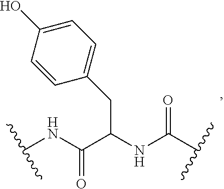 [SMILES: *NC(=O)C(Cc1ccc(O)cc1)NC(*)=O]

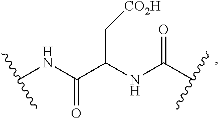 [SMILES: *NC(=O)C(CC(=O)O)NC(*)=O]

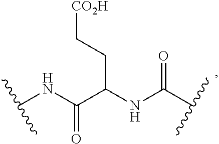 [SMILES: *NC(=O)C(CCC(=O)O)NC(*)=O]

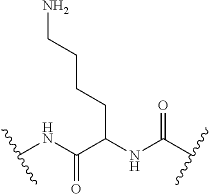 [SMILES: *NC(=O)C(CCCCN)NC(*)=O]

or

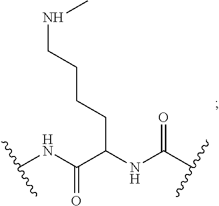 [SMILES: *NC(=O)C(CCCCNC)NC(*)=O]

each J is independently:

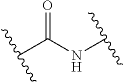 [SMILES: *NC(*)=O]

or

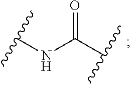 [SMILES: *NC(*)=O]

each alkylene is independently selected from the group consisting of:
—(CH₂)—, —(CH₂)₂—, —(CH₂)₃—, —(CH₂)₄—, —(CH₂)₅—, —(CH₂)₆—, —(CH₂)₇—,
—(CH₂)₈—, —(CH₂)₉—, —(CH₂)₁₀—, —(CH₂)₁₁— and —(CH₂)₁₂—;
each n is independently an integer from 1 to 100; optionally, each n is
independently an integer from 1 to 10; and
* denotes the connection to drug; and
wherein each linker is optionally substituted with at least one water-soluble polymer.

In some embodiments, each n of Table 8 is independently an integer from 1 to 10. In some embodiments, each n of Table 8 is independently 1, 2 or 3.

In some embodiments, each alkylene of Table 8 is independently —(CH₂)—, —(CH2)₂— or —(CH2)₃—In some embodiments, each linker of Table 8 is substituted with one or more water-soluble polymer. In some embodiments, each U of Table 8 is substituted with one or more water-soluble polymers. In some embodiments, each U of Table 8 is substituted with one water-soluble polymer.

In some embodiments, a water-soluble polymer is conjugated to the amino acid side chain of group U. In some embodiments, a water-soluble polymer is conjugated to the amino acid side chain of group U via a spacer element.

In some embodiments, the linker is: *—P(═O)(OH)—O—P(═O)(OH)—(O)-alkylene-U-alkylene-+,

wherein:
U is selected from the group consisting of:

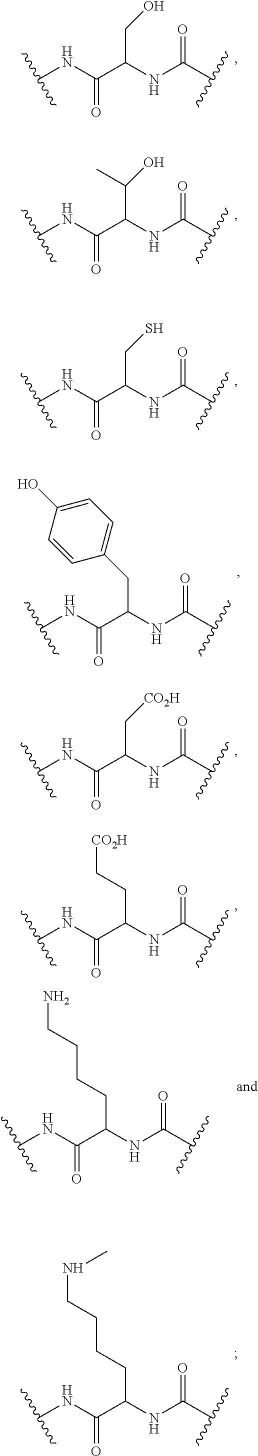

each alkylene is independently selected from the group consisting of:

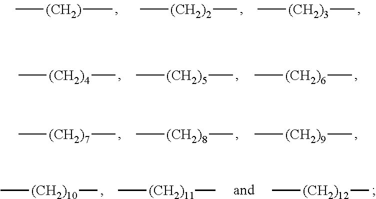

* denotes a point of connection to a drug, for example, the —O— atom of Formula (I), or Formula (Ia), or Formula (Ib), or Formula (Ic) or Formula I(d); and
+ denotes a point of connection to a moiety, such as a reactive moiety;
   wherein each said linker is optionally substituted with one or more water-soluble polymers.

In some embodiments, the linker is substituted with the one or more water-soluble polymers.

In some embodiments, the linker comprises one water-soluble polymer. In some embodiments, the one water-soluble polymer is conjugated to an amino acid side chain of group U. In some embodiments, the one water-soluble polymer is conjugated to the amino acid side chain of group U via a spacer element.

In some embodiments, the water-soluble polymer is a polysaccharide.

In some embodiments, the water-soluble polymer is a PEG moiety. In some embodiments, the PEG moiety has a molecular weight within a range of about 100 Da to about 100,000 Da. In some embodiments, the PEG moiety has a molecular weight within a range of about 100 Da to about 10,000 Da. In some embodiments, the PEG moiety has a molecular weight within a range of about 100 Da to about 5,000 Da. In some embodiments, the PEG moiety has a molecular weight within a range of about 100 Da to about 1,000 Da.

In some embodiments, the PEG moiety is —(CH₂CH₂O)ₙCH₃, wherein n is an integer from 1 to 100. In some embodiments, the PEG moiety is —(CH₂CH₂O)ₙCH₃, wherein n is an integer from 1 to 24. In some embodiments, the PEG moiety is —(CH₂CH₂O)ₙCH₃, wherein n is an integer from 6 to 12. In some embodiments, the PEG moiety is —(CH₂CH₂O)ₙCH₃, wherein n is an integer from 8 to 12. In some embodiments, the PEG moiety is —(CH₂CH₂O)ₙCH₃, wherein n is 8. In some embodiments, the PEG moiety is —(CH₂CH₂O)ₙCH₃, wherein n is 12.

In some embodiments, the PEG is linear. In some embodiments, the PEG is branched, multi-armed or dendritic.

In some embodiments, U is:

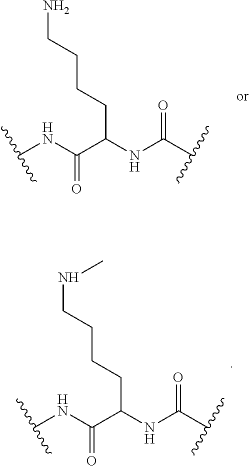 [SMILES: *NC(=O)C(CCCCN)NC(*)=O ; *NC(=O)C(CCCCNC)NC(*)=O]

In some embodiments, U is

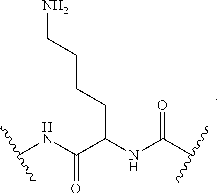 [SMILES: *NC(=O)C(CCCCN)NC(*)=O]

In some other embodiments, U is

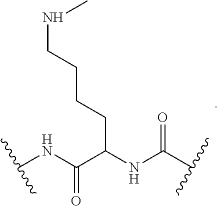 [SMILES: *NC(=O)C(CCCCNC)NC(*)=O]

In some embodiments each said U is conjugated to a water-soluble polymer. In some embodiments, the water-soluble polymer is conjugated to the amino acid side chain of group U.

In some embodiments, the water-soluble polymer is conjugated to the amino acid side chain of group U via a spacer element. In some embodiments, when the water-soluble polymer is conjugated to the amino acid side chain of group U via a spacer element, the spacer element is a carbonyl group.

In some embodiments, the linker has the following structure:

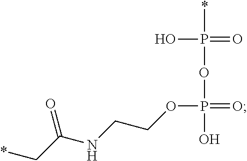 [SMILES: *CC(=O)NCCOP(=O)(O)OP(*)(=O)O]

wherein: * denotes a point of connection to a drug, for example, the —O— atom of Formula (I), or Formula (Ia), or Formula (Ib), or Formula (Ic) or Formula I(d); and + denotes a point of connection to a reactive moiety.

In some embodiments, the linker has the following structure:

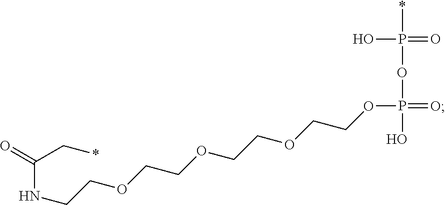 [SMILES: *CC(=O)NCCOCCOCCOCCOP(=O)(O)OP(*)(=O)O]

wherein: * denotes a point of connection to a drug; for example, the —O— atom of Formula (I), or Formula (Ia), or Formula (Ib), or Formula (Ic) or Formula I(d); and + denotes a point of connection to a reactive moiety.

In some other embodiments, the linker has the following structure:

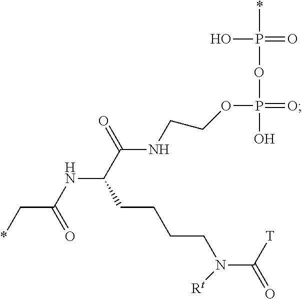

wherein: T is the water-soluble polymer; R^{t} is H or methyl; * denotes a point of connection to a drug; for example, the —O— atom of Formula (I), or Formula (Ia), or Formula (Ib), or Formula (Ic) or Formula I(d); and + denotes a point of connection to a reactive moiety.

In some embodiments, the one or more water-soluble polymer conjugated to a linker is a (polyethylene)glycol (PEG) moiety.

In some embodiments, the linker one water-soluble polymer is conjugated to an amino acid side chain of a linker comprising group U. In some embodiments, the one water-soluble polymer is conjugated to the amino acid side chain of group U via a spacer element.

In some embodiments, the water-soluble polymer is a polysaccharide.

In some embodiments, the water-soluble polymer is a PEG moiety. In some embodiments, the PEG moiety has a molecular weight within a range of about 100 Da to about 100,000 Da. In some embodiments, the PEG moiety has a molecular weight within a range of about 100 Da to about 10,000 Da. In some embodiments, the PEG moiety has a molecular weight within a range of about 100 Da to about 5,000 Da. In some embodiments, the PEG moiety has a molecular weight within a range of about 100 Da to about 1,000 Da.

In some embodiments, the PEG moiety is —(CH₂CH₂O)ₙCH₃, wherein n is an integer from 1 to 100. In some embodiments, the PEG moiety is —(CH₂CH₂O)ₙCH₃, wherein n is an integer from 1 to 24. In some embodiments, the PEG moiety is —(CH₂CH₂O)ₙCH₃, wherein n is an integer from 6 to 12. In some embodiments, the PEG moiety is —(CH₂CH₂O)ₙCH₃, wherein n is an integer from 8 to 12. In some embodiments, the PEG moiety is —(CH₂CH₂O)ₙCH₃, wherein n is 8. In some embodiments, the PEG moiety is —(CH₂CH₂O)ₙCH₃, wherein n is 12.

In some embodiments, the PEG is linear. In some embodiments, the PEG is branched, multi-armed or dendritic.

In some embodiments, a phosphate-based linker of the present disclosure is connected to a drug, and is also connected to a reactive moiety. Thus, the linker bridges the drug and the reactive moiety. The reactive moiety can be one that can react with another moiety of a natural amino acid or non-natural amino acid of a polypeptide, such as an antibody, antibody fragment or variant thereof of the present disclosure, as disclosed herein.

In some other embodiments, a phosphate-based linker as disclosed herein is connected to a drug, and is also connected to an antibody, antibody fragment or variant thereof, via a linkage or adduct moiety. Thus, the linker bridges the drug and the antibody, antibody fragment or variant thereof.

### Drugs and Drug-Linkers

In some aspects, the present disclosure provides a drug or drug-linker, wherein the drug is a cytotoxic drug or agent. In some aspects of the disclosure, the cytotoxic drug is a duocarmycin. In some aspects, the cytotoxic drug is a duocarmycin analog. In some embodiments the drug or drug-linker is a drug or drug-linker generated as described in the Examples herein, wherein the linker, when present, can be derivatized with a reactive or other moiety; or a metabolite thereof.

In some aspects, there is provided a cytotoxic drug which is a duocarmycin analog of Formula (X) having the following general structure:

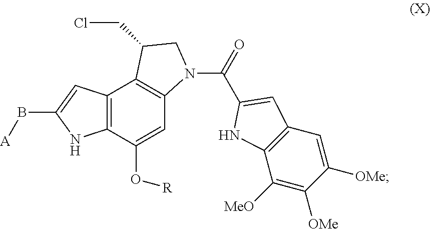

wherein:
A is an optionally substituted bicyclic ring system containing one or more nitrogen ring atoms; B is a carbonyl group; and R is H or L-W, wherein L is a linker and W is a reactive moiety; or a salt thereof. In some embodiments, the salt can be a pharmaceutically acceptable salt. In some embodiments, the bicyclic ring system A is joined to the carbonyl group B via one of the one or more nitrogen ring atoms of the bicyclic ring system A.

In some embodiments, the bicyclic ring system A can contain 9 ring atoms.

In some other embodiments, the bicyclic ring system A can contain 10 ring atoms.

In some embodiments, the bicyclic ring system A can contain 9 or 10 ring atoms, wherein the ring atoms are selected from the group consisting of carbon atoms and nitrogen atoms.

In some embodiments, the bicyclic ring system A contains a 5-membered ring fused to a 6-membered ring, wherein the 5-membered ring contains the nitrogen atom that joins the bicyclic ring system A to the carbonyl group B. In some embodiments, the 5-membered ring is a pyrrolidine ring. In some embodiments, the 6-membered ring is an aromatic ring containing 0 or 1 nitrogen atoms.

In some embodiments, the bicyclic ring system A contains a first 6-membered ring fused to a second 6-membered ring, wherein the first 6-membered ring contains the nitrogen atom that joins the bicyclic ring system A to the carbonyl group B. In some embodiments, the first 6-membered ring is a piperidine ring. In some embodiments, the second 6-membered ring is an aromatic ring containing 0 or 1 nitrogen atoms.

In some embodiments, bicyclic ring system A is hydrophobic. As is understood by a person of ordinary skill in the art, the hydrophobicity of a compound can be estimated from its C Log P value, which can be calculated from its structure; lower C Log P values are indicative of a more hydrophilic molecule, and higher C Log P values are indicative of a more hydrophobic molecule. Thus, in some embodiments, the hydrophobicity of A is characterized by its C log P value. In some embodiments, the hydrophobicity of A is characterized by the C log P value of its corresponding amine “A-H.”

In some embodiments, A-H (the corresponding amine of A) has a C log P value of at least about 1. C Log P values can be calculated using tools such as ChemDraw Professional Software (PerkinElmer Informatics). ChemDraw Professional Version 20.1.1.125 was used to calculate the C Log P values reported below for some non-limiting examples of A-H groups of the present disclosure.

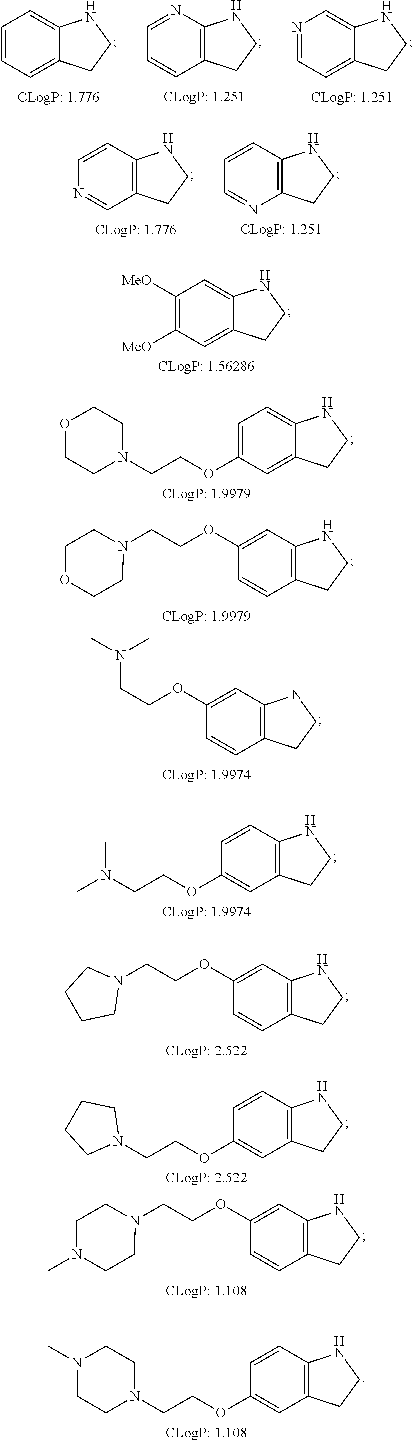

In some embodiments, R is H.

In some embodiments, the compound is a drug-linker compound, and R is L-W. In some embodiments, L is a phosphate-based linker.

Thus, in some embodiments, there is provided a compound of Formula (I), having the following structure:

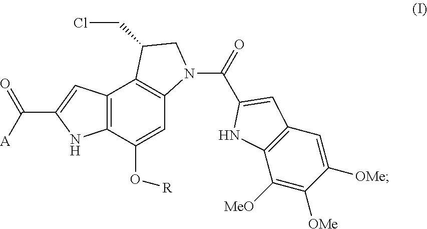

wherein:
R is H or L-W, wherein L is a linker and W is a reactive moiety;
A is a bicyclic ring system selected from the group consisting of formula (a), (b), (c) and (d), having the following structures:

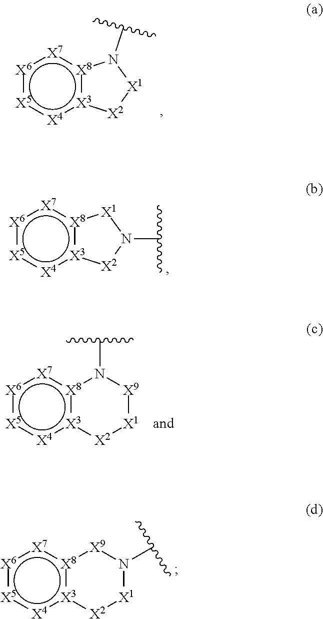

wherein:
   each X¹ is C(R^{1a})(R^{1b}); wherein each R^{1a} and R^{1b} is independently H, halogen, alkyl, alkenyl or alkynyl;
   each X² is C(R^{2a})(R^{2b}); wherein each R^{2a} and R^{2b} is independently H, halogen, alkyl, alkenyl or alkynyl;
   each X³ is C;
   each X⁴ is C(R⁴) or N, wherein R⁴ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
   each X⁵ is C(R⁵) or N, wherein R⁵ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
   each X⁶ is C(R⁶) or N, wherein R⁶ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
   each X⁷ is C(R⁷) or N, wherein R⁷ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
   each X⁸ is C; and
   each X⁹, when present, is C(R^{9a})(R^{9b}); wherein each R^{9a} and R^{9b} is independently H, halogen, alkyl, alkenyl or alkynyl;
      wherein:
each R^{a} and R^{b} is independently H, alkyl, alkenyl or alkynyl;
each R^{c} is independently H, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl or heteroarylalkyl;
each R^{s} is independently H, halogen, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl or heteroarylalkyl; and
each m is independently 0, 1, 2 or 3;
   or a salt thereof.

In some embodiments, A-H (the corresponding amine of moiety A) has a C log P value of at least about 1. In some embodiments, A has the structure of formula (a), and A-H has a C log P value of at least about 1. In some embodiments, A has the structure of formula (b), and A-H has a C log P value of at least about 1. In some embodiments, A has the structure of formula (c), and A-H has a C log P value of at least about 1. In some embodiments, A has the structure of formula (d), and A-H has a C log P value of at least about 1.

In some embodiments, X¹ is C(R^{1a})(R^{1b}); wherein each R^{1a} and R^{1b} is independently H, halogen or unsubstituted alkyl; X² is C(R^{2a})(R^{2b}); wherein each R^{2a} and R^{2b} is independently H, halogen or unsubstituted alkyl; X³ is C; X⁴ is C(R⁴) or N, wherein R⁴ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl; X⁵ is C(R⁵) or N, wherein R⁵ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl; X⁶ is C(R⁶) or N, wherein R⁶ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl; X⁷ is C(R⁷) or N, wherein R⁷ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl; X⁸ is C; and X⁹, when present, is C(R^{9a})(R^{9b}); wherein each R^{9a} and R^{9b} is independently H, halogen or unsubstituted alkyl.

In some embodiments, each X¹ and X² is CH₂; and X⁹, when present, is CH₂.

In some embodiments, X⁴ is C(R⁴) or N, wherein R⁴ is H, halogen or heteroalkyl; X⁵ is C(R⁵) or N, wherein R⁵ is H, halogen or heteroalkyl; X⁶ is C(R⁶) or N, wherein R⁶ is H, halogen or heteroalkyl; and X⁷ is C(R⁷) or N, wherein R⁷ is H, halogen or heteroalkyl.

In some embodiments, X⁴ is C(R⁴) or N, wherein R⁴ is H or heteroalkyl; X⁵ is C(R⁵) or N, wherein R⁵ is H or heteroalkyl; X⁶ is C(R⁶) or N, wherein R⁶ is H or heteroalkyl; and X⁷ is C(R⁷) or N, wherein R⁷ is H or heteroalkyl.

In some embodiments, each heteroalkyl is an alkoxy. In some embodiments, each said alkoxy is independently —OR^{k}, wherein each R^{k} is independently alkyl optionally substituted with —N(R^{d})(R^{e}) or heterocyclyl; wherein each R^{d} and R^{c} is independently H, alkyl, alkenyl or alkynyl.

In some embodiments, each heterocyclyl contains at least one nitrogen atom.

In some embodiments, X⁴ is N, X⁵ is C(R⁵), X⁶ is C(R⁶) and X⁷ is C(R⁷). In some embodiments, X⁷ is CH.

In some embodiments, X⁴ is C(R⁴), X⁵ is N, X⁶ is C(R⁶) and X⁷ is C(R⁷). In some embodiments, at least one of X⁴ and X⁷ is CH.

In some embodiments, X⁴ is C(R⁴), X⁵ is C(R⁵), X⁶ is N and X⁷ is C(R⁷). In some embodiments, at least one of X⁴ and X⁷ is CH.

In some embodiments, X⁴ is C(R⁴), X⁵ is C(R⁵), X⁶ is C(R⁶) and X⁷ is N. In some embodiments, X⁴ is CH.

In some embodiments, X⁴ is C(R⁴), X⁵ is C(R⁵), X⁶ is C(R⁶) and X⁷ is C(R⁷).

In some embodiments, X⁷ is CH. In some embodiments, X⁴ is CH. In some embodiments, each of X⁴ and X⁷ is CH.

In some embodiments, R is H.

In some embodiments, there is provided a compound of Formula (Ia) having the following structure:

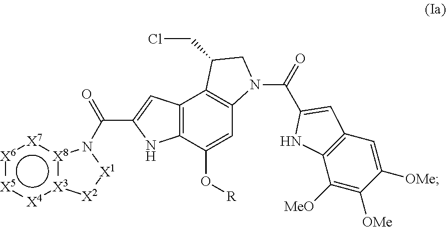

wherein:
R is H or L-W, wherein L is a linker and W is a reactive moiety;
X¹ is C(R^{1a})(R^{1b}); wherein each R^{1a} and R^{1b} is independently H, halogen, alkyl, alkenyl or alkynyl;
X² is C(R^{2a})(R^{2b}); wherein each R^{2a} and R^{2b} is independently H, halogen, alkyl, alkenyl or alkynyl;
X³ is C;
X⁴ is C(R⁴) or N, wherein R⁴ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
X⁵ is C(R⁵) or N, wherein R⁵ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
X⁶ is C(R⁶) or N, wherein R⁶ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
X⁷ is C(R⁷) or N, wherein R⁷ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s}); and
X⁸ is C;
   wherein:
each R^{a} and R^{b} is independently H, alkyl, alkenyl or alkynyl;
each R^{c} is independently H, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl or heteroarylalkyl;
each R^{s} is independently H, halogen, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl or heteroarylalkyl; and
each m is independently 0, 1, 2 or 3.

In some embodiments, X¹ is C(R^{1a})(R^{1b}); wherein each R^{1a} and R^{1b} is independently H, halogen or unsubstituted alkyl; X² is C(R^{2a})(R^{2b}); wherein each R^{2a} and R^{2b} is independently H, halogen or unsubstituted alkyl; X³ is C; X⁴ is C(R⁴) or N, wherein R⁴ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl; X⁵ is C(R⁵) or N, wherein R⁵ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl; X⁶ is C(R⁶) or N, wherein R⁶ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl; X⁷ is C(R⁷) or N, wherein R⁷ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl; X⁸ is C; and X⁹, when present, is C(R^{9a})(R^{9b}); wherein each R^{9a} and R^{9b} is independently H, halogen or unsubstituted alkyl.

In some embodiments, each X¹ and X² is CH₂; and X⁹, when present, is CH₂.

In some embodiments, X⁴ is C(R⁴) or N, wherein R⁴ is H, halogen or heteroalkyl; X⁵ is C(R⁵) or N, wherein R⁵ is H, halogen or heteroalkyl; X⁶ is C(R⁶) or N, wherein R⁶ is H, halogen or heteroalkyl; and X⁷ is C(R⁷) or N, wherein R⁷ is H, halogen or heteroalkyl.

In some embodiments, X⁴ is C(R⁴) or N, wherein R⁴ is H or heteroalkyl; X⁵ is C(R⁵) or N, wherein R⁵ is H or heteroalkyl; X⁶ is C(R⁶) or N, wherein R⁶ is H or heteroalkyl; and X⁷ is C(R⁷) or N, wherein R⁷ is H or heteroalkyl.

In some embodiments, each heteroalkyl is an alkoxy. In some embodiments, each said alkoxy is independently —OR^{k}, wherein each R^{k} is independently alkyl optionally substituted with —N(R^{d})(R^{e}) or heterocyclyl; wherein each R^{d} and R^{c} is independently H, alkyl, alkenyl or alkynyl.

In some embodiments, each heterocyclyl contains at least one nitrogen atom.

In some embodiments, X⁴ is N, X⁵ is C(R⁵), X⁶ is C(R⁶) and X⁷ is C(R⁷). In some embodiments, X⁷ is CH.

In some embodiments, X⁴ is C(R⁴), X⁵ is N, X⁶ is C(R⁶) and X⁷ is C(R⁷). In some embodiments, at least one of X⁴ and X⁷ is CH.

In some embodiments, X⁴ is C(R⁴), X⁵ is C(R⁵), X⁶ is N and X⁷ is C(R⁷). In some embodiments, at least one of X⁴ and X⁷ is CH.

In some embodiments, X⁴ is C(R⁴), X⁵ is C(R⁵), X⁶ is C(R⁶) and X⁷ is N. In some embodiments, X⁴ is CH.

In some embodiments, X⁴ is C(R⁴), X⁵ is C(R⁵), X⁶ is C(R⁶) and X⁷ is C(R⁷).

In some embodiments, X⁷ is CH. In some embodiments, X⁴ is CH. In some embodiments, each of X⁴ and X⁷ is CH.

In some embodiments, R is H.

In some embodiments, there is provided a compound of Formula (Ib) having the following structure:

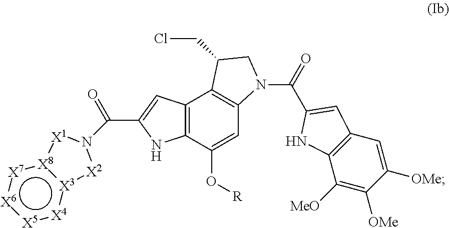

wherein:
R is H or L-W, wherein L is a linker and W is a reactive moiety;
X¹ is C(R^{1a})(R^{1b}); wherein each R^{1a} and R^{1b} is independently H, halogen, alkyl, alkenyl or alkynyl;
X² is C(R^{2a})(R^{2b}); wherein each R^{2a} and R^{2b} is independently H, halogen, alkyl, alkenyl or alkynyl;
X³ is C;
X⁴ is C(R⁴) or N, wherein R⁴ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
X⁵ is C(R⁵) or N, wherein R⁵ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
X⁶ is C(R⁶) or N, wherein R⁶ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
X⁷ is C(R⁷) or N, wherein R⁷ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s}); and
X⁸ is C;
   wherein:
each R^{a} and R^{b} is independently H, alkyl, alkenyl or alkynyl;
each R^{c} is independently H, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl or heteroarylalkyl;
each R^{s} is independently H, halogen, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl or heteroarylalkyl; and
each m is independently 0, 1, 2 or 3.

In some embodiments, X¹ is C(R^{1a})(R^{1b}); wherein each R^{1a} and R^{1b} is independently H, halogen or unsubstituted alkyl; X² is C(R^{2a})(R^{2b}); wherein each R^{2a} and R^{2b} is independently H, halogen or unsubstituted alkyl; X³ is C; X⁴ is C(R⁴) or N, wherein R⁴ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl; X⁵ is C(R⁵) or N, wherein R⁵ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl; X⁶ is C(R⁶) or N, wherein R⁶ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl; X⁷ is C(R⁷) or N, wherein R⁷ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl; X⁸ is C; and X⁹, when present, is C(R^{9a})(R^{9b}); wherein each R^{9a} and R^{9b} is independently H, halogen or unsubstituted alkyl.

In some embodiments, each X¹ and X² is CH₂; and X⁹, when present, is CH₂.

In some embodiments, X⁴ is C(R⁴) or N, wherein R⁴ is H, halogen or heteroalkyl; X⁵ is C(R⁵) or N, wherein R⁵ is H, halogen or heteroalkyl; X⁶ is C(R⁶) or N, wherein R⁶ is H, halogen or heteroalkyl; and X⁷ is C(R⁷) or N, wherein R⁷ is H, halogen or heteroalkyl.

In some embodiments, X⁴ is C(R⁴) or N, wherein R⁴ is H or heteroalkyl; X⁵ is C(R⁵) or N, wherein R⁵ is H or heteroalkyl; X⁶ is C(R⁶) or N, wherein R⁶ is H or heteroalkyl; and X⁷ is C(R⁷) or N, wherein R⁷ is H or heteroalkyl.

In some embodiments, each heteroalkyl is an alkoxy. In some embodiments, each said alkoxy is independently —OR^{k}, wherein each R^{k} is independently alkyl optionally substituted with —N(R^{d})(R^{e}) or heterocyclyl; wherein each R^{d} and R^{e} is independently H, alkyl, alkenyl or alkynyl.

In some embodiments, each heterocyclyl contains at least one nitrogen atom.

In some embodiments, X⁴ is N, X⁵ is C(R⁵), X⁶ is C(R⁶) and X⁷ is C(R⁷). In some embodiments, X⁷ is CH.

In some embodiments, X⁴ is C(R⁴), X⁵ is N, X⁶ is C(R⁶) and X⁷ is C(R⁷). In some embodiments, at least one of X⁴ and X⁷ is CH.

In some embodiments, X⁴ is C(R⁴), X⁵ is C(R⁵), X⁶ is N and X⁷ is C(R⁷). In some embodiments, at least one of X⁴ and X⁷ is CH.

In some embodiments, X⁴ is C(R⁴), X⁵ is C(R⁵), X⁶ is C(R⁶) and X⁷ is N. In some embodiments, X⁴ is CH.

In some embodiments, X⁴ is C(R⁴), X⁵ is C(R⁵), X⁶ is C(R⁶) and X⁷ is C(R⁷).

In some embodiments, X⁷ is CH. In some embodiments, X⁴ is CH. In some embodiments, each of X⁴ and X⁷ is CH.

In some embodiments, R is H.

In some embodiments, there is provided a compound of Formula (Ic) having the following structure:

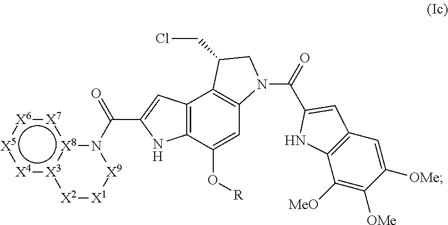

wherein:
R is H or L-W, wherein L is a linker and W is a reactive moiety;
X¹ is C(R^{1a})(R^{1b}); wherein each R^{1a} and R^{1b} is independently H, halogen, alkyl, alkenyl or alkynyl;
X² is C(R^{2a})(R^{2b}); wherein each R^{2a} and R^{2b} is independently H, halogen, alkyl, alkenyl or alkynyl;
X³ is C;
X⁴ is C(R⁴) or N, wherein R⁴ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
X⁵ is C(R⁵) or N, wherein R⁵ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
X⁶ is C(R⁶) or N, wherein R⁶ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
X⁷ is C(R⁷) or N, wherein R⁷ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
X⁸ is C; and
X⁹ is C(R^{9a})(R^{9b}); wherein each R^{9a} and R^{9b} is independently H, halogen, alkyl, alkenyl or alkynyl;
   wherein:
each R^{a} and R^{b} is independently H, alkyl, alkenyl or alkynyl;
each R^{c} is independently H, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl or heteroarylalkyl;
each R^{s} is independently H, halogen, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl or heteroarylalkyl; and
each m is independently 0, 1, 2 or 3.

In some embodiments, X¹ is C(R^{1a})(R^{1b}); wherein each R^{1a} and R^{1b} is independently H, halogen or unsubstituted alkyl; X² is C(R^{2a})(R^{2b}); wherein each R^{2a} and R^{2b} is independently H, halogen or unsubstituted alkyl; X³ is C; X⁴ is C(R⁴) or N, wherein R⁴ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl; X⁵ is C(R⁵) or N, wherein R⁵ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl; X⁶ is C(R⁶) or N, wherein R⁶ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl; X⁷ is C(R⁷) or N, wherein R⁷ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl; X⁸ is C; and X⁹, when present, is C(R^{9a})(R^{9b}); wherein each R^{9a} and R^{9b} is independently H, halogen or unsubstituted alkyl.

In some embodiments, each X¹ and X² is CH₂; and X⁹, when present, is CH₂.

In some embodiments, X⁴ is C(R⁴) or N, wherein R⁴ is H, halogen or heteroalkyl; X⁵ is C(R⁵) or N, wherein R⁵ is H, halogen or heteroalkyl; X⁶ is C(R⁶) or N, wherein R⁶ is H, halogen or heteroalkyl; and X⁷ is C(R⁷) or N, wherein R⁷ is H, halogen or heteroalkyl.

In some embodiments, X⁴ is C(R⁴) or N, wherein R⁴ is H or heteroalkyl; X⁵ is C(R⁵) or N, wherein R⁵ is H or heteroalkyl; X⁶ is C(R⁶) or N, wherein R⁶ is H or heteroalkyl; and X⁷ is C(R⁷) or N, wherein R⁷ is H or heteroalkyl.

In some embodiments, each heteroalkyl is an alkoxy. In some embodiments, each said alkoxy is independently —OR^{k}, wherein each R^{k} is independently alkyl optionally substituted with —N(R^{d})(R^{e}) or heterocyclyl; wherein each R^{d} and R^{e} is independently H, alkyl, alkenyl or alkynyl.

In some embodiments, each heterocyclyl contains at least one nitrogen atom.

In some embodiments, X⁴ is N, X⁵ is C(R⁵), X⁶ is C(R⁶) and X⁷ is C(R⁷). In some embodiments, X⁷ is CH.

In some embodiments, X⁴ is C(R⁴), X⁵ is N, X⁶ is C(R⁶) and X⁷ is C(R⁷). In some embodiments, at least one of X⁴ and X⁷ is CH.

In some embodiments, X⁴ is C(R⁴), X⁵ is C(R⁵), X⁶ is N and X⁷ is C(R⁷). In some embodiments, at least one of X⁴ and X⁷ is CH.

In some embodiments, X⁴ is C(R⁴), X⁵ is C(R⁵), X⁶ is C(R⁶) and X⁷ is N. In some embodiments, X⁴ is CH.

In some embodiments, X⁴ is C(R⁴), X⁵ is C(R⁵), X⁶ is C(R⁶) and X⁷ is C(R⁷).

In some embodiments, X⁷ is CH. In some embodiments, X⁴ is CH. In some embodiments, each of X⁴ and X⁷ is CH.

In some embodiments, R is H.

In some embodiments, there is provided a compound of Formula (Id) having the following structure:

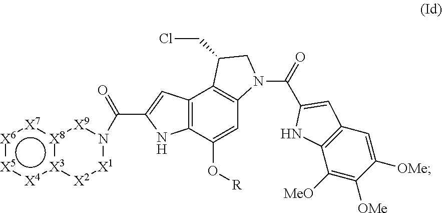

wherein:
R is H or L-W, wherein L is a linker and W is a reactive moiety;
X¹ is C(R^{1a})(R^{1b}); wherein each R^{1a} and R^{1b} is independently H, halogen, alkyl, alkenyl or alkynyl;
X² is C(R^{2a})(R^{2b}); wherein each R^{2a} and R^{2b} is independently H, halogen, alkyl, alkenyl or alkynyl;
X³ is C;
X⁴ is C(R⁴) or N, wherein R⁴ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
X⁵ is C(R⁵) or N, wherein R⁵ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
X⁶ is C(R⁶) or N, wherein R⁶ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
X⁷ is C(R⁷) or N, wherein R⁷ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
X⁸ is C; and
X⁹ is C(R^{9a})(R^{9b}); wherein each R^{9a} and R^{9b} is independently H, halogen, alkyl, alkenyl or alkynyl;
   wherein:
each R^{a} and R^{b} is independently H, alkyl, alkenyl or alkynyl;
each R^{c} is independently H, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl or heteroarylalkyl;
each R^{s} is independently H, halogen, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl or heteroarylalkyl; and
each m is independently 0, 1, 2 or 3.

In some embodiments, X¹ is C(R^{1a})(R^{1b}); wherein each R^{1a} and R^{1b} is independently H, halogen or unsubstituted alkyl; X² is C(R^{2a})(R^{2b}); wherein each R^{2a} and R^{2b} is independently H, halogen or unsubstituted alkyl; X³ is C; X⁴ is C(R⁴) or N, wherein R⁴ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl; X⁵ is C(R⁵) or N, wherein R⁵ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl; X⁶ is C(R⁶) or N, wherein R⁶ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl; X⁷ is C(R⁷) or N, wherein R⁷ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl; X⁸ is C; and X⁹, when present, is C(R^{9a})(R^{9b}); wherein each R^{9a} and R^{9b} is independently H, halogen or unsubstituted alkyl.

In some embodiments, each X¹ and X² is CH₂; and X⁹, when present, is CH₂.

In some embodiments, X⁴ is C(R⁴) or N, wherein R⁴ is H, halogen or heteroalkyl; X⁵ is C(R⁵) or N, wherein R⁵ is H, halogen or heteroalkyl; X⁶ is C(R⁶) or N, wherein R⁶ is H, halogen or heteroalkyl; and X⁷ is C(R⁷) or N, wherein R⁷ is H, halogen or heteroalkyl.

In some embodiments, X⁴ is C(R⁴) or N, wherein R⁴ is H or heteroalkyl; X⁵ is C(R⁵) or N, wherein R⁵ is H or heteroalkyl; X⁶ is C(R⁶) or N, wherein R⁶ is H or heteroalkyl; and X⁷ is C(R⁷) or N, wherein R⁷ is H or heteroalkyl.

In some embodiments, each heteroalkyl is an alkoxy. In some embodiments, each said alkoxy is independently —OR^{k}, wherein each R^{k} is independently alkyl optionally substituted with —N(R^{d})(R^{e}) or heterocyclyl; wherein each R^{d} and R^{e} is independently H, alkyl, alkenyl or alkynyl.

In some embodiments, each heterocyclyl contains at least one nitrogen atom.

In some embodiments, X⁴ is N, X⁵ is C(R⁵), X⁶ is C(R⁶) and X⁷ is C(R⁷). In some embodiments, X⁷ is CH.

In some embodiments, X⁴ is C(R⁴), X⁵ is N, X⁶ is C(R⁶) and X⁷ is C(R⁷). In some embodiments, at least one of X⁴ and X⁷ is CH.

In some embodiments, X⁴ is C(R⁴), X⁵ is C(R⁵), X⁶ is N and X⁷ is C(R⁷). In some embodiments, at least one of X⁴ and X⁷ is CH.

In some embodiments, X⁴ is C(R⁴), X⁵ is C(R⁵), X⁶ is C(R⁶) and X⁷ is N. In some embodiments, X⁴ is CH.

In some embodiments, X⁴ is C(R⁴), X⁵ is C(R⁵), X⁶ is C(R⁶) and X⁷ is C(R⁷).

In some embodiments, X⁷ is CH. In some embodiments, X⁴ is CH. In some embodiments, each of X⁴ and X⁷ is CH.

In some embodiments, R is H.

In some embodiments, there is provided a compound of Formula (I) wherein R is H. In some embodiments, there is provided a compound of Formula (Ia), wherein R is H. In some embodiments, the compound is selected from the group consisting of:

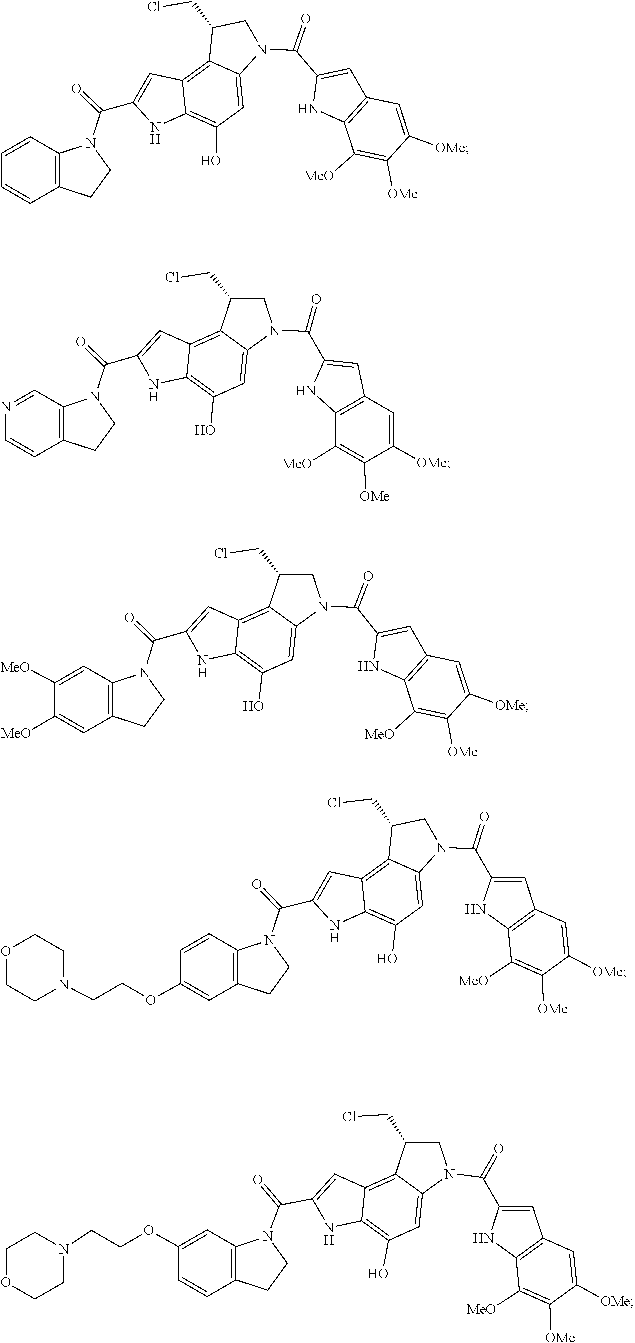 [SMILES: COc1cc2cc(C(=O)N3C[C@@H](CCl)c4c3cc(O)c3[nH]c(C(=O)N5CCc6ccccc65)cc43)[nH]c2c(OC)c1OC ; COc1cc2cc(C(=O)N3C[C@@H](CCl)c4c3cc(O)c3[nH]c(C(=O)N5CCc6ccncc65)cc43)[nH]c2c(OC)c1OC ; COc1cc2c(cc1OC)N(C(=O)c1cc3c4c(cc(O)c3[nH]1)N(C(=O)c1cc3cc(OC)c(OC)c(OC)c3[nH]1)C[C@H]4CCl)CC2 ; COc1cc2cc(C(=O)N3C[C@@H](CCl)c4c3cc(O)c3[nH]c(C(=O)N5CCc6cc(OCCN7CCOCC7)ccc65)cc43)[nH]c2c(OC)c1OC ; COc1cc2cc(C(=O)N3C[C@@H](CCl)c4c3cc(O)c3[nH]c(C(=O)N5CCc6ccc(OCCN7CCOCC7)cc65)cc43)[nH]c2c(OC)c1OC]

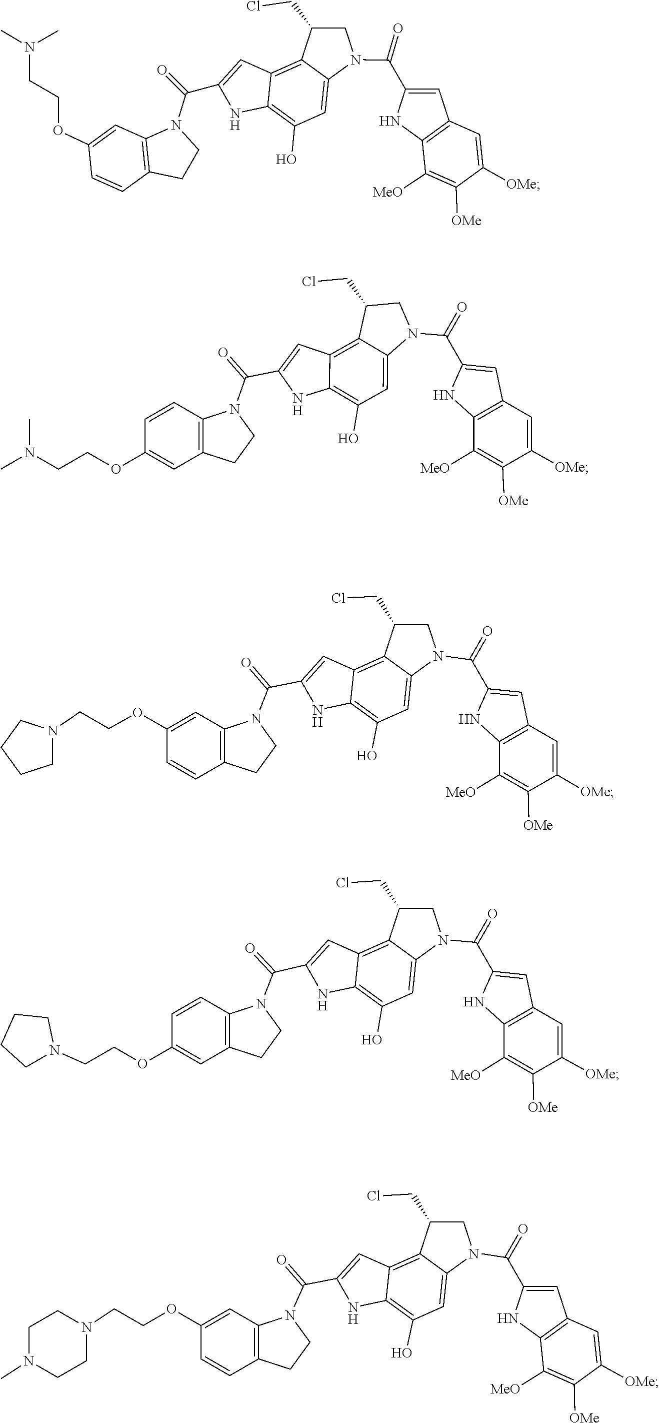 [SMILES: COc1cc2cc(C(=O)N3C[C@@H](CCl)c4c3cc(O)c3[nH]c(C(=O)N5CCc6ccc(OCCN(C)C)cc65)cc43)[nH]c2c(OC)c1OC ; COc1cc2cc(C(=O)N3C[C@@H](CCl)c4c3cc(O)c3[nH]c(C(=O)N5CCc6cc(OCCN(C)C)ccc65)cc43)[nH]c2c(OC)c1OC ; COc1cc2cc(C(=O)N3C[C@@H](CCl)c4c3cc(O)c3[nH]c(C(=O)N5CCc6ccc(OCCN7CCCC7)cc65)cc43)[nH]c2c(OC)c1OC ; COc1cc2cc(C(=O)N3C[C@@H](CCl)c4c3cc(O)c3[nH]c(C(=O)N5CCc6cc(OCCN7CCCC7)ccc65)cc43)[nH]c2c(OC)c1OC ; COc1cc2cc(C(=O)N3C[C@@H](CCl)c4c3cc(O)c3[nH]c(C(=O)N5CCc6ccc(OCCN7CCN(C)CC7)cc65)cc43)[nH]c2c(OC)c1OC]

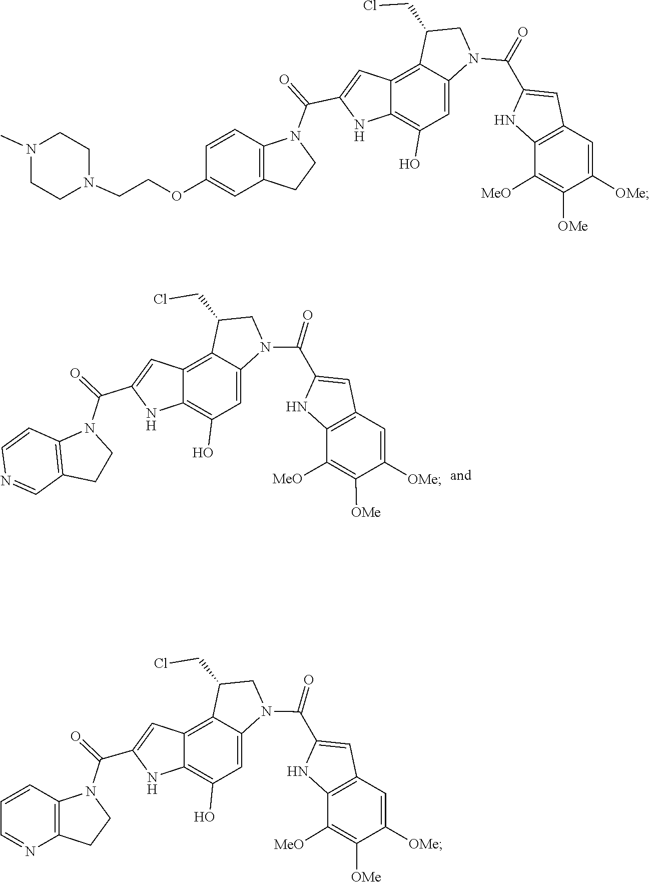 [SMILES: COc1cc2cc(C(=O)N3C[C@@H](CCl)c4c3cc(O)c3[nH]c(C(=O)N5CCc6cc(OCCN7CCN(C)CC7)ccc65)cc43)[nH]c2c(OC)c1OC ; COc1cc2cc(C(=O)N3C[C@@H](CCl)c4c3cc(O)c3[nH]c(C(=O)N5CCc6cnccc65)cc43)[nH]c2c(OC)c1OC ; COc1cc2cc(C(=O)N3C[C@@H](CCl)c4c3cc(O)c3[nH]c(C(=O)N5CCc6ncccc65)cc43)[nH]c2c(OC)c1OC]

and salts thereof. In some embodiments, the salt is a pharmaceutically acceptable salt.

In some embodiments, there is provided a compound of Formula (Ia) having the following structure:

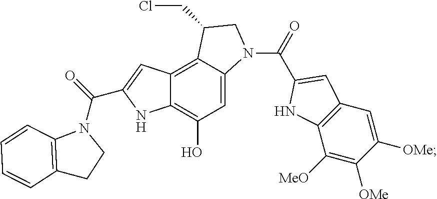 [SMILES: COc1cc2cc(C(=O)N3C[C@@H](CCl)c4c3cc(O)c3[nH]c(C(=O)N5CCc6ccccc65)cc43)[nH]c2c(OC)c1OC]

or a salt thereof.

In some embodiments, there is provided a compound of Formula (Ia) having the following structure:

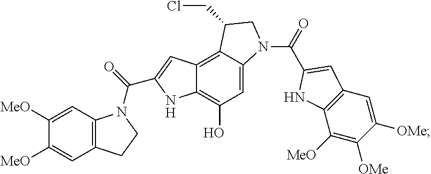 [SMILES: COc1cc2c(cc1OC)N(C(=O)c1cc3c4c(cc(O)c3[nH]1)N(C(=O)c1cc3cc(OC)c(OC)c(OC)c3[nH]1)C[C@H]4CCl)CC2]

or a salt thereof.

In some embodiments, there is provided a compound of Formula (Ia) having the following structure:

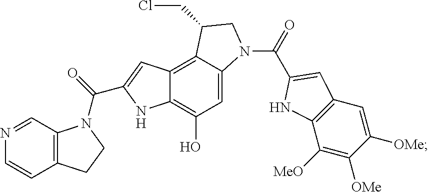 [SMILES: COc1cc2cc(C(=O)N3C[C@@H](CCl)c4c3cc(O)c3[nH]c(C(=O)N5CCc6ccncc65)cc43)[nH]c2c(OC)c1OC]

or a salt thereof.

In some embodiments, there is provided a compound of Formula (Ia) having the following structure:

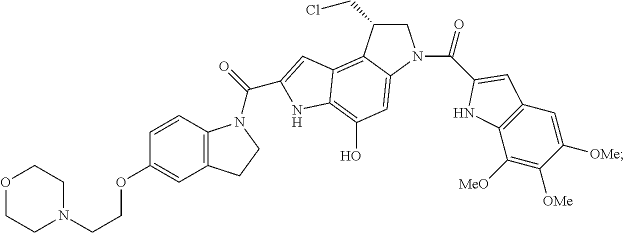 [SMILES: COc1cc2cc(C(=O)N3C[C@@H](CCl)c4c3cc(O)c3[nH]c(C(=O)N5CCc6cc(OCCN7CCOCC7)ccc65)cc43)[nH]c2c(OC)c1OC]

or a salt thereof.

In some aspects, there is provided a drug-linker compound of Formula (I) wherein R is L-W. Thus, in some embodiments, there is provided a compound of Formula (IL) having the following structure:

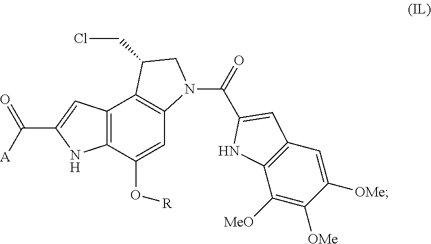

wherein:
R is L-W, wherein L is a linker and W is a reactive moiety;
A is a bicyclic ring system selected from the group consisting of formula (a), (b), (c) and (d), having the following structures:

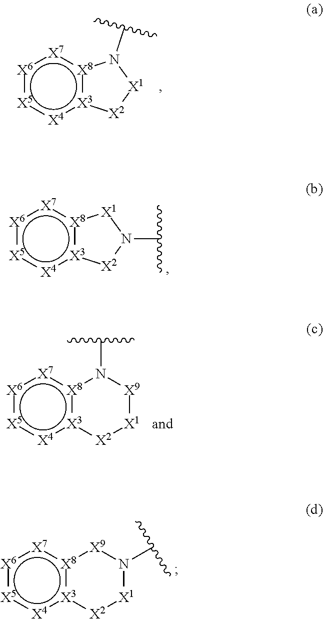

wherein:
   each X¹ is C(R^{1a})(R^{1b}); wherein each R^{1a} and R^{1b} is independently H, halogen, alkyl, alkenyl or alkynyl;
each X² is C(R^{2a})(R^{2b}); wherein each R^{2a} and R^{2b} is independently H, halogen, alkyl, alkenyl or alkynyl;
each X³ is C;
each X⁴ is C(R⁴) or N, wherein R⁴ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
each X⁵ is C(R⁵) or N, wherein R⁵ is H, halogen, —OH, —SH, —NO₂, —CN, —N₃, —N(R^{a})(R^{b}) acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
each X⁶ is C(R⁶) or N, wherein R⁶ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
each X⁷ is C(R⁷) or N, wherein R⁷ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
each X⁸ is C; and
each X⁹, when present, is C(R^{9a})(R^{9b}); wherein each R^{9a} and R^{9b} is independently H, halogen, alkyl, alkenyl or alkynyl;
   wherein:
each R^{a} and R^{b} is independently H, alkyl, alkenyl or alkynyl;
each R^{c} is independently H, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl or heteroarylalkyl;
each R^{s} is independently H, halogen, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl or heteroarylalkyl; and
each m is independently 0, 1, 2 or 3;
   or a salt thereof.

In some embodiments, A-H (the corresponding amine of moiety A) has a C log P value of at least about 1. In some embodiments, A has the structure of formula (a), and A-H has a C log P value of at least about 1. In some embodiments, A has the structure of formula (b), and A-H has a C log P value of at least about 1. In some embodiments, A has the structure of formula (c), and A-H has a C log P value of at least about 1. In some embodiments, A has the structure of formula (d), and A-H has a C log P value of at least about 1.

In some embodiments, X¹ is C(R^{1a})(R^{1b}); wherein each R^{1a} and R^{1b} is independently H, halogen or unsubstituted alkyl; X² is C(R^{2a})(R^{2b}); wherein each R^{2a} and R^{2b} is independently H, halogen or unsubstituted alkyl; X³ is C; X⁴ is C(R⁴) or N, wherein R⁴ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl; X⁵ is C(R⁵) or N, wherein R⁵ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl; X⁶ is C(R⁶) or N, wherein R⁶ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl; X⁷ is C(R⁷) or N, wherein R⁷ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl; X⁸ is C; and X⁹, when present, is C(R^{9a})(R^{9b}); wherein each R^{9a} and R^{9b} is independently H, halogen or unsubstituted alkyl.

In some embodiments, each X¹ and X² is CH₂; and X⁹, when present, is CH₂.

In some embodiments, X⁴ is C(R⁴) or N, wherein R⁴ is H, halogen or heteroalkyl; X⁵ is C(R⁵) or N, wherein R⁵ is H, halogen or heteroalkyl; X⁶ is C(R⁶) or N, wherein R⁶ is H, halogen or heteroalkyl; and X⁷ is C(R⁷) or N, wherein R⁷ is H, halogen or heteroalkyl.

In some embodiments, X⁴ is C(R⁴) or N, wherein R⁴ is H or heteroalkyl; X⁵ is C(R⁵) or N, wherein R⁵ is H or heteroalkyl; X⁶ is C(R⁶) or N, wherein R⁶ is H or heteroalkyl; and X⁷ is C(R⁷) or N, wherein R⁷ is H or heteroalkyl.

In some embodiments, each heteroalkyl is an alkoxy. In some embodiments, each said alkoxy is independently —OR^{k}, wherein each R^{k} is independently alkyl optionally substituted with —N(R^{d})(R^{e}) or heterocyclyl; wherein each R^{d} and R^{e} is independently H, alkyl, alkenyl or alkynyl.

In some embodiments, each heterocyclyl contains at least one nitrogen atom.

In some embodiments, X⁴ is N, X⁵ is C(R⁵), X⁶ is C(R⁶) and X⁷ is C(R⁷). In some embodiments, X⁷ is CH.

In some embodiments, X⁴ is C(R⁴), X⁵ is N, X⁶ is C(R⁶) and X⁷ is C(R⁷). In some embodiments, at least one of X⁴ and X⁷ is CH.

In some embodiments, X⁴ is C(R⁴), X⁵ is C(R⁵), X⁶ is N and X⁷ is C(R⁷). In some embodiments, at least one of X⁴ and X⁷ is CH.

In some embodiments, X⁴ is C(R⁴), X⁵ is C(R⁵), X⁶ is C(R⁶) and X⁷ is N. In some embodiments, X⁴ is CH.

In some embodiments, X⁴ is C(R⁴), X⁵ is C(R⁵), X⁶ is C(R⁶) and X⁷ is C(R⁷).

In some embodiments, X⁷ is CH. In some embodiments, X⁴ is CH. In some embodiments, each of X⁴ and X⁷ is CH.

In some embodiments, L is a phosphate-based linker comprising a phosphate-based moiety. In some embodiments, the phosphate-based moiety is selected from the group consisting of a phosphate ester, a pyrophosphate ester, a triphosphate ester, a tetraphosphate ester, a phosphonate, a diphosphonate, a phosporamidate, a pyrophosporamidate, a triphosphoramidate, a tetraphosphoramidate, a phosphorthioate and a diphosphorthioate.

In some embodiments, the drug-linker comprises a phosphate-based moiety. In some embodiments, the phosphate-based moiety is selected from the group consisting of a phosphate ester, a pyrophosphate ester, a triphosphate ester, a tetraphosphate ester, a phosphonate, a diphosphonate, a phosporamidate, a pyrophosporamidate, a triphosphoramidate, a tetraphosphoramidate, a phosphorthioate and a diphosphorthioate. In some embodiments, the phosphate-based moiety is a pyrophosphate ester. In some embodiments, the phosphate-based moiety is a diphosphonate.

In some embodiments, the phosphate-based linker is a bivalent linker.

In some embodiments, the phosphate-based moiety of the phosphate-based linker is covalently bound to an —O— atom of the drug. Accordingly, in some embodiments, the phosphate-based moiety of the phosphate-based linker is covalently bound to an —O— atom of a compound of Formula (X) or Formula (I), as disclosed herein, via a phosphorous atom of the phosphate-based moiety.

In some embodiments, the phosphate-based linker further comprises at least one additional moiety. In some embodiments, each at least one additional moiety is independently selected from the group consisting of unsubstituted alkylene, substituted alkylene, -(alkylene-O)—, optionally substituted arylene, —O—, —C(O)—, —N(R_{w})—, —S(O)₀₋₂—, a water-soluble polymer and an amino acid; wherein each R_{w} is independently H or alkyl, alkenyl or alkynyl; and combinations thereof. In some embodiments, each R_{w} is independently H or unsubstituted C₁-C₈ alkyl, C₁-C₈ alkenyl or C₁-C₈ alkyl alkynyl. In some further embodiments, each R_{w} is independently H or unsubstituted C₁-C₈ alkyl. In yet some further embodiments, each R_{w} in independently H or methyl.

In some embodiments, each at least one additional moiety is independently selected from the group consisting of unsubstituted alkylene, -(alkylene-O)—, —C(O)—, —N(R_{w})—, a water-soluble polymer and an amino acid; wherein each R_{w} is independently H or alkyl, alkenyl or alkynyl; and combinations thereof. In some embodiments, each R_{w} is independently H or unsubstituted C₁-C₈ alkyl, C₁-C₈ alkenyl or C₁-C₈ alkyl alkynyl. In some further embodiments, each R_{w} is independently H or unsubstituted C₁-C₈ alkyl. In yet some further embodiments, each R_{w} in independently H or methyl.

It is understood that each at least one additional moiety that can be present in a phosphate-based linker of the present disclosure can occur one or more times within said linker. In a non-limiting example, a phosphate-based linker of the present disclosure can comprise one or more unsubstituted alkylene group, wherein, each said unsubstituted alkylene group can be the same or different. In another non-limiting example, a phosphate-based linker of the present disclosure can comprise one or more amino acids, wherein each amino acid is the same or different.

In some embodiments, L comprises at least one alkylene group.

In some embodiments, L comprises at least one amino acid. In some embodiments, L comprises one amino acid. In some embodiments, the amino acid is selected from the group consisting of serine, threonine, cysteine, tyrosine, aspartic acid, glutamic acid, lysine and N_{ϵ}-methyl-lysine. In some embodiments, the amino acid is lysine or N_{ϵ}-methyl-lysine.

In some embodiments, L comprises one or more water-soluble polymer. In some embodiments, L comprises one water-soluble polymer.

In some embodiments, L comprises a water-soluble polymer and an amino acid, wherein the water-soluble polymer is conjugated to the amino acid. In some embodiments, the water-soluble polymer is conjugated to a side chain of the amino acid. In some embodiments, the water-soluble polymer is conjugated to the amino acid via a spacer element.

In some embodiments, L is selected from the group of linkers listed in Table 6.

In some embodiments, L is selected from the group of linkers listed in Table 7.

In some embodiments, L is selected from the group of linkers listed in Table 8.

In some embodiments, L is selected from the group consisting of:

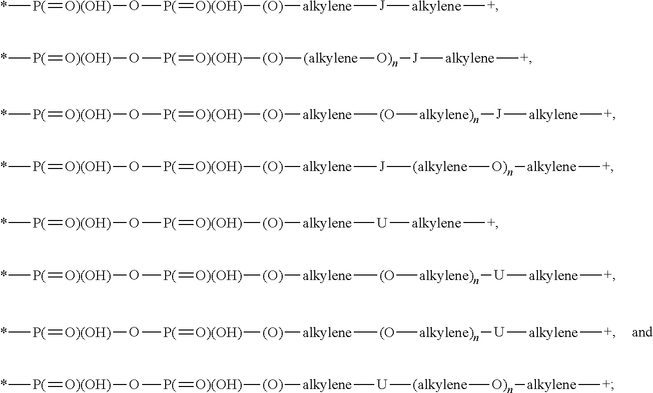

wherein:
each U is independently selected from the group consisting of:

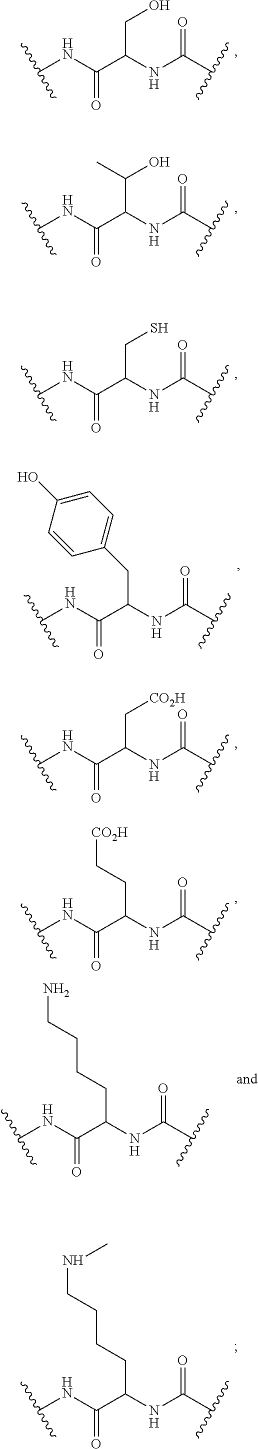

each J is independently

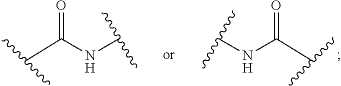 [SMILES: *NC(*)=O ; *NC(*)=O]

each alkylene is independently selected from the group consisting of:

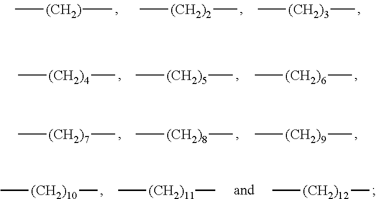

each n is independently an integer from 1 to 100;
* denotes the connection to the —O— atom of Formula (IL); and
+ denotes the connection to W;
   wherein each linker L is optionally substituted with one or more water-soluble polymers.

In some embodiments, each n is independently an integer from 1 to 10. In some embodiments, each n is independently 1, 2 or 3.

In some embodiments, L is substituted with the one or more water-soluble polymer.

In some embodiments, L comprises group U, and one water-soluble polymer is conjugated to an amino acid side chain of group U. In some embodiments, the water-soluble polymer is conjugated to the amino acid side chain of group U via a spacer element.

In some embodiments, L is:

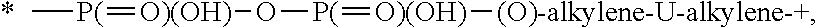

wherein:
U is selected from the group consisting of:

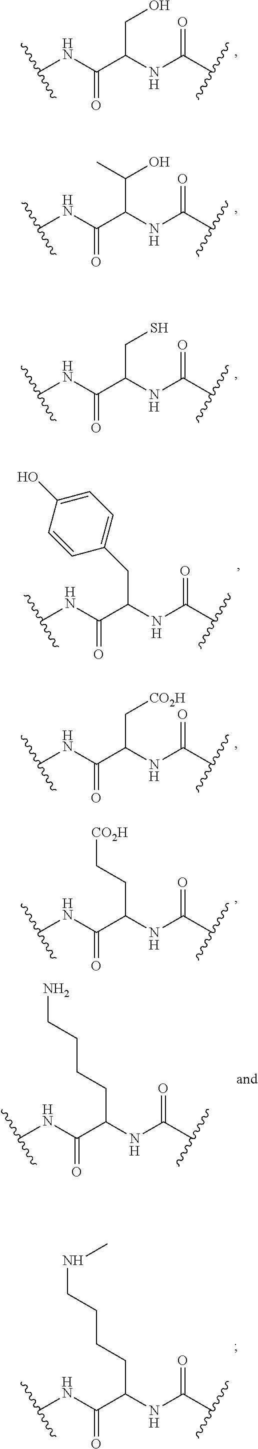

each alkylene is independently selected from the group consisting of:

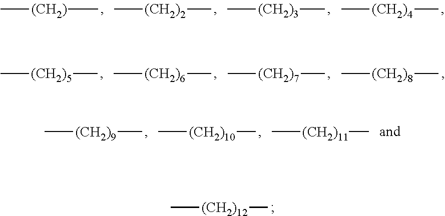

* denotes the connection to the —O— atom of Formula (IL); and
+ denotes the connection to W;
   wherein L is optionally substituted with one or more water-soluble polymers.

In some embodiments, L is substituted with the one or more water-soluble polymers. In some embodiments, one water-soluble polymer is conjugated to an amino acid side chain of group U. In some embodiments, the one water-soluble polymer is conjugated to the amino acid side chain of group U via a spacer element. In some embodiments, the spacer element is a carbonyl group.

In some embodiments, U is:

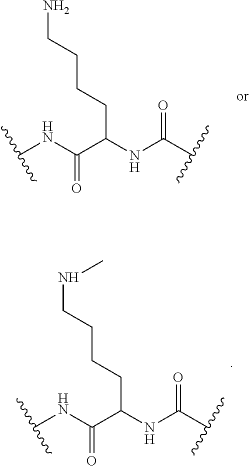 [SMILES: *NC(=O)C(CCCCN)NC(*)=O ; *NC(=O)C(CCCCNC)NC(*)=O]

In some embodiments, L has the following structure:

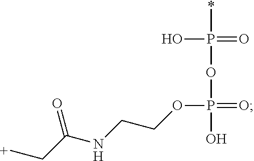

wherein: * denotes the connection to the —O— atom of Formula (IL); and + denotes the connection to W.

In some other embodiments, L has the following structure:

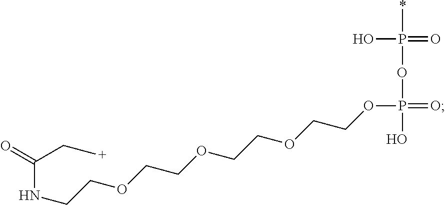

wherein: * denotes the connection to the —O— atom of Formula (IL); and + denotes the connection to W.

In some other embodiments, L has the following structure:

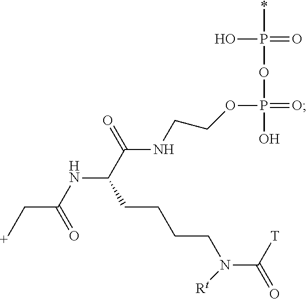

wherein: T is the water-soluble polymer; R^{t} is H or methyl; * denotes the connection to the —O— atom of Formula (IL); and + denotes the connection to W.

In some embodiments, the water-soluble polymer is a polysaccharide.

In some embodiments, the water-soluble polymer is a (polyethylene)glycol (PEG) moiety. In some embodiments, the PEG moiety has a molecular weight within a range of about 100 Da to about 100,000 Da. In some embodiments, the PEG moiety has a molecular weight within a range of about 100 Da to about 10,000 Da. In some embodiments, the PEG moiety has a molecular weight within a range of about 100 Da to about 5,000 Da. In some embodiments, the PEG moiety has a molecular weight within a range of about 100 Da to about 1,000 Da.

In some embodiments, the PEG moiety is —(CH₂CH₂O)ₙCH₃, wherein n is an integer from 1 to 100. In some embodiments, the PEG moiety is —(CH₂CH₂O)ₙCH₃, wherein n is an integer from 1 to 24. In some embodiments, the PEG moiety is —(CH₂CH₂O)ₙCH₃, wherein n is an integer from 6 to 12. In some embodiments, the PEG moiety is —(CH₂CH₂O)ₙCH₃, wherein n is an integer from 8 to 12. In some embodiments, the PEG moiety is —(CH₂CH₂O)ₙCH₃, wherein n is 8. In some embodiments, the PEG moiety is —(CH₂CH₂O)ₙCH₃, wherein n is 12.

In some embodiments, the PEG is linear. In some embodiments, the PEG is branched, multi-armed or dendritic.

In some embodiments, the reactive moiety W comprises —N₃, —OH, —SH, —NH(R^{j}), —C(O)R^{q}, —C(O)OR^{x}, —C(O)CH₂NH₂, an activated ester, —O—NH₂, a maleimide, a tetrazine, an alkyne, a cyclooctyne or an (E)-cyclooctene; wherein R^{j} is H or unsubstituted alkyl, R^{q} is unsubstituted alkyl, and R^{x} is H, unsubstituted alkyl or a carboxylic acid protecting group.

In some embodiments, the reactive moiety W is selected from the group consisting of:

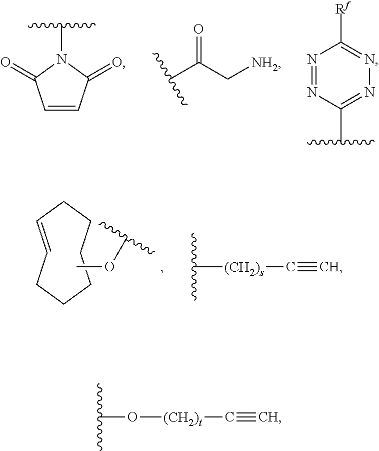

—N₃, —OH, —SH, —NH(Ri), —C(O)R^{q}, —C(O)OR^{x}, an activated ester, —O—NH₂ and an optionally substituted monocyclic or polycyclic group comprising the cyclooctyne;
wherein:
   R^{j} is H or unsubstituted C₁-C₆ alkyl,
   R^{q} is unsubstituted C₁-C₆ alkyl,
   R^{x} is H, unsubstituted C₁-C₆ alkyl or a carboxylic acid protecting group,
   R^{f} is H or unsubstituted C₁-C₆ alkyl,
   s is 0, 1, 2, 3, 4, 5 or 6, and
   t is 0, 1, 2, 3, 4, 5 or 6.

In some embodiments, the optionally substituted monocyclic or polycyclic group comprising the cyclooctyne is selected from the group consisting of:

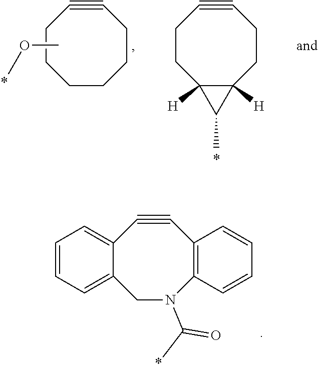

In some embodiments, W is —ONH₂.

In some aspects, there is provided a drug-linker compound of Formula (Ia) wherein R is L-W. Thus, in some embodiments, there is provided a compound of Formula (ILa) having the following structure:

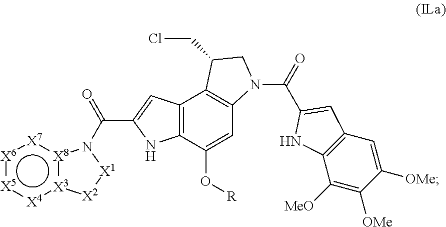

wherein:
R is L-W, wherein L is a linker and W is a reactive moiety;
X^{f} is C(R^{1a})(R^{1b}); wherein each R^{1a} and R^{1b} is independently H, halogen, alkyl, alkenyl or alkynyl;
X² is C(R^{2a})(R^{2b}); wherein each R^{2a} and R^{2b} is independently H, halogen, alkyl, alkenyl or alkynyl;
X³ is C;
X⁴ is C(R⁴) or N, wherein R⁴ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
X⁵ is C(R⁵) or N, wherein R⁵ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
X⁶ is C(R⁶) or N, wherein R⁶ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
X⁷ is C(R⁷) or N, wherein R⁷ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{S}); and
X⁸ is C;
   wherein:
each R^{a} and R^{b} is independently H, alkyl, alkenyl or alkynyl;
each R^{c} is independently H, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl or heteroarylalkyl;
each R^{s} is independently H, halogen, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl or heteroarylalkyl; and
each m is independently 0, 1, 2 or 3.

In some embodiments, X¹ is C(R^{1a})(R^{1b}); wherein each R^{1a} and R^{1b} is independently H, halogen or unsubstituted alkyl; X² is C(R^{2a})(R^{2b}); wherein each R^{2a} and R^{2b} is independently H, halogen or unsubstituted alkyl; X³ is C; X⁴ is C(R⁴) or N, wherein R⁴ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl; X⁵ is C(R⁵) or N, wherein R⁵ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl; X⁶ is C(R⁶) or N, wherein R⁶ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl; X⁷ is C(R⁷) or N, wherein R⁷ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl; X⁸ is C; and X⁹, when present, is C(R^{9a})(R^{9b}); wherein each R^{9a} and R^{9b} is independently H, halogen or unsubstituted alkyl.

In some embodiments, each X¹ and X² is CH₂; and X⁹, when present, is CH₂.

In some embodiments, X⁴ is C(R⁴) or N, wherein R⁴ is H, halogen or heteroalkyl; X⁵ is C(R⁵) or N, wherein R⁵ is H, halogen or heteroalkyl; X⁶ is C(R⁶) or N, wherein R⁶ is H, halogen or heteroalkyl; and X⁷ is C(R⁷) or N, wherein R⁷ is H, halogen or heteroalkyl.

In some embodiments, X⁴ is C(R⁴) or N, wherein R⁴ is H or heteroalkyl; X⁵ is C(R⁵) or N, wherein R⁵ is H or heteroalkyl; X⁶ is C(R⁶) or N, wherein R⁶ is H or heteroalkyl; and X⁷ is C(R⁷) or N, wherein R⁷ is H or heteroalkyl.

In some embodiments, each heteroalkyl is an alkoxy. In some embodiments, each said alkoxy is independently —OR^{k}, wherein each R^{k} is independently alkyl optionally substituted with —N(R^{d})(R^{e}) or heterocyclyl; wherein each R^{d} and R^{e} is independently H, alkyl, alkenyl or alkynyl.

In some embodiments, each heterocyclyl contains at least one nitrogen atom.

In some embodiments, X⁴ is N, X⁵ is C(R⁵), X⁶ is C(R⁶) and X⁷ is C(R⁷). In some embodiments, X⁷ is CH.

In some embodiments, X⁴ is C(R⁴), X⁵ is N, X⁶ is C(R⁶) and X⁷ is C(R⁷). In some embodiments, at least one of X⁴ and X⁷ is CH.

In some embodiments, X⁴ is C(R⁴), X⁵ is C(R⁵), X⁶ is N and X⁷ is C(R⁷). In some embodiments, at least one of X⁴ and X⁷ is CH.

In some embodiments, X⁴ is C(R⁴), X⁵ is C(R⁵), X⁶ is C(R⁶) and X⁷ is N. In some embodiments, X⁴ is CH.

In some embodiments, X⁴ is C(R⁴), X⁵ is C(R⁵), X⁶ is C(R⁶) and X⁷ is C(R⁷).

In some embodiments, X⁷ is CH. In some embodiments, X⁴ is CH. In some embodiments, each of X⁴ and X⁷ is CH.

In some embodiments, L is a phosphate-based linker comprising a phosphate-based moiety. In some embodiments, the phosphate-based moiety is selected from the group consisting of a phosphate ester, a pyrophosphate ester, a triphosphate ester, a tetraphosphate ester, a phosphonate, a diphosphonate, a phosporamidate, a pyrophosporamidate, a triphosphoramidate, a tetraphosphoramidate, a phosphorthioate and a diphosphorthioate. In some embodiments, the phosphate-based moiety is a pyrophosphate ester. In some other embodiments, the phosphate-based moiety is a diphosphonate.

In some embodiments, the phosphate-based linker is a bivalent linker.

In some embodiments, the phosphate-based moiety of the phosphate-based linker is covalently bound to an —O— atom of the drug. Accordingly, in some embodiments, the phosphate-based moiety of the phosphate-based linker is covalently bound to an —O— atom of a compound of Formula (X) or Formula (I), as disclosed herein, via a phosphorous atom of the phosphate-based moiety.

In some embodiments, the phosphate-based linker further comprises at least one additional moiety. In some embodiments, each at least one additional moiety is independently selected from the group consisting of unsubstituted alkylene, substituted alkylene, -(alkylene-O)—, optionally substituted arylene, —O—, —C(O)—, —N(R_{w})—, —S(O)₀₋₂—, a water-soluble polymer and an amino acid; wherein each R_{w} is independently H or alkyl, alkenyl or alkynyl; and combinations thereof. In some embodiments, each R_{w} is independently H or unsubstituted C₁-C₈ alkyl, C₁-C₈ alkenyl or C₁-C₈ alkyl alkynyl. In some further embodiments, each R_{w} is independently H or unsubstituted C₁-C₈ alkyl. In yet some further embodiments, each R_{w} in independently H or methyl.

In some embodiments, each at least one additional moiety is independently selected from the group consisting of unsubstituted alkylene, -(alkylene-O)—, —C(O)—, —N(R_{w})—, a water-soluble polymer and an amino acid; wherein each R_{w} is independently H or alkyl, alkenyl or alkynyl; and combinations thereof. In some embodiments, each R_{w} is independently H or unsubstituted C₁-C₈ alkyl, C₁-C₈ alkenyl or C₁-C₈ alkyl alkynyl. In some further embodiments, each R_{w} is independently H or unsubstituted C₁-C₈ alkyl. In yet some further embodiments, each R_{w} in independently H or methyl.

It is understood that each at least one additional moiety that can be present in a phosphate-based linker of the present disclosure can occur one or more times within said linker. In a non-limiting example, a phosphate-based linker of the present disclosure can comprise one or more unsubstituted alkylene group, wherein, each said unsubstituted alkylene group can be the same or different. In another non-limiting example, a phosphate-based linker of the present disclosure can comprise one or more amino acids, wherein each amino acid is the same or different.

In some embodiments, L comprises at least one alkylene group.

In some embodiments, L comprises at least one amino acid. In some embodiments, L comprises one amino acid. In some embodiments, the amino acid is selected from the group consisting of serine, threonine, cysteine, tyrosine, aspartic acid, glutamic acid, lysine and N_{ϵ}-methyl-lysine. In some embodiments, the amino acid is lysine or N_{ϵ}-methyl-lysine.

In some embodiments, L comprises one or more water-soluble polymer. In some embodiments, L comprises one water-soluble polymer.

In some embodiments, L comprises a water-soluble polymer and an amino acid, wherein the water-soluble polymer is conjugated to the amino acid. In some embodiments, the water-soluble polymer is conjugated to a side chain of the amino acid. In some embodiments, the water-soluble polymer is conjugated to the amino acid via a spacer element.

In some embodiments, L is selected from the group of linkers listed in Table 6.

In some embodiments, L is selected from the group of linkers listed in Table 7.

In some embodiments, L is selected from the group of linkers listed in Table 8.

In some embodiments, L is selected from the group consisting of:

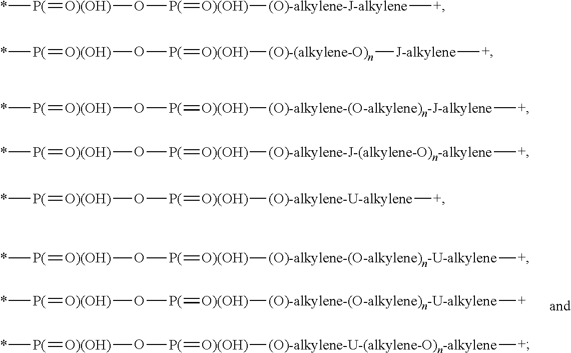

wherein:
each U is independently selected from the group consisting of:

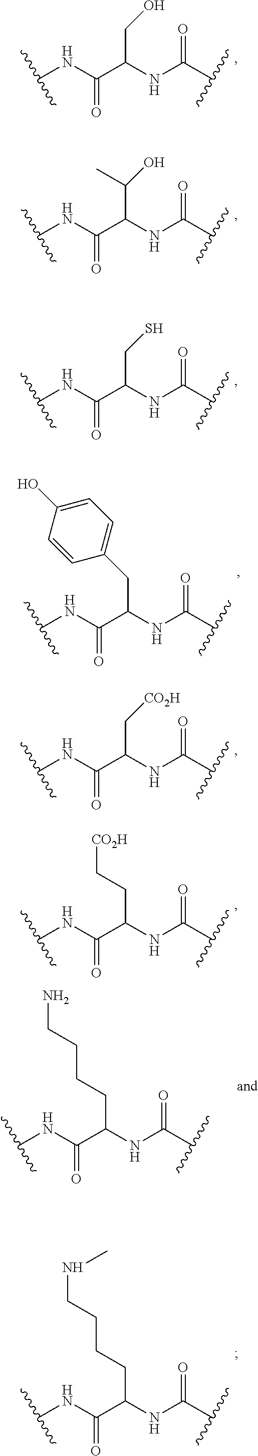

each J is independently

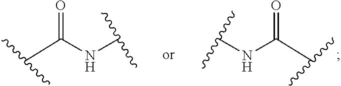 [SMILES: *NC(*)=O ; *NC(*)=O]

each alkylene is independently selected from the group consisting of:

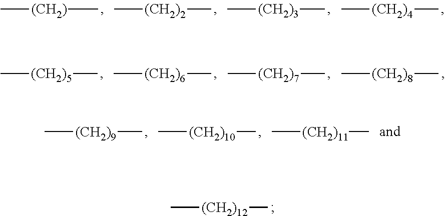

each n is independently an integer from 1 to 100;
* denotes the connection to the —O— atom of Formula (ILa); and
+ denotes the connection to W;
wherein each linker L is optionally substituted with one or more water-soluble polymers.

In some embodiments, each n is independently an integer from 1 to 10. In some embodiments, each n is independently 1, 2 or 3.

In some embodiments, L is substituted with the one or more water-soluble polymer.

In some embodiments, L comprises group U, and one water-soluble polymer is conjugated to an amino acid side chain of group U. In some embodiments, the water-soluble polymer is conjugated to the amino acid side chain of group U via a spacer element.

In some embodiments, L is:

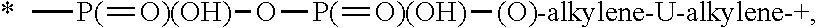

wherein:
U is selected from the group consisting of:

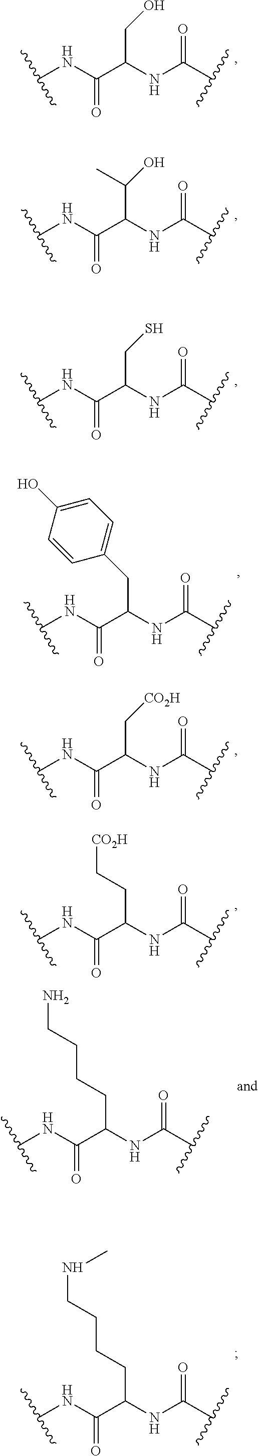

each alkylene is independently selected from the group consisting of:

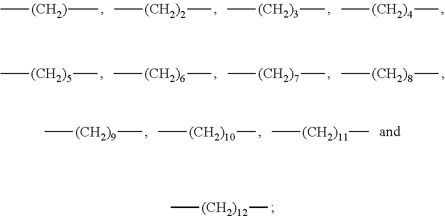

* denotes the connection to the —O— atom of Formula (ILa); and
+ denotes the connection to W;
   wherein L is optionally substituted with one or more water-soluble polymers.

In some embodiments, L is substituted with the one or more water-soluble polymers. In some embodiments, one water-soluble polymer is conjugated to an amino acid side chain of group U. In some embodiments, the one water-soluble polymer is conjugated to the amino acid side chain of group U via a spacer element. In some embodiments, the spacer element is a carbonyl group.

In some embodiments, U is:

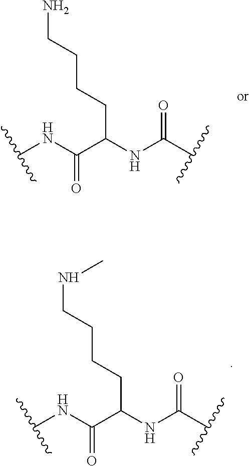 [SMILES: *NC(=O)C(CCCCN)NC(*)=O ; *NC(=O)C(CCCCNC)NC(*)=O]

In some embodiments, L has the following structure:

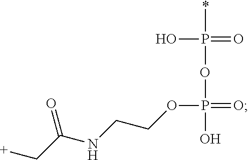

wherein: * denotes the connection to the —O— atom of Formula (ILa); and + denotes the connection to W.

In some other embodiments, L has the following structure:

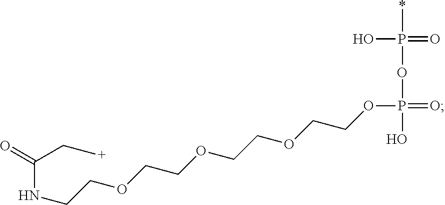

wherein: * denotes the connection to the —O— atom of Formula (ILa); and + denotes the connection to W.

In some other embodiments, L has the following structure:

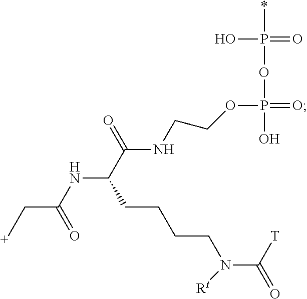

wherein: T is the water-soluble polymer; R^{t} is H or methyl; * denotes the connection to the —O— atom of Formula (ILa); and + denotes the connection to W.

In some embodiments, the water-soluble polymer is a polysaccharide.

In some embodiments, the water-soluble polymer is a (polyethylene)glycol (PEG) moiety. In some embodiments, the PEG moiety has a molecular weight within a range of about 100 Da to about 100,000 Da. In some embodiments, the PEG moiety has a molecular weight within a range of about 100 Da to about 10,000 Da. In some embodiments, the PEG moiety has a molecular weight within a range of about 100 Da to about 5,000 Da. In some embodiments, the PEG moiety has a molecular weight within a range of about 100 Da to about 1,000 Da.

In some embodiments, the PEG moiety is —(CH₂CH₂O)ₙCH₃, wherein n is an integer from 1 to 100. In some embodiments, the PEG moiety is —(CH₂CH₂O)ₙCH₃, wherein n is an integer from 1 to 24. In some embodiments, the PEG moiety is —(CH₂CH₂O)ₙCH₃, wherein n is an integer from 6 to 12. In some embodiments, the PEG moiety is —(CH₂CH₂O)ₙCH₃, wherein n is an integer from 8 to 12. In some embodiments, the PEG moiety is —(CH₂CH₂O)ₙCH₃, wherein n is 8. In some embodiments, the PEG moiety is —(CH₂CH₂O)ₙCH₃, wherein n is 12.

In some embodiments, the PEG is linear. In some embodiments, the PEG is branched, multi-armed or dendritic.

In some embodiments, the reactive moiety W comprises —N₃, —OH, —SH, —NH(R^{j}), —C(O)R^{q}, —C(O)OR^{x}, —C(O)CH₂NH₂, an activated ester, —O—NH₂, a maleimide, a tetrazine, an alkyne, a cyclooctyne or an (E)-cyclooctene; wherein Ri is H or unsubstituted alkyl, R^{q} is unsubstituted alkyl, and R^{x} is H, unsubstituted alkyl or a carboxylic acid protecting group.

In some embodiments, the reactive moiety W is selected from the group consisting of:

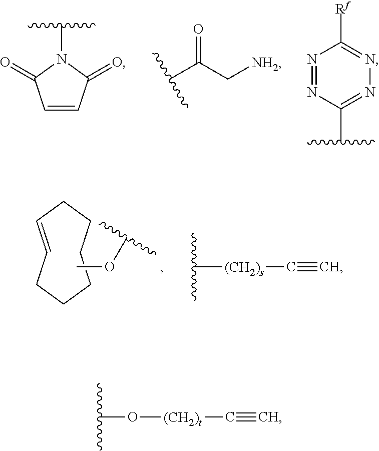

—N₃, —OH, —SH, —NH(Ri), —C(O)R^{q}, —C(O)OR^{x}, an activated ester, —O—NH₂ and an optionally substituted monocyclic or polycyclic group comprising the cyclooctyne;
wherein:
   R^{j} is H or unsubstituted C₁-C₆ alkyl,
   R^{q} is unsubstituted C₁-C₆ alkyl,
   R^{x} is H, unsubstituted C₁-C₆ alkyl or a carboxylic acid protecting group,
   R^{f} is H or unsubstituted C₁-C₆ alkyl,
   s is 0, 1, 2, 3, 4, 5 or 6, and
   t is 0, 1, 2, 3, 4, 5 or 6.

In some embodiments, the optionally substituted monocyclic or polycyclic group comprising the cyclooctyne is selected from the group consisting of:

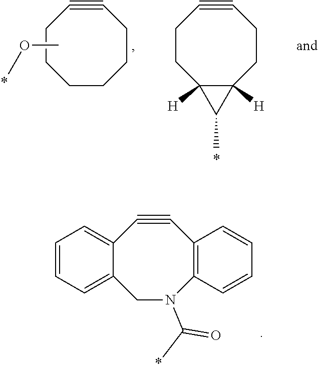

In some embodiments, W is —ONH₂.

In some aspects, there is provided a drug-linker compound of Formula (Ib) wherein R is L-W. Thus, in some embodiments, there is provided a compound of Formula (ILb) having the following structure:

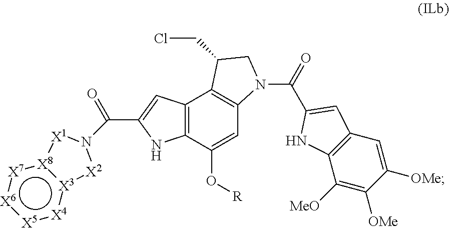

wherein:
R is L-W, wherein L is a linker and W is a reactive moiety;
X¹ is C(R^{1a})(R^{1b}); wherein each R^{1a} and R^{1b} is independently H, halogen, alkyl, alkenyl or alkynyl;
X² is C(R^{2a})(R^{2b}); wherein each R^{2a} and R^{2b} is independently H, halogen, alkyl, alkenyl or alkynyl;
X³ is C;
X⁴ is C(R⁴) or N, wherein R⁴ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
X⁵ is C(R⁵) or N, wherein R⁵ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
X⁶ is C(R⁶) or N, wherein R⁶ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
X⁷ is C(R⁷) or N, wherein R⁷ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s}); and
X⁸ is C;
   wherein:
each R^{a} and R^{b} is independently H, alkyl, alkenyl or alkynyl;
each R^{c} is independently H, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl or heteroarylalkyl;
each R^{s} is independently H, halogen, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl or heteroarylalkyl; and
each m is independently 0, 1, 2 or 3.

In some embodiments, X¹ is C(R^{1a})(R^{1b}); wherein each R^{1a} and R^{1b} is independently H, halogen or unsubstituted alkyl; X² is C(R^{2a})(R^{2b}); wherein each R^{2a} and R^{2b} is independently H, halogen or unsubstituted alkyl; X³ is C; X⁴ is C(R⁴) or N, wherein R⁴ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl; X⁵ is C(R⁵) or N, wherein R⁵ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl; X⁶ is C(R⁶) or N, wherein R⁶ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl; X⁷ is C(R⁷) or N, wherein R⁷ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl; X⁸ is C; and X⁹, when present, is C(R^{9a})(R^{9b}); wherein each R^{9a} and R^{9b} is independently H, halogen or unsubstituted alkyl.

In some embodiments, each X¹ and X² is CH₂; and X⁹, when present, is CH₂.

In some embodiments, X⁴ is C(R⁴) or N, wherein R⁴ is H, halogen or heteroalkyl; X⁵ is C(R⁵) or N, wherein R⁵ is H, halogen or heteroalkyl; X⁶ is C(R⁶) or N, wherein R⁶ is H, halogen or heteroalkyl; and X⁷ is C(R⁷) or N, wherein R⁷ is H, halogen or heteroalkyl.

In some embodiments, X⁴ is C(R⁴) or N, wherein R⁴ is H or heteroalkyl; X⁵ is C(R⁵) or N, wherein R⁵ is H or heteroalkyl; X⁶ is C(R⁶) or N, wherein R⁶ is H or heteroalkyl; and X⁷ is C(R⁷) or N, wherein R⁷ is H or heteroalkyl.

In some embodiments, each heteroalkyl is an alkoxy. In some embodiments, each said alkoxy is independently —OR^{k}, wherein each R^{k} is independently alkyl optionally substituted with —N(R^{d})(R^{e}) or heterocyclyl; wherein each R^{d} and R^{e} is independently H, alkyl, alkenyl or alkynyl.

In some embodiments, each heterocyclyl contains at least one nitrogen atom.

In some embodiments, X⁴ is N, X⁵ is C(R⁵), X⁶ is C(R⁶) and X⁷ is C(R⁷). In some embodiments, X⁷ is CH.

In some embodiments, X⁴ is C(R⁴), X⁵ is N, X⁶ is C(R⁶) and X⁷ is C(R⁷). In some embodiments, at least one of X⁴ and X⁷ is CH.

In some embodiments, X⁴ is C(R⁴), X⁵ is C(R⁵), X⁶ is N and X⁷ is C(R⁷). In some embodiments, at least one of X⁴ and X⁷ is CH.

In some embodiments, X⁴ is C(R⁴), X⁵ is C(R⁵), X⁶ is C(R⁶) and X⁷ is N. In some embodiments, X⁴ is CH.

In some embodiments, X⁴ is C(R⁴), X⁵ is C(R⁵), X⁶ is C(R⁶) and X⁷ is C(R⁷).

In some embodiments, X⁷ is CH. In some embodiments, X⁴ is CH. In some embodiments, each of X⁴ and X⁷ is CH.

In some embodiments, L is a phosphate-based linker comprising a phosphate-based moiety. In some embodiments, the phosphate-based moiety is selected from the group consisting of a phosphate ester, a pyrophosphate ester, a triphosphate ester, a tetraphosphate ester, a phosphonate, a diphosphonate, a phosporamidate, a pyrophosporamidate, a triphosphoramidate, a tetraphosphoramidate, a phosphorthioate and a diphosphorthioate. In some embodiments, the phosphate-based moiety is a pyrophosphate ester. In some other embodiments, the phosphate-based moiety is a diphosphonate.

In some embodiments, the phosphate-based linker is a bivalent linker.

In some embodiments, the phosphate-based moiety of the phosphate-based linker is covalently bound to an —O— atom of the drug. Accordingly, in some embodiments, the phosphate-based moiety of the phosphate-based linker is covalently bound to an —O— atom of a compound of Formula (X) or Formula (I), as disclosed herein, via a phosphorous atom of the phosphate-based moiety.

In some embodiments, the phosphate-based linker further comprises at least one additional moiety. In some embodiments, each at least one additional moiety is independently selected from the group consisting of unsubstituted alkylene, substituted alkylene, -(alkylene-O)—, optionally substituted arylene, —O—, —C(O)—, —N(R_{w})—, —S(O)₀₋₂—, a water-soluble polymer and an amino acid; wherein each R_{w} is independently H or alkyl, alkenyl or alkynyl; and combinations thereof. In some embodiments, each R_{w} is independently H or unsubstituted C₁-C₈ alkyl, C₁-C₈ alkenyl or C₁-C₈ alkyl alkynyl. In some further embodiments, each R_{w} is independently H or unsubstituted C₁-C₈ alkyl. In yet some further embodiments, each R_{w} in independently H or methyl.

In some embodiments, each at least one additional moiety is independently selected from the group consisting of unsubstituted alkylene, -(alkylene-O)—, —C(O)—, —N(R_{w})—, a water-soluble polymer and an amino acid; wherein each R_{w} is independently H or alkyl, alkenyl or alkynyl; and combinations thereof. In some embodiments, each R_{w} is independently H or unsubstituted C₁-C₈ alkyl, C₁-C₈ alkenyl or C₁-C₈ alkyl alkynyl. In some further embodiments, each R_{w} is independently H or unsubstituted C₁-C₈ alkyl. In yet some further embodiments, each R_{W} in independently H or methyl.

It is understood that each at least one additional moiety that can be present in a phosphate-based linker of the present disclosure can occur one or more times within said linker. In a non-limiting example, a phosphate-based linker of the present disclosure can comprise one or more unsubstituted alkylene group, wherein, each said unsubstituted alkylene group can be the same or different. In another non-limiting example, a phosphate-based linker of the present disclosure can comprise one or more amino acids, wherein each amino acid is the same or different.

In some embodiments, L comprises at least one alkylene group.

In some embodiments, L comprises at least one amino acid. In some embodiments, L comprises one amino acid. In some embodiments, the amino acid is selected from the group consisting of serine, threonine, cysteine, tyrosine, aspartic acid, glutamic acid, lysine and N_{ϵ}-methyl-lysine. In some embodiments, the amino acid is lysine or N_{ϵ}-methyl-lysine.

In some embodiments, L comprises one or more water-soluble polymer. In some embodiments, L comprises one water-soluble polymer.

In some embodiments, L comprises a water-soluble polymer and an amino acid, wherein the water-soluble polymer is conjugated to the amino acid. In some embodiments, the water-soluble polymer is conjugated to a side chain of the amino acid. In some embodiments, the water-soluble polymer is conjugated to the amino acid via a spacer element.

In some embodiments, L is selected from the group of linkers listed in Table 6.

In some embodiments, L is selected from the group of linkers listed in Table 7.

In some embodiments, L is selected from the group of linkers listed in Table 8.

In some embodiments, L is selected from the group consisting of:

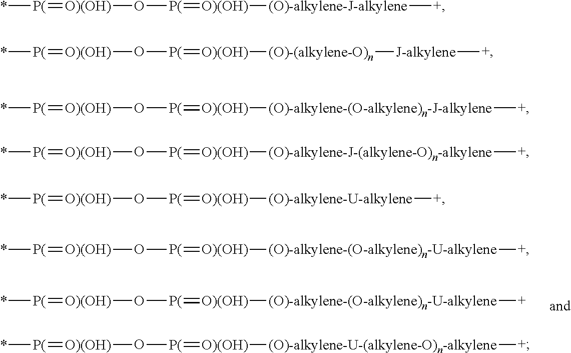

wherein:
each U is independently selected from the group consisting of:

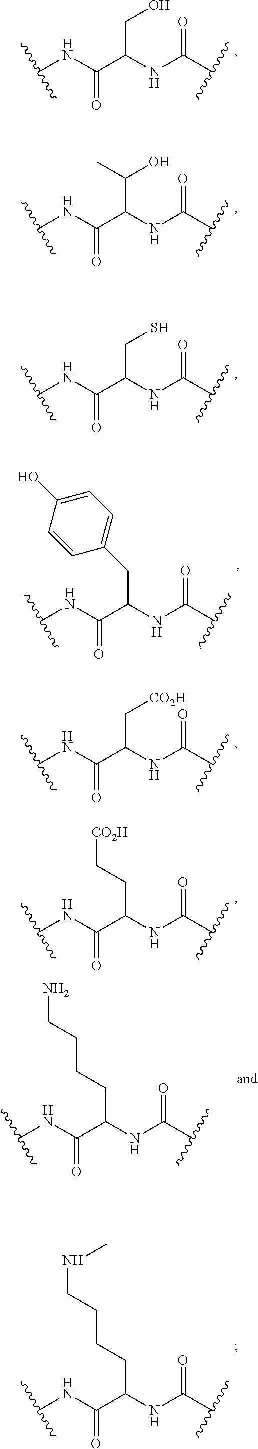

each J is independently

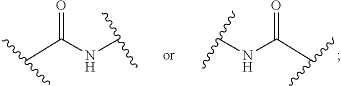 [SMILES: *NC(*)=O ; *NC(*)=O]

each alkylene is independently selected from the group consisting of:

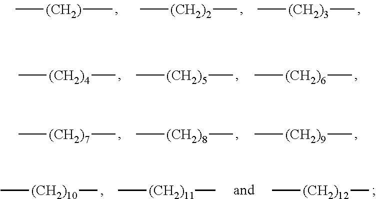

each n is independently an integer from 1 to 100;
* denotes the connection to the —O— atom of Formula (ILb); and
+ denotes the connection to W;
   wherein each linker L is optionally substituted with one or more water-soluble polymers.

In some embodiments, each n is independently an integer from 1 to 10. In some embodiments, each n is independently 1, 2 or 3.

In some embodiments, L is substituted with the one or more water-soluble polymer.

In some embodiments, L comprises group U, and one water-soluble polymer is conjugated to an amino acid side chain of group U. In some embodiments, the water-soluble polymer is conjugated to the amino acid side chain of group U via a spacer element.

In some embodiments, L is:

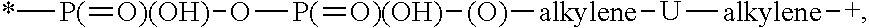

wherein:
U is selected from the group consisting of:

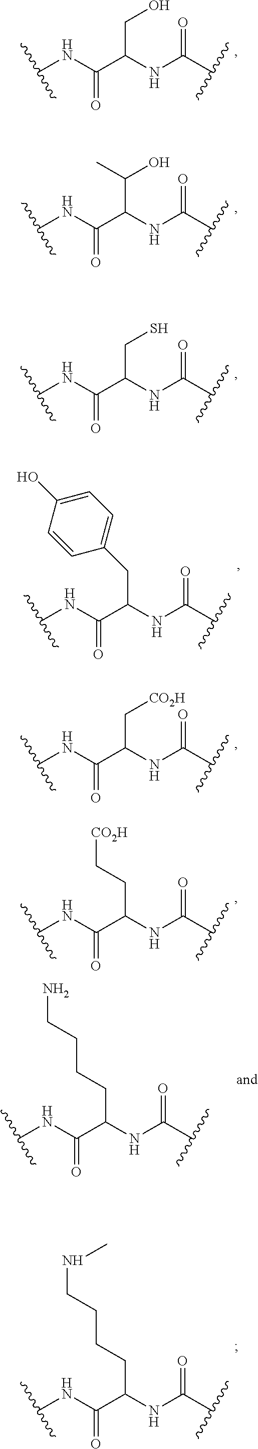

each alkylene is independently selected from the group consisting of:

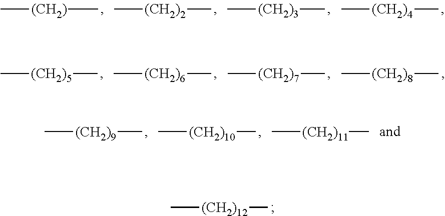

* denotes the connection to the —O— atom of Formula (ILb); and
+ denotes the connection to W;
   wherein L is optionally substituted with one or more water-soluble polymers.

In some embodiments, L is substituted with the one or more water-soluble polymers. In some embodiments, one water-soluble polymer is conjugated to an amino acid side chain of group U. In some embodiments, the one water-soluble polymer is conjugated to the amino acid side chain of group U via a spacer element. In some embodiments, the spacer element is a carbonyl group.

In some embodiments, U is:

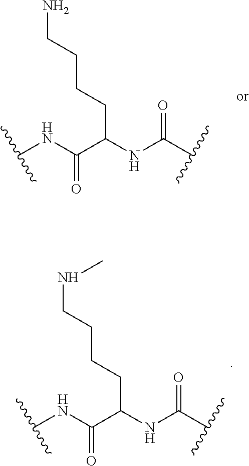 [SMILES: *NC(=O)C(CCCCN)NC(*)=O ; *NC(=O)C(CCCCNC)NC(*)=O]

In some embodiments, L has the following structure:

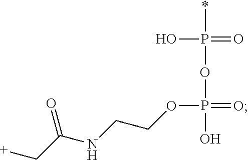

wherein: * denotes the connection to the —O— atom of Formula (ILb); and + denotes the connection to W.

In some other embodiments, L has the following structure:

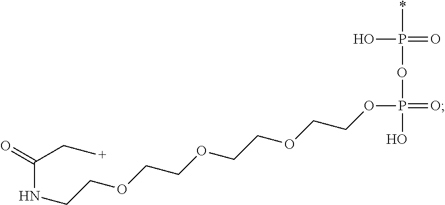

wherein: * denotes the connection to the —O— atom of Formula (ILb); and + denotes the connection to W.

In some other embodiments, L has the following structure:

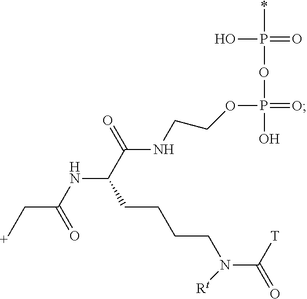

wherein: T is the water-soluble polymer; R^{t} is H or methyl; * denotes the connection to the —O— atom of Formula (ILb); and + denotes the connection to W.

In some embodiments, the water-soluble polymer is a polysaccharide.

In some embodiments, the water-soluble polymer is a (polyethylene)glycol (PEG) moiety. In some embodiments, the PEG moiety has a molecular weight within a range of about 100 Da to about 100,000 Da. In some embodiments, the PEG moiety has a molecular weight within a range of about 100 Da to about 10,000 Da. In some embodiments, the PEG moiety has a molecular weight within a range of about 100 Da to about 5,000 Da. In some embodiments, the PEG moiety has a molecular weight within a range of about 100 Da to about 1,000 Da.

In some embodiments, the PEG moiety is —(CH₂CH₂O)ₙCH₃, wherein n is an integer from 1 to 100. In some embodiments, the PEG moiety is —(CH₂CH₂O)ₙCH₃, wherein n is an integer from 1 to 24. In some embodiments, the PEG moiety is —(CH₂CH₂O)ₙCH₃, wherein n is an integer from 6 to 12. In some embodiments, the PEG moiety is —(CH₂CH₂O)ₙCH₃, wherein n is an integer from 8 to 12. In some embodiments, the PEG moiety is —(CH₂CH₂O)ₙCH₃, wherein n is 8. In some embodiments, the PEG moiety is —(CH₂CH₂O)ₙCH₃, wherein n is 12.

In some embodiments, the PEG is linear. In some embodiments, the PEG is branched, multi-armed or dendritic.

In some embodiments, the reactive moiety W comprises —N₃, —OH, —SH, —NH(Ri), —C(O)R^{q}, —C(O)OR^{x}, —C(O)CH₂NH₂, an activated ester, —O—NH₂, a maleimide, a tetrazine, an alkyne, a cyclooctyne or an (E)-cyclooctene; wherein Ri is H or unsubstituted alkyl, R^{q} is unsubstituted alkyl, and R^{x} is H, unsubstituted alkyl or a carboxylic acid protecting group.

In some embodiments, the reactive moiety W is selected from the group consisting of:

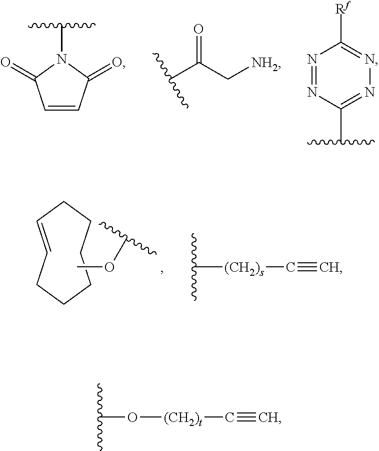

—N₃, —OH, —SH, —NH(R^{j}), —C(O)R^{q}, —C(O)OR^{x}, an activated ester, —O—NH₂ and an optionally substituted monocyclic or polycyclic group comprising the cyclooctyne;
wherein:
   R^{j} is H or unsubstituted C₁-C₆ alkyl,
   R^{q} is unsubstituted C₁-C₆ alkyl,
   R^{x} is H, unsubstituted C₁-C₆ alkyl or a carboxylic acid protecting group,
   R^{f} is H or unsubstituted C₁-C₆ alkyl,
   s is 0, 1, 2, 3, 4, 5 or 6, and
   t is 0, 1, 2, 3, 4, 5 or 6.

In some embodiments, the optionally substituted monocyclic or polycyclic group comprising the cyclooctyne is selected from the group consisting of:

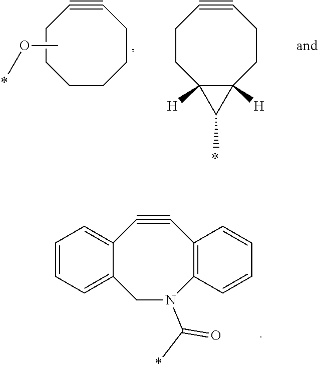

In some embodiments, W is —ONH₂.

In some aspects, there is provided a drug-linker compound of Formula (Ic) wherein R is L-W. Thus, in some embodiments, there is provided a compound of Formula (ILc) having the following structure:

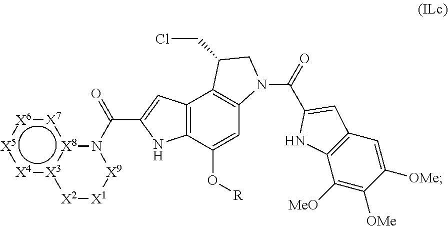

wherein:
R is L-W, wherein L is a linker and W is a reactive moiety;
X¹ is C(R^{1a})(R^{1b}); wherein each R^{1a} and R^{1b} is independently H, halogen, alkyl, alkenyl or alkynyl;
X² is C(R^{2a})(R^{2b}); wherein each R^{2a} and R^{2b} is independently H, halogen, alkyl, alkenyl or alkynyl;
X³ is C;
X⁴ is C(R⁴) or N, wherein R⁴ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
X⁵ is C(R⁵) or N, wherein R⁵ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
X⁶ is C(R⁶) or N, wherein R⁶ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
X⁷ is C(R⁷) or N, wherein R⁷ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
X⁸ is C; and
X⁹ is C(R^{9a})(R^{9b}); wherein each R^{9a} and R^{9b} is independently H, halogen, alkyl, alkenyl or alkynyl;
   wherein:
each R^{a} and R^{b} is independently H, alkyl, alkenyl or alkynyl;
each R^{c} is independently H, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl or heteroarylalkyl;
each R^{s} is independently H, halogen, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl or heteroarylalkyl; and
each m is independently 0, 1, 2 or 3.

In some embodiments, X¹ is C(R^{1a})(R^{1b}); wherein each R^{1a} and R^{1b} is independently H, halogen or unsubstituted alkyl; X² is C(R^{2a})(R^{2b}); wherein each R^{2a} and R^{2b} is independently H, halogen or unsubstituted alkyl; X³ is C; X⁴ is C(R⁴) or N, wherein R⁴ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl; X⁵ is C(R⁵) or N, wherein R⁵ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl; X⁶ is C(R⁶) or N, wherein R⁶ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl; X⁷ is C(R⁷) or N, wherein R⁷ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl; X⁸ is C; and X⁹, when present, is C(R^{9a})(R^{9b}); wherein each R^{9a} and R^{9b} is independently H, halogen or unsubstituted alkyl.

In some embodiments, each X¹ and X² is CH₂; and X⁹, when present, is CH₂.

In some embodiments, X⁴ is C(R⁴) or N, wherein R⁴ is H, halogen or heteroalkyl; X⁵ is C(R⁵) or N, wherein R⁵ is H, halogen or heteroalkyl; X⁶ is C(R⁶) or N, wherein R⁶ is H, halogen or heteroalkyl; and X⁷ is C(R⁷) or N, wherein R⁷ is H, halogen or heteroalkyl.

In some embodiments, X⁴ is C(R⁴) or N, wherein R⁴ is H or heteroalkyl; X⁵ is C(R⁵) or N, wherein R⁵ is H or heteroalkyl; X⁶ is C(R⁶) or N, wherein R⁶ is H or heteroalkyl; and X⁷ is C(R⁷) or N, wherein R⁷ is H or heteroalkyl.

In some embodiments, each heteroalkyl is an alkoxy. In some embodiments, each said alkoxy is independently —OR^{k}, wherein each R^{k} is independently alkyl optionally substituted with —N(R^{d})(R^{e}) or heterocyclyl; wherein each R^{d} and R^{e} is independently H, alkyl, alkenyl or alkynyl.

In some embodiments, each heterocyclyl contains at least one nitrogen atom.

In some embodiments, X⁴ is N, X⁵ is C(R⁵), X⁶ is C(R⁶) and X⁷ is C(R⁷). In some embodiments, X⁷ is CH.

In some embodiments, X⁴ is C(R⁴), X⁵ is N, X⁶ is C(R⁶) and X⁷ is C(R⁷). In some embodiments, at least one of X⁴ and X⁷ is CH.

In some embodiments, X⁴ is C(R⁴), X⁵ is C(R⁵), X⁶ is N and X⁷ is C(R⁷). In some embodiments, at least one of X⁴ and X⁷ is CH.

In some embodiments, X⁴ is C(R⁴), X⁵ is C(R⁵), X⁶ is C(R⁶) and X⁷ is N. In some embodiments, X⁴ is CH.

In some embodiments, X⁴ is C(R⁴), X⁵ is C(R⁵), X⁶ is C(R⁶) and X⁷ is C(R⁷).

In some embodiments, X⁷ is CH. In some embodiments, X⁴ is CH. In some embodiments, each of X⁴ and X⁷ is CH.

In some embodiments, L is a phosphate-based linker comprising a phosphate-based moiety. In some embodiments, the phosphate-based moiety is selected from the group consisting of a phosphate ester, a pyrophosphate ester, a triphosphate ester, a tetraphosphate ester, a phosphonate, a diphosphonate, a phosporamidate, a pyrophosporamidate, a triphosphoramidate, a tetraphosphoramidate, a phosphorthioate and a diphosphorthioate. In some embodiments, the phosphate-based moiety is a pyrophosphate ester. In some other embodiments, the phosphate-based moiety is a diphosphonate.

In some embodiments, the phosphate-based linker is a bivalent linker.

In some embodiments, the phosphate-based moiety of the phosphate-based linker is covalently bound to an —O— atom of the drug. Accordingly, in some embodiments, the phosphate-based moiety of the phosphate-based linker is covalently bound to an —O— atom of a compound of Formula (X) or Formula (I), as disclosed herein, via a phosphorous atom of the phosphate-based moiety.

In some embodiments, the phosphate-based linker further comprises at least one additional moiety. In some embodiments, each at least one additional moiety is independently selected from the group consisting of unsubstituted alkylene, substituted alkylene, -(alkylene-O)—, optionally substituted arylene, —O—, —C(O)—, —N(R_{w})—, —S(O)₀₋₂—, a water-soluble polymer and an amino acid; wherein each R_{w} is independently H or alkyl, alkenyl or alkynyl; and combinations thereof. In some embodiments, each R_{w} is independently H or unsubstituted C₁-C₈ alkyl, C₁-C₈ alkenyl or C₁-C₈ alkyl alkynyl. In some further embodiments, each R_{w} is independently H or unsubstituted C₁-C₈alkyl. In yet some further embodiments, each R_{w} in independently H or methyl.

In some embodiments, each at least one additional moiety is independently selected from the group consisting of unsubstituted alkylene, -(alkylene-O)—, —C(O)—, —N(R_{w})—, a water-soluble polymer and an amino acid; wherein each R_{w} is independently H or alkyl, alkenyl or alkynyl; and combinations thereof. In some embodiments, each R_{w} is independently H or unsubstituted C₁-C₈ alkyl, C₁-C₈ alkenyl or C₁-C₈ alkyl alkynyl. In some further embodiments, each R_{w} is independently H or unsubstituted C₁-C₈ alkyl. In yet some further embodiments, each R_{w} in independently H or methyl.

It is understood that each at least one additional moiety that can be present in a phosphate-based linker of the present disclosure can occur one or more times within said linker. In a non-limiting example, a phosphate-based linker of the present disclosure can comprise one or more unsubstituted alkylene group, wherein, each said unsubstituted alkylene group can be the same or different. In another non-limiting example, a phosphate-based linker of the present disclosure can comprise one or more amino acids, wherein each amino acid is the same or different.

In some embodiments, L comprises at least one alkylene group.

In some embodiments, L comprises at least one amino acid. In some embodiments, L comprises one amino acid. In some embodiments, the amino acid is selected from the group consisting of serine, threonine, cysteine, tyrosine, aspartic acid, glutamic acid, lysine and N_{ϵ}-methyl-lysine. In some embodiments, the amino acid is lysine or N_{ϵ}-methyl-lysine.

In some embodiments, L comprises one or more water-soluble polymer. In some embodiments, L comprises one water-soluble polymer.

In some embodiments, L comprises a water-soluble polymer and an amino acid, wherein the water-soluble polymer is conjugated to the amino acid. In some embodiments, the water-soluble polymer is conjugated to a side chain of the amino acid. In some embodiments, the water-soluble polymer is conjugated to the amino acid via a spacer element.

In some embodiments, L is selected from the group of linkers listed in Table 6.

In some embodiments, L is selected from the group of linkers listed in Table 7.

In some embodiments, L is selected from the group of linkers listed in Table 8.

In some embodiments, L is selected from the group consisting of:

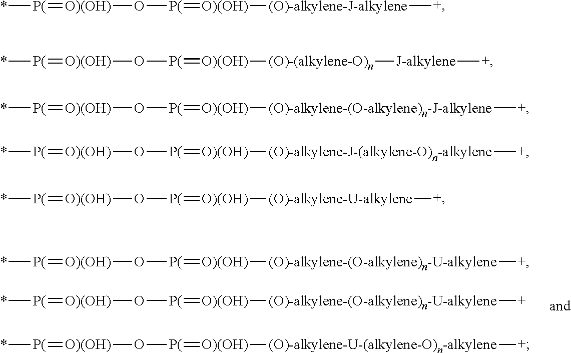

wherein:
each U is independently selected from the group consisting of:

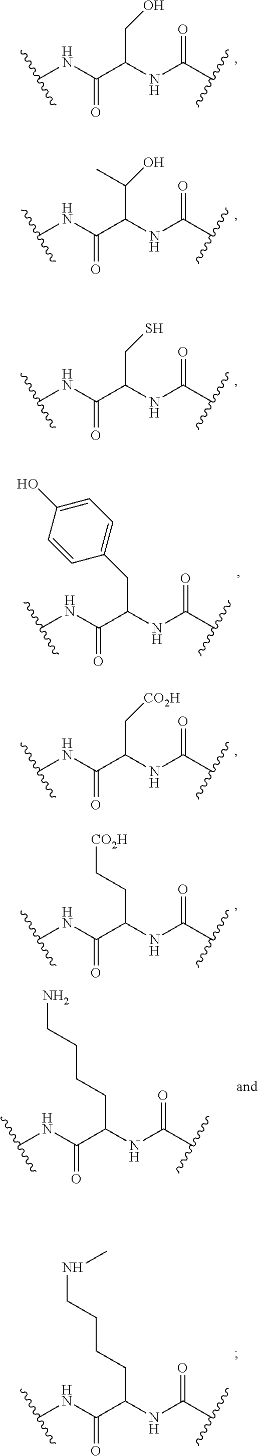

each J is independently

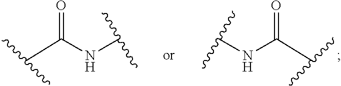 [SMILES: *NC(*)=O ; *NC(*)=O]

each alkylene is independently selected from the group consisting of:

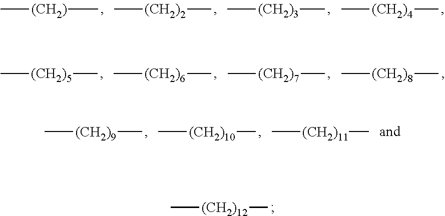

each n is independently an integer from 1 to 100;
* denotes the connection to the —O— atom of Formula (ILc); and
+ denotes the connection to W;
   wherein each linker L is optionally substituted with one or more water-soluble polymers.

In some embodiments, each n is independently an integer from 1 to 10. In some embodiments, each n is independently 1, 2 or 3.

In some embodiments, L is substituted with the one or more water-soluble polymer.

In some embodiments, L comprises group U, and one water-soluble polymer is conjugated to an amino acid side chain of group U. In some embodiments, the water-soluble polymer is conjugated to the amino acid side chain of group U via a spacer element.

In some embodiments L is:

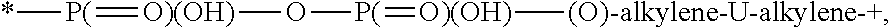

wherein:
U is selected from the group consisting of:

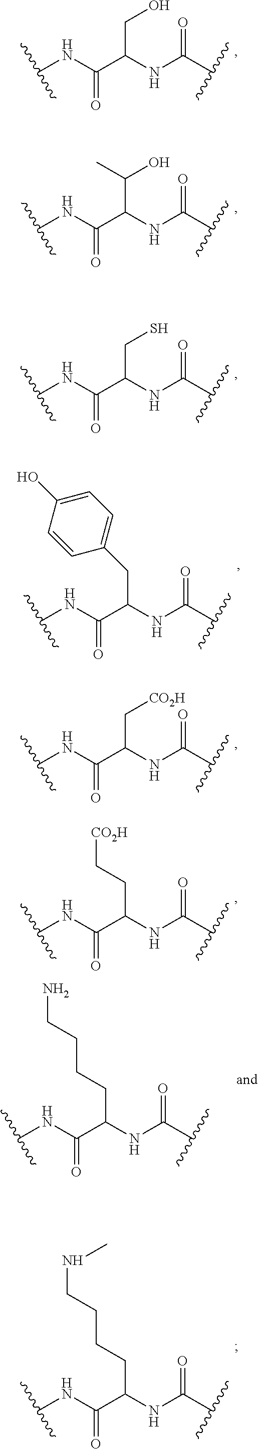

each alkylene is independently selected from the group consisting of:

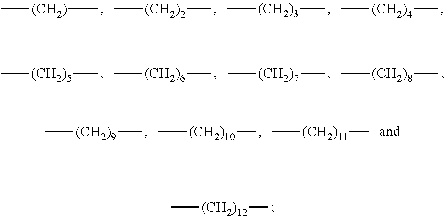

* denotes the connection to the —O— atom of Formula (ILc); and
+ denotes the connection to W;
   wherein L is optionally substituted with one or more water-soluble polymers.

In some embodiments, L is substituted with the one or more water-soluble polymers. In some embodiments, one water-soluble polymer is conjugated to an amino acid side chain of group U. In some embodiments, the one water-soluble polymer is conjugated to the amino acid side chain of group U via a spacer element. In some embodiments, the spacer element is a carbonyl group.

In some embodiments, U is:

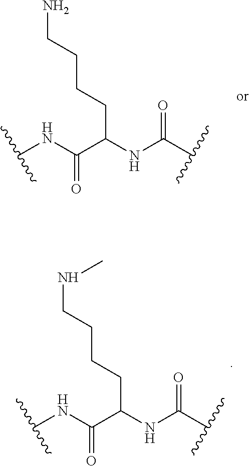 [SMILES: *NC(=O)C(CCCCN)NC(*)=O ; *NC(=O)C(CCCCNC)NC(*)=O]

In some embodiments, L has the following structure:

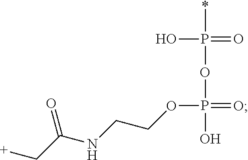

wherein: * denotes the connection to the —O— atom of Formula (ILc); and + denotes the connection to W.

In some other embodiments, L has the following structure:

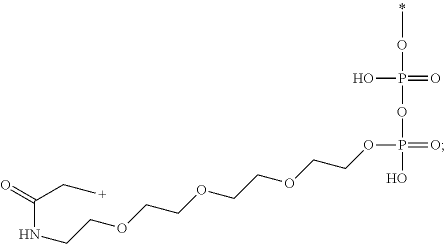

wherein: * denotes the connection to the —O— atom of Formula (ILc); and + denotes the connection to W.

In some other embodiments, L has the following structure:

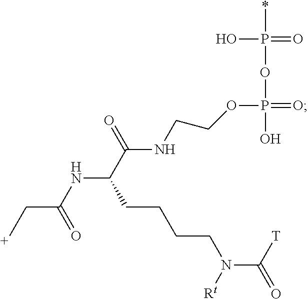

wherein: T is the water-soluble polymer; R^{t} is H or methyl; * denotes the connection to the —O— atom of Formula (ILc); and + denotes the connection to W.

In some embodiments, the water-soluble polymer is a polysaccharide.

In some embodiments, the water-soluble polymer is a (polyethylene)glycol (PEG) moiety. In some embodiments, the PEG moiety has a molecular weight within a range of about 100 Da to about 100,000 Da. In some embodiments, the PEG moiety has a molecular weight within a range of about 100 Da to about 10,000 Da. In some embodiments, the PEG moiety has a molecular weight within a range of about 100 Da to about 5,000 Da. In some embodiments, the PEG moiety has a molecular weight within a range of about 100 Da to about 1,000 Da.

In some embodiments, the PEG moiety is —(CH₂CH₂O)ₙCH₃, wherein n is an integer from 1 to 100. In some embodiments, the PEG moiety is —(CH₂CH₂O)ₙCH₃, wherein n is an integer from 1 to 24. In some embodiments, the PEG moiety is —(CH₂CH₂O)ₙCH₃, wherein n is an integer from 6 to 12. In some embodiments, the PEG moiety is —(CH₂CH₂O)ₙCH₃, wherein n is an integer from 8 to 12. In some embodiments, the PEG moiety is —(CH₂CH₂O)ₙCH₃, wherein n is 8. In some embodiments, the PEG moiety is —(CH₂CH₂O)ₙCH₃, wherein n is 12.

In some embodiments, the PEG is linear. In some embodiments, the PEG is branched, multi-armed or dendritic.

In some embodiments, the reactive moiety W comprises —N₃, —OH, —SH, —NH(Ri), —C(O)R^{q}, —C(O)OR^{x}, —C(O)CH₂NH₂, an activated ester, —O—NH₂, a maleimide, a tetrazine, an alkyne, a cyclooctyne or an (E)-cyclooctene; wherein Ri is H or unsubstituted alkyl, R^{q} is unsubstituted alkyl, and R^{x} is H, unsubstituted alkyl or a carboxylic acid protecting group.

In some embodiments, the reactive moiety W is selected from the group consisting of:

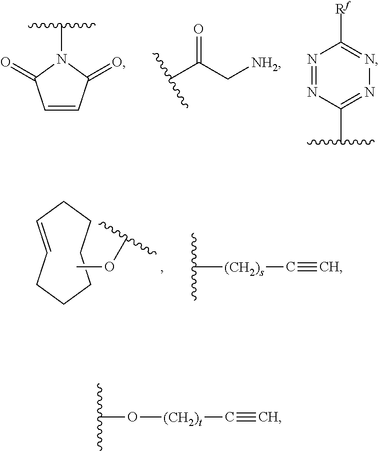

—N₂, —OH, —SH, —NH(R^{j}), —C(O)R^{q}, —C(O)OR^{x}, an activated ester, —O—NH₂ and an optionally substituted monocyclic or polycyclic group comprising the cyclooctyne;
wherein:
   R^{j} is H or unsubstituted C₁-C₆ alkyl,
   R^{q} is unsubstituted C₁-C₆ alkyl,
   R^{x} is H, unsubstituted C₁-C₆ alkyl or a carboxylic acid protecting group,
   R^{f} is H or unsubstituted C₁-C₆ alkyl,
   s is 0, 1, 2, 3, 4, 5 or 6, and
   t is 0, 1, 2, 3, 4, 5 or 6.

In some embodiments, the optionally substituted monocyclic or polycyclic group comprising the cyclooctyne is selected from the group consisting of:

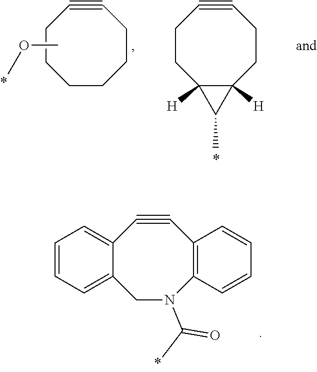

In some embodiments, W is —ONH₂.

In some aspects, there is provided a drug-linker compound of Formula (Id) wherein R is L-W. Thus, in some embodiments, there is provided a compound of Formula (ILd) having the following structure:

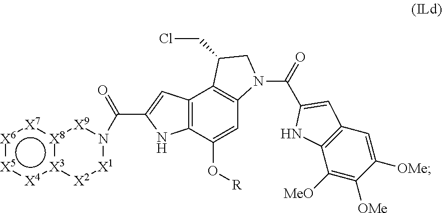

wherein:
R is L-W wherein L is a linker and W is a reactive moiety;
X¹ is C(R^{1a})(R^{1b}); wherein each R^{1a} and R^{1b} is independently H, halogen, alkyl, alkenyl or alkynyl;
X² is C(R^{2a})(R^{2b}); wherein each R^{2a} and R^{2b} is independently H, halogen, alkyl, alkenyl or alkynyl;
X³ is C;
X⁴ is C(R⁴) or N, wherein R⁴ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
X⁵ is C(R⁵) or N, wherein R⁵ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
X⁶ is C(R⁶) or N, wherein R⁶ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
X⁷ is C(R⁷) or N, wherein R⁷ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{S});
X⁸ is C; and
X⁹ is C(R^{9a})(R^{9b}); wherein each R^{9a} and R^{9b} is independently H, halogen, alkyl, alkenyl or alkynyl;
   wherein:
each R^{a} and R^{b} is independently H, alkyl, alkenyl or alkynyl;
each R^{c} is independently H, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl or heteroarylalkyl;
each R^{s} is independently H, halogen, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl or heteroarylalkyl; and
each m is independently 0, 1, 2 or 3.

In some embodiments, X¹ is C(R^{1a})(R^{1b}); wherein each R^{1a} and R^{1b} is independently H, halogen or unsubstituted alkyl; X² is C(R^{2a})(R^{2b}); wherein each R^{2a} and R^{2b} is independently H, halogen or unsubstituted alkyl; X³ is C; X⁴ is C(R⁴) or N, wherein R⁴ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl; X⁵ is C(R⁵) or N, wherein R⁵ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl; X⁶ is C(R⁶) or N, wherein R⁶ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl; X⁷ is C(R⁷) or N, wherein R⁷ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl; X⁸ is C; and X⁹, when present, is C(R^{9a})(R^{9b}); wherein each R^{9a} and R^{9b} is independently H, halogen or unsubstituted alkyl.

In some embodiments, each X¹ and X² is CH₂; and X⁹, when present, is CH₂.

In some embodiments, X⁴ is C(R⁴) or N, wherein R⁴ is H, halogen or heteroalkyl; X⁵ is C(R⁵) or N, wherein R⁵ is H, halogen or heteroalkyl; X⁶ is C(R⁶) or N, wherein R⁶ is H, halogen or heteroalkyl; and X⁷ is C(R⁷) or N, wherein R⁷ is H, halogen or heteroalkyl.

In some embodiments, X⁴ is C(R⁴) or N, wherein R⁴ is H or heteroalkyl; X⁵ is C(R⁵) or N, wherein R⁵ is H or heteroalkyl; X⁶ is C(R⁶) or N, wherein R⁶ is H or heteroalkyl; and X⁷ is C(R⁷) or N, wherein R⁷ is H or heteroalkyl.

In some embodiments, each heteroalkyl is an alkoxy. In some embodiments, each said alkoxy is independently —OR^{k}, wherein each R^{k} is independently alkyl optionally substituted with —N(R^{d})(R^{e}) or heterocyclyl; wherein each R^{d} and R^{e} is independently H, alkyl, alkenyl or alkynyl.

In some embodiments, each heterocyclyl contains at least one nitrogen atom.

In some embodiments, X⁴ is N, X⁵ is C(R⁵), X⁶ is C(R⁶) and X⁷ is C(R⁷). In some embodiments, X⁷ is CH.

In some embodiments, X⁴ is C(R⁴), X⁵ is N, X⁶ is C(R⁶) and X⁷ is C(R⁷). In some embodiments, at least one of X⁴ and X⁷ is CH.

In some embodiments, X⁴ is C(R⁴), X⁵ is C(R⁵), X⁶ is N and X⁷ is C(R⁷). In some embodiments, at least one of X⁴ and X⁷ is CH.

In some embodiments, X⁴ is C(R⁴), X⁵ is C(R⁵), X⁶ is C(R⁶) and X⁷ is N. In some embodiments, X⁴ is CH.

In some embodiments, X⁴ is C(R⁴), X⁵ is C(R⁵), X⁶ is C(R⁶) and X⁷ is C(R⁷).

In some embodiments, X⁷ is CH. In some embodiments, X⁴ is CH. In some embodiments, each of X⁴ and X⁷ is CH.

In some embodiments, L is a phosphate-based linker comprising a phosphate-based moiety. In some embodiments, the phosphate-based moiety is selected from the group consisting of a phosphate ester, a pyrophosphate ester, a triphosphate ester, a tetraphosphate ester, a phosphonate, a diphosphonate, a phosporamidate, a pyrophosporamidate, a triphosphoramidate, a tetraphosphoramidate, a phosphorthioate and a diphosphorthioate. In some embodiments, the phosphate-based moiety is a pyrophosphate ester. In some other embodiments, the phosphate-based moiety is a diphosphonate.

In some embodiments, the phosphate-based linker is a bivalent linker.

In some embodiments, the phosphate-based moiety of the phosphate-based linker is covalently bound to an —O— atom of the drug. Accordingly, in some embodiments, the phosphate-based moiety of the phosphate-based linker is covalently bound to an —O— atom of a compound of Formula (X) or Formula (I), as disclosed herein, via a phosphorous atom of the phosphate-based moiety.

In some embodiments, the phosphate-based linker further comprises at least one additional moiety. In some embodiments, each at least one additional moiety is independently selected from the group consisting of unsubstituted alkylene, substituted alkylene, -(alkylene-O)—, optionally substituted arylene, —O—, —C(O)—, —N(R_{w})—, —S(O)₀₋₂—, a water-soluble polymer and an amino acid; wherein each R_{w} is independently H or alkyl, alkenyl or alkynyl; and combinations thereof. In some embodiments, each R_{w} is independently H or unsubstituted C₁-C₈ alkyl, C₁-C₈ alkenyl or C₁-C₈ alkyl alkynyl. In some further embodiments, each R_{w} is independently H or unsubstituted C₁-C₈ alkyl. In yet some further embodiments, each R_{w} in independently H or methyl.

In some embodiments, each at least one additional moiety is independently selected from the group consisting of unsubstituted alkylene, -(alkylene-O)—, —C(O)—, —N(R_{w})—, a water-soluble polymer and an amino acid; wherein each R_{w} is independently H or alkyl, alkenyl or alkynyl; and combinations thereof. In some embodiments, each R_{w} is independently H or unsubstituted C₁-C₈ alkyl, C₁-C₈ alkenyl or C₁-C₈ alkyl alkynyl. In some further embodiments, each R_{w} is independently H or unsubstituted C₁-C₈ alkyl. In yet some further embodiments, each R_{w} in independently H or methyl.

It is understood that each at least one additional moiety that can be present in a phosphate-based linker of the present disclosure can occur one or more times within said linker. In a non-limiting example, a phosphate-based linker of the present disclosure can comprise one or more unsubstituted alkylene group, wherein, each said unsubstituted alkylene group can be the same or different. In another non-limiting example, a phosphate-based linker of the present disclosure can comprise one or more amino acids, wherein each amino acid is the same or different.

In some embodiments, L comprises at least one alkylene group.

In some embodiments, L comprises at least one amino acid. In some embodiments, L comprises one amino acid. In some embodiments, the amino acid is selected from the group consisting of serine, threonine, cysteine, tyrosine, aspartic acid, glutamic acid, lysine and N_{ϵ}-methyl-lysine. In some embodiments, the amino acid is lysine or N_{ϵ}-methyl-lysine.

In some embodiments, L comprises one or more water-soluble polymer. In some embodiments, L comprises one water-soluble polymer.

In some embodiments, L comprises a water-soluble polymer and an amino acid, wherein the water-soluble polymer is conjugated to the amino acid. In some embodiments, the water-soluble polymer is conjugated to a side chain of the amino acid. In some embodiments, the water-soluble polymer is conjugated to the amino acid via a spacer element.

In some embodiments, L is selected from the group of linkers listed in Table 6.

In some embodiments, L is selected from the group of linkers listed in Table 7.

In some embodiments, L is selected from the group of linkers listed in Table 8.

In some embodiments, L is selected from the group consisting of:
-P(=O)(OH)-O-P(=O)(OH)-(O)-alkylene-J-alkylene-+,
-P(=O)(OH)-O-P(=O)(OH)-(O)-(alkylene-O)ₙ-J-alkylene-+,
-P(=O)(OH)-O-P(=O)(OH)-(O)-alkylene-(O-alkylene)ₙ-J-alkylene-+,
-P(=O)(OH)-O-P(=O)(OH)-(O)-alkylene-J-(alkylene-O)ₙ-alkylene-+,
-P(=O)(OH)-O-P(=O)(OH)-(O)-alkylene-U-alkylene-+,
-P(=O)(OH)-O-P(=O)(OH)-(O)-alkylene-(O-alkylene)ₙ-U-alkylene-+,
-P(=O)(OH)-O-P(=O)(OH)-(O)-alkylene-(O-alkylene)ₙ-U-alkylene-+and
-P(=O)(OH)-O-P(=O)(OH)-(O)-alkylene-U-(alkylene-O)ₙ-alkylene-+;
   wherein:
each U is independently selected from the group consisting of:

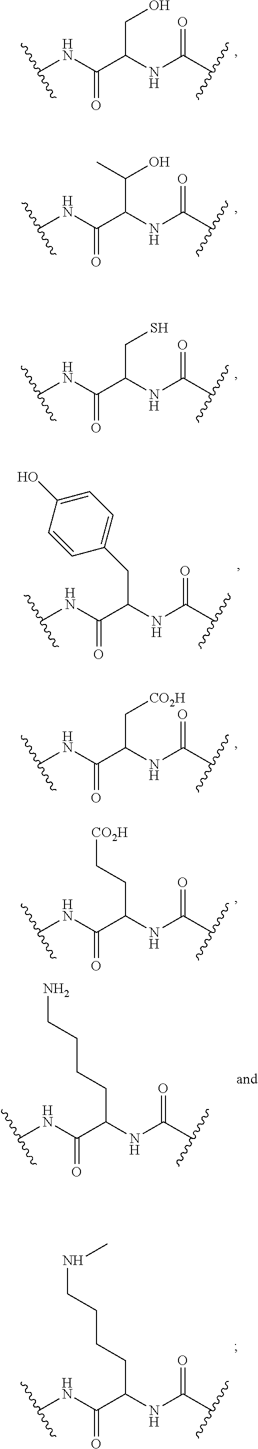

each J is independently

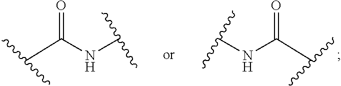 [SMILES: *NC(*)=O ; *NC(*)=O]

each alkylene is independently selected from the group consisting of:

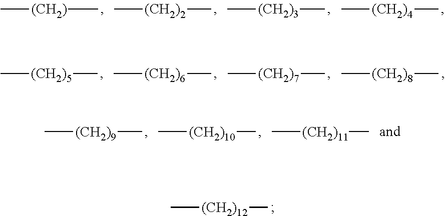

each n is independently an integer from 1 to 100;
* denotes the connection to the —O— atom of Formula (ILd); and
+ denotes the connection to W;
   wherein each linker L is optionally substituted with one or more water-soluble polymers.

In some embodiments, each n is independently an integer from 1 to 10. In some embodiments, each n is independently 1, 2 or 3.

In some embodiments, L is substituted with the one or more water-soluble polymer.

In some embodiments, L comprises group U, and one water-soluble polymer is conjugated to an amino acid side chain of group U. In some embodiments, the water-soluble polymer is conjugated to the amino acid side chain of group U via a spacer element.

In some embodiments, L is:

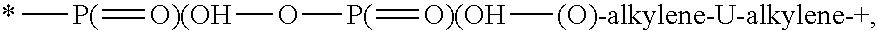

wherein:
U is selected from the group consisting of:

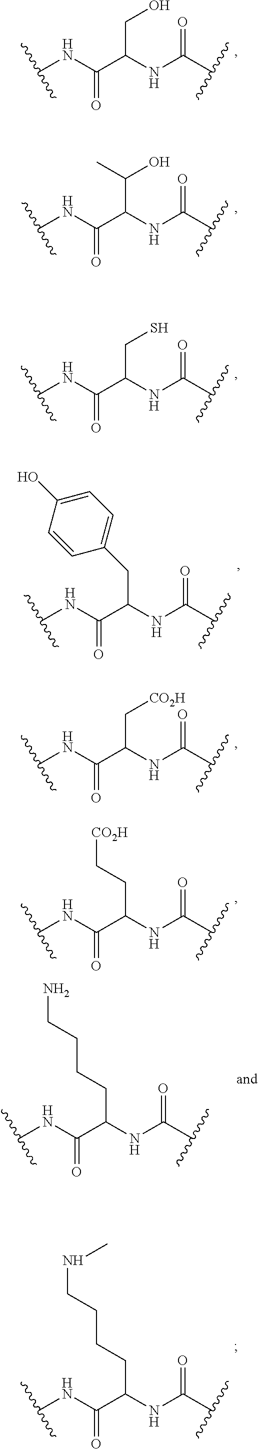

each alkylene is independently selected from the group consisting of:

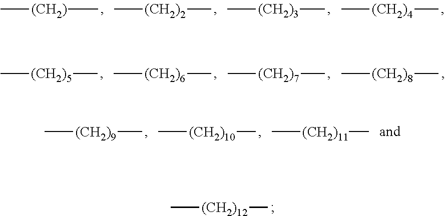

* denotes the connection to the —O— atom of Formula (ILd); and
+ denotes the connection to W;
   wherein L is optionally substituted with one or more water-soluble polymers.

In some embodiments, L is substituted with the one or more water-soluble polymers. In some embodiments, one water-soluble polymer is conjugated to an amino acid side chain of group U. In some embodiments, the one water-soluble polymer is conjugated to the amino acid side chain of group U via a spacer element. In some embodiments, the spacer element is a carbonyl group.

In some embodiments, U is:

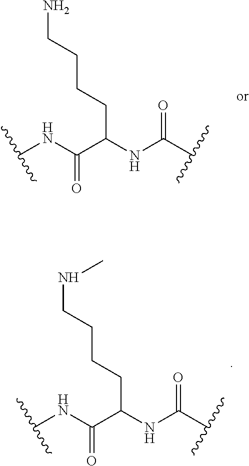 [SMILES: *NC(=O)C(CCCCN)NC(*)=O ; *NC(=O)C(CCCCNC)NC(*)=O]

In some embodiments, L has the following structure:

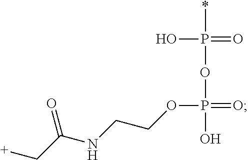

wherein: * denotes the connection to the —O— atom of Formula (ILd); and + denotes the connection to W.

In some other embodiments, L has the following structure:

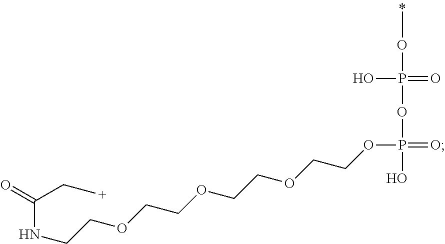

wherein: * denotes the connection to the —O— atom of Formula (ILd); and + denotes the connection to W.

In some other embodiments, L has the following structure:

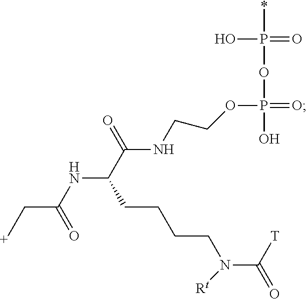

wherein: T is the water-soluble polymer; R^{t} is H or methyl; * denotes the connection to the —O— atom of Formula (ILd); and + denotes the connection to W.

In some embodiments, the water-soluble polymer is a polysaccharide.

In some embodiments, the water-soluble polymer is a (polyethylene)glycol (PEG) moiety. In some embodiments, the PEG moiety has a molecular weight within a range of about 100 Da to about 100,000 Da. In some embodiments, the PEG moiety has a molecular weight within a range of about 100 Da to about 10,000 Da. In some embodiments, the PEG moiety has a molecular weight within a range of about 100 Da to about 5,000 Da. In some embodiments, the PEG moiety has a molecular weight within a range of about 100 Da to about 1,000 Da.

In some embodiments, the PEG moiety is —(CH₂CH₂O)ₙCH₃, wherein n is an integer from 1 to 100. In some embodiments, the PEG moiety is —(CH₂CH₂O)ₙCH₃, wherein n is an integer from 1 to 24. In some embodiments, the PEG moiety is —(CH₂CH₂O)ₙCH₃, wherein n is an integer from 6 to 12. In some embodiments, the PEG moiety is —(CH₂CH₂O)ₙCH₃, wherein n is an integer from 8 to 12. In some embodiments, the PEG moiety is —(CH₂CH₂O)ₙCH₃, wherein n is 8. In some embodiments, the PEG moiety is —(CH₂CH₂O)ₙCH₃, wherein n is 12.

In some embodiments, the PEG is linear. In some embodiments, the PEG is branched, multi-armed or dendritic.

In some embodiments, the reactive moiety W comprises —N₃, —OH, —SH, —NH(R^{j}), —C(O)R^{q}, —C(O)OR^{x}, —C(O)CH₂NH₂, an activated ester, —O—NH₂, a maleimide, a tetrazine, an alkyne, a cyclooctyne or an (E)-cyclooctene; wherein R^{j} is H or unsubstituted alkyl, R^{q} is unsubstituted alkyl, and R^{x} is H, unsubstituted alkyl or a carboxylic acid protecting group.

In some embodiments, the reactive moiety W is selected from the group consisting of:

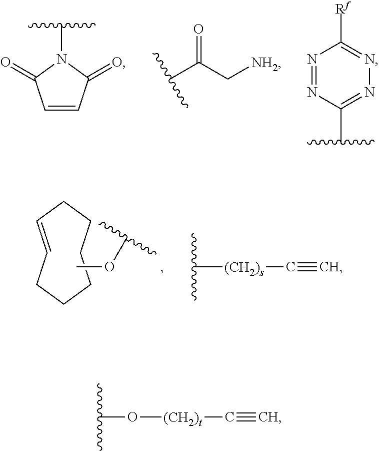

—N₃, —OH, —SH, —NH(R^{j}), —C(O)R^{q}, —C(O)OR^{x}, an activated ester, —O—NH₂ and an optionally substituted monocyclic or polycyclic group comprising the cyclooctyne;
wherein:
   R^{j} is H or unsubstituted C₁-C₆ alkyl,
   R^{q} is unsubstituted C₁-C₆ alkyl,
   R^{x} is H, unsubstituted C₁-C₆ alkyl or a carboxylic acid protecting group,
   R^{f} is H or unsubstituted C₁-C₆ alkyl,
   s is 0, 1, 2, 3, 4, 5 or 6, and
   t is 0, 1, 2, 3, 4, 5 or 6.

In some embodiments, the optionally substituted monocyclic or polycyclic group comprising the cyclooctyne is selected from the group consisting of:

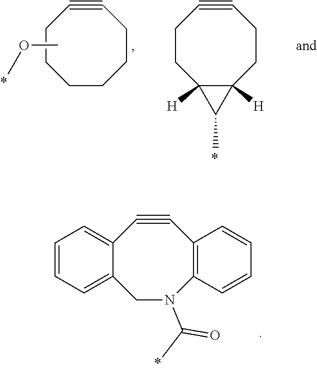

In some embodiments, W is —ONH₂.

Table 9 provides exemplary drug-linker compounds that can be employed or conjugated with any targeting ligand such as an antibody or antibody fragment, that is selected based in its specificity for an antigen expressed on a target cell or at a target site of interest. The drug-linkers of the invention can be employed with antibody or antibody fragments to a variety of antigens including but not limited to tumor associated antigens, tumor specific antigens, cancer antigens or diseases specific antigens. Non-limiting examples of such antigens include PD-1, PD-L1, PSMA, CD70, CD3, HER2, HER3, TROP2, GPC3, VEGFR, EGFR, c-Met (HGFR), CD4, CD33, CD19, CD22, CD25 (IL-2R alpha), CD30, CD37, CD38, CD40L, CD44, CD46, CD47, CD48, CD52, CD56 (NCAM-1), CD71 (Transferrin R), CD74, CD79b, CD80, CD123 (IL-3R alpha), CD138 (syndecan-1), CD142, CD163, CD166 (ALCAM), CD203c (ENPP3), CD205 (LY75), CD221 (IGF-1R), CD262 (TRAIL R2), CD276 (B7-H3), CTLA4, integrins, mesothelin, EpCAM, CEACAM5, CEACAM6, DLL3, FOLR1, ROR1, ROR2, GPNMB, GCC, GUCY2c, NaPi2b, Flt-1, Flt-3, folate receptor alpha, Tissue Factor (TF), CA6, MUC1, MUC16 (CA-125), BCMA, SLAMF7 (CS1), TIM1, CanAg, Ckit (CD117), EphA2, Nectin4, SLTRK6, FGFR2, LYPD3 (C4.4a), Cadherin 3, 5T4 (TPBG), STEAP1, PTK7, Ephrin-A4, LIV-1 (SLC39A6 or ZIP6), SLC1A5, TENB2, ETBR, integrin v3, Cripto, AGS-5 (SLC44A4), LY6E, AXL, LAMP1, LRRC15, TNF-alpha, and MN/CA IX.

In some embodiments, drug-linker compounds disclosed in Table 9 can be employed with anti-HER2 antibody, antibody fragments or antibody drug conjugates of the invention. In some embodiments, drug-linker compounds disclosed in Table 9 can be employed with an anti-CD3 antibody, antibody fragments or antibody drug conjugates of the present disclosure. In some embodiments, drug-linker compounds disclosed in Table 9 can be employed with anti-CD70 antibody, antibody fragments or antibody drug conjugates of the invention. In some embodiments, drug-linker compounds disclosed in Table 9 can be employed with anti-PSMA antibody, antibody fragments or antibody drug conjugates of the invention. In some embodiments, drug-linker compounds disclosed in Table 9 can be employed with an anti-TROP2 antibody, antibody fragments or antibody drug conjugates of the present disclosure. In some embodiments, drug-linker compounds disclosed in Table 9 can be employed with an anti-HER3 antibody, antibody fragments or antibody drug conjugates of the present disclosure. In some embodiments, drug-linker compounds disclosed in Table 9 can be employed with an anti-GPC3 antibody, antibody fragments or antibody drug conjugates of the present disclosure.

TABLE 9
Non-limiting Drug-linker Compounds of the Invention

| Compound | Structure |
|---|---|
| 12 | 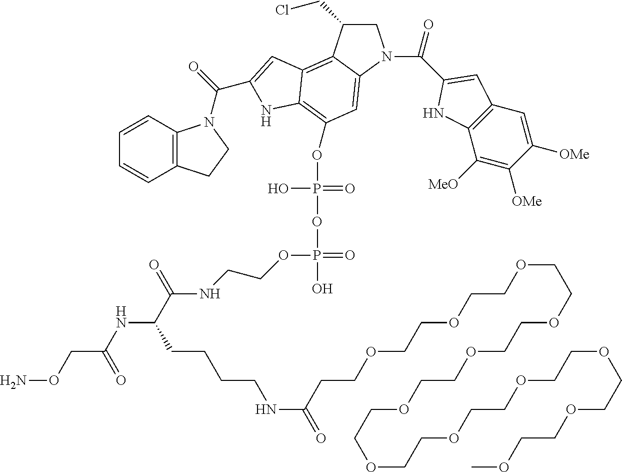 [SMILES: COCCOCCOCCOCCOCCOCCOCCOCCOCCOCCOCCOCCC(=O)NCCCC[C@H](NC(=O)CON)C(=O)NCCOP(=O)(O)OP(=O)(O)Oc1cc2c(c3cc(C(=O)N4CCc5ccccc54)[nH]c13)[C@H](CCl)CN2C(=O)c1cc2cc(OC)c(OC)c(OC)c2[nH]1] |
| 14 | 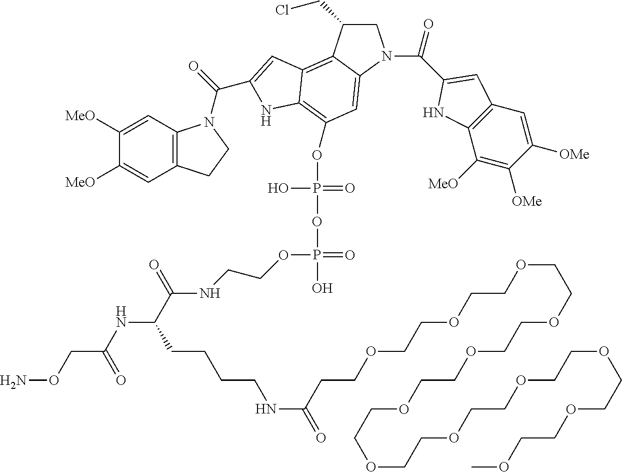 [SMILES: COCCOCCOCCOCCOCCOCCOCCOCCOCCOCCOCCOCCC(=O)NCCCC[C@H](NC(=O)CON)C(=O)NCCOP(=O)(O)OP(=O)(O)Oc1cc2c(c3cc(C(=O)N4CCc5cc(OC)c(OC)cc54)[nH]c13)[C@H](CCl)CN2C(=O)c1cc2cc(OC)c(OC)c(OC)c2[nH]1] |
| 16 | 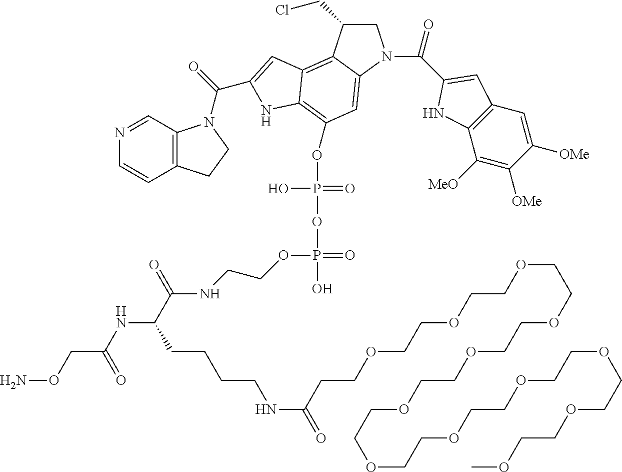 [SMILES: COCCOCCOCCOCCOCCOCCOCCOCCOCCOCCOCCOCCC(=O)NCCCC[C@H](NC(=O)CON)C(=O)NCCOP(=O)(O)OP(=O)(O)Oc1cc2c(c3cc(C(=O)N4CCc5ccncc54)[nH]c13)[C@H](CCl)CN2C(=O)c1cc2cc(OC)c(OC)c(OC)c2[nH]1] |
| 18 | 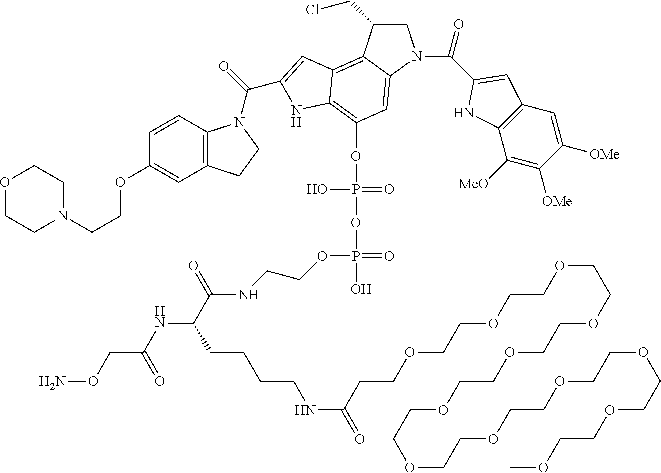 [SMILES: COCCOCCOCCOCCOCCOCCOCCOCCOCCOCCOCCOCCC(=O)NCCCC[C@H](NC(=O)CON)C(=O)NCCOP(=O)(O)OP(=O)(O)Oc1cc2c(c3cc(C(=O)N4CCc5cc(OCCN6CCOCC6)ccc54)[nH]c13)[C@H](CCl)CN2C(=O)c1cc2cc(OC)c(OC)c(OC)c2[nH]1] |
| 19 | 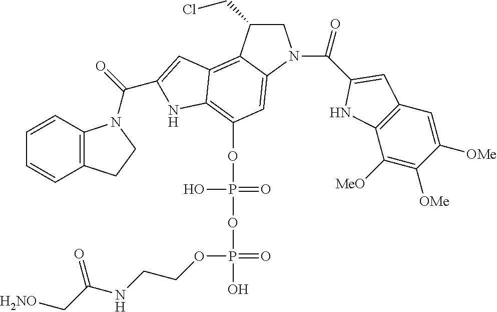 |
| 20 | 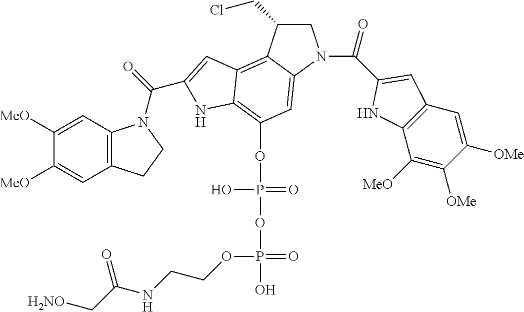 |
| 21 | 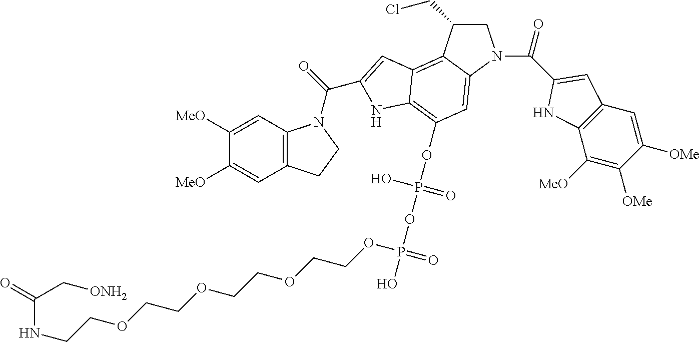 |
| 22 | 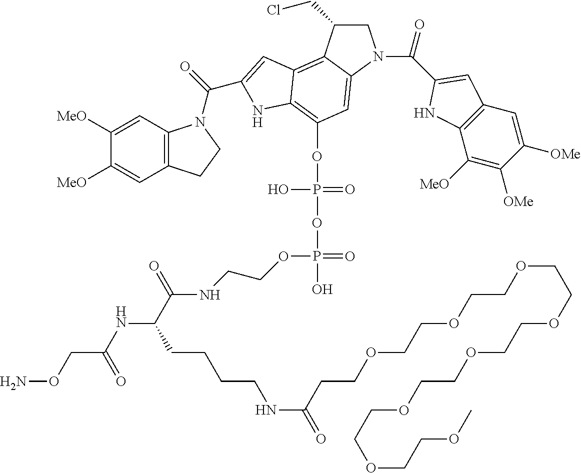 [SMILES: COCCOCCOCCOCCOCCOCCOCCOCCC(=O)NCCCC[C@H](NC(=O)CON)C(=O)NCCOP(=O)(O)OP(=O)(O)Oc1cc2c(c3cc(C(=O)N4CCc5cc(OC)c(OC)cc54)[nH]c13)[C@H](CCl)CN2C(=O)c1cc2cc(OC)c(OC)c(OC)c2[nH]1] |
| 23 | 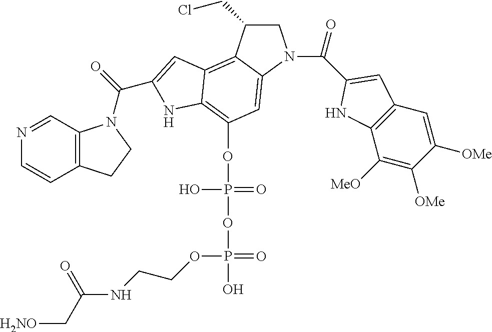 |
| 24 | 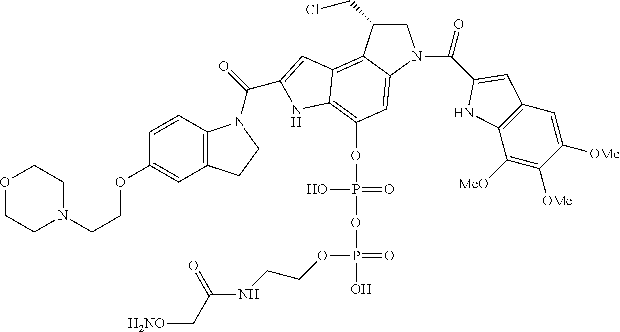 |

In some embodiments, the disclosure provides a compound selected from the group consisting of compounds listed in Table 9, and salts thereof. In some further embodiments, there is provided a compound selected from the group consisting of compound 12, 14, 16, 18 and 22, and salts thereof. In yet further embodiments, there is provided a compound selected from the group consisting of compound 12, 14, 16 and 18, and salts thereof.

In some embodiments the present invention provides additional drug-linkers prepared using similar procedures as described herein, including the schemes disclosed in the Examples. Additional drug-linker compounds are engineered by linkage of any possible linker group known in the art or elsewhere. The drug-linker compounds are engineered by linkage of one or more phosphate-based linkers via any chemical or functional reactive positions in the drug, for example a nitrogen, halogen, boron, phosphorus, silicon, carbon or oxygen of the cytotoxic agent. Selection of the nitrogen, halogen, boron, phosphorus, silicon, sulfur, carbon or oxygen position in the drug for linkage to a phosphate-based linker is assessed as disclosed elsewhere herein, based on structure of the cytotoxic agent, and using the process known in the art or elsewhere to generate a phosphate-drug linkage. In some embodiments, drug-linkers of the invention include a phosphate-based linker attached or linked at a hydroxyl group of the cytotoxic agent or analogue thereof, such as a topoisomerase inhibitor. In other embodiments, drug-linkers of the invention include a phosphate-based linker attached or linked at a methyl or methylene group of the cytotoxic agent or analogue thereof. In some embodiments, such additional drug-linker compounds can comprise a branched linker, which connects to two identical or different drugs. In some embodiments, drug-linkers of the present invention include drug-linkers generated via linkage of one or more phosphate-based linkers at one or more a nitrogen, halogen, boron, phosphorus, silicon, sulfur, carbon, or oxygen of the cytotoxic agent.

The present disclosure provides drug moieties with linkers that reduce the toxicity of the moiety in vivo while retaining pharmacological activity. In some embodiments, the toxicity of the linked drug, when administered to an animal or human, is reduced or eliminated compared to the free toxic group or toxic group derivatives comprising labile linkages, while retaining pharmacological activity. In some embodiments, increased doses of the linked toxic group may be administered to animals or humans with greater safety. In certain embodiments, the non-natural amino acid polypeptides linked to a drug moiety (e.g., a duocarmycin or a duocarmycin derivative or analog) provides in vitro and in vivo stability. In some embodiments, the non-natural amino acid polypeptides linked to a drug moiety are efficacious and less toxic compared to the free drug moiety.

In some embodiments, at least one post-translational modification at some position on the polypeptide may occur. In some embodiments the co-translational or post-translational modification occurs via the cellular machinery (e.g., glycosylation, acetylation, acylation, lipid-modification, palmitoylation, palmitate addition, phosphorylation, glycolipid-linkage modification, and the like), in many instances, such cellular-machinery-based co-translational or post-translational modifications occur at the naturally occurring amino acid sites on the polypeptide, however, in certain embodiments, the cellular-machinery-based co-translational or post-translational modifications occur on the non-natural amino acid site(s) on the polypeptide.

In other embodiments, the post-translational modification does not utilize the cellular machinery, but the functionality is instead provided by attachment of a molecule (a polymer; a water-soluble polymer; a derivative of polyethylene glycol; a second protein or polypeptide or polypeptide analog; an antibody or antibody fragment; and any combination thereof) comprising a second reactive group to the at least one non-natural amino acid comprising a first reactive group (including but not limited to, non-natural amino acid containing a ketone, aldehyde, acetal, hemiacetal, alkyne, cycloalkyne, azide, oxime, or hydroxylamine functional group) utilizing chemistry methodology described herein, or others suitable for the particular reactive groups. In certain embodiments, the co-translational or post-translational modification is made in vivo in a eukaryotic cell or in a non-eukaryotic cell. In certain embodiments, the post-translational modification is made in vitro not utilizing the cellular machinery. Also included with this aspect are methods for producing, purifying, characterizing and using such a drug-linker containing at least one such co-translationally or post-translationally modified non-natural amino acids.

Also included within the scope of the methods, compositions, strategies and techniques described herein are reagents capable of reacting with a drug-linker (containing a carbonyl or dicarbonyl group, alkyne, cycloalkyne, azide, hydroxylamine group, or masked or protected forms thereof) that is part of a polypeptide so as to produce any of the aforementioned post-translational modifications. In certain embodiments, the resulting post-translationally modified drug-linker will contain at least one oxime group; the resulting modified oxime-containing drug-linker may undergo subsequent modification reactions. Also included with this aspect are methods for producing, purifying, characterizing and using such reagents that are capable of any such post-translational modifications of such drug-linkers.

In certain embodiments, the polypeptide or non-natural amino acid linked composition includes at least one co-translational or post-translational modification that is made in vivo by one host cell, where the post-translational modification is not normally made by another host cell type. In certain embodiments, the polypeptide includes at least one co-translational or post-translational modification that is made in vivo by a eukaryotic cell, where the co-translational or post-translational modification is not normally made by a non-eukaryotic cell. Examples of such co-translational or post-translational modifications include, but are not limited to, glycosylation, acetylation, acylation, lipid-modification, palmitoylation, palmitate addition, phosphorylation, glycolipid-linkage modification, and the like. In one embodiment, the co-translational or post-translational modification comprises attachment of an oligosaccharide to an asparagine by a GlcNAc-asparagine linkage (including but not limited to, where the oligosaccharide comprises (GlcNAc-Man)₂-Man-GlcNAc-GlcNAc, and the like). In another embodiment, the co-translational or post-translational modification comprises attachment of an oligosaccharide (including but not limited to, Gal-GalNAc, Gal-GlcNAc, etc.) to a serine or threonine by a GalNAc-serine, a GalNAc-threonine, a GlcNAc-serine, or a GlcNAc-threonine linkage. In certain embodiments, a protein or polypeptide can comprise a secretion or localization sequence, an epitope tag, a FLAG tag, a polyhistidine tag, a GST fusion, and/or the like. Also included with this aspect are methods for producing, purifying, characterizing and using such polypeptides containing at least one such co-translational or post-translational modification. In other embodiments, the glycosylated non-natural amino acid polypeptide is produced in a non-glycosylated form. Such a non-glycosylated form of a glycosylated non-natural amino acid may be produced by methods that include chemical or enzymatic removal of oligosaccharide groups from an isolated or substantially purified or unpurified glycosylated non-natural amino acid polypeptide; production of the non-natural amino acid in a host that does not glycosylate such a non-natural amino acid polypeptide (such a host including, prokaryotes or eukaryotes engineered or mutated to not glycosylate such a polypeptide), the introduction of a glycosylation inhibitor into the cell culture medium in which such a non-natural amino acid polypeptide is being produced by a eukaryote that normally would glycosylate such a polypeptide, or a combination of any such methods. Also described herein are such non-glycosylated forms of normally-glycosylated non-natural amino acid polypeptides (by normally-glycosylated is meant a polypeptide that would be glycosylated when produced under conditions in which naturally-occurring polypeptides are glycosylated). Of course, such non-glycosylated forms of normally-glycosylated non-natural amino acid polypeptides (or indeed any polypeptide described herein) may be in an unpurified form, a substantially purified form, or in an isolated form.

In some instances, incorporation of a non-natural amino acid into the antibody or antibody fragment will be combined with other additions, substitutions, or deletions within the polypeptide to affect other chemical, physical, pharmacologic and/or biological traits. In some cases, the other additions, substitutions or deletions may increase the stability (including but not limited to, resistance to proteolytic degradation) of the polypeptide or increase affinity of the polypeptide for its appropriate receptor, ligand and/or binding proteins. In some cases, the other additions, substitutions or deletions may increase the solubility (including but not limited to, when expressed in E. coli or other host cells) of the polypeptide. In some embodiments, sites are selected for substitution with a naturally encoded or non-natural amino acid in addition to another site for incorporation of a non-natural amino acid for the purpose of increasing the polypeptide solubility following expression in E. coli, or other recombinant host cells. In some embodiments, the polypeptides comprise another addition, substitution, or deletion that modulates affinity for the associated ligand, binding proteins, and/or receptor, modulates (including but not limited to, increases or decreases) receptor dimerization, stabilizes receptor dimers, modulates circulating half-life, modulates release or bio-availability, facilitates purification, or improves or alters a particular route of administration. Similarly, the non-natural amino acid polypeptide can comprise chemical or enzyme cleavage sequences, protease cleavage sequences, reactive groups, antibody-binding domains (including but not limited to, FLAG or poly-His) or other affinity based sequences (including but not limited to, FLAG, poly-His, GST, etc.) or linked molecules (including but not limited to, biotin) that improve detection (including but not limited to, GFP), purification, transport thru tissues or cell membranes, prodrug release or activation, size reduction, or other traits of the polypeptide.

### Antibody Drug Conjugates (ADCs)

Antibody drug conjugates (ADCs) of the present disclosure provide novel therapeutics or anti-cancer drugs by combining the selectivity of antibodies comprising one or more non-natural amino acids and a cytotoxic agent. Targeted cytotoxic drug delivery into tumor tissue increases the therapeutic window of these agents considerably. ADCs of the present disclosure comprise of an antibody bound to a cytotoxic drug via a linker. Stability of the linker between the antibody and the cytotoxic drug is essential for the ADC integrity in circulation. The successful ADC development for a given target antigen depends on optimization of antibody selection, linker design and stability, drug potency and mode of drug and linker conjugation to the antibody. Linker properties of pH and redox sensitivities and protease susceptibility influence circulatory stability and release of the drug moiety.

In some embodiments of the disclosure, the antibody of the ADC comprises a full length antibody or fragment thereof that binds to an antigen, and is conjugated to a cytotoxic agent or an immunosuppressive agent, wherein the antibody-drug conjugate exerts: (a) a cytotoxic or cytostatic effect on the antigen-expressing or antigen targeting cell line, or (b) a cytotoxic, cytostatic, or immunosuppressive/immune activating effect on an antigen-expressing immune cell, wherein the conjugation occurs at a non-natural amino acid in the antibody. In some embodiments, the antigen, antigen-expressing cell, or antigen-targeting cell, or antigen-expressing immune cell is PD-1, PD-Li, PSMA, CD70, CD3, HER2, HER3, TROP2, GPC3, VEGFR, EGFR, c-Met (HGFR), CD19, CD22, CD25 (IL-2R alpha), CD30, CD33, CD37, CD46, CD48, CD56 (NCAM-1), CD71 (Transferrin R), CD74, CD79b, C-D123 (IL-3R alpha), CD138 (syndecan-1), CD142, CD166 (ALCAM), CD203c (ENPP3), CD205 (LY75), CD221 (IGF-1R), CD262 (TRAIL R2), CD276 (B7-H3), mesothelin, EpCAM, CEACAM5, CEACAM6, DLL3, ROR1, ROR2, GPNMB, GCC, GUCY2c, NaPi2b, Flt-1, Flt-3, folate receptor alpha, Tissue Factor (TF), CA6, MUC1, MUC16 (CA-125), BCMA, SLAMF7 (CS1), TIM1, CanAg, Ckit (CD117), EphA2, Nectin4, SLTRK6, FGFR2, LYPD3 (C4.4a), Cadherin 3, 5T4 (TPBG), STEAP1, PTK7, Ephrin-A4, LIV-1 (SLC39A6 or ZIP6), SLC1A5, TENB2, ETBR, integrin v3, Cripto, AGS-5 (SLC44A4), LY6E, AXL, LAMP1, LRRC15, TNF-alpha, or MN/CA IX, but is not limited to such. In some embodiments, the antigen, antigen-expressing cell, or antigen-targeting cell, or antigen-expressing immune cell is a TROP2, or HER2 or CD70 antigen, or antigen-targeting cell, or antigen-expressing cell or antigen-expressing immune cell. In some embodiments of the disclosure, the antibody of the ADC comprises a full length antibody or fragment thereof that: binds to CD70 and is conjugated to a cytotoxic agent or an immunosuppressive agent, wherein the antibody-drug conjugate exerts: (a) a cytotoxic or cytostatic effect on a CD70-expressing cancer cell line, or (b) a cytotoxic, cytostatic, or immunosuppressive/immune activating effect on a CD70-expressing immune cell, wherein the conjugation occurs at a non-natural amino acid in the antibody.

In some embodiments, the antibody, variant, or composition of the present disclosure may be an antibody, variant, or composition that binds to an antigen receptor. In other embodiments the antibody, variant, or composition may be an antibody, variant, or composition that binds to extracellular surface of an antigen receptor. In some embodiments the antibody, variant, or composition of the present disclosure may be an antibody, variant, or composition that has CDRs grafted onto the framework region of the variable region. In other embodiments the antibody, variant, or composition of the present disclosure may be an antibody, variant, or composition that has a non-natural amino acid. In some embodiments the antibody, variant, or composition may be an antibody, variant, or composition that is described by more than one of the embodiments elsewhere herein the present disclosure. In some embodiments the antibody, antibody variant or antibody composition(s) disclosed herein may be fully humanized. In other embodiments the antibody, antibody variant or antibody composition(s) disclosed herein may be chimeric. In some embodiments the antibody may be an antibody that is full length antibody (Variable+Fc regions), Fab, bispecific, Fab-dimers, Fab-bispecific, Fab-trispecific, bispecific T-cell engagers, dual-affinity re-targeting antibody, IgG1/IgG3 bispecific antibody, diabody, bispecific diabody, scFv-Fc, minibody.

In some embodiments, the ADC comprises an antibody conjugated to a drug wherein the conjugation occurs via a non-natural amino acid in the antibody. The antibody comprises at least one non-natural amino acid; non-limiting examples of non-natural amino acids are disclosed herein.

In some embodiment, the ADC comprises an antibody conjugated to a drug wherein the conjugation occurs via a non-natural amino acid in the heavy chain of the antibody. In some embodiments, the ADC comprises an antibody conjugated to a drug wherein the conjugation occurs via a non-natural amino acid in the light chain of the antibody. In some embodiments, the ADC comprises a full-length antibody conjugated to a drug wherein the conjugation occurs via a non-natural amino acid in the antibody. In some embodiments, the ADC comprises a full-length antibody conjugated to a drug wherein the conjugation occurs via a non-natural amino acid in the heavy chain of the antibody. In some embodiments, the ADC comprises a full-length antibody conjugated to a drug wherein the conjugation occurs via a non-natural amino acid in the light chain of the antibody. In some embodiments, the ADC comprises a full-length antibody conjugated to a drug wherein a first conjugation occurs via a non-natural amino acid in the heavy chain of the antibody, and a second conjugation occurs via a non-natural amino acid in the light chain of the antibody. In some embodiments, the full-length antibody comprises two full-length heavy chains and two full-length light chains, wherein a first pair of conjugations occur via a non-natural amino acid in each heavy chain of the antibody, and a second pair of conjugations occur via a non-natural amino acid in each light chain of the antibody.

In some embodiments, the drug of the ADC is a cytotoxic drug or agent. In some aspects of the invention, the cytotoxic drug is a duocarmycin analog, such as a duocarmycin analog of the present disclosure. In some embodiments the drug is a drug generated as described in the Examples herein. In some embodiments, the ADC comprises an antibody, antibody fragment or variant thereof engineered to have one or more non-natural amino acids site specifically incorporated in the heavy and/or light chain amino acid sequence conjugated to drug via a phosphate-based linker.

In some aspects, the present disclosure provides an ADC of Formula (II):

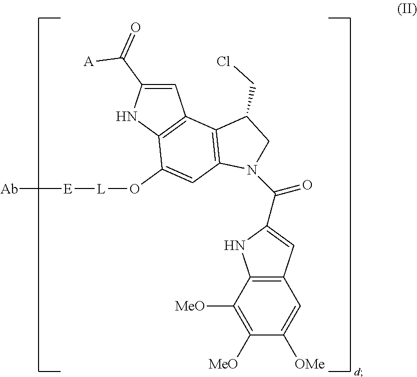

wherein:
Ab is an antibody, wherein Ab comprises one or more non-natural amino acids;
L is a linker;
E is a moiety joining Ab and L;
d is an integer from 1 to 100; and
A is a bicyclic ring system selected from the group consisting of formula (a), (b), (c) and (d), having the following structures:

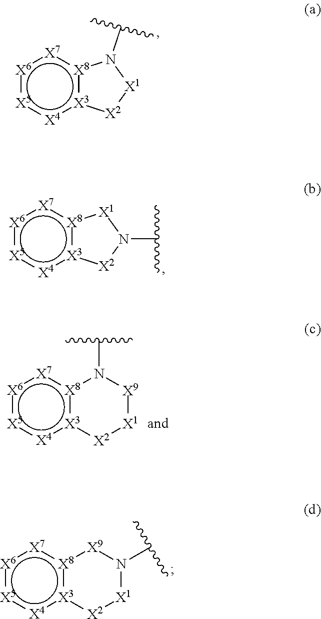

wherein:
   each X¹ is C(R^{1a})(R^{1b}); wherein each R^{1a} and R^{1b} is independently H, halogen, alkyl, alkenyl or alkynyl;
   each X² is C(R^{2a})(R^{2b}); wherein each R^{2a} and R^{2b} is independently H, halogen, alkyl, alkenyl or alkynyl;
   each X³ is C;
   each X⁴ is C(R⁴) or N, wherein R⁴ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
   each X⁵ is C(R⁵) or N, wherein R⁵ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
   each X⁶ is C(R⁶) or N, wherein R⁶ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
   each X⁷ is C(R⁷) or N, wherein R⁷ is H, halogen, —OH, —SH, —NO₂, —CN, —N₃, —N(R^{a})(R^{b}) acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
   each X⁸ is C; and
   each X⁹, when present, is C(R^{9a})(R^{9b}); wherein each R^{9a} and R^{9b} is independently H, halogen, alkyl, alkenyl or alkynyl;
wherein:
   each R^{a} and R^{b} is independently H, alkyl, alkenyl or alkynyl;
   each R^{c} is independently H, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl or heteroarylalkyl;
   each R^{s} is independently H, halogen, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl or heteroarylalkyl; and
   each m is independently 0, 1, 2 or 3;
or a pharmaceutically acceptable salt thereof.

In some embodiments, A-H (the corresponding amine of moiety A) has a C log P value of at least about 1.

In some embodiments, A has the structure of formula (a):

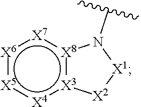

In some embodiments, the corresponding amine of formula (a) has a C log P value of at least about 1.

In some embodiments, A has the structure of formula (b):

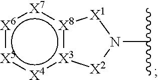

In some embodiments, the corresponding amine of formula (b) has a C log P value of at least about 1.

In some embodiments, A has the structure of formula (c):

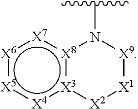

In some embodiments, the corresponding amine of formula (c) has a C log P value of at least about 1.

In some embodiments, A has the structure of formula (d):

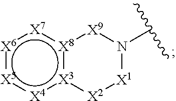

In some embodiments, the corresponding amine of formula (d) has a C log P value of at least about 1.

In some embodiments, the antibody Ab comprises one or more non-natural amino acids.

In some embodiments, d is an integer from 1 to 100. In some embodiments, d is an integer from 1 to 10. In some embodiments, d is 1, 2, 3, 4, 5, 6, 7, 8, 9 or 10. In some embodiments, d is 1, 2, 3 or 4. In some embodiments, d is 1. In some embodiments, d is 2. In some embodiments, d is 3. In some embodiments, d is 4.

In some further embodiments of Formula (II):
X¹ is C(R^{1a})(R^{1b}); wherein each R^{1a} and R^{1b} is independently H, halogen or unsubstituted alkyl;
X² is C(R^{2a})(R^{2b}); wherein each R^{2a} and R^{2b} is independently H, halogen or unsubstituted alkyl;
X³ is C;
X⁴ is C(R⁴) or N, wherein R⁴ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl;
X⁵ is C(R⁵) or N, wherein R⁵ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl;
X⁶ is C(R⁶) or N, wherein R⁶ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl;
X⁷ is C(R⁷) or N, wherein R⁷ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl;
X⁸ is C; and
X⁹, when present, is C(R^{9a})(R^{9b}); wherein each R^{9a} and R^{9b} is independently H, halogen or unsubstituted alkyl.

In some embodiments, each X¹ and X² is CH₂; and X⁹, when present, is CH₂.

In some embodiments, X⁴ is C(R⁴) or N, wherein R⁴ is H, halogen or heteroalkyl; X⁵ is C(R⁵) or N, wherein R⁵ is H, halogen or heteroalkyl; X⁶ is C(R⁶) or N, wherein R⁶ is H, halogen or heteroalkyl; and X⁷ is C(R⁷) or N, wherein R⁷ is H, halogen or heteroalkyl.

In some embodiments, each said heteroalkyl is an alkoxy. In some embodiments, each said alkoxy is independently —OR^{k}, wherein each R^{k} is independently alkyl, wherein said alkyl is optionally substituted with —N(R^{d})(R^{e}) or heterocyclyl; wherein each R^{d} and R^{e} is independently H, alkyl, alkenyl or alkynyl, and wherein each heterocyclyl contains at least one nitrogen atom.

In some embodiments, X⁴ is N, X⁵ is C(R⁵), X⁶ is C(R⁶) and X⁷ is C(R⁷). In some other embodiments, X⁴ is C(R⁴), X⁵ is N, X⁶ is C(R⁶) and X⁷ is C(R⁷). In some other embodiments, X⁴ is C(R⁴), X⁵ is C(R⁵), X⁶ is N and X⁷ is C(R⁷). In some embodiments, X⁷ is CH. In some other embodiments, at least one of X⁴ and X⁷ is CH.

In some embodiments, X⁴ is C(R⁴), X⁵ is C(R⁵), X⁶ is C(R⁶) and X⁷ is N. In some embodiments, X⁴ is CH.

In some embodiments, X⁴ is C(R⁴), X⁵ is C(R⁵), X⁶ is C(R⁶) and X⁷ is C(R⁷). In some embodiments, X⁴ is CH, X⁷ is CH, or both.

In some embodiments, d is an integer from 1 to 100. In some embodiments, d is an integer from 1 to 10. In some embodiments, d is 1, 2, 3 or 4. In some embodiments, d is 1. In some embodiments, d is 2. In some embodiments, d is 3. In some embodiments, d is 4.

In some embodiments, L is a phosphate-based linker. In some embodiments, the phosphate-based linker comprises a phosphate-based moiety selected from the group consisting of a phosphate ester, a pyrophosphate ester, a triphosphate ester, a tetraphosphate ester, a phosphonate, a diphosphonate, a phosporamidate, a pyrophosporamidate, a triphosphoramidate, a tetraphosphoramidate, a phosphorthioate and a diphosphorthioate. In some embodiments, the phosphate-based moiety is a pyrophosphate ester. In some embodiments, the phosphate-based moiety is a diphosphonate.

In some embodiments, L further comprises at least one additional moiety, wherein each at least one additional moiety is independently selected from the group consisting of unsubstituted alkylene, substituted alkylene, -(alkylene-O)—, optionally substituted arylene, —O—, —C(O)—, —N(R_{w})—, —S(O)₀₋₂—, a water-soluble polymer and an amino acid; wherein each R_{w} is independently H or C₁-C₈ alkyl; and combinations thereof. In some embodiments, the phosphate-based moiety is covalently bound to the —O— atom of Formula (II) via a phosphorous atom of the phosphate-based moiety.

In some embodiments, each at least one additional moiety is independently selected from the group consisting of unsubstituted alkylene, -(alkylene-O)—, —C(O)—, —N(R_{w})—, a water-soluble polymer and an amino acid; wherein each R_{w} is independently H or C₁-C₈ alkyl; and combinations thereof.

In some embodiments, L comprises at least one alkylene group. In some embodiments, L comprises an amino acid. In some embodiments, the amino acid is selected from the group consisting of serine, threonine, cysteine, tyrosine, aspartic acid, glutamic acid, lysine and N_{ϵ}-methyl-lysine. In some embodiments, the amino acid is lysine or N_{ϵ}-methyl-lysine. In some embodiments, the amino acid is lysine. In some embodiments, the amino acid is N_{ϵ}-methyl-lysine.

In some embodiments, L comprises a water-soluble polymer. In some embodiments, L comprises a water-soluble polymer and an amino acid, wherein the water-soluble polymer is conjugated to the amino acid. In some embodiments, the water-soluble polymer is conjugated to a side chain of the amino acid. In some embodiments, the water-soluble polymer is conjugated to the amino acid via a spacer element.

In some embodiments, L is selected from the group of linkers of Table 6. In some embodiments, L is selected from the group of linkers of Table 7. In some embodiments, L is selected from the group consisting of:

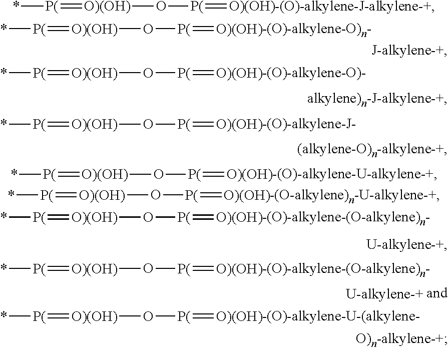

wherein:
each U is independently selected from the group consisting of:

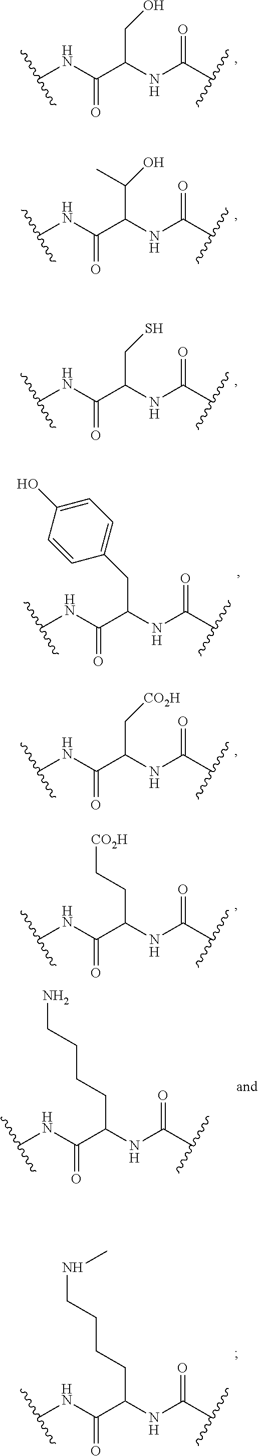

each J is independently

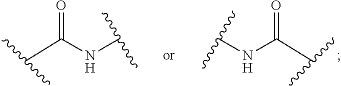 [SMILES: *NC(*)=O ; *NC(*)=O]

each alkylene is independently selected from the group consisting of:

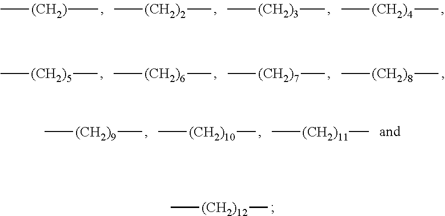

each n is independently an integer from 1 to 100;
* denotes the connection to the —O— atom of Formula (II); and
+ denotes the connection to E;
   wherein each linker L is optionally substituted with one or more water-soluble polymers.

In some embodiments, each n is independently an integer from 1 to 10. In some other embodiments, each n is independently 1, 2 or 3.

In some embodiments, L is substituted with one or more water-soluble polymers.

In some embodiments, L comprises group U, and one water-soluble polymer is conjugated to an amino acid side chain of group U. In some embodiments, the water-soluble polymer is conjugated to the amino acid side chain of group U via a spacer element.

In some embodiments, L is a bivalent linker. In some embodiments, L is a bivalent linker optionally substituted with one or more water-soluble polymers. In some embodiments, L is a linker substituted with one or more water-soluble polymers. In some embodiments, L is a linker substituted with one water-soluble polymer.

In some embodiments, L is:

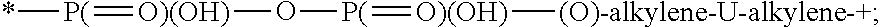

wherein:
U is selected from the group consisting of:

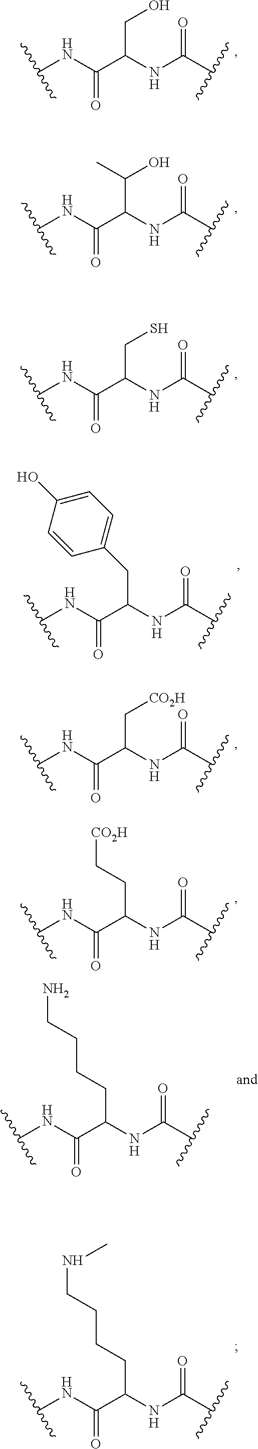

each alkylene is independently selected from the group consisting of:

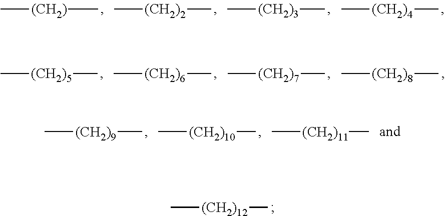

* denotes the connection to the —O— atom of Formula (II); and
+ denotes the connection to E;
   wherein L is optionally substituted with one or more water-soluble polymers. In some embodiments, L is substituted with one or more water-soluble polymers. In some embodiments, one water-soluble polymer is conjugated to an amino acid side chain of group U. In some embodiments, the one water-soluble polymer is conjugated to the amino acid side chain of group U via a spacer element. In some embodiments, the spacer element is a carbonyl group.

In some embodiments, U is:

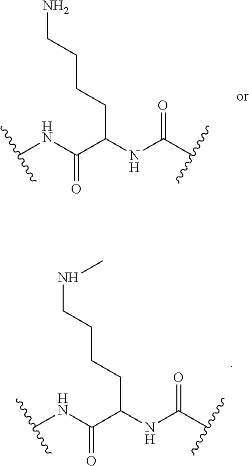 [SMILES: *NC(=O)C(CCCCN)NC(*)=O ; *NC(=O)C(CCCCNC)NC(*)=O]

In some embodiments, L has the following structure:

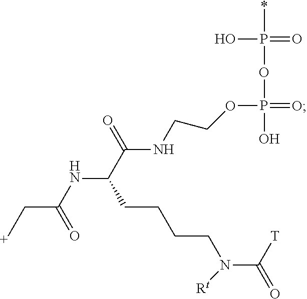

wherein: T is the water-soluble polymer; R^{t} is H or methyl; * denotes the connection to the —O— atom of Formula (II); and + denotes the connection to E.

In some embodiments, the water-soluble polymer is a polysaccharide.

In some embodiments, the water-soluble polymer is a (polyethylene)glycol (PEG) moiety.

In some embodiments, the PEG moiety has a molecular weight within a range of about 100 Da to about 100,000 Da. In some embodiments, the PEG moiety has a molecular weight within a range of about 100 Da to about 10,000 Da. In some embodiments, the PEG moiety has a molecular weight within a range of about 100 Da to about 5,000 Da. In some embodiments, the PEG moiety has a molecular weight within a range of about 100 Da to about 1,000 Da.

In some embodiments, the PEG moiety is —(CH₂CH₂O)ₙCH₃, wherein n is an integer from 1 to 100. In some embodiments, the PEG moiety is —(CH₂CH₂O)ₙCH₃, wherein n is an integer from 1 to 24. In some embodiments, the PEG moiety is —(CH₂CH₂O)ₙCH₃, wherein n is an integer from 6 to 12. In some embodiments, the PEG moiety is —(CH₂CH₂O)ₙCH₃, wherein n is an integer from 8 to 12. In some embodiments, the PEG moiety is —(CH₂CH₂O)ₙCH₃, wherein n is 8. In some embodiments, the PEG moiety is —(CH₂CH₂O)ₙCH₃, wherein n is 12.

In some embodiments, the PEG is linear. In some other embodiments, the PEG is branched.

In some embodiments, a drug linker compound containing a reactive moiety is conjugated to an antibody or antibody fragment by reacting a drug-linker compound with an antibody, antibody fragment or variant thereof (or simply “antibody”) containing one or more natural or non-natural amino acids. The conjugation reaction provides an ADC, wherein drug-linker is conjugated to a natural or non-natural amino acid of the antibody via a covalent linkage. The covalent linkage can be a product of the reactive moiety of the drug-linker and an additional moiety present in the natural or non-natural amino acid, wherein the additional moiety can react to form the covalent linkage with the reactive moiety. Methods of conjugating drug-linkers to antibodies are known in the art (see, e.g., Johann, K. et al., Polymer Chemistry, 27(11):4396-4407 (2020); Bioconjug Chem., 27(12):2791-2807 (2016); Northrop, B. H. et al., Polymer Chemistry, 18(6):3415-3430 (2015); Axup, J. Y. et al., Proc. Natl. Acad. Sci., 109(40):16101-16016 (2012); Hartmuth, C. et al., Angew. Chem. Int. Ed., 40(11):2004-2021 (2001); Sletten, E. M. and Bertozzi, C. R., Angew. Chem. Int. Ed., 48(38):6974-6998 (2009); WO2006/050262A2; and WO2013/185177A1; the contents of each of which are hereby incorporated by reference in their entirety. Non-limiting examples of reactions and linkages formed between drug-linker compounds and natural or non-natural amino acids incorporated into an antibody of the present disclosure include the following.

A. (i) Reaction of a drug-linker comprising —N₃ with a non-natural amino acid comprising an alkynyl group, thereby providing a linkage comprising a 1,2,3-triazolyl moiety; or (ii) reaction of a drug-linker comprising an alkynyl group with a non-natural amino acid comprising —N₃, thereby providing a linkage comprising a 1,2,3-triazolyl moiety. In some embodiments, the alkynyl group is a cyclooctynyl group. In some embodiments, the non-natural amino acid is p-azido-L-phenylalanine. In some embodiments, the linkage comprising the 1,2,3-triazolyl moiety has the following structure:

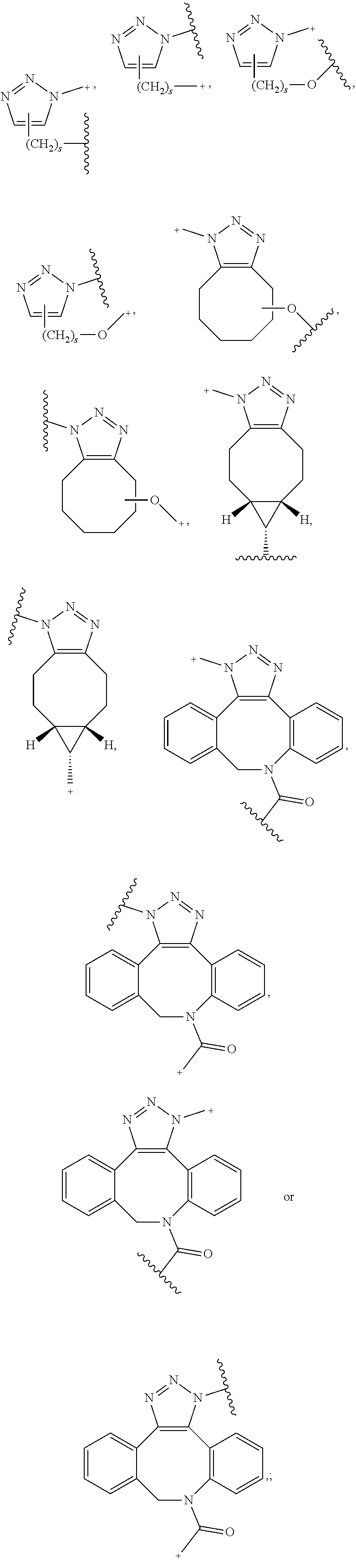

wherein:
each s is independently 0 or an integer from 1 to 50; optionally, each s is independently 0, 1, 2, 3, 4, 5 or 6;
each t is independently 0 or an integer from 1 to 50; optionally, each t is independently 0, 1, 2, 3, 4, 5 or 6;
each + denotes connection to a linker of the drug-linker; and
each wavy line denotes connection to the antibody.

B. (i) Reaction of a drug-linker comprising a tetrazinyl group with a non-natural amino acid comprising an (E)-cyclooctenyl group, thereby providing a linkage comprising a 1,4-dihydropyridazinyl moiety; or (ii) reaction of a drug-linker comprising a tetrazinyl group with a non-natural amino acid comprising an (E)-cyclooctenyl, thereby providing a linkage comprising a 1,4-dihydropyridazinyl moiety. In some embodiments, the linkage comprising the 1,4-dihydropyridazinyl moiety has the following structure:

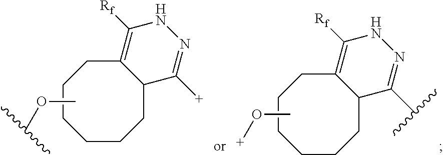

wherein:
each R^{f} is independently H or alkyl, optionally unsubstituted C₁-C₆ alkyl;
each + denotes connection to a linker of the drug-linker; and
each wavy line denotes connection to the antibody.

C. (i) Reaction of a drug-linker comprising an —ONH₂ group with a non-natural amino acid comprising a carbonyl or ketone group, thereby providing a linkage comprising an oxime moiety; or (ii) reaction of a drug-linker comprising a carbonyl or ketone group with a non-natural amino acid comprising an —ONH₂ group, thereby providing a linkage comprising an oxime moiety. In some embodiments, the carbonyl or ketone group is —C(O)R^{q}, wherein R^{q} is unsubstituted C₁-C₆ alkyl. In some embodiments, R^{q} is methyl. In some embodiments, the linkage comprising the oxime moiety has the following structure:

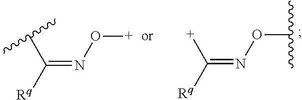

wherein:
each R^{q} is independently unsubstituted C₁-C₆ alkyl; optionally, each R^{q} is methyl;
each + denotes connection to a linker of the drug-linker; and
each wavy line denotes connection to the antibody.

D. (i) Reaction of a drug-linker comprising a maleimide group with a natural or non-natural amino acid comprising a thiol (—SH), thereby providing a linkage comprising a pyrrolidine-2,5-dione moiety, such as a 3-(λ1-sulfaneyl)pyrrolidine-2,5-dione moiety; or (ii) reaction of a drug-linker comprising a thiol (—SH) group with a non-natural amino acid comprising a maleimide group, thereby providing a linkage comprising a pyrrolidine-2,5-dione moiety, such as a 3-(λ1-sulfaneyl)pyrrolidine-2,5-dione moiety. In some embodiments, the natural amino acid is cysteine. In some embodiments, the linkage comprising the a pyrrolidine-2,5-dione moiety, such as a 3-(λ1-sulfaneyl)pyrrolidine-2,5-dione moiety, has the following structure:

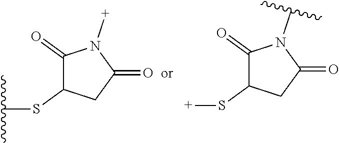

wherein:
each + denotes connection to a linker of the drug-linker; and
each wavy line denotes connection to the antibody.

E. (i) Reaction of a drug-linker comprising a primary or secondary amine with a natural or non-natural amino acid comprising a carboxylic acid group, a protected carboxylic acid, or an activated ester group, thereby providing a linkage comprising an amide moiety; or (ii) reaction of a drug-linker comprising a carboxylic acid group, a protected carboxylic acid, or an activated ester group with a natural or non-natural amino acid comprising a primary or secondary amine group, thereby providing a linkage comprising a amide moiety. In some embodiments, the natural amino acid is aspartic acid or glutamic acid. In some other embodiments, the natural amino acid is lysine.

In some embodiments, the reaction is a peptide coupling reaction or other well-known method of forming an amide, each of which can be performed using methods readily understood by a person of ordinary skill in the art. In some embodiments, the linkage comprising the amide moiety has the following structure:

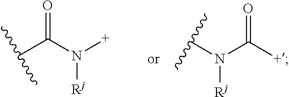

wherein:
each R^{j} is independently H or alkyl; optionally unsubstituted C₁-C₆ alkyl;
each + denotes connection to a linker of the drug-linker; and
each wavy line denotes connection to the antibody.

F. (i) Reaction of a drug-linker comprising a hydroxyl group (—OH) with a natural or non-natural amino acid comprising a carboxylic acid group, a protected carboxylic acid, or an activated ester group, thereby providing a linkage comprising an ester moiety; or (ii) reaction of a drug-linker comprising a carboxylic acid group, a protected carboxylic acid, or an activated ester group with a natural or non-natural amino acid comprising a hydroxyl group (—OH), thereby providing a linkage comprising an ester moiety. In some embodiments, the natural amino acid is aspartic acid or glutamic acid. In some other embodiments, the natural amino acid is serine, threonine or tyrosine. Methods of forming such esters linkages can be performed using methods readily understood by a person of ordinary skill in the art. In some embodiments, the linkage comprising the ester moiety has the following structure:

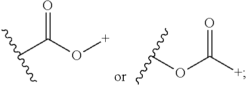

wherein:
each + denotes connection to a linker of the drug-linker; and
each wavy line denotes connection to the antibody.

G. (i) Reaction of a drug-linker comprising a thiol group (—SH) with a natural or non-natural amino acid comprising a carboxylic acid group, a protected carboxylic acid, or an activated ester group, thereby providing a linkage comprising a thioester moiety; or (ii) reaction of a drug-linker comprising a carboxylic acid group, a protected carboxylic acid, or an activated ester group with a natural or non-natural amino acid comprising a thiol group (—SH), thereby providing a linkage comprising a thioester moiety. In some embodiments, the natural amino acid is aspartic acid or glutamic acid. In some other embodiments, the natural amino acid is cysteine. Methods of forming such thioesters linkages can be performed using methods readily understood by a person of ordinary skill in the art. In some embodiments, the linkage comprising the ester moiety has the following structure:

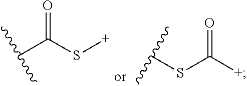

wherein:
each + denotes connection to a linker of the drug-linker; and
each wavy line denotes connection to the antibody.

H. Reaction of a drug-linker comprising a —C(O)CH₂NH₂ group with a natural or non-natural amino acid comprising a carboxylic acid group, a protected carboxylic acid, or an activated ester group, thereby providing a linkage comprising a —C(O)CH₂NHC(O)— moiety; or (ii) reaction of a drug-linker comprising a carboxylic acid group, a protected carboxylic acid, or an activated ester group with a non-natural amino acid comprising a —C(O)CH₂NH₂ group, thereby providing a linkage comprising a —C(O)CH₂NHC(O)— moiety. In some embodiments, the natural amino acid is aspartic acid or glutamic acid. Methods of forming such linkages can be performed using methods readily understood by a person of ordinary skill in the art. In some embodiments, the linkage has the following structure:

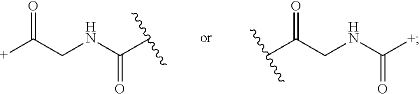

wherein:
each + denotes connection to a linker of the drug-linker; and
each wavy line denotes connection to the antibody.

I. Reaction of a drug-linker comprising a thiol group (—SH) with a natural or non-natural amino acid comprising a thiol group, thereby providing a linkage comprising a disulfide. In some embodiments, the natural amino acid is cysteine. Methods of forming disulfide linkages can be performed using methods readily understood by a person of ordinary skill in the art.

Thus, in some embodiments, E comprises an amide, an ester, a thioester, a pyrrolidine-2,5-dione, an oxime, a 1,2,3-triazole or a 1,4-dihydropyridazine, wherein the 1,2,3-triazole and the 1,4-dihydropyridazine are each optionally fused to an 8-membered ring. In some embodiments, E is selected from the group consisting of:

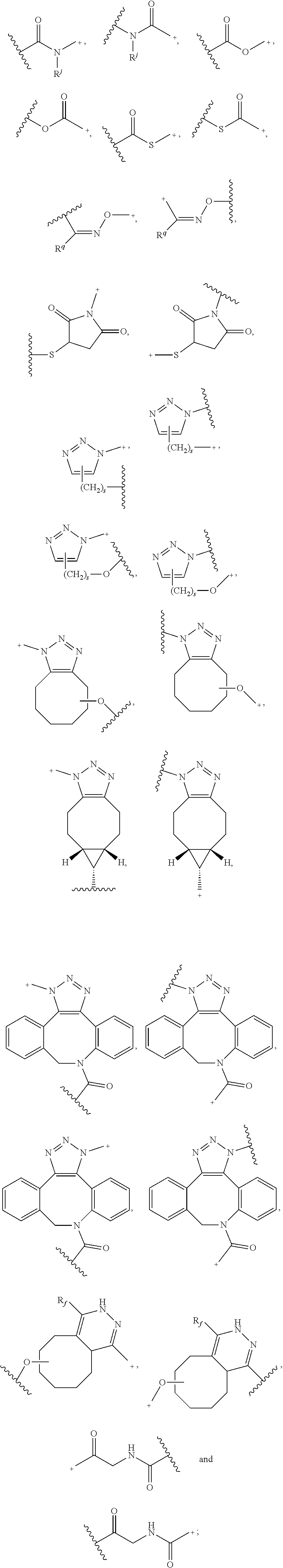

wherein:
   each R^{j} is independently H or unsubstituted C₁-C₆ alkyl;
   each R^{q} is independently unsubstituted C₁-C₆ alkyl;
   each R^{f} is independently H or unsubstituted C₁-C₆ alkyl;
   each s is independently 0, 1, 2, 3, 4, 5 or 6;
   each t is independently 0, 1, 2, 3, 4, 5 or 6;
   each + denotes connection to L; and
   each wavy line denotes connection to Ab.

In some embodiments, E is:

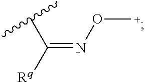

wherein R^{q} is unsubstituted C₁-C₆ alkyl. In some embodiments, R^{q} is methyl.

In some embodiments, Ab comprises 1 to 10 non-natural amino acids. In some embodiments, Ab comprises 1, 2, 3, 4, 5, 6, 7, 8, 9 or 10 non-natural amino acids. In some embodiments, Ab comprises 1, 2, 3 or 4 non-natural amino acids. In some embodiments, Ab comprises 1 non-natural amino acid. In some embodiments, Ab comprises 2 non-natural amino acids. In some embodiments, Ab comprises 3 non-natural amino acids. In some embodiments, Ab comprises 4 non-natural amino acid.

In some embodiments, Ab is configured to bind to an antigen.

In some embodiments, Ab is configured to bind to a tumor-associated antigen (TAA) or cancer antigen.

In some embodiments, Ab binds to a tumor-associated antigen (TAA) selected from the group consisting of PD-1, PD-L1, PSMA, CD70, CD3, HER2, HER3, TROP2, GPC3, VEGFR, EGFR, c-Met (HGFR), CD19, CD22, CD25 (IL-2R alpha), CD30, CD33, CD37, CD46, CD48, CD56 (NCAM-1), CD71 (Transferrin R), CD74, CD79b, CD123 (IL-3R alpha), CD138 (syndecan-1), CD142, CD166 (ALCAM), CD203c (ENPP3), CD205 (LY75), CD221 (IGF-1R), CD262 (TRAIL R2), CD276 (B7-H3), mesothelin, EpCAM, CEACAM5, CEACAM6, DLL3, ROR1, ROR2, GPNMB, GCC, GUCY2c, NaPi2b, Flt-1, Flt-3, folate receptor alpha, Tissue Factor (TF), CA6, MUC1, MUC16 (CA-125), BCMA, SLAMF7 (CS1), TIM1, CanAg, Ckit (CD117), EphA2, Nectin4, SLTRK6, FGFR2, LYPD3 (C4.4a), Cadherin 3, 5T4 (TPBG), STEAP1, PTK7, Ephrin-A4, LIV-1 (SLC39A6 or ZIP6), SLC1A5, TENB2, ETBR, integrin v3, Cripto, AGS-5 (SLC44A4), LY6E, AXL, LAMP1, LRRC15, TNF-alpha, and MN/CA IX.

In some embodiments, Ab binds to an antigen selected from the group consisting of TROP2, CD70, HER2, CD3, PSMA, HER3 and GPC3. In some embodiments, Ab binds to CD70. In some embodiments, Ab binds to GPC3.

In some embodiments, the one or more non-natural amino acids is selected from the group consisting of para-acetyl phenylalanine, 4-acetyl-L-phenylalanine (para-acetyl-L-phenylalanine (pAF)), 3-O—(N-acetyl-beta-D-glucosaminyl)-L-threonine, N4-(β-N-Acetyl-D-glucosaminyl)-L-asparagine, 0-allyl-L-tyrosine, alpha-N-acetylgalactosamine-O-L-serine, alpha-N-acetylgalactosamine-O-L-threonine, 2-aminooctanoic acid, 2-amino-L-phenylalanine, 3-amino-L-phenylalanine, 4-amino-L-phenylalanine, 2-amino-L-tyrosine, 3-amino-L-tyrosine, 4-azido-L-phenylalanine, 4-benzoyl-L-phenylalanine, (2,2-bipyridin-5yl)-L-alanine, 3-borono-L-phenylalanine, 4-borono-L-phenylalanine, 4-bromo-L-phenylalanine, p-carboxymethyl-L-phenylalanine, 4-carboxy-L-phenylalanine, p-cyano-L-phenylalanine, 3,4-dihydroxy-L-phenylalanine (L-DOPA), 4-ethynyl-L-phenylalanine, 2-fluoro-L-phenylalanine, 3-fluoro-L-phenylalanine, 4-fluoro-L-phenylalanine, 0-(3-O-D-galactosyl-N-acetyl-beta-D-galactosaminyl)-L-serine, L-homoglutamine, (8-hydroxyquinolin-3-yl)-L-alanine, 4-iodo-L-phenylalanine, 4-isopropyl-L-phenylalanine, O-i-propyl-L-tyrosine, 3-isopropyl-L-tyrosine, O-mannopyranosyl-L-serine, 2-methoxy-L-phenylalanine, 3-methoxy-L-phenylalanine, 4-methoxy-L-phenylalanine, 3-methyl-L-phenylalanine, O-methyl-L-tyrosine, 3-(2-naphthyl)-L-alanine, 5-nitro-L-histidine, 4-nitro-L-histidine, 4-nitro-L-leucine, 2-nitro-L-phenylalanine, 3-nitro-L-phenylalanine, 4-nitro-L-phenylalanine, 4-nitro-L-tryptophan, 5-nitro-L-tryptophan, 6-nitro-L-tryptophan, 7-nitro-L-tryptophan, 2-nitro-L-tyrosine, 3-nitro-L-tyrosine, O-phospho-L-serine, O-phospho-L-tyrosine, 4-propargyloxy-L-phenylalanine, O-2-propyn-1-yl-L-tyrosine, 4-sulfo-L-phenylalanine and O-sulfo-L-tyrosine.

In some embodiments, at least one of the one or more non-natural amino acids is para-acetyl phenylalanine. In some more particular embodiments, at least one of the one or more non-natural amino acid is 4-acetyl-L-phenylalanine (para-acetyl-L-phenylalanine (pAF)). In some embodiments, each of the one or more non-natural amino acids is para-acetyl phenylalanine. In some more particular embodiments, each of the one or more non-natural amino acid is 4-acetyl-L-phenylalanine (para-acetyl-L-phenylalanine (pAF)).

In some embodiments, Ab comprises a heavy chain having a heavy chain sequence, a light chain having a light chain sequence, or both.

In some embodiments, the heavy chain sequence comprises at least one of the one or more non-natural amino acids.

In some embodiments, the light chain sequence comprises at least one of the one or more non-natural amino acids.

In some embodiments, Ab comprises two heavy chains, each heavy chain having a heavy chain sequence, wherein each heavy chain sequence comprises at least one of the one or more non-natural amino acid.

In some embodiments, Ab comprises two light chains, each light chain having a light chain sequence, wherein each light chain sequence comprises at least one of the one or more non-natural amino acid.

In some embodiments, Ab comprises two heavy chains and two light chains, wherein each heavy chain sequence and each light chain sequence comprises at least one of the one or more non-natural amino acid.

In some embodiments, the one or more non-natural amino acids are solvent accessible.

In some embodiments, each of the one or more non-natural amino acids is the same. In some embodiments, each of the one or more non-natural amino acids is para-acetyl phenylalanine. In some more particular embodiments, each of the one or more non-natural amino acid is 4-acetyl-L-phenylalanine (para-acetyl-L-phenylalanine (pAF)).

In some embodiments, Ab comprises 1, 2, 3 or 4 non-natural amino acids.

In some embodiments, Ab comprises two heavy chains and two light chains, each said heavy chain having a heavy chain sequence and each said light chain having a light chain sequence, wherein each said heavy chain sequence and each said light chain sequence comprises one non-natural amino acid.

In some other embodiments, Ab is an anti-CD70 antibody (i.e., Ab is an anti-CD70 Ab), antibody fragment or variant thereof. In some embodiments, the anti-CD70 Ab comprises at least one sequence listed in Table 2.

In some embodiments, the anti-CD70 Ab comprises a heavy chain sequence and a light chain sequence, wherein the heavy chain sequence comprises an amino acid sequence selected from the group consisting of the heavy chain sequences listed in Table 2, and the light chain sequence comprises an amino acid sequence selected from the group consisting of the light chain sequences listed in Table 2, wherein at least one of the heavy chain and light chain sequences comprises the one or more non-natural amino acids.

In some embodiments, the anti-CD70 Ab heavy chain sequence comprises at least one of the one or more non-natural amino acids, and the position occupied by the at least one non-natural amino acid in the heavy chain sequence is selected from the group consisting of Kabat position 114, 115, 129, 136, 159 and 160. In some embodiments, the position occupied by the at least one non-natural amino acid in the heavy chain sequence is Kabat position 114.

In some embodiments, the anti-CD70 Ab light chain sequence comprises at least one of the one or more non-natural amino acids, and the position occupied by the at least one non-natural amino acid in the light chain sequence is selected from the group consisting of Kabat position 110, 112, 114 and 121.

In some embodiments, the anti-CD70 Ab comprises two heavy chain sequences, wherein each heavy chain sequence comprises at least one of the one or more non-natural amino acids, and the position occupied by each at least one non-natural amino acid in each heavy chain sequence is independently selected from the group consisting of Kabat position 114, 115, 129, 136, 159 and 160.

In some embodiments, each heavy chain sequence comprises a single non-natural amino acid.

In some embodiments, the position occupied by each at least one non-natural amino acid in each heavy chain sequence is Kabat position 114. In some embodiments, each anti-CD70 Ab heavy chain sequence comprises a single non-natural amino acid.

In some embodiments, the anti-CD70 Ab comprises two light chain sequences, wherein each light chain sequence comprises at least one of the one or more non-natural amino acids, and the position occupied by each at least one non-natural amino acid in each light chain sequence is independently selected from the group consisting of Kabat position 110, 112, 114 and 121.

In some embodiments, the anti-CD70 Ab comprises two heavy chain sequences and two light chain sequences, wherein: (i) each heavy chain sequence contains one non-natural amino acid, and the position occupied by each non-natural amino acid is Kabat position 114; and (ii) each light chain sequence contains one non-natural amino acid, and the position occupied by each non-natural amino acid is selected from the group consisting of Kabat position 110, 112, 114 and 121. In some embodiments, each non-natural amino acid is para-acetyl phenylalanine. In some more particular embodiments, each non-natural amino acid is 4-acetyl-L-phenylalanine (para-acetyl-L-phenylalanine (pAF)).

In some embodiments, the anti-CD70 antibody comprises a heavy chain variable region having the amino acid sequence of SEQ ID NO: 26.

In some embodiments, the anti-CD70 antibody comprises a light chain variable region having the amino acid sequence of SEQ ID NO: 27.

In some embodiments, the anti-CD70 antibody comprises a heavy chain having the amino acid sequence of SEQ ID NO: 25.

In some embodiments, the anti-CD70 antibody comprises a heavy chain having the amino acid sequence of SEQ ID NO: 20.

In some embodiments, the anti-CD70 antibody comprises a light chain having the amino acid sequence of SEQ ID NO: 19.

In some embodiments, the anti-CD70 antibody comprises two heavy chains, each having the amino acid sequence of SEQ ID NO: 20, and two light chains, each having the amino acid sequence of SEQ ID NO: 19.

In some embodiments, the anti-CD70 antibody comprises two heavy chains, each having the amino acid sequence of SEQ ID NO: 25, and two light chains, each having the amino acid sequence of SEQ ID NO: 19.

In some other embodiments, Ab is an anti-TROP2 antibody (i.e., Ab is an anti-TROP2 Ab), antibody fragment or variant thereof. In some embodiments, the anti-TROP2 Ab comprises at least one sequence listed in Table 1. In some other embodiments, the anti-TROP2 antibody comprises a heavy chain having the amino acid sequence of SEQ ID NO: 5. In some other embodiments, the anti-TROP2 antibody comprises a light chain having the amino acid sequence of SEQ ID NO: 4.

In some other embodiments, Ab is an anti-HER2 antibody (i.e., Ab is an anti-HER2 Ab), antibody fragment or variant thereof. In some embodiments, the anti-TROP2 Ab comprises at least one sequence listed in Table 3. In some other embodiments, the anti-HER2 antibody comprises a heavy chain having the amino acid sequence of SEQ ID NO: 29. In some other embodiments, the anti-HER2 antibody comprises a light chain having the amino acid sequence of SEQ ID NO: 30.

In some other embodiments, Ab is an anti-PSMA antibody (i.e., Ab is an anti-PSMA Ab), antibody fragment or variant thereof. In some embodiments, the anti-PSMA Ab comprises at least one sequence listed in Table 4. In some embodiments, the anti-PSMA antibody comprises a heavy chain having the amino acid sequence of SEQ ID NO: 39. In some embodiments, the anti-PSMA antibody comprises a light chain having the amino acid sequence of SEQ ID NO: 40.

In some other embodiments, Ab is an anti-HER3 antibody (i.e., Ab is an anti-HER3 Ab), antibody fragment or variant thereof. In some embodiments, the anti-HER3 Ab comprises at least one sequence listed in Table 5. In some embodiments, the anti-HER3 antibody comprises a heavy chain having the amino acid sequence of SEQ ID NO: 58. In some embodiments, the anti-HER3 antibody comprises a light chain having the amino acid sequence of SEQ ID NO: 47. In some other embodiments, the anti-HER3 antibody comprises a light chain having the amino acid sequence of SEQ ID NO: 51.

It is understood that an ADC is typically produced as a composition containing a population of ADCs, i.e., a mixture of ADCs that are essentially identical, except for the drug load. As disclosed herein, an ADC composition can be characterized by a drug-to-antibody ratio (DAR), which reports on the average number of drugs conjugated to antibody in the ADC composition.

Thus, in some aspects, the present disclosure provides an ADC composition comprising a mixture of ADCs, wherein each ADC in the mixture is identical, except that the number of drugs or drug-linkers that are conjugated to each ADC can vary. In a non-limiting example, an ADC of the present disclosure comprises a first ADC, a second ADC, a third ADC and a fourth ADC, wherein the first ADC, the second ADC, the third ADC and the fourth ADC are identical, except that the first ADC comprises one drug or drug-linker, the second ADC comprises two drugs or drug-linkers, the third ADC comprises three drugs or drug-linkers, and the fourth ADC comprises four drugs or drug-linkers.

In some other embodiments, there is provided an ADC composition, comprising:
(a) an ADC of Formula (II), wherein d is 1;
(b) an ADC of Formula (II), wherein the ADC is identical to (a), except that d is 2;
(c) an ADC of Formula (II), wherein the ADC is identical to (a), except that d is 3;
(d) an ADC of Formula (II), wherein the ADC is identical to (a), except that d is 4;
(e) an ADC of Formula (II), wherein the ADC is identical to (a), except that d is 5;
(f) an ADC of Formula (II), wherein the ADC is identical to (a), except that d is 6;
(g) an ADC of Formula (II), wherein the ADC is identical to (a), except that d is 7;
(h) an ADC of Formula (II), wherein the ADC is identical to (a), except that d is 8;
(i) an ADC of Formula (II), wherein the ADC is identical to (a), except that d is 9; or
(j) an ADC of Formula (II), wherein the ADC is identical to (a), except that d is 10;
or a combination of any two or more of the foregoing; wherein the composition is characterized as having a DAR of at least about 1 and at most about 10.

In some embodiments, the ADC composition is characterized as having a DAR of at least about 1 and at most about 8. In some embodiments, the ADC composition is characterized as having a DAR of at least about 2 and at most about 8, at least about 2 and at most about 6, or at least about 2 and at most about 4. In some embodiments, the ADC composition is characterized as having a DAR of at least about 3 and at most about 4. In some embodiments, the ADC composition is characterized as having a DAR of at least about 1 and at most about 2. In some embodiments, the ADC composition is characterized as having a DAR of at least about 1 and at most about 3. In some embodiments, the ADC composition is characterized as having a DAR of at least about 2 and at most about 4. In some embodiments, the ADC composition is characterized as having a DAR of at least about 3 and at most about 4. In some embodiments, the ADC composition is characterized as having a DAR of about 2. In some other embodiments, the ADC composition is characterized as having a DAR of about 4.

In some aspects of the disclosure, an antibody, antibody fragment, variant or drug conjugate with increased serum half-life, water solubility, bioavailability, therapeutic half-life or circulation time, or with modulated immunogenicity, or with modulated biological activity, is desired. One method of achieving such desired features of the compositions disclosed herein, is by covalent attachment of the polymer polyethylene glycol, (PEG). To maximize the desired properties of PEG, the total molecular weight and hydration state of the polymer or polymers attached to the biologically active molecule must be sufficiently high to impart the advantageous characteristics typically associated with such polymer attachment, such as increased water solubility and circulating half-life, while not adversely impacting the bioactivity of the molecule to which the PEG is attached. PEG derivatives are frequently linked to biologically active molecules through reactive chemical functionalities, such as amino acid residues, the N-terminus, and/or carbohydrate moieties. In some aspects of the present invention, PEG derivatives are linked to biologically active molecules through reactive chemical functionalities to improve biophysical properties of the resulting ADC. WO99/67291 discloses a process for conjugating a protein with PEG, wherein at least one amino acid residue on the protein is substituted with a synthetic amino acid and the protein is contacted with PEG under conditions sufficient to achieve conjugation to the protein.

In some aspects of the disclosure antibody, antibody fragments, variant or drug conjugate with increase serum half-life, water solubility, bioavailability, therapeutic half-life, or circulation time, or to modulate immunogenicity, or biological activity is desired. One method of achieving such desired features of the composition disclosed herein, is by covalent attachment of the polymer polyethylene glycol, (PEG). To maximize the desired properties of PEG, the total molecular weight and hydration state of the polymer or polymers attached to the biologically active molecule must be sufficiently high to impart the advantageous characteristics typically associated with such polymer attachment, such as increased water solubility and circulating half-life, while not adversely impacting the bioactivity of the molecule to which the PEG is attached. PEG derivatives are frequently linked to biologically active molecules through reactive chemical functionalities, such as amino acid residues, the N-terminus, and/or carbohydrate moieties. In some aspects of the present invention, PEG derivatives are linked to biologically active molecules through reactive chemical functionalities to improve biophysical properties of the resulting ADC. WO99/67291 discloses a process for conjugating a protein with PEG, wherein at least one amino acid residue on the protein is substituted with a synthetic amino acid and the protein is contacted with PEG under conditions sufficient to achieve conjugation to the protein.

Proteins and other molecules often have a limited number of reactive sites available for polymer attachment. The sites most suitable for modification via polymer attachment may play a significant role in receptor binding, and such sites may be necessary for retention of the biological activity of the molecule therefore making them inappropriate for polymer attachment. As a result, indiscriminate attachment of polymer chains to such reactive sites on a biologically active molecule often leads to a significant reduction or even total loss of biological activity of the polymer-modified molecule, PEG attachment can be directed to a particular position within a protein such that the PEG moiety does not interfere with the function of that protein. One method of directing PEG attachment is to introduce a synthetic amino acid into the protein sequence. The protein biosynthetic machinery of the prokaryote Escherichia coli (E. coli) can be altered in order to incorporate synthetic amino acids efficiently and with high fidelity into proteins in response to the amber codon, UAG. See, e.g., J. W. Chin et al., J. Amer. Chem. Soc. 124: 9026-9027, 2002; J. W. Chin, & P. G. Schultz, ChemBioChem 3(11): 1135-1137, 2002; J. W. Chin, et al., PNAS USA 99: 11020-11024, 2002; and, L. Wang, & P. G. Schultz, Chem. Comm., 1: 1-11, 2002. A similar method can be accomplished with the eukaryote, Saccharomyces cerevisiae (S. cerevisiae) (e.g., J. Chin et al., Science 301: 964-7, 2003). Using this method, a non-natural amino acid can be incorporated into an antibody, variant or drug conjugate of the present disclosure, providing an attachment site for PEG. See, for example WO2010/011735 and WO2005/074650.

### Methodology and Techniques

The present disclosure encompasses methodologies and technologies well known in the art. These include conventional methods of mass spectroscopy, NMR, HPLC, protein chemistry, biochemistry, recombinant DNA techniques and pharmacology, within the skill of the art. Compounds of the present disclosure can be synthesized using several processes or schemes employed in the art. See for example, Dubowchik et al., Bioconjugate Chem. 13: 855-869, 2002; Doronina et al., Nature Biotechnology 21(7): 778-784, 2003; WO2012/166560; WO2013/185117, each incorporated herein by reference. Many methodologies and techniques for synthesis of pharmaceutical, diagnostic or therapeutic compounds are well known to one of ordinary skill in the art.

The present disclosure, unless otherwise indicated, also encompass conventional techniques of molecular biology (including recombinant techniques), cell biology, biochemistry and immunology, all within the skill of the art. Such techniques are explained fully in the literature, such as, Molecular Cloning: A Laboratory Manual, Third Edition, Cold Spring Harbor Press, Cold Spring Harbor, NY (Sambrook et al. Eds., 2001); Oligonucleotide Synthesis: Methods And Applications (Methods in Molecular Biology), Herdewijn, P., Ed., Humana Press, Totowa, NJ; Oligonucleotide Synthesis (Gait, M. J., Ed., 1984); Methods In Molecular Biology, Humana Press, Totowa, NJ; Cell Biology: A Laboratory Notebook, Academic Press, New York, NY (Cellis, J. E., Ed., 1998); Animal Cell Culture (Freshney, R. I., Ed., 1987); Introduction To Cell And Tissue Culture Plenum Press, New York, NY, (Mather, J. P. and Roberts, P. E., Eds., 1998); Cell And Tissue Culture: Laboratory Procedures John Wiley and Sons, Hoboken, NJ, (Doyle, A. et al., Eds., 1993-8); Methods In Enzymology (Academic Press, Inc.) New York, NY; Weir's Handbook Of Experimental Immunology Wiley-Blackwell Publishers, New York, NY, (Herzenberg, L. A. et al. Eds., 1997); Gene Transfer Vectors For Mammalian Cells Cold Spring Harbor Press, Cold Spring Harbor, NY, (Miller, J. M. et al. Eds., 1987); Current Protocols In Molecular Biology, Greene Pub. Associates, New York, NY, (Ausubel, F. M. et al., Eds., 1987); PCR: The Polymerase Chain Reaction, Birkhauser, Boston, MA, (Mullis, K. et al., Eds., 1994); Current Protocols In Immunology, John Wiley and Sons, Hoboken, NJ, (Coligan, J. E. et al., eds., 1991); Short Protocols In Molecular Biology, Hoboken, NJ, (John Wiley and Sons, 1999); Immunobiology 7 Garland Science, London, UK, (Janeway, C. A. et al., 2007); Antibodies. Stride Publications, Devoran, UK, (P. Finch, 1997); Antibodies: A Practical Approach Oxford University Press, USA, New York, NY, (D. Catty., ed., 1989); Monoclonal Antibodies: A Practical Approach Oxford University Press, USA, New York NY, (Shepherd, P. et al. Eds., 2000); Using Antibodies: A Laboratory Manual Cold Spring Harbor Laboratory Press, Cold Spring Harbor, NY, (Harlow, E. et al. Eds., 1998); The Antibodies Harwood Academic Publishers, London, UK, (Zanetti, M. et al. Eds. 1995).

### Therapeutic Uses of ADCs

The antibodies or ADCs of the disclosure are useful for treating a wide range of diseases. disorders, conditions, or cancers. Compositions disclosed herein may be used to modulate an immune response. Modulation of an immune response may comprise stimulating, activating, increasing, enhancing, or up-regulating an immune response. Modulation of an immune response may comprise suppressing, inhibiting, preventing, reducing, or downregulating an immune response. In some embodiments, the ADCs of the present invention may be used for reducing or inhibiting tumor growth or progression in an antigen-expressing cancer or cancer cell comprising an effective amount of the ADC.

Disclosed herein are methods of treating a subject for a condition with an ADC or pharmaceutical composition of the disclosure. In some cancers, overexpression of specific cell surface receptors can allow selective targeting of cancerous cells with small molecules or drugs, while minimizing effects on healthy cells. The invention provides a method of treating cancer by administering to a patient a therapeutically-effective amount of an ADC of the invention comprising an antibody or antibody fragment conjugated to a drug-linker disclosed herein. The cancer to be treated by an ADC of the present invention may be, a breast cancer including triple negative breast cancer (TNBC), a brain cancer, a pancreatic cancer, a skin cancer, a lung cancer, a liver cancer, a gall bladder cancer, a colon cancer, an ovarian cancer, a prostate cancer, a uterine cancer, a bone cancer, a blood cancer or a cancer or disease or conditions related to any of these cancers. In some embodiments, the cancer is a blood cancer. In some embodiments, the blood cancer is a leukemia, a lymphoma or a myeloma. In some embodiments, the blood cancer is a leukemia, wherein the leukemia is acute myeloid leukemia (AML). In some embodiments, the AML is responsive to treatment with an ADC of the present disclosure comprising an anti-CD70 antibody. In some other embodiments, the cancer is a liver cancer. In some embodiments, the liver cancer is hepatocellular carcinoma (HCC). In some embodiments, the liver cancer is responsive to treatment with an ADC of the present disclosure comprising an anti-GPC3 antibody.

In some embodiments, the invention provides a method of treating cancer by administering to a patient a therapeutically-effective amount of an ADC of the invention. The cancer may be an antigen expressing cancer. The cancer may be ovarian cancer including, but not limited to, an epithelial, stromal and germ cell tumor. The ovarian cancer may comprise a fallopian tube cancer or primary peritoneal carcinoma. The cancer may be characterized by high expression of an antigen receptor. The cancer may be treated by recruiting cytotoxic T cells to the antigen receptor expressing tumor cells. In some embodiments, the disclosure provides a method of treating any cancer, disease or condition associated with high expression of antigen receptors by administering to a patient a therapeutically-effective amount of an antibody or ADC of the disclosure. In some embodiments, the invention provides a method of treating a disorder, or condition, or disease, or cancer by administering to a patient a therapeutically-effective amount of an antibody or ADC of the invention. In some embodiments, the antibody, antibody fragment or variant thereof binds to a tumor-associated antigen (TAA) selected from the group consisting of PD-1, PD-Li, PSMA, CD70, CD3, HER2, HER3, TROP2, GPC3, VEGFR, EGFR, c-Met (HGFR), CD33, CD19, CD22, CD25 (IL-2R alpha), CD30, CD37, CD46, CD48, CD56 (NCAM-1), CD71 (Transferrin R), CD74, CD79b, C-D123 (IL-3R alpha), CD138 (syndecan-1), CD142, CD166 (ALCAM), CD203c (ENPP3), CD205 (LY75), CD221 (IGF-1R), CD262 (TRAIL R2), CD276 (B7-H3), mesothelin, EpCAM, CEACAM5, CEACAM6, DLL3, ROR1, ROR2, GPNMB, GCC, GUCY2c, NaPi2b, Flt-1, Flt-3, folate receptor alpha, Tissue Factor (TF), CA6, MUC1, MUC16 (CA-125), BCMA, SLAMF7 (CSi), TIM1, CanAg, Ckit (CD117), EphA2, Nectin4, SLTRK6, FGFR2, LYPD3 (C4.4a), Cadherin 3, 5T4 (TPBG), STEAP1, PTK7, Ephrin-A4, LIV-1 (SLC39A6 or ZIP6), SLC1A5, TENB2, ETBR, integrin v3, Cripto, AGS-5 (SLC44A4), LY6E, AXL, LAMP1, LRRC15, TNF-alpha, MN/CA IX but not limited to such. In some embodiments, the invention provides a method of treating a disorder, or condition, or disease, or cancer by administering to a patient a therapeutically-effective amount of an anti-TROP2 antibody or ADC of the invention. In some embodiments, the invention provides a method of treating a disorder, or condition, or disease, or cancer by administering to a patient a therapeutically-effective amount of an anti-HER2 antibody or ADC of the invention. In some embodiments, the invention provides a method of treating a disorder, or condition, or disease, or cancer by administering to a patient a therapeutically-effective amount of an anti-CD3 antibody or ADC of the invention. In some embodiments, the invention provides a method of treating a disorder, or condition, or disease, or cancer by administering to a patient a therapeutically-effective amount of an anti-PSMA antibody or ADC of the invention. In some embodiments, the invention provides a method of treating a disorder, or condition, or disease, or cancer by administering to a patient a therapeutically-effective amount of an anti-CD70 antibody or ADC of the invention.

In some aspects, the disclosure provides ADCs for use in treating a disease or condition in a cell expressing high TROP2 receptor number. In some aspects, the disclosure provides ADCs for use in treating a disease or condition in a cell expressing high HER2 receptor number. In some aspects, the disclosure provides ADCs for use in treating a disease or condition in a cell expressing high CD3 receptor number. In some aspects, the disclosure provides ADCs for use in treating a disease or condition in a cell expressing high PSMA receptor number. In some aspects, the disclosure provides ADCs for use in treating a disease or condition in a cell expressing high CD70 receptor number. The antibodies and ADCs of the disclosure are for use in treating cancer including, but not limited to, ovarian cancer ovarian cancer including, but not limited to, an epithelial, stromal and germ cell tumor. The ovarian cancer may comprise a fallopian tube cancer or primary peritoneal carcinoma. The cancer may be characterized by high expression of antigen receptors, such as ovarian cancer, for example. The cancer may be treated by recruiting cytotoxic T cells to high expressing antigen receptor tumor cells. The antibodies of the disclosure are for use in treating inherited diseases, AIDS, or diabetes but is not limited to such. The antibodies, compounds or composition or conjugates of the disclosure can be used in the manufacture of a medicament for treating a disease or condition in a cell expressing high receptor number. The antibodies, compounds or composition or conjugates of the disclosure can be used in the manufacture of a medicament for treating cancer including, but not limited to, breast cancer including triple negative breast cancer, ovarian cancer including, but not limited to, an epithelial, stromal and germ cell tumor. The antibodies of the invention can be used in the manufacture of a medicament for treating diseases, conditions or cancers related to or associated with expression of an antigen receptor such as TROP2, HER2, CD3, PSMA, CD70, HER3 or GPC3 or antigen receptor for example. The anti-TROP2 antibodies of the invention can be used in the manufacture of a medicament for treating diseases, conditions or cancers related to or associated with high TROP2 receptor numbers. In other embodiments, the anti-HER2 antibodies of the disclosure can be used in the manufacture of a medicament for treating diseases, conditions or cancers related to or associated with HER2 expression. In other embodiments, the anti-CD3 antibodies of the disclosure can be used in the manufacture of a medicament for treating diseases, conditions or cancers related to or associated with CD3 expression. In other embodiments, the anti-PSMA antibodies of the disclosure can be used in the manufacture of a medicament for treating diseases, conditions or cancers related to or associated with PSMA expression. In other embodiments, the anti-CD70 antibodies of the disclosure can be used in the manufacture of a medicament for treating diseases, conditions or cancers related to or associated with CD70 expression. In other embodiments, the anti-CD70 antibodies of the disclosure can be used in the manufacture of a medicament for treating diseases, conditions or cancers related to or associated with HER3 expression. In other embodiments, the anti-CD70 antibodies of the disclosure can be used in the manufacture of a medicament for treating diseases, conditions or cancers related to or associated with GPC3 expression.

In some embodiments the condition to be treated is a cancer. The cancer may be, but is non-limited to, a breast cancer including triple negative breast cancer (TNBC), a brain cancer, a pancreatic cancer, a skin cancer, a lung cancer, a liver cancer, a gall bladder cancer, a colon cancer, an ovarian cancer, a prostate cancer, a uterine cancer, a bone cancer, and a blood cancer or a cancer or disease or conditions related to any of these cancers. A blood cancer can be a leukemia, a lymphoma or a myeloma. In some embodiments, the cancer is leukemia and the leukemia is AML. Carcinomas are cancers that begin in the epithelial cells, which are cells that cover the surface of the body, produce hormones, and make up glands. By way of non-limiting example, carcinomas include breast cancer, pancreatic cancer, lung cancer, colon cancer, colorectal cancer, rectal cancer, kidney cancer, bladder cancer, stomach cancer, prostate cancer, liver cancer, ovarian cancer, brain cancer, vaginal cancer, vulvar cancer, uterine cancer, oral cancer, penile cancer, testicular cancer, esophageal cancer, skin cancer, cancer of the fallopian tubes, head and neck cancer, gastrointestinal stromal cancer, adenocarcinoma, cutaneous or intraocular melanoma, cancer of the anal region, cancer of the small intestine, cancer of the endocrine system, cancer of the thyroid gland, cancer of the parathyroid gland, cancer of the adrenal gland, cancer of the urethra, cancer of the renal pelvis, cancer of the ureter, cancer of the endometrium, cancer of the cervix, cancer of the pituitary gland, neoplasms of the central nervous system (CNS), primary CNS lymphoma, brain stem glioma, and spinal axis tumors. In some instances, the cancer is a skin cancer, such as a basal cell carcinoma, squamous, melanoma, nonmelanoma, or actinic (solar) keratosis. In some embodiments the cancer is any cancer with highly expressed antigen receptor numbers such as, for example, TROP2 antigen receptor numbers, HER2 antigen receptor numbers, CD3 antigen receptor numbers, PSMA antigen receptor numbers, CD70 antigen receptor numbers, HER3 antigen receptor numbers, or GPC3 antigen receptor numbers. In some embodiments the condition to be treated is a disease or condition associated with or having a high antigen receptor number such as, for example, TROP2 antigen receptor number, HER2 antigen receptor number, CD3 antigen receptor number, PSMA antigen receptor numbers, CD70 antigen receptor number, HER3 antigen receptor number or GPC3 receptor number. The disease or condition may be a pathogenic infection. The pathogenic infection may be a bacterial infection. The pathogenic infection may be a viral infection. The disease or condition may be an inflammatory disease. The disease or condition may be an autoimmune disease. The autoimmune disease may be diabetes. The disease or condition may be a cancer. In some embodiments the disease or condition is any disease or condition with highly expressed antigen receptor numbers such as, for example, TROP2 antigen receptor numbers. The disease or condition may be a pathogenic infection. The biologically active molecule may interact with a cell surface molecule on an infected cell. The biologically active molecule may interact with a molecule on a bacterium, a virus, or a parasite. Pathogenic infections may be caused by one or more pathogens. In some instances, the pathogen is a bacterium, fungi, virus, or protozoan. Exemplary pathogens include but are not limited to: Bordetella, Borrelia, Brucella, Campylobacter, Chlamydia, Chlamydophila, Clostridium, Corynebacterium, Enterococcus, Escherichia, Francisella, Haemophilus, Helicobacter, Legionella, Leptospira, Listeria, Mycobacterium, Mycoplasma, Neisseria, Pseudomonas, Rickettsia, Salmonella, Shigella, Staphylococcus, Streptococcus, Treponema, Vibrio, or Yersinia. The pathogen may be a virus. Examples of viruses include, but are not limited to, adenovirus, coxsackievirus, Epstein-Barr virus, Hepatitis virus (e.g., Hepatitis A, B, and C), herpes simplex virus (type 1 and 2), cytomegalovirus, herpes virus, HIV, influenza virus, measles virus, mumps virus, papillomavirus, parainfluenza virus, poliovirus, respiratory syncytial virus, rubella virus, and varicella-zoster virus. Examples of diseases or conditions caused by viruses include, but are not limited to, cold, flu, hepatitis, AIDS, chicken pox, rubella, mumps, measles, warts, and poliomyelitis. The disease or condition may be an autoimmune disease or autoimmune related disease. An autoimmune disorder may be a malfunction of the body's immune system that causes the body to attack its own tissues. Examples of autoimmune diseases and autoimmune related diseases include, but are not limited to, Addison's disease, alopecia areata, ankylosing spondylitis, antiphospholipid syndrome (APS), autoimmune aplastic anemia, autoimmune hemolytic anemia, autoimmune hepatitis, autoimmune myocarditis, Behcet's disease, celiac sprue, Crohn's disease, dermatomyositis, eosinophilic fasciitis, erythema nodosum, giant cell arteritis (temporal arteritis), Goodpasture's syndrome, Graves' disease, Hashimoto's disease, idiopathic thrombocytopenic purpura (ITP), IgA nephropathy, juvenile arthritis, diabetes, juvenile diabetes, Kawasaki syndrome, Lambert-Eaton syndrome, lupus (SLE), mixed connective tissue disease (MCTD), multiple sclerosis, myasthenia gravis, pemphigus, polyarteritis nodosa, type I, II, & III autoimmune polyglandular syndromes, polymyalgia rheumatica, polymyositis, psoriasis, psoriatic arthritis, Reiter's syndrome, relapsing polychondritis, rheumatoid arthritis, sarcoidosis, scleroderma, Sjogren's syndrome, sperm & testicular autoimmunity, stiff person syndrome, Takayasu's arteritis, temporal arteritis/giant cell arteritis, ulcerative colitis, uveitis, vasculitis, vitiligo, and Wegener's granulomatosis.

The disease or condition may be an inflammatory disease. Examples of inflammatory diseases include, but are not limited to, alveolitis, amyloidosis, angiitis, ankylosing spondylitis, avascular necrosis, Basedow's disease, Bell's palsy, bursitis, carpal tunnel syndrome, celiac disease, cholangitis, chondromalacia patella, chronic active hepatitis, chronic fatigue syndrome, Cogan's syndrome, congenital hip dysplasia, costochondritis, Crohn's Disease, cystic fibrosis, De Quervain's tendinitis, diabetes associated arthritis, diffuse idiopathic skeletal hyperostosis, discoid lupus, Ehlers-Danlos syndrome, familial mediterranean fever, fascitis, fibrositis/fibromyalgia, frozen shoulder, ganglion cysts, giant cell arteritis, gout, Graves' Disease, HIV-associated rheumatic disease syndromes, hyperparathyroid associated arthritis, infectious arthritis, inflammatory bowel syndrome/irritable bowel syndrome, juvenile rheumatoid arthritis, Lyme disease, Marfan's Syndrome, Mikulicz's Disease, mixed connective tissue disease, multiple sclerosis, myofascial pain syndrome, osteoarthritis, osteomalacia, osteoporosis and corticosteroid-induced osteoporosis, Paget's Disease, palindromic rheumatism, Parkinson's Disease, Plummer's Disease, polymyalgia rheumatica, polymyositis, pseudogout, psoriatic arthritis, Raynaud's Phenomenon/Syndrome, Reiter's Syndrome, rheumatic fever, rheumatoid arthritis, sarcoidosis, sciatica (lumbar radiculopathy), scleroderma, scurvy, sickle cell arthritis, Sjogren's Syndrome, spinal stenosis, spondyloisthesis, Still's Disease, systemic lupus erythematosis, Takayasu's (Pulseless) Disease, Tendinitis, tennis elbow/golf elbow, thyroid associated arthritis, trigger finger, ulcerative colitis, Wegener's Granulomatosis, and Whipple's Disease.

The pharmaceutical compositions containing an antibody or ADC of the invention may be formulated at a strength effective for administration by various means to a human patient experiencing disorders that may be affected by antibody agonists or antagonists, such as but not limited to, anti-proliferatives, anti-inflammatory, or anti-virals are used, either alone or as part of a condition or disease. Average quantities of an antibody or ADC may vary and in particular should be based upon the recommendations and prescription of a qualified physician. The exact amount of an antibody or ADC is a matter of preference subject to such factors as the exact type of condition being treated, the condition of the patient being treated, as well as the other ingredients in the composition. The disclosure also provides for administration of a therapeutically effective amount of another active agent such as an anti-cancer chemotherapeutic agent or immunotherapeutic agent but is not limited to such. The amount to be given may be readily determined by one skilled in the art based upon therapy with the antibody or ADCs of the invention.

### Pharmaceutical Compositions

In other aspects of the present invention the antibody, antibody fragments, variants or ADCs further comprise a pharmaceutical composition or formulation. Such a pharmaceutical composition can employ various pharmaceutically acceptable excipients, stabilizers, buffers, and other components for administration to animals. See, for example, Remington, The Science and Practice of Pharmacy, 19th ed., Gennaro, ed., Mack Publishing Co., Easton, PA, 1995. Identifying suitable composition or formulations for stability, administration to a subject, and activity varies with each compound as a number of components, (for example, purifying, stabilizing components), need to be considered. Suitable salts for inclusion into the composition or formulation can include, but not limited to, sodium chloride, potassium chloride or calcium chloride. Buffering and/or stabilizing agents such as sodium acetate can be used. Suitable buffers can include phosphate-citrate buffer, phosphate buffer, citrate buffer, L-histidine, L-arginine hydrochloride, bicarbonate buffer, succinate buffer, citrate buffer, and TRIS buffer, either alone or in combination. Surfactants can also be employed, including polysorbates (e.g., polysorbate 80), dodecyl sulfate (SDS), lecithin either alone or in combination.

In some aspects of the present invention, the pharmaceutical composition or formulation can be an aqueous composition or in the form of a reconstituted liquid composition or as a powder. The composition or formulation can have a pH range from about 4.0 to about 7.0 or from about 4.5 to about 6.5 when the formulation is in a liquid form. However, the pH can be adjusted to provide acceptable stability and administration by the skilled medical practitioner.

The composition can be stored in a vial or cartridge, a pen delivery device, a syringe, intravenous administration tubing or an intravenous administration bag but is not limited to such. In other embodiments a pharmaceutical composition of the invention can be administered as a single dose or followed by one or more subsequent dose(s) minutes, days, or weeks after the first dose. Further administrations may be contemplated as needed to treat, reduce or prevent a cancer, condition, disorder or disease.

In some instances, the antibodies, antibody fragments, variants, or ADCs of the present invention disclosure may be used in conjunction with an additional therapy or treatment including but not limited to surgery, radiation, cryosurgery, thermotherapy, hormone treatment, chemotherapy, vaccines and other immunotherapies. In some embodiments such additional treatment can include a therapeutic agent such as chemotherapeutic agent, hormonal agent, antitumor agent, immunostimulatory agent, immunomodulator, corticosteroid or combination thereof.

In other embodiments the antibodies, antibody fragments, variants, or ADCs of the invention can be administered with one or more immunostimulatory agents to induce or enhance an immune response. Immunostimulatory agents that can stimulate specific arms of the immune system, such as natural killer (NK) cells that mediate antibody-dependent cell cytotoxicity (ADCC). Such immunostimulatory agents include, but are not limited to, IL-2, immunostimulatory oligonucleotides (for example, CpG motifs), α-interferon, γ-interferon, tumor necrosis factor alpha (TNFα). In other embodiments the ADCs of the invention can be administered with one or more immunomodulators including, but not limited to, cytokines, chemokines (including, but are not limited to, SLC5 ELC, MIP3α, MIP3β, IP—IO, MIG, and combinations thereof). Other therapeutic agents can be a vaccine that immunizes a subject against an antigen such as, for example, TROP2, HER2, CD3, PSMA, CD70, HER3 or GPC3. Such vaccines, in some embodiments, include antigens, with, optionally, one or more adjuvants to induce or enhance an immune response. Adjuvants of many kinds are well known in the art.

The chemotherapeutic agent or any agent involved in treating, reducing or preventing a disease, condition or cancer in a subject in need thereof can also be administered in combination with an ADC of the invention disclosure. Chemotherapeutic agents may include, but are not limited to, erlotinib (TARCEVA®, Genentech/OSI Pharm.), bortezomib (VELCADE®, Millennium Pharm.), fulvestrant (FASLODEX®, AstraZeneca), sutent (SU11248, Pfizer), letrozole (FEMARA®, Novartis), imatinib mesylate (GLEEVEC®, Novartis), PTK787/ZK 222584 (Novartis), oxaliplatin (Eloxatin®, Sanofi), 5-FU (5-fluorouracil), leucovorin, Rapamycin (Sirolimus, RAPAMUNE®, Wyeth), lapatinib (TYKERB®, GSK572016, GlaxoSmithKline), lonafarnib (SCH 66336), sorafenib (BAY43-9006, Bayer Labs.), and gefitinib (IRESSA®, AstraZeneca), AG1478, AG1571 (SU 5271; Sugen), alkylating agents such as thiotepa and CYTOXAN® cyclosphosphamide; alkyl sulfonates such as busulfan, improsulfan and piposulfan; antifolate antineoplastic such as pemetrexed (ALIMTA®, Eli Lilly), aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics, calicheamicin, calicheamicin gamma and calicheamicin omega; dynemicin, including dynemicin A; bisphosphonates, such as clodronate; an esperamicin; as well as neocarzinostatin chromophore and related chromoprotein enediyne antibiotic chromophores, aclacinomysins, actinomycin, anthramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; antiandrogens or androgen deprivation therapy; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, OR); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel (TAXOL®, Bristol-Myers Squibb Oncology, Princeton, N.J.), ABRAXANE™ Cremophor-free, albumin, nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; CPT-11; topoisomerase inhibitor RFS 2000; difluoromethylornithine (DMFO); retinoids such as retinoic acid; capecitabine; and pharmaceutically acceptable salts, acids or derivatives of any of the above.

### EXAMPLES

It is understood that the examples and embodiments described herein are for illustrative purposes only and that various modifications or changes in light thereof will be suggested to persons skilled in the art and are to be included within the spirit and purview of this application and scope of the appended claims.

### Example 1: General Experimental Procedures

All commercially available anhydrous solvents were used without further purification and were stored under a nitrogen atmosphere. TLC was performed on Merck Silica gel 60 F254 plates using UV light and/or staining with aqueous KMnO₄ solution for visualization. Chromatographic purification was performed on CombiFlash Rf from Teledyne ISCO using conditions detailed in the experimental procedure. Analytical HPLC was performed on Shimadzu system using Phenomenex Gemini —NX C18 5 μm 50×4.6 mm column, which was eluted at 1 ml/min with a linear gradient of acetonitrile in water containing 0.05% TFA. (Mobile phase A: 0.05% TFA in water; mobile phase B: 0.05% TFA in 90% acetonitrile (ACN) aqueous solution) or Water BEH 1.7 μM v2.1×50 mm column. Analytic Method 1: 0% B in 1 min, 0-50% B in 11 min, 50-100% B in 0.5 min, 100% B for 1.5 min, 100-0% B in 1 min, 0% B for 2 min; Method 2: 10-20% B in 1 min, 20-70% B in 11 min, 70-100% B in 0.5 min, 100% B for 1.5 min, 100-10% B in 1 min, 10% B for 2 min; Method 3: Method 2: 0-40% B in 1 min, 40-90% B in 11 min, 90-100% B in 0.5 min, 100% B for 1.5 min, 100-10% B in 1 min, 10% B for 2 min. Preparative HPLC was performed on Shimadzu system using Gemini —NX C18 5 μm 100×30 mm, 150×30 mm or 250×50 mm column, depending on the scale. Mass spectra (MS) were recorded on a Shimadzu LCMS-2020 system and data were processed using Shimadzu LabSolutions software. Agilent 1260 Infinity Binary LC coupled with 6230 Accurate-Mass TOFMS system was used for HR-ESI-TOF analysis. NMR spectral data were collected on a 500 MHz Bruker NMR spectrometer. Chemical shifts (δ) were reported in ppm and referenced off the deuterium solvent signal. Coupling constants (J) are reported in hertz (Hz). Spin multiplicities are described as: s (singlet), br (broad), d (doublet), dd (doublet of doublets), t (triplet), q (quartet), or m (multiplet).

Chemical names of compounds were derived from chemical structures using ChemDraw 20.1.1 (CambridgeSoft).

Abbreviations used in the Examples herein: ACN: acetonitrile, CDI: 1,1′-carbonyldiimidazole, DCM: dichloromethane, DIPEA: N,N-diisopropylethylamine, DIAD: diisopropyl azodicarboxylate, DMF: dimethylformamide, EDC: 1-ethyl-3-(3-dimethylaminopropyl)carbodiimide HCl, EtOAc: ethyl acetate, EtOH: ethanol, HATU: 1-[bis(dimethylamino)methylene]-1H-1,2,3-triazolo[4,5-b]pyridinium 3-oxide hexafluorophosphate, HPLC: high performance liquid chromatography, MeOH: methanol, MS: mass spectrometry, TFA: trifluoroacetic acid, THF: tetrahydrofuran, TLC: thin layer chromatography.

### Example 2: Synthesis of Compound 4

The general scheme for the synthesis of compound 4 is shown below.

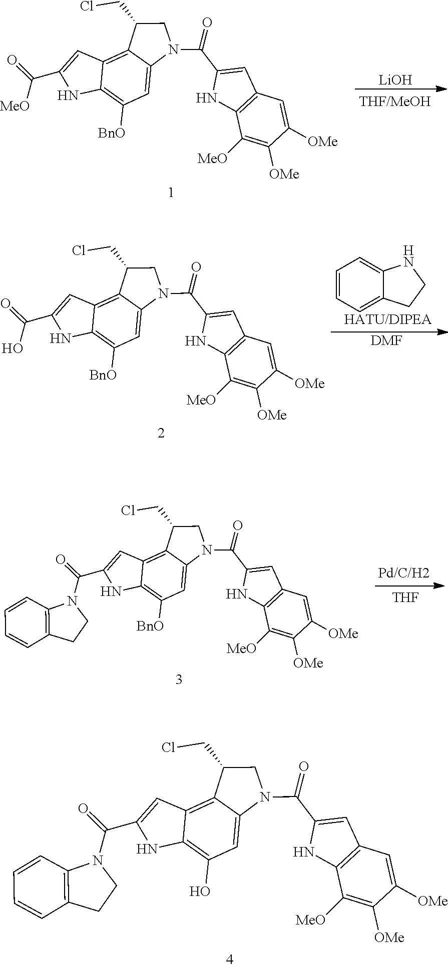

(S)-methyl 4-(benzyloxy)-8-(chloromethyl)-6-(5,6,7-trimethoxy-1H-indole-2-carbonyl)-3,6,7,8-tetrahydropyrrolo[3,2-e]indole-2-carboxylate (Compound 2). Methyl (S)-4-(benzyloxy)-8-(chloromethyl)-6-(5,6,7-trimethoxy-1H-indole-2-carbonyl)-3,6,7,8-tetrahydropyrrolo[3,2-e]indole-2-carboxylate (compound 1 (obtained by benzylation of seco-duocarmycin SA, CAS no. 152785-82-5, MedChemExpress, Monmouth Junction, NJ, USA); 1.25 g, 2.07 mmol) was dissolved in THF/MeOH (20 mL/10 mL) and treated with LiOH solution (1M, 2 mL). The reaction was run at room temperature for 1 hour, and then additional LiOH (2 mL) was added; after another 1 hour, additional LiOH (2 mL) was added. The solution was then kept at −20° C. overnight. On the second day, the volatile was removed, and water (20 mL) was added when cooled by ice-water bath. HCl (0.5N) solution was added until pH=1, and precipitate was collected by filtration, and the cake was washed with water (5 mL×3) and dried over high vacuum to give greenish solid as compound 2 (1.2 g, 98%). HPLC (Method 2): 7.0 min, MS m/z 590 (M+H)⁺.

(S)-(5-(benzyloxy)-1-(chloromethyl)-7-(indoline-1-carbonyl)-1,2-dihydropyrrolo[3,2-e]indol-3(6H)-yl)(5,6,7-trimethoxy-1H-indol-2-yl)methanone (Compound 3). The acid 2 (140 mg, 0.237 mmol) and indoline (CAS no. 496-15-1), 30 μL, 0.264 mmol; Sigma Aldrich) were dissolved in DMF (2 mL) and treated with HATU (110 mg, 0.289 mmol) followed by DIPEA (62.1 μL, 0.36 mmol). The reaction was kept at room temperature for 1 hour. Water (18 mL) was added into the solution, and the precipitate was collected by filtration. The cake was washed with water (3 mL×3) and dried under high vacuum to give yellowish solid (180 mg, >100%) directly for next step. HPLC (Method 2): 12.4 min, MS m/z 713 (M+Na)⁺.

(S)-(8-(chloromethyl)-4-hydroxy-6-(5,6,7-trimethoxy-1H-indole-2-carbonyl)-3,6,7,8-tetrahydropyrrolo[3,2-e]indol-2-yl)(indolin-1-yl)methanone (Compound 4). The substrate 3 (180 mg, 0.237 mmol) was dissolved in THF (30 ml, clear solution) and degassed and purged with N₂. Pd/C (120 mg) was added and re-degassed and purged with H₂ two times. The reaction was stirred at room temperature for 4 h. The reaction was quenched with Celite and filtrated, concentrated to get compound (4) (130 mg, 91%) as brown solid. HPLC (Method 2): 10.8 min, MS m/z 601 (M+H)⁺.

### Example 3: Synthesis of Compound 8

The general scheme for the synthesis of compound 8 is shown below.

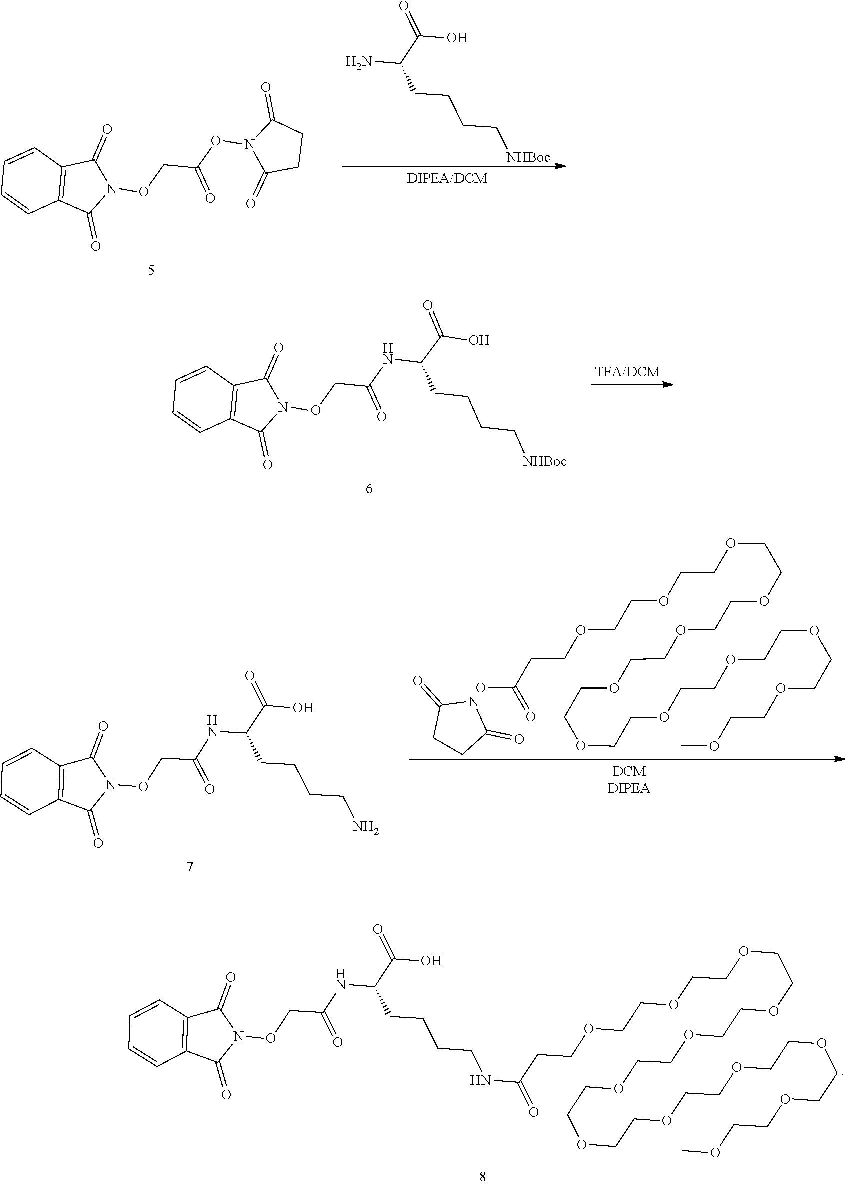

(S)-6-(tert-Butoxycarbonylamino)-2-(2-(1,3-dioxoisoindolin-2-yloxy)acetamido)hexanoic acid (Compound 6). The suspension of 2,5-dioxopyrrolidin-1-yl 2-(1,3-dioxoisoindolin-2-yloxy)acetate (compound 5 (CAS no. 1333377-78-8), 1 g, 4.17 mmol; Combi-Blocks, Catalog no. QD-9979), (S)-2-amino-6-(tert-butoxycarbonylamino)hexanoic acid (1.15 g, 4.67 mmol) and DIPEA (800.9 μL, 4.59 mmol) in DCM (40 ml) were stirred at room temperature for 1 hour (became almost clear solution). HPLC showed most of starting material was consumed. Removed the solvent via rotavap and the residue was dissolved in ACN/H₂O/TFA (adjusted to pH 1-2) and purified by HPLC and freeze-dried to give white powder as compound 6 (460 mg, 25%). HPLC (Method 2): 6.6 min, MS m/z 450 (M+H)⁺.

(S)-6-Amino-2-(2-(1,3-dioxoisoindolin-2-yloxy)acetamido)hexanoic acid (Compound 7). The Boc-protected compound (6) was dissolved in DCM (6 mL) and treated with TFA (2 mL) at room temperature for 15 min, and the volatile was removed in vacuo and co-evaporated with DCM (3 mL×2) to give a residue 7 (480 mg, >100%), which was used directly for the next step. HPLC (Method 1): 5.1 min.

(S)-44-(2-(1,3-Dioxoisoindolin-2-yloxy)acetamido)-38-oxo-2,5,8,11,14,17,20,23,26,29,32,35-dodecaoxa-39-azapentatetracontan-45-oic acid (Compound 8). Acid (7) (480 mg, calc. 1.02 mmol), m-dPEG12-Osuccinimide (780 mg, 1.14 mmol) and DIPEA (800.9 μL, 4.59 mmol) were dissolved in DCM (20 mL) and stirred at room temperature for 3 hours. Removed the solvent via rotavap and the residue was dissolved in ACN/H₂O/TFA (adjusted to pH 1-2) and purified by HPLC and freeze-dried to give white powder as compound 8 (575 mg, 3-step 61%). HPLC (Method 1): 8.9 min, MS m/z 920 (M+H)⁺.

### Example 4: Synthesis of Compound 12

The general scheme for the synthesis of compound 12 is shown below.

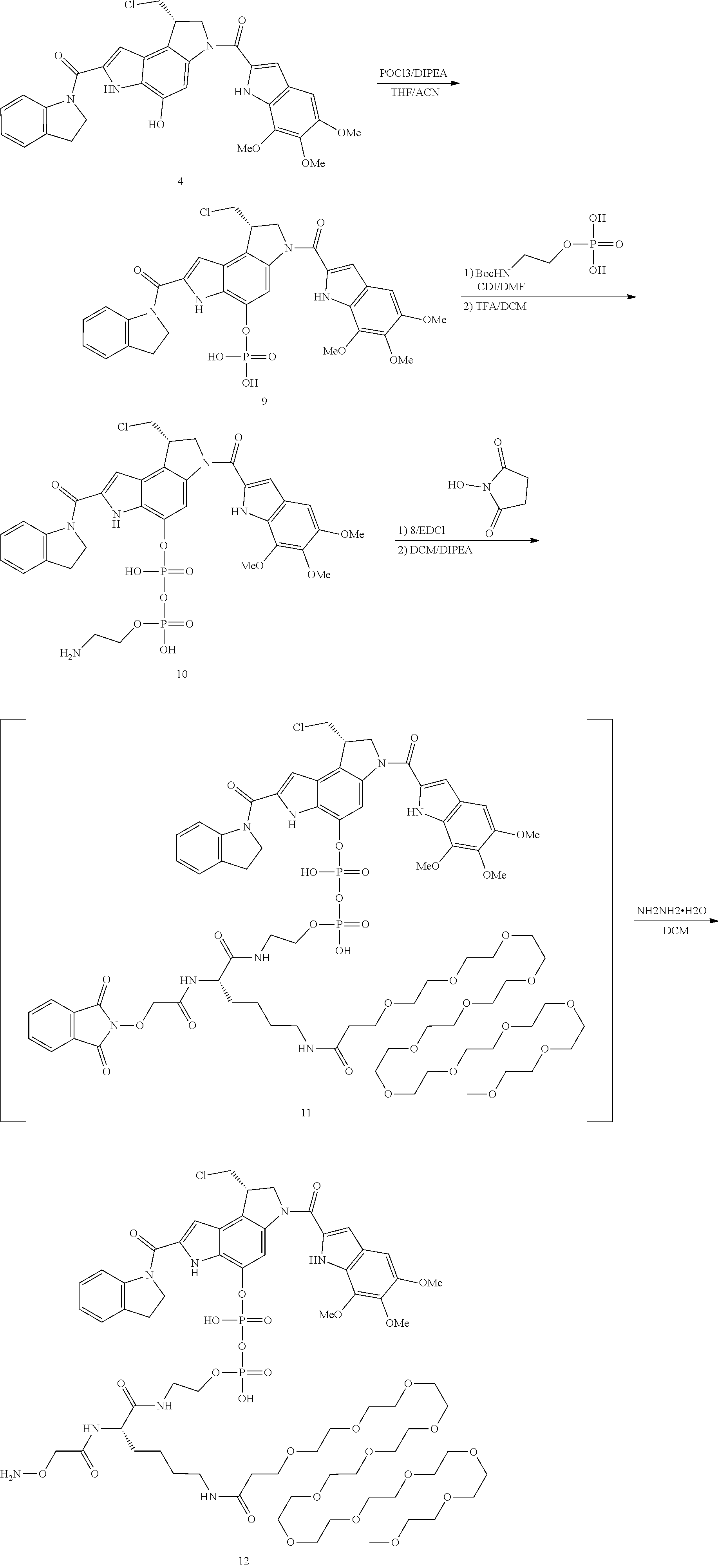

(S)-8-(chloromethyl)-2-(indoline-1-carbonyl)-6-(5,6,7-trimethoxy-1H-indole-2-carbonyl)-3,6,7,8-tetrahydropyrrolo[3,2-e]indol-4-yl dihydrogen phosphate (9). Compound 4 (130 mg, 0.216 mmol) in THF/ACN (30 mL/20 mL; with some suspension) was cooled to −20° C. (ACN-dry ice bath) and treated with POCl₃ (205 μL, 2.2 mmol) slowly, followed by DIPEA (227 μL, 1.3 mmol). The reaction mixture was maintained at −20° C. for 30 min, then 4° C. (ice-water bath) for 30 min. HPLC showed starting material was still present. The reaction mixture was re-cooled to −20° C., and additional POCl₃ (205 μL, 2.2 mmol) and DIPEA (227 μL, 1.3 mmol) were added and the reaction mixture was warmed up to 4° C. for 30 min. HPLC showed there was no starting material, and the reaction was quenched with 1M NaH₂PO4 and left at 4° C. overnight. On the second day, intermediate was still present. The reaction mixture was allowed to warm up to room temperature for 3 hours. After removing all volatile, the residue was suspended in water (20 mL) and filtrated. The cake was washed with water (10 mL×3) to give a light brown solid, which was dried over high vacuum as crude compound 9 (160 mg, >100%) and directly used for the next step. HPLC (Method 3): 5.7 min, MS m/z 679 (M−H)⁺.

(S)-4-(((2-aminoethoxy)(hydroxy)phosphoryloxy)(hydroxy)phosphoryloxy)-8-(chloromethyl)-2-(indoline-1-carbonyl)-6-(5,6,7-trimethoxy-1H-indole-2-carbonyl)-3,6,7,8-tetrahydropyrrolo[3,2-e]indole (10). tert-Butyl 2-(phosphonooxy)ethylcarbamate (147 mg, 0.43 mmol) was dissolved in DMF (1 mL) and treated with CDI (215 mg, 1.33 mmol) at room temperature for 30 min. After quenching with MeOH (3 drops) for 15 min, removed all the volatile to give a syrup. To the above syrup in DMF (2 mL) solution was added compound 9 (160 mg, calc. 0.216 mmol). The reaction was kept at room temperature overnight. After diluting with DCM (4 mL) and cooling to 4° C. on ice-bath, TFA (4 mL) was added for 30 min. DCM was removed and the residue was purified by HPLC and desired fractions were collected and concentrated using a rotavap to give the target molecule TFA salt (10) (67 mg, 34%). HPLC (Method 2): 4.9 min, MS m/z 802 (M−H)⁺.

(8S)-4-(((44-(2-(aminooxy)acetamido)-38,45-dioxo-2,5,8,11,14,17,20,23,26,29,32,35-dodecaoxa-39,46-diazaoctatetracontan-48-yl) hydrooxyhydroxylphosphoryloxy)(hydroxy)phosphoryloxy)-8-(chloromethyl)-2-(indolin-1-carbonyl)-6-(5,6,7-trimethoxy-1H-indole-2-carbonyl)-3,6,7,8-tetrahydropyrrolo[3,2-e]indole (11). Acid (8) (95 mg, 0.103 mmol) was dissolved in DCM (3 mL) and treated with N-hydroxysuccinimide (16.8 mg, 0.146 mmol) and EDC (32.2 mg, 0.168 mmol). The reaction was kept at room temperature for 6 h and was added into a suspension of 10 (67 mg, 0.073 mmol) and DIPEA (63.7 μL, 0.365 mmol) in DCM (3 mL). The mixture was kept at room temperature for 2 h, then at 4° C. overnight (16 h). After removing some DCM (~2 mL remaining), NH₂NH₂ (36.6 μL, 0.73 mmol) was added to the crude product 11 at room temperature for 10 min. Completed removal of volatile and re-dissolved in ACN/H₂O (1/1.5 mL) and adjusted pH to ~2 by 10% citric acid solution. The clear solution was then injected into HPLC and purified. Collected desired fractions and freeze-dried to obtain target compound as white powder (11) (42 mg, 34%). HPLC (Method 1): 8.9 min, MS m/z 1575 (M+H)⁺.

### Example 5: Synthesis of Compound 14

The general scheme for the synthesis of compound 14 is shown below.

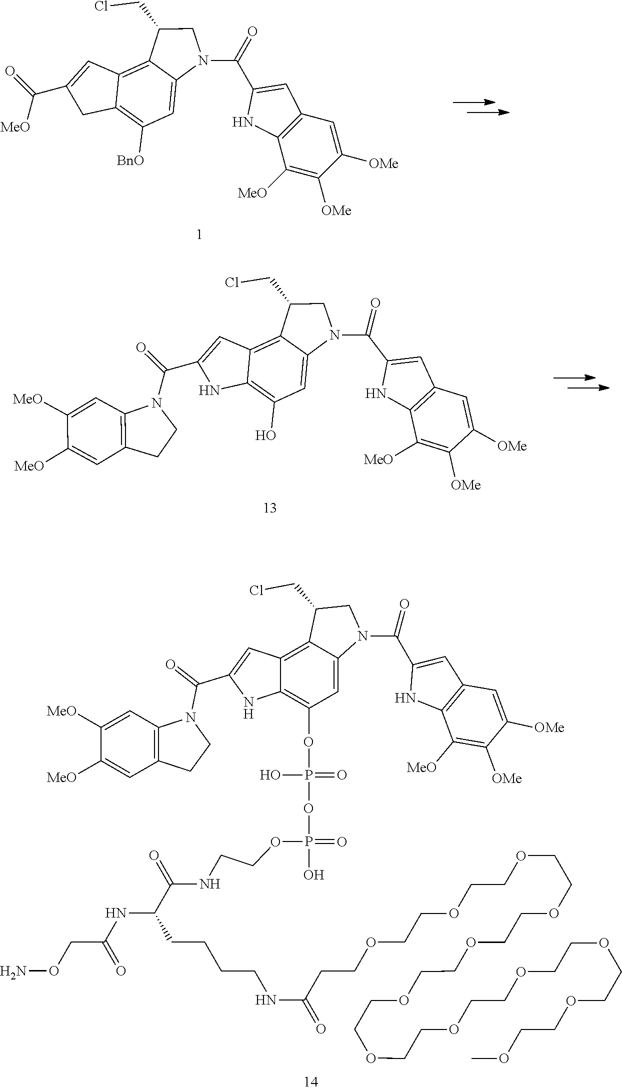 [SMILES: COC(=O)C1=Cc2c(c(OCc3ccccc3)cc3c2[C@H](CCl)CN3C(=O)c2cc3cc(OC)c(OC)c(OC)c3[nH]2)C1 ; COc1cc2c(cc1OC)N(C(=O)c1cc3c4c(cc(O)c3[nH]1)N(C(=O)c1cc3cc(OC)c(OC)c(OC)c3[nH]1)C[C@H]4CCl)CC2 ; COCCOCCOCCOCCOCCOCCOCCOCCOCCOCCOCCOCCC(=O)NCCCC[C@H](NC(=O)CON)C(=O)NCCOP(=O)(O)OP(=O)(O)Oc1cc2c(c3cc(C(=O)N4CCc5cc(OC)c(OC)cc54)[nH]c13)[C@H](CCl)CN2C(=O)c1cc2cc(OC)c(OC)c(OC)c2[nH]1]

(8S)-4-(((44-(2-(aminooxy)acetamido)-38,45-dioxo-2,5,8,11,14,17,20,23,26,29,32,35-dodecaoxa-39,46-diazaoctatetracontan-48-yl) hydrooxyhydroxylphosphoryloxy)(hydroxy)phosphoryloxy)-8-(chloromethyl)-2-(5,6-dimethoxyindolin-1-carbonyl)-6-(5,6,7-trimethoxy-1H-indole-2-carbonyl)-3,6,7,8-tetrahydropyrrolo[3,2-e]indole (14) Compound 13 was prepared using a similar method as described for compound 4 (see Example 2). Then, compound 14 was prepared via compound 13 using a similar method as described for compound 12 (see Example 4) to get the target compound 14 (15 mg, 6.9% from 1). HPLC (Method 3): 4.1 min, MS m/z 1633 (M−H)⁺.

### Example 6: Synthesis of Compound 16

The general scheme for the synthesis of compound 16 is shown below.

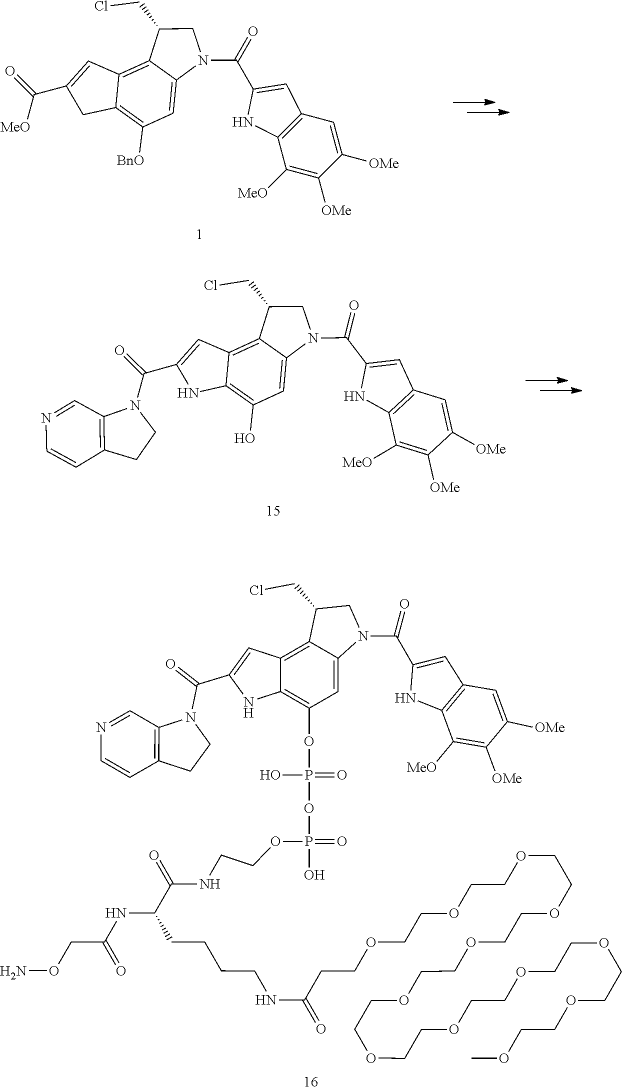 [SMILES: COC(=O)C1=Cc2c(c(OCc3ccccc3)cc3c2[C@H](CCl)CN3C(=O)c2cc3cc(OC)c(OC)c(OC)c3[nH]2)C1 ; COc1cc2cc(C(=O)N3C[C@@H](CCl)c4c3cc(O)c3[nH]c(C(=O)N5CCc6ccncc65)cc43)[nH]c2c(OC)c1OC ; COCCOCCOCCOCCOCCOCCOCCOCCOCCOCCOCCOCCC(=O)NCCCC[C@H](NC(=O)CON)C(=O)NCCOP(=O)(O)OP(=O)(O)Oc1cc2c(c3cc(C(=O)N4CCc5ccncc54)[nH]c13)[C@H](CCl)CN2C(=O)c1cc2cc(OC)c(OC)c(OC)c2[nH]1]

(8S)-4-(((44-(2-(aminooxy)acetamido)-38,45-dioxo-2,5,8,11,14,17,20,23,26,29,32,35-dodecaoxa-39,46-diazaoctatetracontan-48-yl) hydrooxyhydroxylphosphoryloxy)(hydroxy)phosphoryloxy)-8-(chloromethyl)-2-(2,3-dihydro-1H-pyrrolo[2,3-c]pyridin-1-carbonyl)-6-(5,6,7-trimethoxy-1H-indole-2-carbonyl)-3,6,7,8-tetrahydropyrrolo[3,2-e]indole (Compound 16). Compound 15 was prepared using a similar method as described for compound 4 (see Example 2). Then, compound 16 was prepared via compound 15 using similar method as described for compound 12 (see Example 4) to get the target compound 16 (16 mg, 0.64% from 1). HPLC (Method 2): 5.6 min, MS m/z 1577 (M+H)⁺, 1575 (M−H)⁺.

### Example 7: Synthesis of Compound 18

The general scheme for the synthesis of compound 18 is shown below.

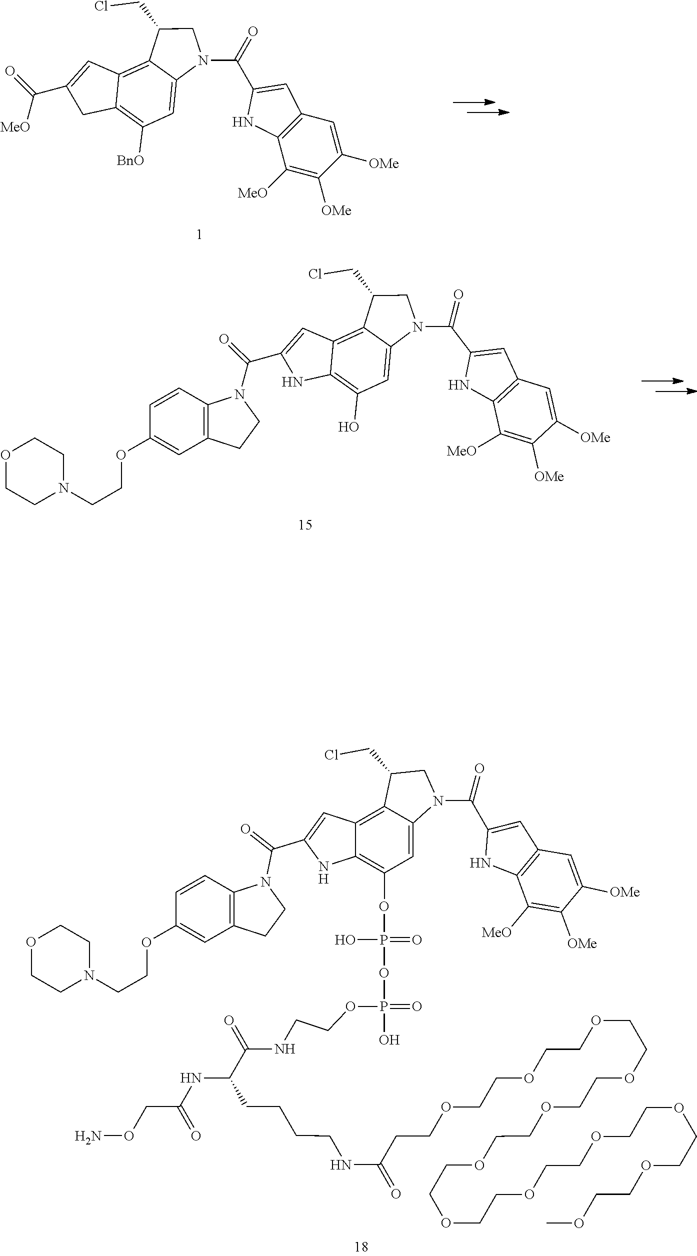 [SMILES: COC(=O)C1=Cc2c(c(OCc3ccccc3)cc3c2[C@H](CCl)CN3C(=O)c2cc3cc(OC)c(OC)c(OC)c3[nH]2)C1 ; COc1cc2cc(C(=O)N3C[C@@H](CCl)c4c3cc(O)c3[nH]c(C(=O)N5CCc6cc(OCCN7CCOCC7)ccc65)cc43)[nH]c2c(OC)c1OC ; COCCOCCOCCOCCOCCOCCOCCOCCOCCOCCOCCOCCC(=O)NCCCC[C@H](NC(=O)CON)C(=O)NCCOP(=O)(O)OP(=O)(O)Oc1cc2c(c3cc(C(=O)N4CCc5cc(OCCN6CCOCC6)ccc54)[nH]c13)[C@H](CCl)CN2C(=O)c1cc2cc(OC)c(OC)c(OC)c2[nH]1]

(8S)-4-(((44-(2-(aminooxy)acetamido)-38,45-dioxo-2,5,8,11,14,17,20,23,26,29,32,35-dodecaoxa-39,46-diazaoctatetracontan-48-yl) hydrooxyhydroxylphosphoryloxy)(hydroxy)phosphoryloxy)-8-(chloromethyl)-2-(5-(2-morpholinoethoxy)indolin-1-carbonyl)-6-(5,6,7-trimethoxy-1H-indole-2-carbonyl)-3,6,7,8-tetrahydropyrrolo[3,2-e]indole (18). Compound 17 was prepared using a similar method as described for compound 4 (see Example 2). Then, compound 18 was prepared via compound 17 using similar method as described for compound 12 (see Example 4) to get the target compound 18 (15 mg, 7.8% from 1). HPLC (Method 2): 6.0 min, MS m/z 1705 (M+H)⁺, 1703 (M−H)⁺.

### Example 8: Synthesis of Compound 19

The general scheme for the synthesis of compound 19 is shown below.

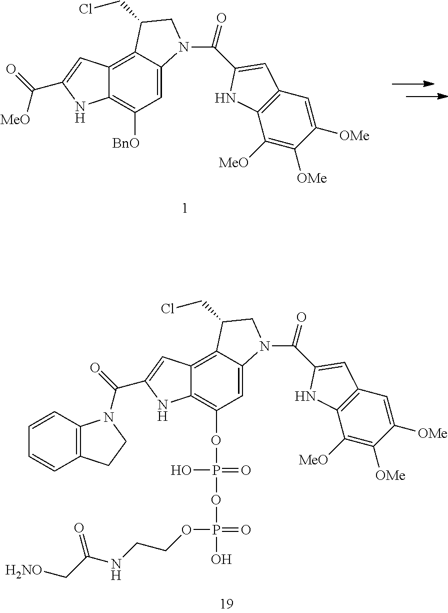

(S)-4-(((2-(2-(Aminooxy)acetamido)ethyl) hydrooxyhydroxylphosphoryloxy)(hydroxy)phosphoryloxy)-8-(chloromethyl)-2-(indolin-1-carbonyl)-6-(5,6,7-trimethoxy-1H-indole-2-carbonyl)-3,6,7,8-tetrahydropyrrolo[3,2-e]indole (19). Compound 19 was prepared using a similar method as described for compound 12 (see Example 4), using 2,5-dioxopyrrolidin-1-yl 2-(((tert-butoxycarbonyl)amino)oxy)acetate instead of compound 8 to get target compound 19 (10 mg, 7.5% from 1). HPLC (Method 3): 4.0 min, MS m/z 877 (M−H)⁺.

### Example 9: Synthesis of Compound 20

The general scheme for the synthesis of compound 20 is shown below.

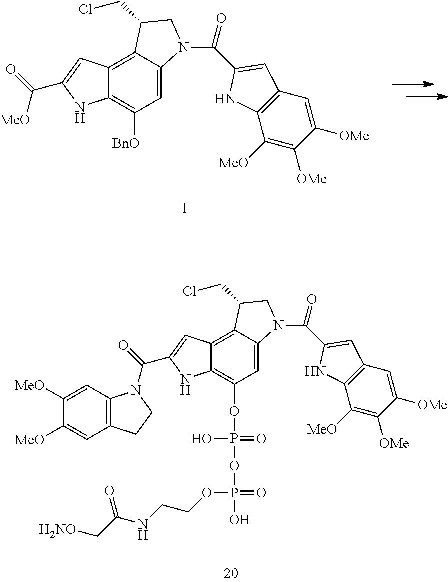

(S)-4-(((2-(2-(Aminooxy)acetamido)ethyl) hydrooxyhydroxylphosphoryloxy)(hydroxy)phosphoryloxy)-8-(chloromethyl)-2-(5,6-dimethoxyindolin-1-carbonyl)-6-(5,6,7-trimethoxy-1H-indole-2-carbonyl)-3,6,7,8-tetrahydropyrrolo[3,2-e]indole (20). Compound 20 was prepared using a similar method as described for compound 12 (see Example 4), using 2,5-dioxopyrrolidin-1-yl 2-(((tert-butoxycarbonyl)amino)oxy)acetate instead of compound 8 to get target compound 20 (14 mg, 4.0% from 1). HPLC (Method 3): 3.7 min, MS m/z 935 (M−H)⁺.

### Example 10: Synthesis of Compound 21

The general scheme for the synthesis of compound 21 is shown below.

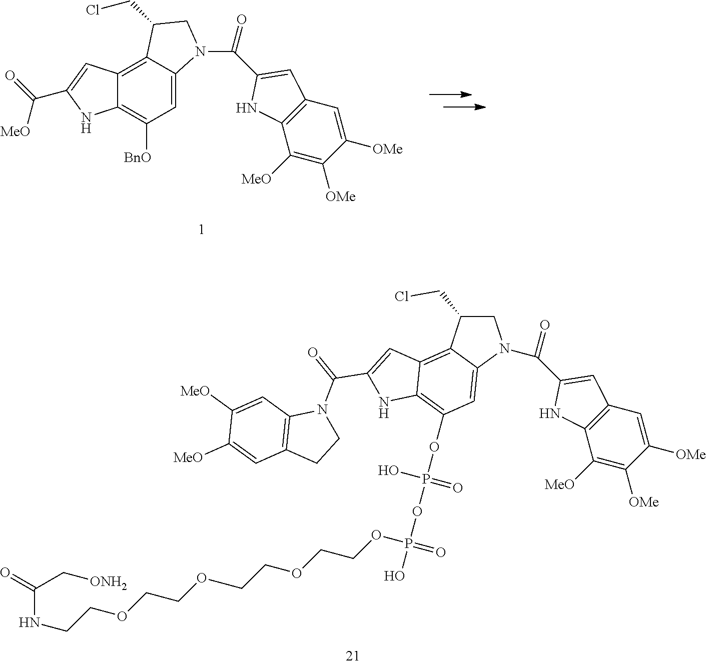

(S)-4-(((1-(aminooxy)-2-oxo-6,9,12-trioxa-3-azatetradecan-14-yl)hydrooxyhydroxylphosphoryloxy)(hydroxy)phosphoryloxy)-8-(chloromethyl)-2-(5,6-dimethoxyindolin-1-carbonyl)-6-(5,6,7-trimethoxy-1H-indole-2-carbonyl)-3,6,7,8-tetrahydropyrrolo[3,2-e]indole (21). Compound 21 was prepared using a similar method as described for compound 12 (see Example 4) to get target compound 21 (10 mg, 2.3% from 1). HPLC (Method 3): 4.2 min, MS m/z 1067 (M−H)⁺.

### Example 11: Synthesis of Compound 22

The general scheme for the synthesis of compound 22 is shown below.

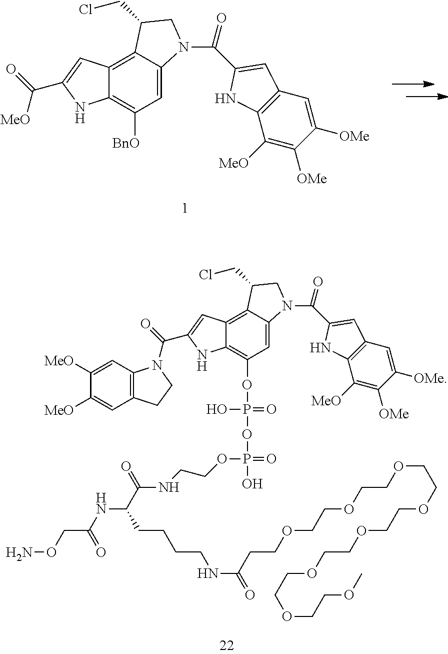 [SMILES: COC(=O)c1cc2c3c(cc(OCc4ccccc4)c2[nH]1)N(C(=O)c1cc2cc(OC)c(OC)c(OC)c2[nH]1)C[C@H]3CCl ; COCCOCCOCCOCCOCCOCCOCCOCCC(=O)NCCCC[C@H](NC(=O)CON)C(=O)NCCOP(=O)(O)OP(=O)(O)Oc1cc2c(c3cc(C(=O)N4CCc5cc(OC)c(OC)cc54)[nH]c13)[C@H](CCl)CN2C(=O)c1cc2cc(OC)c(OC)c(OC)c2[nH]1]

(8S)-4-(((32-(2-(aminooxy)acetamido)-26,33-dioxo-2,5,8,11,14,17,20,23-octaoxa-27,34-diazahexatriacontan-36-yl) hydrooxyhydroxylphosphoryloxy)(hydroxy)phosphoryloxy)-8-(chloromethyl)-2-(5,6-dimethoxyindolin-1-carbonyl)-6-(5,6,7-trimethoxy-1H-indole-2-carbonyl)-3,6,7,8-tetrahydropyrrolo[3,2-e]indole (22). Compound 22 was prepared using a similar method as described for compound 12 (see Example 4) to get target compound 22 (0.9 mg, 2.2% from 1). HPLC (Method 3): 4.0 min, MS m/z 1459 (M+H)⁺, 1457 (M−H)⁺.

### Example 12: Synthesis of Compound 23

The general scheme for the synthesis of compound 23 is shown below.

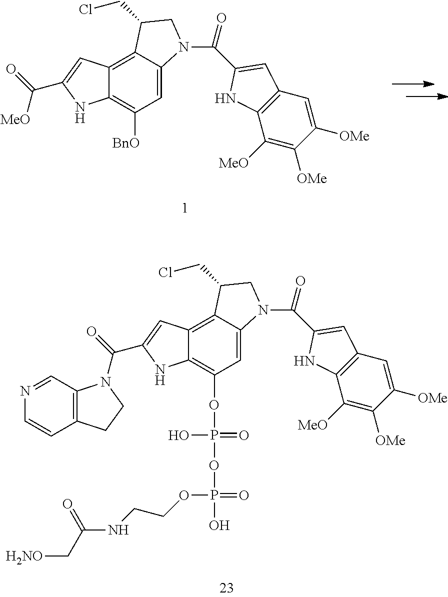

(S)-4-(((2-(2-(aminooxy)acetamido)ethyl) hydrooxyhydroxylphosphoryloxy)(hydroxy)phosphoryloxy)-8-(chloromethyl)-2-(2,3-dihydro-1H-pyrrolo[2,3-c]pyridin-1-carbonyl)-6-(5,6,7-trimethoxy-1H-indole-2-carbonyl)-3,6,7,8-tetrahydropyrrolo[3,2-e]indole (23). Compound 23 was prepared using a similar method as described for compound 12 (see Example 4) to get target compound 23. HPLC (Method 2): 5.0 min, MS m/z 876 (M−H)⁺.

### Example 13: Synthesis of Compound 24

The general scheme for the synthesis of compound 24 is shown below.

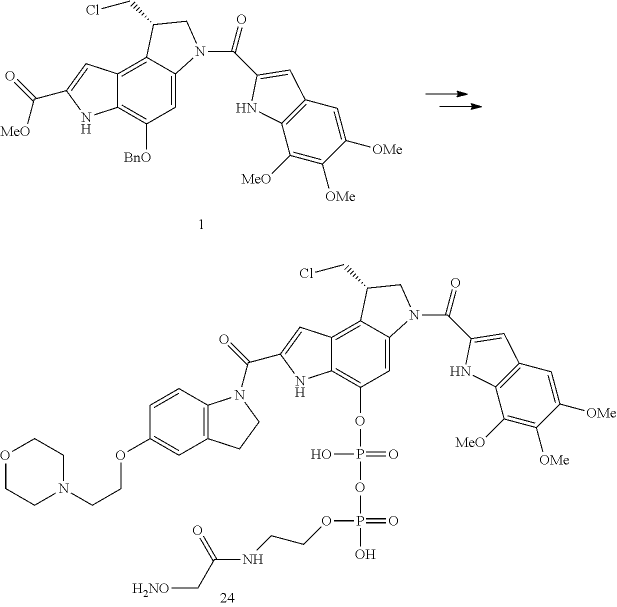

(8S)-4-(((2-(2-(aminooxy)acetamido)ethyl) hydrooxyhydroxylphosphoryloxy)(hydroxy)phosphoryloxy)-8-(chloromethyl)-2-(5-(2-morpholinoethoxy)indolin-1-carbonyl)-6-(5,6,7-trimethoxy-1H-indole-2-carbonyl)-3,6,7,8-tetrahydropyrrolo[3,2-e]indole (24). Compound 24 was prepared using a similar method as described for compound 12 (see Example 4) to get the target compound 24 (10 mg, 8.1% from 1).

HPLC (Method 2): 5.7 min, MS m/z 1006 (M+H)⁺, 1004 (M−H)⁺.

### Example 14: Synthesis of Compound 25

The general scheme for the synthesis of compound 25 is shown below.

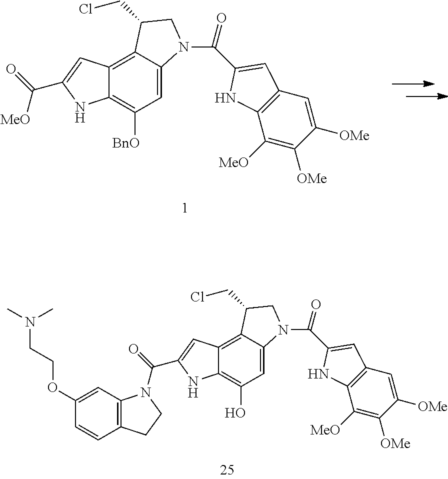 [SMILES: COC(=O)c1cc2c3c(cc(OCc4ccccc4)c2[nH]1)N(C(=O)c1cc2cc(OC)c(OC)c(OC)c2[nH]1)C[C@H]3CCl ; COc1cc2cc(C(=O)N3C[C@@H](CCl)c4c3cc(O)c3[nH]c(C(=O)N5CCc6ccc(OCCN(C)C)cc65)cc43)[nH]c2c(OC)c1OC]

(S)-(8-(chloromethyl)-4-hydroxy-6-(5,6,7-trimethoxy-1H-indole-2-carbonyl)-3,6,7,8-tetrahydropyrrolo[3,2-e]indol-2-yl)(6-(2-(dimethylamino)ethoxy)indolin-1-yl)methanone (25) Compound 25 was prepared using a similar method as described for compound 4 (see Example 2) to get the target compound 25 (4 mg, 11% from 1). HPLC (Method 2): 6.9 min, MS m/z 688 (M+H)⁺.

### Example 15: Synthesis of Compound 26

The general scheme for the synthesis of compound 26 is shown below.

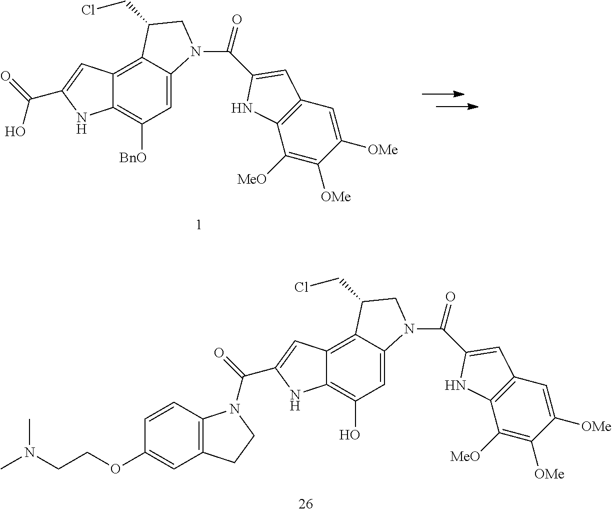 [SMILES: COc1cc2cc(C(=O)N3C[C@@H](CCl)c4c3cc(OCc3ccccc3)c3[nH]c(C(=O)O)cc43)[nH]c2c(OC)c1OC ; COc1cc2cc(C(=O)N3C[C@@H](CCl)c4c3cc(O)c3[nH]c(C(=O)N5CCc6cc(OCCN(C)C)ccc65)cc43)[nH]c2c(OC)c1OC]

(S)-(8-(chloromethyl)-4-hydroxy-6-(5,6,7-trimethoxy-1H-indole-2-carbonyl)-3,6,7,8-tetrahydropyrrolo[3,2-e]indol-2-yl)(5-(2-(dimethylamino)ethoxy)indolin-1-yl)methanone (26) Compound 26 was prepared using a similar method as described for compound 4 (see Example 2) to get the target compound 26 (2 mg, 10% from 1). HPLC (Method 2): 6.4 min, MS m/z 688 (M+H)⁺.

### Example 16: Synthesis of Compound 27

The general scheme for the synthesis of compound 27 is shown below.

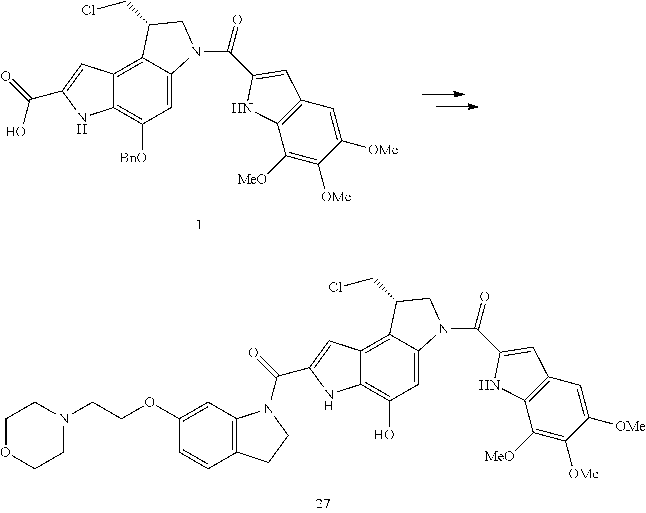 [SMILES: COc1cc2cc(C(=O)N3C[C@@H](CCl)c4c3cc(OCc3ccccc3)c3[nH]c(C(=O)O)cc43)[nH]c2c(OC)c1OC ; COc1cc2cc(C(=O)N3C[C@@H](CCl)c4c3cc(O)c3[nH]c(C(=O)N5CCc6ccc(OCCN7CCOCC7)cc65)cc43)[nH]c2c(OC)c1OC]

(S)-(8-(chloromethyl)-4-hydroxy-6-(5,6,7-trimethoxy-1H-indole-2-carbonyl)-3,6,7,8-tetrahydropyrrolo[3,2-e]indol-2-yl)(6-(2-morpholinoethoxy)indolin-1-yl)methanone (27). Compound 27 was prepared using a similar method as described for compound 4 (see Example 2) to get the target compound 27 (2 mg, 10% from 1). HPLC (Method 2): 7.0 min, MS m/z 730 (M+H)⁺.

### Example 17: Synthesis of Compound 28

The general scheme for the synthesis of compound 28 is shown below.

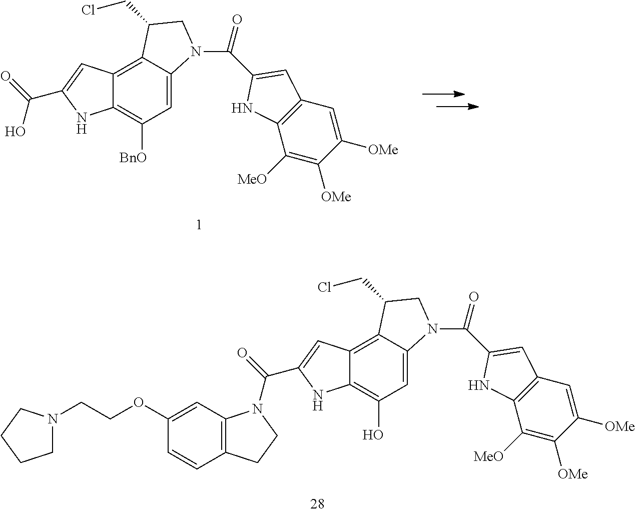 [SMILES: COc1cc2cc(C(=O)N3C[C@@H](CCl)c4c3cc(OCc3ccccc3)c3[nH]c(C(=O)O)cc43)[nH]c2c(OC)c1OC ; COc1cc2cc(C(=O)N3C[C@@H](CCl)c4c3cc(O)c3[nH]c(C(=O)N5CCc6ccc(OCCN7CCCC7)cc65)cc43)[nH]c2c(OC)c1OC]

(S)-(8-(chloromethyl)-4-hydroxy-6-(5,6,7-trimethoxy-1H-indole-2-carbonyl)-3,6,7,8-tetrahydropyrrolo[3,2-e]indol-2-yl)(6-(2-(pyrrolidin-1-yl)ethoxy)indolin-1-yl)methanone (28). Compound 28 was prepared using a similar method as described for compound 4 (see Example 2) to get the target compound 28 (2 mg, 10% from 1). HPLC (Method 2): 7.1 min, MS m/z 714 (M+H)⁺.

### Example 18: Synthesis of Compound 29

The general scheme for the synthesis of compound 29 is shown below.

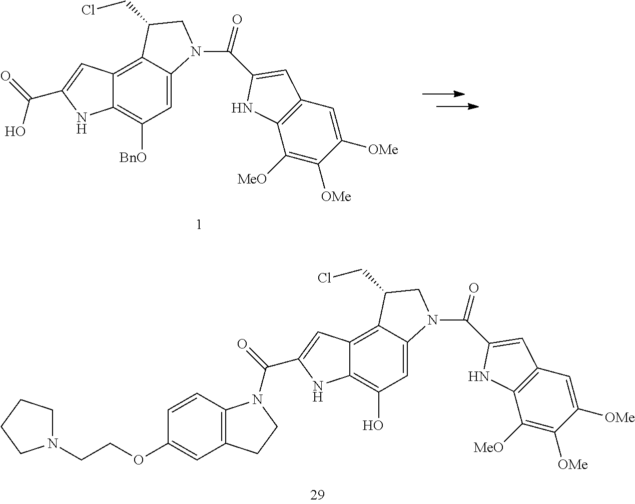 [SMILES: COc1cc2cc(C(=O)N3C[C@@H](CCl)c4c3cc(OCc3ccccc3)c3[nH]c(C(=O)O)cc43)[nH]c2c(OC)c1OC ; COc1cc2cc(C(=O)N3C[C@@H](CCl)c4c3cc(O)c3[nH]c(C(=O)N5CCc6cc(OCCN7CCCC7)ccc65)cc43)[nH]c2c(OC)c1OC]

(S)-(8-(chloromethyl)-4-hydroxy-6-(5,6,7-trimethoxy-1H-indole-2-carbonyl)-3,6,7,8-tetrahydropyrrolo[3,2-e]indol-2-yl)(5-(2-(pyrrolidin-1-yl)ethoxy)indolin-1-yl)methanone (29). Compound 28 was prepared using a similar method as described for compound 4 (see Example 2) to get the target compound 29 (4 mg, 17% from 1). HPLC (Method 2): 7.0 min, MS m/z 714 (M+H)⁺.

### Example 19: Synthesis of Compound 30

The general scheme for the synthesis of compound 30 is shown below.

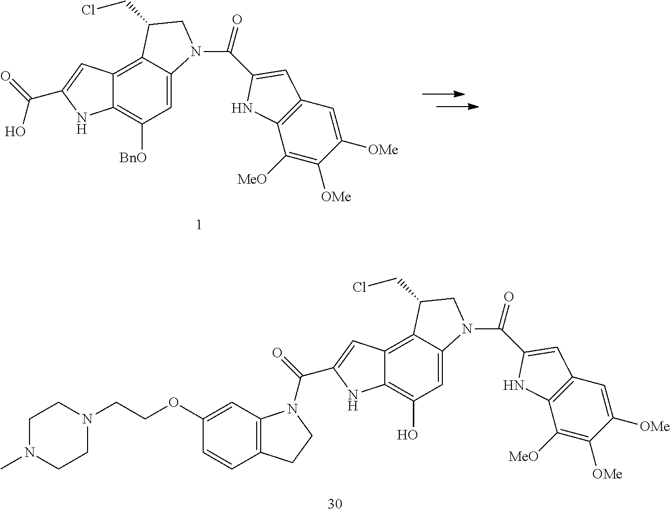 [SMILES: COc1cc2cc(C(=O)N3C[C@@H](CCl)c4c3cc(OCc3ccccc3)c3[nH]c(C(=O)O)cc43)[nH]c2c(OC)c1OC ; COc1cc2cc(C(=O)N3C[C@@H](CCl)c4c3cc(O)c3[nH]c(C(=O)N5CCc6ccc(OCCN7CCN(C)CC7)cc65)cc43)[nH]c2c(OC)c1OC]

(S)-(8-(chloromethyl)-4-hydroxy-6-(5,6,7-trimethoxy-1H-indole-2-carbonyl)-3,6,7,8-tetrahydropyrrolo[3,2-e]indol-2-yl)(6-(2-(4-methylpiperazin-1-yl)ethoxy)indolin-1-yl)methanone (30). Compound 30 was prepared using similar method as described for compound 4 to get the target compound 30 (6 mg, 30% from 1). HPLC (Method 2): 6.0 min, MS m/z 743 (M+H)⁺.

### Example 20: Synthesis of Compound 31

The general scheme for the synthesis of compound 31 is shown below.

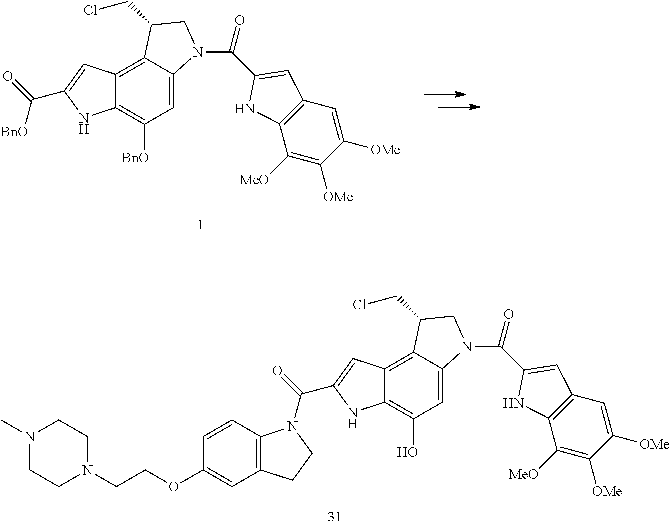 [SMILES: COc1cc2cc(C(=O)N3C[C@@H](CCl)c4c3cc(OCc3ccccc3)c3[nH]c(C(=O)OCc5ccccc5)cc43)[nH]c2c(OC)c1OC ; COc1cc2cc(C(=O)N3C[C@@H](CCl)c4c3cc(O)c3[nH]c(C(=O)N5CCc6cc(OCCN7CCN(C)CC7)ccc65)cc43)[nH]c2c(OC)c1OC]

(S)-(8-(chloromethyl)-4-hydroxy-6-(5,6,7-trimethoxy-1H-indole-2-carbonyl)-3,6,7,8-tetrahydropyrrolo[3,2-e]indol-2-yl)(5-(2-(4-methylpiperazin-1-yl)ethoxy)indolin-1-yl)methanone (31). Compound 31 was prepared using a similar method as described for compound 4 (see Example 2) to get the target compound 31 (7.8 mg, 34% from 1). HPLC (Method 2): 5.7 min, MS m/z 743 (M+H)⁺.

### Example 21: Synthesis of Compound 35

The general scheme for the synthesis of compound 35 is shown below.

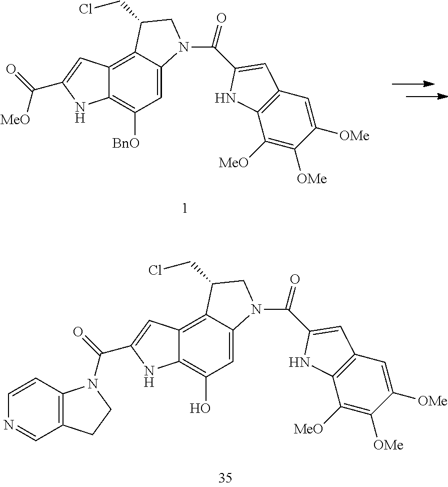 [SMILES: COC(=O)c1cc2c3c(cc(OCc4ccccc4)c2[nH]1)N(C(=O)c1cc2cc(OC)c(OC)c(OC)c2[nH]1)C[C@H]3CCl ; COc1cc2cc(C(=O)N3C[C@@H](CCl)c4c3cc(O)c3[nH]c(C(=O)N5CCc6cnccc65)cc43)[nH]c2c(OC)c1OC]

Compound 35 was prepared using a similar method as described for compound 4 (see Example 2). MS m/z 602 (M+H)⁺.

### Example 22: Synthesis of Compound 36

The general scheme for the synthesis of compound 36 is shown below.

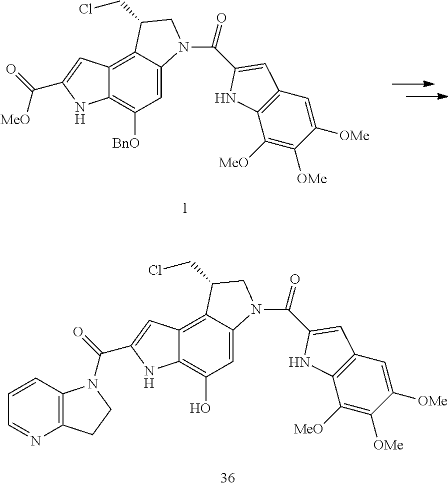 [SMILES: COC(=O)c1cc2c3c(cc(OCc4ccccc4)c2[nH]1)N(C(=O)c1cc2cc(OC)c(OC)c(OC)c2[nH]1)C[C@H]3CCl ; COc1cc2cc(C(=O)N3C[C@@H](CCl)c4c3cc(O)c3[nH]c(C(=O)N5CCc6ncccc65)cc43)[nH]c2c(OC)c1OC]

Compound 36 was prepared using a similar method as described for compound 4 (see Example 2).

### Example 23: Compounds 37, 38 and 39

Compound 37 was prepared as described in WO 2015/153401 A1, the entire contents of which are hereby incorporated by reference in their entirety.

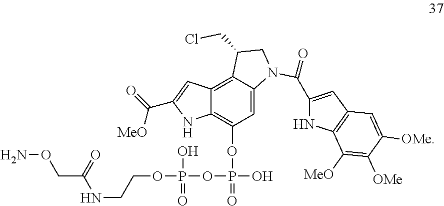 [SMILES: COC(=O)c1cc2c3c(cc(OP(=O)(O)OP(=O)(O)OCCNC(=O)CON)c2[nH]1)N(C(=O)c1cc2cc(OC)c(OC)c(OC)c2[nH]1)C[C@H]3CCl]

Compound 38 (CAS Registry No. 157922-77-5) and compound 39 (CAS Registry No. 152785-82-5, also known as (S)-seco-duocarmycin SA), each commercially available (MedChemExpress, Monmouth Junction, NJ, USA), have the following structures, respectively:

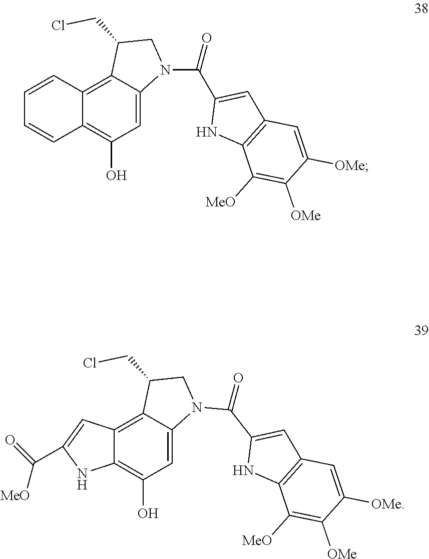 [SMILES: COc1cc2cc(C(=O)N3C[C@@H](CCl)c4c3cc(O)c3ccccc43)[nH]c2c(OC)c1OC ; COC(=O)c1cc2c3c(cc(O)c2[nH]1)N(C(=O)c1cc2cc(OC)c(OC)c(OC)c2[nH]1)C[C@H]3CCl]

### Example 24: Site Specific Conjugation of Drug-Linkers to Anti-CD70 Antibodies Containing Non-Natural Amino Acid pAF

Anti-CD70 antibodies with para-acetyl phenylalanine (pAF) incorporated into each of the two heavy chains at Kabat position 114 were buffer exchanged into 50 mM sodium acetate; 2.5% trehalose; 0-20% DMSO, pH 4.0-4.3 and concentrated to 1-20 mg/mL. Acetic hydrazide (50-100 mM) and amino-oxy drug-linker-payload compounds 12, 14, 16 and 18, prepared as described above (10-15 molar equivalents), were added and reacted for 16-72 hours at 30° C. The antibody-drug conjugates (ADCs) were purified over a Capto SP Impres column (Cytiva) to remove excess reagents. The ADCs were buffer exchanged into 50 mM histidine; 100 mM NaCl; 2.5% trehalose; pH 6.0, 0.22 μm filtered, and stored at 4° C.

Specifically, anti-CD70 ADCs were prepared as described above by conjugating each of compounds 12, 14, 16 and 18 with anti-CD70 mAbs, wherein each of the two heavy chains have the amino acid sequence of SEQ ID NO: 20, with pAF at Kabat position 114, and each of the two light chains have the amino acid sequence of SEQ ID NO: 19. The corresponding ADCs (hereinafter, anti-CD70-12, anti-CD70-14, anti-CD70-16 and anti-CD70-18 respectively) were tested for cytotoxicity (see Example 25).

As a benchmark, an anti-CD70 ADC containing the drug (S)-seco-duocarmycin SA (compound 39) was prepared as described above by conjugating drug-linker compound 37 with anti-CD70 mAb, wherein each of the two heavy chains have the amino acid sequence of SEQ ID NO: 25, with pAF at Kabat position 114, and each of the two light chains have the amino acid sequence of SEQ ID NO: 19 (hereinafter, anti-CD70-37). SEQ ID NOs: 20 and 25 share the same heavy chain variable region amino acid sequence (i.e., SEQ ID NO: 26). As shown in Table 2, SEQ ID NO: 25 contains the tripeptide amino acid sequence “DEL” within the Fc constant region, which is replaced with “EEM” in the Fc constant region of SEQ ID NO: 20; SEQ ID NOs: 20 and 25 are otherwise identical.

### Example 25: In Vitro Cytotoxicity of Anti-CD70 ADCs

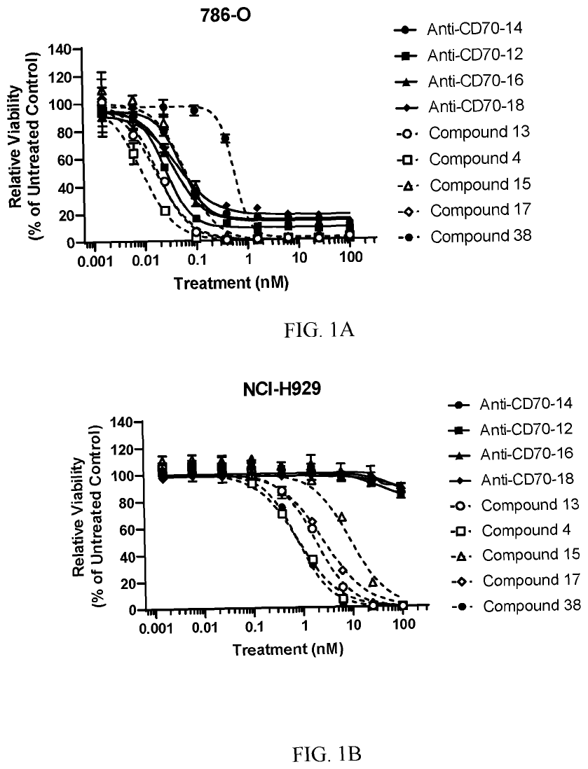

Small molecule duocarmycin analogs (compounds 4, 13, 15, 17 and 38) and anti-CD70 ADCs (anti-CD70-12, anti-CD70-14, anti-CD70-16 and anti-CD70-18) were evaluated for cytotoxicity. Briefly, 786-0 (CD70 positive) cells were seeded into 96-well clear bottom white plate at 1,000 cells/well and incubated overnight in a 37° C. and 5% CO₂ incubator. NCI-H929 (CD70 negative) cells were seeded 30,000 cells/well just before the time of treatment of test articles. The serially diluted small molecule drugs or anti-CD70 ADCs were added to the wells and the plates were incubated for 4 days in a 37° C. and 5% CO₂ incubator. At the end of incubation, CellTiter-Glo2.0 Reagent (Promega, Madison, WI) was added to the room temperature equilibrated plates and luminescence was measured in a SpectraMax M5E plate reader. The relative viability was calculated as a percentage of untreated cells. The half-maximal inhibitory concentration (IC₅₀) was determined by a nonlinear 4-parameter dose-response curve fitting using GraphPad Prism (GraphPad Software, San Diego, CA). The maximal killing (Emax) was determined by subtracting the % viability at the indicated dose from 100%. The results are shown in FIGS. 1A and 1B and summarized in Table 10.

TABLE 10
Cytotoxicity data for duocarmycin analogs and anti-CD70 ADCs.

| | ADC or | Anti- | Anti- | Anti- | Anti- | | | | | |
|---|---|---|---|---|---|---|---|---|---|---|
| | Compound/ | CD70- | CD70- | CD70- | CD70- | | | | | |
| | Parameter | 14 | 12 | 16 | 18 | 13 | 4 | 15 | 17 | 38 |
| | DAR | 1.90 | 1.92 | 1.94 | 1.82 | NA | NA | NA | NA | NA |
| 786-O | IC₅₀ (nM) | 0.0518 | 0.0254 | 0.0356 | 0.0429 | 0.0166 | 0.0089 | 0.0626 | 0.0179 | 0.561 |
| (CD70 | Emax (%) | 85.36 | 91.81 | 87.04 | 82.22 | 99.19 | 98.99 | 98.10 | 99.28 | 97.36 |
| Positive) | at 25 nM | | | | | | | | | |
| NCI-H929 | IC₅₀ (nM) | >100 | >100 | >100 | >100 | 1.794 | 0.8045 | 9.119 | 2.592 | 0.791 |
| (CD70 | Emax (%) | 3.53 | 7.64 | 1.48 | 5.11 | 99.48 | 99.70 | 82.36 | 97.62 | 99.53 |
| Negative) | at 25 nM | | | | | | | | | |

(S)-seco-duocarmycin SA (compound 39) and anti-CD70-37 ADC were also tested for cytotoxicity against 786-0 (CD70-positive) cells as described above; the IC₅₀ values for the free drug and the ADC were 0.065 nM and 0.321 nM, respectively. Upon repeat testing under the same assay conditions, anti-CD70-37 ADC exhibited an IC₅₀ value of 0.55 nM. Thus, anti-CD70-37 ADC had a mean IC₅₀ value of 0.436 nM.

In sum, anti-CD70-12, anti-CD70-14, anti-CD70-16 and anti-CD70-18 ADCs were about 10-fold more active than anti-CD70-37 ADC in the 786-0 (CD70-positive) cytotoxicity assay.

### Example 26: Anti-GPC3 ADCs

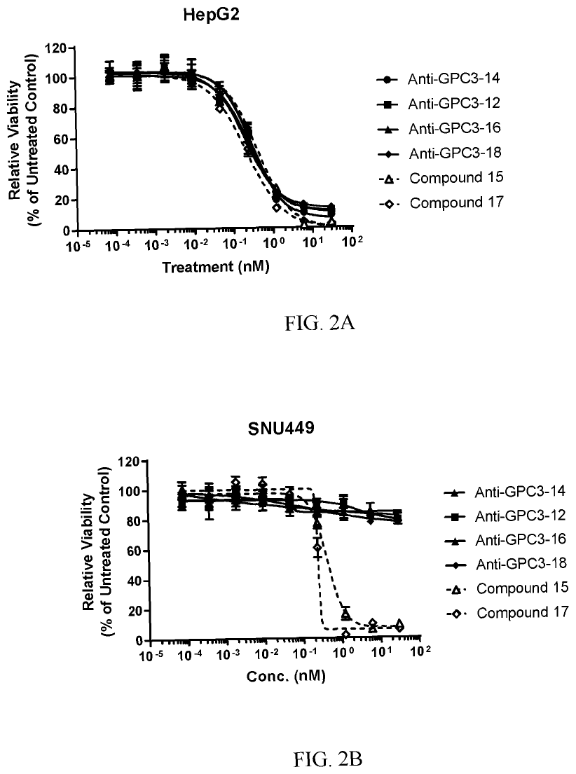

Antibodies that specifically bind glypican-3 (GPC3) are known in the art, including wild-type GPC3 and recombinant monoclonal anti-GPC3 antibodies, and can be prepared essentially as described by Pilia G. et al. (1996) Nature Genetics 12:241-247; Kiyotaka N. et al. (2010) Anti-Cancer Drugs, 21(10:907-916); Terrette J. A. et al. (US 2010/0209432 A1); and Feng M. et al. (2013) Proc. Natl. Acad. Sci. USA, 110(12):E1083-91, the entire contents of each of which are hereby incorporated by reference in their entirety. The wild type amino acid sequence disclosed by Pilia G. et al. (1996) Nature Genetics 12:241-247 (see, e.g., FIG. 2, the entire contents of which are hereby incorporated by reference in their entirety) and any other anti-GPC3 antibody, including those disclosed in the foregoing references (i.e., Kiyotaka N. et al. (2010); Terrette J. A. et al. (US 2010/0209432 A1); and Feng M. et al. (2013)) can be modified to incorporate one or more non-natural amino acid at any desired position using the methods described herein.

Anti-GPC3 ADCs were prepared via conjugation of GPC3 clone 1 mAb containing non-natural amino acid pAF with each of compounds 12, 14, 16 and 18. The corresponding ADCs (hereinafter, anti-GPC3-12, anti-GPC3-14, anti-GPC3-16 and anti-GPC3-18 respectively) and small molecule duocarmycin analogs (compounds 15 and 17) were evaluated for cytotoxicity. Briefly, HepG2 and SNU449 cells were seeded into 96-well white opaque plate at 3,000 cells/well and incubated overnight in a 37° C. and 5% CO₂ incubator. The serially diluted small molecule drugs or anti-GPC3 ADCs were added to the wells and the plates were incubated for 3 days in a 37° C. and 5% CO₂ incubator. At the end of incubation, CellTiter-Glo2.0 Reagent (Promega, Madison, WI) was added to the room temperature equilibrated plates and luminescence was measured in a SpectraMax M5E plate reader. The relative viability was calculated as a percentage of untreated cells. The half-maximal inhibitory concentration (IC₅₀) was determined by a nonlinear 4-parameter dose-response curve fitting using GraphPad Prism (GraphPad Software, San Diego, CA). The maximal killing (Emax) was determined by subtracting the % viability at the indicated dose from 100%. The results are shown in FIGS. 2A and 2B and summarized in Table 11.

TABLE 11
Cytotoxicity data for duocarmycin analogs and anti-GPC3 ADCs.

| | ADC or | Anti- | Anti- | Anti- | Anti- | | |
|---|---|---|---|---|---|---|---|
| | Compound/ | GPC3- | GPC3- | GPC3- | GPC3- | | |
| | Parameter | 14 | 12 | 16 | 18 | 15 | 17 |
| | DAR | 1.74 | 1.92 | 1.89 | 1.84 | NA | NA |
| HepG2 | IC₅₀ (nM) | 0.297 | 0.260 | 0.282 | 0.211 | 0.410 | 0.217 |
| (GPC3 | Emax (%) | 91.96 | 88.50 | 87.93 | 86.60 | 96.54 | 97.59 |
| Positive) | at 30 nM | | | | | | |
| SNU449 | IC₅₀ (nM) | >30 | >30 | >30 | >30 | 0.427 | 0.245 |
| (GPC3 | Emax (%) | 18.74 | 20.88 | 19.02 | 22.96 | 91.11 | 94.23 |
| Negative) | at 30 nM | | | | | | |

### Example 27: Anti-Tumor Activity of Anti-GPC3 ADCs

Anti-GPC3 ADCs (anti-GPC3-12, anti-GPC3-14, anti-GPC3-16 and anti-GPC3-18) were evaluated in a HepG2 xenograft mouse model of human hepatocellular carcinoma (HCC) at a low dose of 1 mg/kg, with a view towards detecting small changes in tumor growth rates and establishing the relative efficacy among the ADCs in this model. The study design is summarized in Table 12. Tumor growth inhibition was observed for all tested ADCs relative to vehicle control, with anti-GPC3-14 exhibiting 66% tumor growth inhibition and a tumor growth delay of 30 days (data not shown).

TABLE 12
Study design for in vivo testing of anti-
GPC3 ADS in HepG2 xenograft model

| Group | Treatment | Dose | Route | Schedule | N |
|---|---|---|---|---|---|
| 1 | Vehicle | NA | IV | QWx3 | 10 |
| 2 | Anti-GPC3-12 | 1 mg/kg | IV | QWx3 | 9 |
| 3 | Anti-GPC3-14 | 1 mg/kg | IV | QWx3 | 10 |
| 4 | Anti-GPC3-16 | 1 mg/kg | IV | QWx3 | 9 |
| 5 | Anti-GPC3-18 | 1 mg/kg | IV | QWx3 | 9 |

### Example 28: Molecular Cloning for Expression of Anti-CD70, Anti-TROP2 and Anti-HER3 Antibodies

For each of CD70, TROP2 and HER3 antibodies, CHO cell codon-optimized antibody heavy chain and light chain cDNA sequences were obtained from a commercial DNA synthesis service (Integrated DNA Technologies (IDT), San Diego, CA). The synthesized DNA fragments were digested with Hind III and EcoR I (both from New England BioLabs, (NEB), Ipswich, MA) and purified using a PCR purification kit (Qiagen, Valencia, CA). Then the digested antibody gene fragments were ligated into the expression vector via a quick ligation kit (NEB) to yield the constructs for expression of wild type antibody heavy chain and light chain. The resulting plasmids were propagated in E. coli and verified by a DNA sequencing service (Eton Biosciences, San Diego, CA).

Generation of amber codon-containing mutants—Based on the crystal structure of an immunoglobulin G1 [IgG1]mAb, heavy chain amino acid A114 (Kabat numbering) was chosen to genetically incorporate non-natural amino acid pAF. For HER3, additional sites within the light chain sequence were also chosen for pAF incorporation (see Table 5). The genetic codon of the chosen site(s) was then mutated to amber codon (TAG) via site-directed mutagenesis to generate expression plasmid for that antibody mutant. Primers were purchased from IDT. All site-directed mutagenesis experiments were carried out using Q5 site-directed mutagenesis kit following instruction manuals (NEB). The expression plasmids for the mutants were propagated in E. coli and verified by DNA sequencing service (Eton Biosciences).

### Example 29: Protocols for Production of Antibodies Containing pAF at Heavy Chain Amino Acid Sequence Position 114 (Kabat Numbering; Anti-CD70, Anti-TROP2 and Anti-HER3 Heavy Chains), and Light Chain Amino Acid Sequence Position 121 (Kabat Numbering; Anti-HER3 Light Chains)

Transient expression—Platform cell lines for anti-CD70, anti-TROP2 and anti-HER3 were maintained in EX-CELL 302 (Sigma) supplemented with 3 mM L-glutamine (Gibco) and 3 mM GlutaMAX (Gibco). Cells were passaged every 3 to 4 days seeded at a density of 0.4 million cells per ml. One day prior to transfection, cells were seeded at 0.6 million cells per ml. On day 0, cells were transfected with antibody expression plasmids encoding the light chain and heavy chain using MaxCyte electroporation platform following the instruction manual. After transfection, cells were rested in an empty 125 ml shake flask and incubated at 37° C. in a static incubator for 30 min. Basal expression media (50% Dynamis:50% ExCELL 302 supplemented with 3 mM L-glutamine and 3 mM GlutaMAX) was added to the transfected cells in the shake flask for a final density of 3×10⁶ cells/ml. The transfected cells were incubated at 37° C., 5% CO₂ on an orbital shaker set to 140 or 155 rpm. The following were added to the culture on day 1: pAF (final concentration in culture: 1 mM), Long R3 IGF-1 (Sigma; final concentration in culture: 120 μg/L), GlutaMAX (final concentration in culture: 2 mM) and either (a) Cell Boost 5 (GE Healthcare; final concentration in culture: 7 g/L), or (b) Cell Boost 4 (GE Healthcare; final concentration in culture: 3.75 g/L) and Cell Boost 7b (GE Healthcare; final concentration in culture: 0.2 g/L). The incubator temperature was shifted from 37° C. to 32° C. Additional GlutaMAX (final concentration: 2 mM) and (a) Cell Boost 5 (final concentration: 7 g/L), were added on day 3, and supernatant was collected on day 5, or (b) Cell Boost 4 (GE Healthcare; final concentration in culture: 2 g/L) and Cell Boost 7b (final concentration in culture: 0.1 g/L) were added on days 3 and 5, and supernatant was collected on day 7. The culture media glucose level was monitored using glucose meters, and additional glucose was added to the culture when the glucose level was below 2 g/L. Viable cell count and viability were measured by Vi-Cell instrument. Antibody production was measured by Octet using Protein G sensors.

Stable bulk pool generation—The expression plasmid was linearized using Pvu I (NEB) digestion for four hours. After linearization, the DNA was purified using phenol:chloroform:isoamyl alcohol extraction and dissolved in endotoxin-free water at the concentration of 2.5 μg/μl. Platform cell line was maintained in EX-CELL 302 supplemented with 3 mM L-glutamine and 3 mM GlutaMAX. Cells were passaged every 3 to 4 days seeded at a density of 0.3×10⁶/ml. One day prior to transfection, cells were seeded at 0.6×10⁶/ml. On day 0, 15×10⁶ cells were transfected with 25 μg of linearized antibody expression plasmids using MaxCyte electroporation (OC-100) platform following the instruction manual. After transfection, cells were rested in an empty 125 ml shake flask and incubated at 37° C. in a static incubator for 30 min. Then 30 ml recovery media (50% Ex-302:50% CD-CHO supplemented with 3 mM L-glutamine and 3 mM GlutaMAX) was added into the flask and shaken overnight. On day one, transfected cells were counted, spun down, washed and re-suspended in selection media (50% ExCELL 302:50% CD-CHO with 50 μM MSX) for stable bulk pool generation. The viable cell numbers and viability were monitored, and media was changed every 3 to 4 days until the viability of the stable bulk pool returned to 90%. When selection ended, frozen cell stocks were made, and the resulting stable bulk pool was used to generate material for fed-batch expression.

Fed-batch expression—Previously generated antibody stable bulk pools were inoculated into basal expression media (50% Dynamis:50% ExCELL 302 supplemented with 50 μM MSX, or 50% Dynamis:50% ExCELL 302 supplemented with 1×GS, 2 μg/ml insulin, 0.5 mM ornithine, 2 g/L glucose and 1× anti clumping agent and 25 μM MSX) at a density of 0.5×10⁶/ml in a shake flask on day 0. The transfected cells were incubated at 37° C., 5% CO₂ on an orbital shaker set to 150 rpm. The following were added to the culture on day 3: pAF (final concentration in culture: 0.5 mM), Cell Boost 4 (GE Healthcare; final concentration in culture: 10 g/L) and Cell Boost 7b (GE Healthcare; final concentration in culture: 0.52 g/L). Long R3 IGF-1 (final concentration in culture: 120 μg/L) was added to the culture on day 5. The culture media glucose level was monitored using glucose meters, and additional glucose was added to the culture when the glucose level was below 2 g/L. Viable cell count and viability were measured by Vi-Cell instrument. The supernatant was collected for purification on day 7 or 10. Antibody production was measured by Octet using Protein G sensors.

### Example 30: Purification of Antibodies from EuCODE Expression System

Clarified Cell culture media containing the target antibody containing non-natural amino acid pAF was loaded over a protein A ProSep Ultra column (EMD Millipore) equilibrated in 20 mM sodium phosphate, 100 mM sodium chloride, pH 7.5. After loading, the column was washed with buffer A (20 mM sodium phosphate, 100 mM sodium chloride, pH 7.5) followed by wash buffer B (5 mM succinic acid, pH 5.8) to remove host cell contaminants. The target antibody was eluted from the column with elution buffer C (50 mM glycine, 10 mM succinic acid, pH 3.2). The target antibody was pooled, and pH adjusted to pH 5.0 with 2.0 M tris base. The target antibody was further purified by loading the conditioned protein A pool over a Capto SP Impres column (GE Healthcare) equilibrated in 30 mM sodium acetate, pH 5.0. The target antibody was eluted from the column with a linear gradient to 100% buffer B (30 mM sodium acetate, 0.5 M sodium chloride, pH 5.0) and fractions containing monomeric antibody were pooled, 0.22 μM filtered, and stored at <65° C. until further use.

### Example 31: Anti-HER2 and Anti-PSMA Antibodies

Anti-HER2 and anti-PSMA antibodies and variants thereof of the present disclosure, and ADCs of the present disclosure containing said antibodies and variants thereof, can be prepared essentially as described in WO2022/212899A1 and WO2019/191728A1, the entire contents of each of which are hereby incorporated by reference in their entirety, and/or by adapting methods expressly disclosed herein.

Additional non-limiting embodiments of the invention are listed below.

A1. A compound of Formula (I):

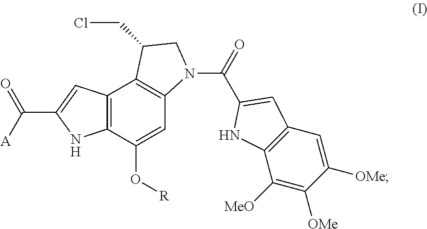

wherein:
R is H or L-W, wherein L is a linker and W is a reactive moiety; and
A is a bicyclic ring system selected from the group consisting of formula (a), (b), (c) and (d), having the following structures:

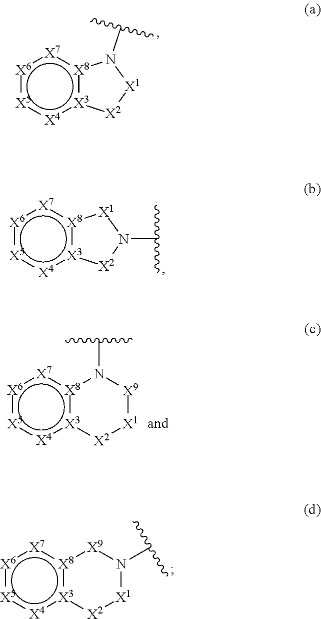

wherein:
   each X¹ is C(R^{1a})(R^{1b}); wherein each R^{1a} and R^{1b} is independently H, halogen, alkyl, alkenyl or alkynyl;
   each X² is C(R^{2a})(R^{2b}); wherein each R^{2a} and R^{2b} is independently H, halogen, alkyl, alkenyl or alkynyl;
   each X³ is C;
   each X⁴ is C(R⁴) or N, wherein R⁴ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
   each X⁵ is C(R⁵) or N, wherein R⁵ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
   each X⁶ is C(R⁶) or N, wherein R⁶ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
   each X⁷ is C(R⁷) or N, wherein R⁷ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
   each X⁸ is C; and
   each X⁹, when present, is C(R^{9a})(R^{9b}); wherein each R^{9a} and R^{9b} is independently H, halogen, alkyl, alkenyl or alkynyl;
      wherein:
each R^{a} and R^{b} is independently H, alkyl, alkenyl or alkynyl;
each R^{c} is independently H, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl or heteroarylalkyl;
each R^{s} is independently H, halogen, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl or heteroarylalkyl; and
each m is independently 0, 1, 2 or 3;
   or a salt thereof.

A2. The compound of embodiment A1, wherein the compound is a compound of Formula (Ia) having the following structure:

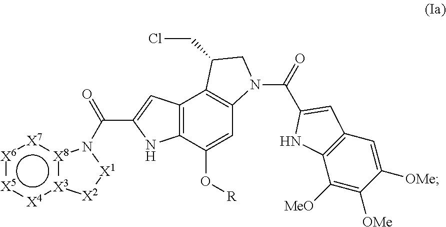

wherein:
R is H or L-W, wherein L is a linker and W is a reactive moiety;
X¹ is C(R^{1a})(R^{1b}); wherein each R^{1a} and R^{1b} is independently H, halogen, alkyl, alkenyl or alkynyl; X² is C(R^{2a})(R^{2b}); wherein each R^{2a} and R^{2b} is independently H, halogen, alkyl, alkenyl or alkynyl;
X³ is C;
X⁴ is C(R⁴) or N, wherein R⁴ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
X⁵ is C(R⁵) or N, wherein R⁵ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
X⁶ is C(R⁶) or N, wherein R⁶ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
X⁷ is C(R⁷) or N, wherein R⁷ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s}); and
X⁸ is C;
   wherein:
each R^{a} and R^{b} is independently H, alkyl, alkenyl or alkynyl;
each R^{c} is independently H, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl or heteroarylalkyl;
each R^{s} is independently H, halogen, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl or heteroarylalkyl; and
each m is independently 0, 1, 2 or 3.

A3. The compound of embodiment A1, wherein the compound is a compound of Formula (Ib) having the following structure:

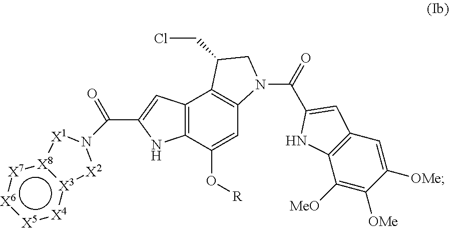

wherein:
R is H or L-W, wherein L is a linker and W is a reactive moiety;
X¹ is C(R^{1a})(R^{1b}); wherein each R^{1a} and R^{1b} is independently H, halogen, alkyl, alkenyl or alkynyl;
X² is C(R^{2a})(R^{2b}); wherein each R^{2a} and R^{2b} is independently H, halogen, alkyl, alkenyl or alkynyl;
X³ is C;
X⁴ is C(R⁴) or N, wherein R⁴ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
X⁵ is C(R⁵) or N, wherein R⁵ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
X⁶ is C(R⁶) or N, wherein R⁶ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
X⁷ is C(R⁷) or N, wherein R⁷ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{S}); and
X⁸ is C;
   wherein:
each R^{a} and R^{b} is independently H, alkyl, alkenyl or alkynyl;
each R^{c} is independently H, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl or heteroarylalkyl;
each R^{s} is independently H, halogen, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl or heteroarylalkyl; and
each m is independently 0, 1, 2 or 3.

A4. The compound of embodiment A1, wherein the compound is a compound of Formula (Ic) having the following structure:

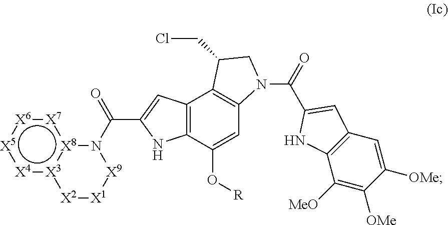

wherein:
R is H or L-W, wherein L is a linker and W is a reactive moiety;
X¹ is C(R^{1a})(R^{1b}); wherein each R^{1a} and R^{1b} is independently H, halogen, alkyl, alkenyl or alkynyl;
X² is C(R^{2a})(R^{2b}); wherein each R^{2a} and R^{2b} is independently H, halogen, alkyl, alkenyl or alkynyl;
X³ is C;
X⁴ is C(R⁴) or N, wherein R⁴ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
X⁵ is C(R⁵) or N, wherein R⁵ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
X⁶ is C(R⁶) or N, wherein R⁶ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
X⁷ is C(R⁷) or N, wherein R⁷ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
X⁸ is C; and
X⁹ is C(R^{9a})(R^{9b}); wherein each R^{9a} and R^{9b} is independently H, halogen, alkyl, alkenyl or alkynyl;
   wherein:
each R^{a} and R^{b} is independently H, alkyl, alkenyl or alkynyl;
each R^{c} is independently H, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl or heteroarylalkyl;
each R^{s} is independently H, halogen, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl or heteroarylalkyl; and
each m is independently 0, 1, 2 or 3.

A5. The compound of embodiment A1, wherein the compound is a compound of Formula (Id) having the following structure:

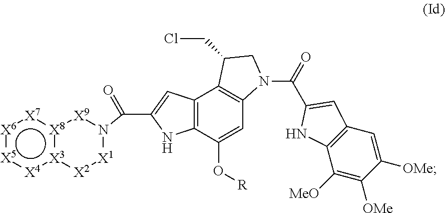

wherein:
R is H or L-W, wherein L is a linker and W is a reactive moiety;
X¹ is C(R^{1a})(R^{1b}); wherein each R^{1a} and R^{1b} is independently H, halogen, alkyl, alkenyl or alkynyl;
X² is C(R^{2a})(R^{2b}); wherein each R^{2a} and R^{2b} is independently H, halogen, alkyl, alkenyl or alkynyl;
X³ is C;
X⁴ is C(R⁴) or N, wherein R⁴ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
X⁵ is C(R⁵) or N, wherein R⁵ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
X⁶ is C(R⁶) or N, wherein R⁶ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
X⁷ is C(R⁷) or N, wherein R⁷ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
X⁸ is C; and
X⁹ is C(R^{9a})(R^{9b}); wherein each R^{9a} and R^{9b} is independently H, halogen, alkyl, alkenyl or alkynyl;
   wherein:
each R^{a} and R^{b} is independently H, alkyl, alkenyl or alkynyl;
each R^{c} is independently H, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl or heteroarylalkyl;
each R^{s} is independently H, halogen, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl or heteroarylalkyl; and
each m is independently 0, 1, 2 or 3.

A6. The compound of any one of embodiments A1 to A5, wherein:
X¹ is C(R^{1a})(R^{1b}); wherein each R^{1a} and R^{1b} is independently H, halogen or unsubstituted alkyl;
X² is C(R^{2a})(R^{2b}); wherein each R^{2a} and R^{2b} is independently H, halogen or unsubstituted alkyl;
X³ is C;
X⁴ is C(R⁴) or N, wherein R⁴ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl;
X⁵ is C(R⁵) or N, wherein R⁵ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl;
X⁶ is C(R⁶) or N, wherein R⁶ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl;
X⁷ is C(R⁷) or N, wherein R⁷ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl;
X⁸ is C; and
X⁹, when present, is C(R^{9a})(R^{9b}); wherein each R^{9a} and R^{9b} is independently H, halogen or unsubstituted alkyl.

A7. The compound of any one of embodiments A1 to A6, wherein each X¹ and X² is CH₂; and X⁹, when present, is CH₂.

A8. The compound of any one of embodiments A1 to A7, wherein:
X⁴ is C(R⁴) or N, wherein R⁴ is H, halogen or heteroalkyl;
X⁵ is C(R⁵) or N, wherein R⁵ is H, halogen or heteroalkyl;
X⁶ is C(R⁶) or N, wherein R⁶ is H, halogen or heteroalkyl; and
X⁷ is C(R⁷) or N, wherein R⁷ is H, halogen or heteroalkyl.

A9. The compound of any one of embodiments A1 to A8, wherein each said heteroalkyl is an alkoxy.

A10. The compound of embodiment A9, wherein each said alkoxy is independently —OR^{k}, wherein each R^{k} is independently alkyl, wherein said alkyl is optionally substituted with —N(R^{d})(R^{e}) or heterocyclyl; wherein each R^{d} and R^{e} is independently H, alkyl, alkenyl or alkynyl, and wherein each heterocyclyl contains at least one nitrogen atom.

A11. The compound of any one of embodiments A1 to A10, wherein X⁴ is N, X⁵ is C(R⁵), X⁶ is C(R⁶) and X⁷ is C(R⁷).

A12. The compound of any one of embodiments A1 to A10, wherein X⁴ is C(R⁴), X⁵ is N, X⁶ is C(R⁶) and X⁷ is C(R⁷).

A13. The compound of any one of embodiments A1 to A10, wherein X⁴ is C(R⁴), X⁵ is C(R⁵), X⁶ is N and X⁷ is C(R⁷).

A14. The compound of any one of embodiments All, A12 and A13, wherein X⁷ is CH.

A15. The compound of embodiment A12 or A13, wherein at least one of X⁴ and X⁷ is CH.

A16. The compound of any one of embodiments A1 to A10, wherein X⁴ is C(R⁴), X⁵ is C(R⁵), X⁶ is C(R⁶) and X⁷ is N.

A17. The compound of embodiment A16, wherein X⁴ is CH.

A18. The compound of any one of embodiments A1 to A10, wherein X⁴ is C(R⁴), X⁵ is C(R⁵), X⁶ is C(R⁶) and X⁷ is C(R⁷).

A19. The compound of embodiment A18, wherein X⁴ is CH, X⁷ is CH, or both.

A20. The compound of any one of embodiments A1 to A19, wherein R is H.

A21. The compound of any one of embodiments A1 to A19, wherein R is L-W.

A22. The compound of embodiment A1 or A21, wherein R is L-W, and L is a phosphate-based linker comprising a phosphate-based moiety selected from the group consisting of a phosphate ester, a pyrophosphate ester, a triphosphate ester, a tetraphosphate ester, a phosphonate, a diphosphonate, a phosporamidate, a pyrophosporamidate, a triphosphoramidate, a tetraphosphoramidate, a phosphorthioate and a diphosphorthioate.

A23. The compound of any one of embodiments A1 to A22, wherein the corresponding amine of moiety A has a C log P value of at least about 1.

A24. The compound of embodiment A22, wherein the phosphate-based moiety is a diphosphonate.

A25. The compound of any one of embodiments A22 to A24, wherein L further comprises at least one additional moiety, wherein each at least one additional moiety is independently selected from the group consisting of unsubstituted alkylene, substituted alkylene, -(alkylene-O)—, optionally substituted arylene, —O—, —C(O)—, —N(R_{w})—, —S(O)₀₋₂—, a water-soluble polymer and an amino acid; wherein each R_{w} is independently H or C₁-C₈ alkyl; and combinations thereof.

A26. The compound of embodiment A25, wherein each at least one additional moiety is independently selected from the group consisting of unsubstituted alkylene, -(alkylene-O)—, —C(O)—, —N(R_{w})—, a water-soluble polymer and an amino acid; wherein each R_{w} is independently H or C₁-C₈ alkyl; and combinations thereof.

A27. The compound of any one of embodiments A22 to A26, wherein the phosphate-based moiety is covalently bound to the —O— atom of Formula (I), or Formula (Ia), or Formula (Ib), or Formula (Ic) or Formula I(d)) via a phosphorous atom of the phosphate-based moiety.

A28. The compound of embodiment A25, A26 or A27, wherein L comprises at least one alkylene group.

A29. The compound of any one of embodiments A25 to A28, wherein L comprises an amino acid.

A30. The compound of any one of embodiments A25 to A29, wherein L comprises a water-soluble polymer.

A31. The compound of any one of embodiments A25 to A30, wherein L comprises a water-soluble polymer and an amino acid, wherein the water-soluble polymer is conjugated to the amino acid.

A32. The compound of embodiment A31, wherein the water-soluble polymer is conjugated to a side chain of the amino acid.

A33. The compound of embodiment A31 or A32, wherein the water-soluble polymer is conjugated to the amino acid via a spacer element.

A34. The compound of any one of embodiments A25 to A33, wherein the amino acid is selected from the group consisting of serine, threonine, cysteine, tyrosine, aspartic acid, glutamic acid, lysine and N_{ϵ}-methyl-lysine.

A35. The compound of any one of embodiments A25 to A34, wherein the amino acid is lysine or N_{ϵ}-methyl-lysine.

A36. The compound of embodiment A21, wherein L is selected from the group of linkers of Table 6.

A37. The compound of embodiment A21, wherein L is selected from the group of linkers of Table 7.

A38. The compound of embodiment A21, wherein L is selected from the group consisting of:

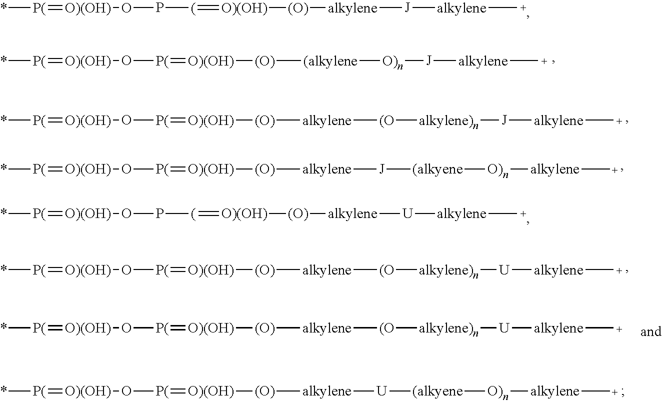

wherein:
each U is independently selected from the group consisting of:

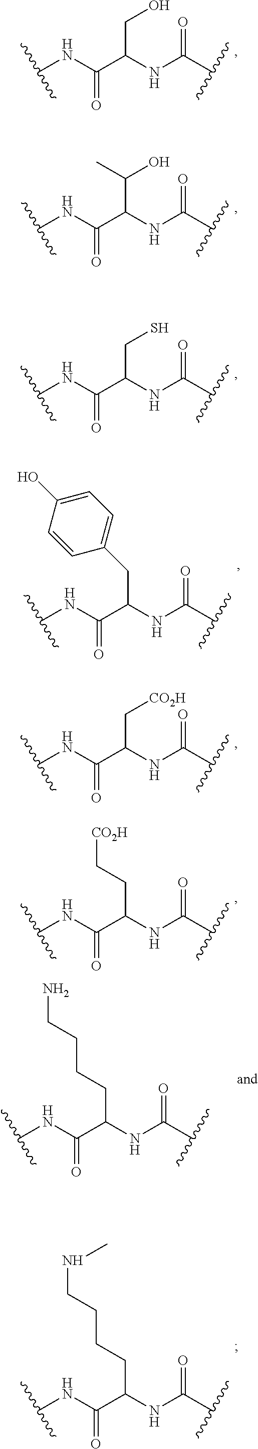

each J is independently

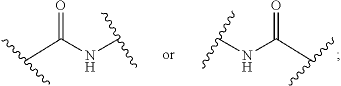 [SMILES: *NC(*)=O ; *NC(*)=O]

each alkylene is independently selected from the group consisting of:

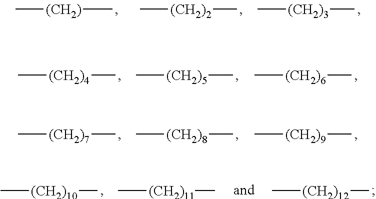

each n is independently an integer from 1 to 100;
* denotes the connection to the —O— atom of Formula (I), or Formula (Ia), or Formula (Ib), or Formula (Ic) or Formula I(d); and
+ denotes the connection to W;
   wherein each linker L is optionally substituted with one or more water-soluble polymers.

A39. The compound of any one of embodiments A36 to A38, wherein each n is independently an integer from 1 to 10, or wherein each n is independently 1, 2 or 3.

A40. The compound of any one of embodiments A36 to A39, wherein L is substituted with the one or more water-soluble polymer.

A41. The compound of any one of embodiments A36 to A40, wherein L comprises group U, and one water-soluble polymer is conjugated to an amino acid side chain of group U.

A42. The compound of embodiment A41, wherein the water-soluble polymer is conjugated to the amino acid side chain of group U via a spacer element.

A43. The compound of any one of embodiments A1 to A5 and A21 to A42, wherein R is L-W and L is a bivalent linker.

A44. The compound of any one of embodiments A1 to A5 and A36 to A43, wherein R is L-W, and L is:

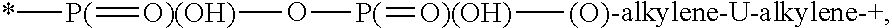

wherein:
U is selected from the group consisting of:

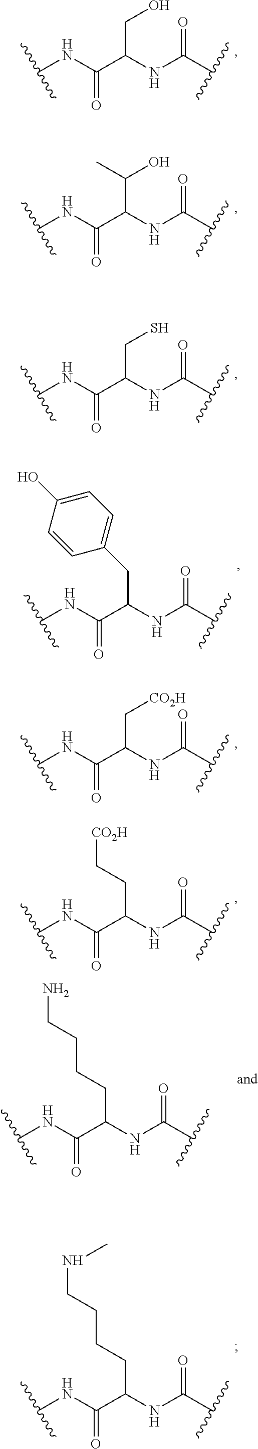

each alkylene is independently selected from the group consisting of:

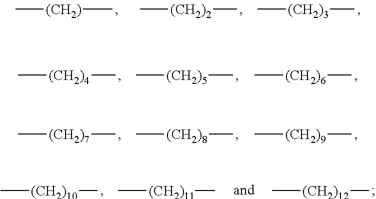

* denotes the connection to the —O— atom of Formula (I), or Formula (Ia), or Formula (Ib), or Formula (Ic) or Formula I(d); and
+ denotes the connection to W;
   wherein L is optionally substituted with one or more water-soluble polymers.

A45. The compound of embodiment A44, wherein L is substituted with the one or more water-soluble polymers.

A46. The compound of A45, wherein one water-soluble polymer is conjugated to an amino acid side chain of group U.

A47. The compound of embodiment A46, wherein the one water-soluble polymer is conjugated to the amino acid side chain of group U via a spacer element.

A48. The compound of any one of embodiments A44 to A47, wherein U is:

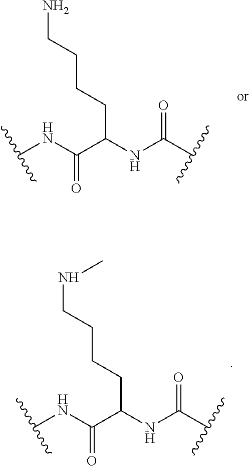 [SMILES: *NC(=O)C(CCCCN)NC(*)=O ; *NC(=O)C(CCCCNC)NC(*)=O]

A49. The compound of embodiment A47 or A48, wherein the spacer element is a carbonyl group.

A50. The compound of any one of embodiments A47 to A49, wherein L has the following structure:

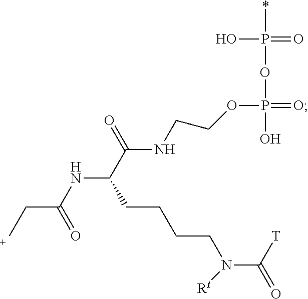

wherein: T is the water-soluble polymer; R^{t} is H or methyl; * denotes the connection to the —O— atom of Formula (I), or Formula (Ia), or Formula (Ib), or Formula (Ic) or Formula I(d); and + denotes the connection to W.

A51. The compound of any one of embodiments A25 to A50, wherein the water-soluble polymer is a (polyethylene)glycol (PEG) moiety.

A52. The compound of embodiment A51, wherein the PEG moiety has a molecular weight within a range of about 100 Da to about 100,000 Da.

A53. The compound of embodiment A52, wherein the PEG moiety has a molecular weight within a range of about 100 Da to about 10,000 Da, about 100 Da to about 5,000 Da, or about 100 Da to about 1,000 Da.

A54. The compound of any one of embodiments A1 to A5 and A21 to A53, wherein R is L-W, and the reactive moiety W comprises —N₃, —OH, —SH, —NH(R^{j}), —C(O)R^{q}, —C(O)OR^{x}, —C(O)CH₂NH₂, an activated ester, —O—NH₂, a maleimide, a tetrazine, an alkyne, a cyclooctyne or an (E)-cyclooctene; wherein R^{j} is H or unsubstituted alkyl, R^{q} is unsubstituted alkyl, and R^{x} is H, unsubstituted alkyl or a carboxylic acid protecting group.

A55. The compound of embodiment A54, wherein reactive moiety W is selected from the group consisting of:

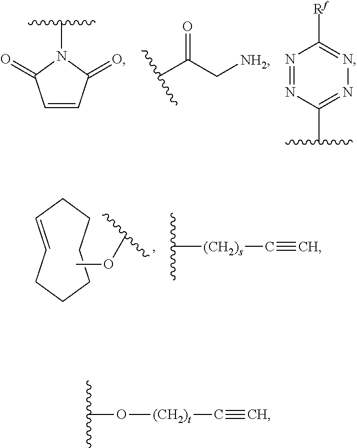

—N₃, —OH, —SH, —NH(Ri), —C(O)R^{q}, —C(O)OR^{x}, an activated ester, —O—NH₂ and an optionally substituted monocyclic or polycyclic group comprising the cyclooctyne;
wherein:
   R^{j} is H or unsubstituted C₁-C₆ alkyl,
   R^{q} is unsubstituted C₁-C₆ alkyl,
   R^{x} is H, unsubstituted C₁-C₆ alkyl or a carboxylic acid protecting group,
   R^{f} is H or unsubstituted C₁-C₆ alkyl,
   s is 0, 1, 2, 3, 4, 5 or 6, and
   t is 0, 1, 2, 3, 4, 5 or 6.

A56. The compound of embodiment A55, wherein the optionally substituted monocyclic or polycyclic group comprising the cyclooctyne is selected from the group consisting of:

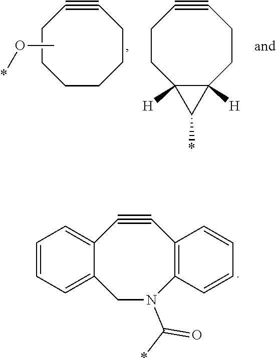

A57. The compound of any one of embodiments A1 to A5 and A21 to A56, wherein R is L-W, and W is —ONH₂.

A58. The compound of embodiment A2, wherein R is H.

A59. The compound of embodiment A58, wherein the compound is selected from the group consisting of compound 4, 13, 15, 17, 25, 26, 27, 28, 29, 30, 31, 35 and 36, each having the structure as disclosed in the Examples herein; and salts thereof.

A60. The compound of embodiment A2, wherein R is L-W, and the compound is selected from the group consisting of the compounds listed in Table 9; and salts thereof.

A61. An antibody-drug conjugate (ADC) of Formula (II):

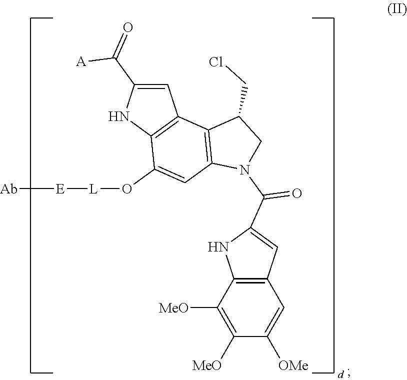

wherein:
Ab is an antibody, wherein Ab comprises one or more non-natural amino acids;
L is a linker;
E is a moiety joining Ab and L;
d is an integer from 1 to 100; and
A is a bicyclic ring system selected from the group consisting of formula (a), (b), (c) and (d), having the following structures:

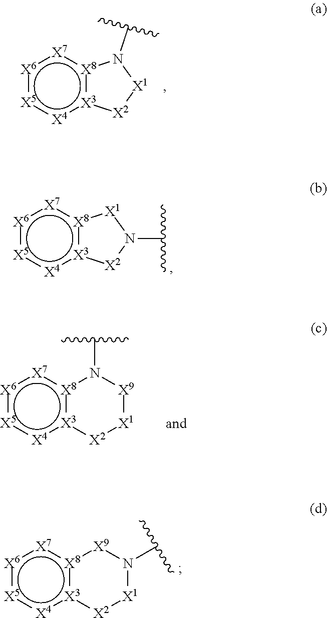

wherein:
   each X¹ is C(R^{1a})(R^{1b}); wherein each R^{1a} and R^{1b} is independently H, halogen, alkyl, alkenyl or alkynyl;
   each X² is C(R^{2a})(R^{2b}); wherein each R^{2a} and R^{2b} is independently H, halogen, alkyl, alkenyl or alkynyl;
   each X³ is C;
   each X⁴ is C(R⁴) or N, wherein R⁴ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
   each X⁵ is C(R⁵) or N, wherein R⁵ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
   each X⁶ is C(R⁶) or N, wherein R⁶ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
   each X⁷ is C(R⁷) or N, wherein R⁷ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
   each X⁸ is C; and
   each X⁹, when present, is C(R^{9a})(R^{9b}); wherein each R^{9a} and R^{9b} is independently H, halogen, alkyl, alkenyl or alkynyl;
wherein:
   each R^{a} and R^{b} is independently H, alkyl, alkenyl or alkynyl;
   each R^{c} is independently H, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl or heteroarylalkyl;
   each R^{s} is independently H, halogen, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl or heteroarylalkyl; and
   each m is independently 0, 1, 2 or 3;
or a pharmaceutically acceptable salt thereof.

A62. The ADC of embodiment A61, wherein A has the following structure:

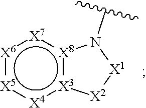

wherein the remaining variables are as defined in embodiment 61.

A63. The ADC of embodiment A61, wherein A has the following structure:

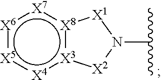

wherein the remaining variables are as defined in embodiment 61.

A64. The ADC of embodiment A61, wherein A has the following structure:

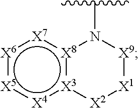

wherein the remaining variables are as defined in embodiment 61.

A65. The ADC of embodiment A61, wherein A has the following structure:

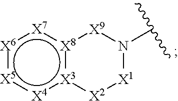

wherein the remaining variables are as defined in embodiment 61.

A66. The ADC of any one of embodiments A61 to A65, wherein:
X¹ is C(R^{1a})(R^{1b}); wherein each R^{1a} and R^{1b} is independently H, halogen or unsubstituted alkyl;
X² is C(R^{2a})(R^{2b}); wherein each R^{2a} and R^{2b} is independently H, halogen or unsubstituted alkyl;
X³ is C;
X⁴ is C(R⁴) or N, wherein R⁴ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl;
X⁵ is C(R⁵) or N, wherein R⁵ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl;
X⁶ is C(R⁶) or N, wherein R⁶ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl;
X⁷ is C(R⁷) or N, wherein R⁷ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl;
X⁸ is C; and
X⁹, when present, is C(R^{9a})(R^{9b}); wherein each R^{9a} and R^{9b} is independently H, halogen or unsubstituted alkyl.

A67. The ADC of any one of embodiments A61 to A66, wherein each X¹ and X² is CH₂; and X⁹, when present, is CH₂.

A68. The ADC of any one of embodiments A61 to A67, wherein:
X⁴ is C(R⁴) or N, wherein R⁴ is H, halogen or heteroalkyl;
X⁵ is C(R⁵) or N, wherein R⁵ is H, halogen or heteroalkyl;
X⁶ is C(R⁶) or N, wherein R⁶ is H, halogen or heteroalkyl; and
X⁷ is C(R⁷) or N, wherein R⁷ is H, halogen or heteroalkyl.

A69. The ADC of any one of embodiments A61 to A68, wherein each said heteroalkyl is an alkoxy.

A70. The ADC of embodiment A69, wherein each said alkoxy is independently —OR^{k}, wherein each R^{k} is independently alkyl, wherein said alkyl is optionally substituted with —N(R^{d})(R^{e}) or heterocyclyl; wherein each R^{d} and R^{e} is independently H, alkyl, alkenyl or alkynyl, and wherein each heterocyclyl contains at least one nitrogen atom.

A71. The ADC of any one of embodiments A61 to A70, wherein X⁴ is N, X⁵ is C(R⁵), X⁶ is C(R⁶) and X⁷ is C(R⁷).

A72. The ADC of any one of embodiments A61 to A70, wherein X⁴ is C(R⁴), X⁵ is N, X⁶ is C(R⁶) and X⁷ is C(R⁷).

A73. The ADC of any one of embodiments A61 to A70, wherein X⁴ is C(R⁴), X⁵ is C(R⁵), X⁶ is N and X⁷ is C(R⁷).

A74. The ADC of any one of embodiments A71, A72 and A73, wherein X⁷ is CH.

A75. The ADC of embodiment A72 or A73, wherein at least one of X⁴ and X⁷ is CH.

A76. The ADC of any one of embodiments A61 to A70, wherein X⁴ is C(R⁴), X⁵ is C(R⁵), X⁶ is C(R⁶) and X⁷ is N.

A77. The ADC of embodiment A76, wherein X⁴ is CH.

A78. The ADC of any one of embodiments A61 to A70, wherein X⁴ is C(R⁴), X⁵ is C(R⁵), X⁶ is C(R⁶) and X⁷ is C(R⁷).

A79. The ADC of embodiment A78, wherein X⁴ is CH, X⁷ is CH, or both.

A80. The ADC of any one of embodiments A61 to A79, wherein d is an integer from 1 to 10, or is 1, 2, 3 or 4.

A81. The ADC of any one of embodiments A61 to A80, wherein L is a phosphate-based linker.

A82. The ADC of any one of embodiments A61 to A81, wherein L is a phosphate-based linker comprising a phosphate-based moiety selected from the group consisting of a phosphate ester, a pyrophosphate ester, a triphosphate ester, a tetraphosphate ester, a phosphonate, a diphosphonate, a phosporamidate, a pyrophosporamidate, a triphosphoramidate, a tetraphosphoramidate, a phosphorthioate and a diphosphorthioate.

A83. The ADC of any one of embodiments A61 to A82, wherein the corresponding amine of moiety A has a C log P value of at least about 1.

A84. The ADC of embodiment A82, wherein the phosphate-based moiety is a diphosphonate.

A85. The ADC of any one of embodiments A82 to A84, wherein L further comprises at least one additional moiety, wherein each at least one additional moiety is independently selected from the group consisting of unsubstituted alkylene, substituted alkylene, -(alkylene-O)—, optionally substituted arylene, —O—, —C(O)—, —N(R_{w})—, —S(O)₀₋₂—, a water-soluble polymer and an amino acid; wherein each R_{w} is independently H or C₁-C₈ alkyl; and combinations thereof.

A86. The ADC of embodiment A85, wherein each at least one additional moiety is independently selected from the group consisting of unsubstituted alkylene, -(alkylene-O)—, —C(O)—, —N(R_{w})—, a water-soluble polymer and an amino acid; wherein each R_{w} is independently H or C₁-C₈ alkyl; and combinations thereof.

A87. The ADC of any one of embodiments A82 to A86, wherein the phosphate-based moiety is covalently bound to the —O— atom of Formula (II) via a phosphorous atom of the phosphate-based moiety.

A88. The ADC of embodiment A85, A86 or A87, wherein L comprises at least one alkylene group.

A89. The ADC of any one of embodiments A85 to A88, wherein L comprises an amino acid.

A90. The ADC of any one of embodiments A85 to A89, wherein L comprises a water-soluble polymer.

A91. The ADC of any one of embodiments A85 to A90, wherein L comprises a water-soluble polymer and an amino acid, wherein the water-soluble polymer is conjugated to the amino acid.

A92. The ADC of embodiment A91, wherein the water-soluble polymer is conjugated to a side chain of the amino acid.

A93. The ADC of embodiment A91 or A92, wherein the water-soluble polymer is conjugated to the amino acid via a spacer element.

A94. The ADC of any one of embodiments A85 to A93, wherein the amino acid is selected from the group consisting of serine, threonine, cysteine, tyrosine, aspartic acid, glutamic acid, lysine and N_{ϵ}-methyl-lysine.

A95. The ADC of any one of embodiments A85 to A94, wherein the amino acid is lysine or N_{ϵ}-methyl-lysine.

A96. The ADC of embodiment A81, wherein L is selected from the group of linkers of Table 6.

A97. The ADC of embodiment A81, wherein L is selected from the group of linkers of Table 7.

A98. The ADC of embodiment A81, wherein L is selected from the group consisting of:

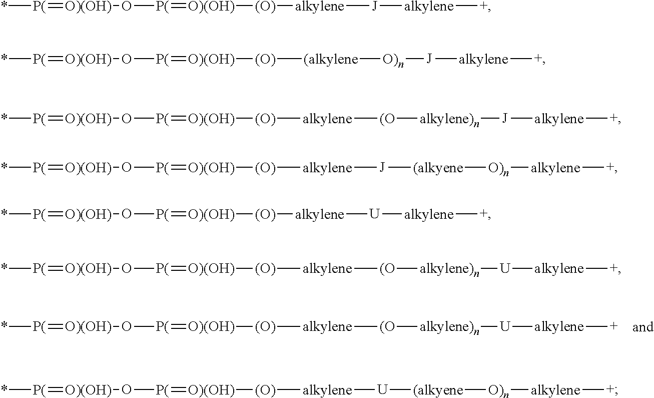

wherein:
each U is independently selected from the group consisting of:

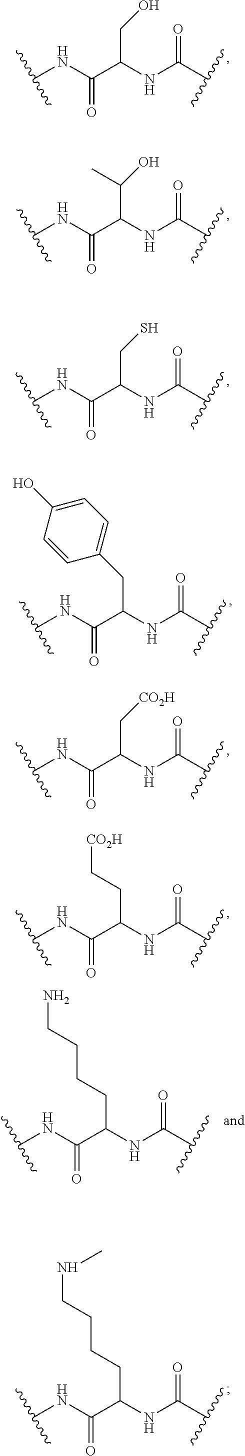

each J is independently

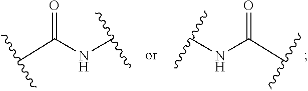 [SMILES: *NC(*)=O ; *NC(*)=O]

each alkylene is independently selected from the group consisting of:

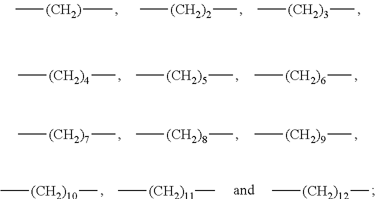

each n is independently an integer from 1 to 100;
* denotes the connection to the —O— atom of Formula (II); and
+ denotes the connection to E;
   wherein each linker L is optionally substituted with one or more water-soluble polymers.

A99. The ADC of any one of embodiments A96 to A98, wherein each n is independently an integer from 1 to 10, or wherein each n is independently 1, 2 or 3.

A100. The ADC of any one of embodiments A96 to A99, wherein L is substituted with one or more water-soluble polymers.

A101. The ADC of any one of embodiments A96 to A100, wherein L comprises group U, and one water-soluble polymer is conjugated to an amino acid side chain of group U.

A102. The ADC of embodiment A101, wherein the water-soluble polymer is conjugated to the amino acid side chain of group U via a spacer element.

A103. The ADC of any one of embodiments A61 to A102, wherein L is a bivalent linker optionally substituted with a water-soluble polymer.

A104. The ADC of any one of embodiments A96 to A103, wherein L is:

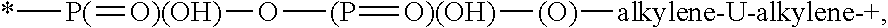

wherein:
U is selected from the group consisting of:

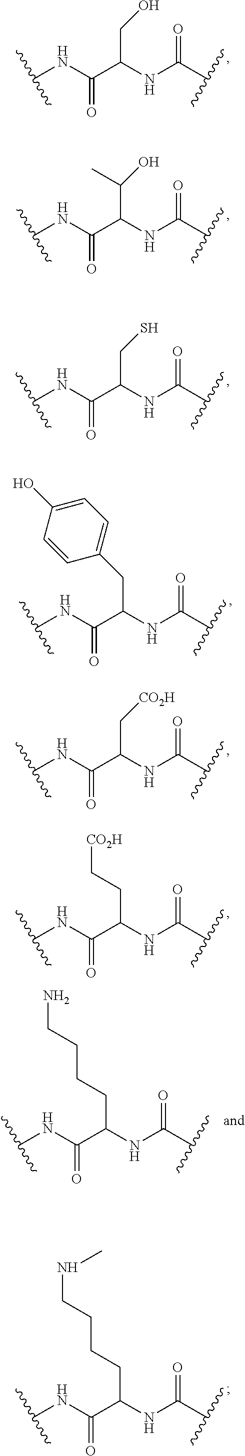

each alkylene is independently selected from the group consisting of:

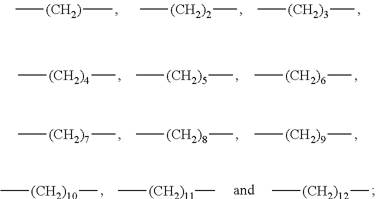

* denotes the connection to the —O— atom of Formula (II); and
+ denotes the connection to E;
   wherein L is optionally substituted with one or more water-soluble polymers.

A105. The ADC of embodiment A104, wherein L is substituted with one or more water-soluble polymers.

A106. The ADC of A105, wherein one water-soluble polymer is conjugated to an amino acid side chain of group U.

A107. The ADC of embodiment A106, wherein the one water-soluble polymer is conjugated to the amino acid side chain of group U via a spacer element.

A108. The ADC of any one of embodiments A104 to A107, wherein U is:

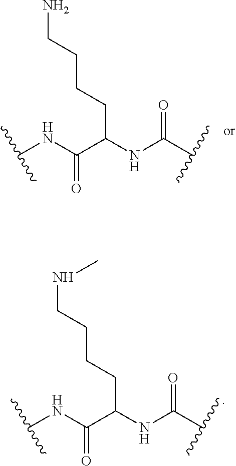 [SMILES: *NC(=O)C(CCCCN)NC(*)=O ; *NC(=O)C(CCCCNC)NC(*)=O]

A109. The ADC of embodiment A107 or A108, wherein the spacer element is a carbonyl group.

A110. The ADC of any one of embodiments A107 to A109, wherein L has the following structure:

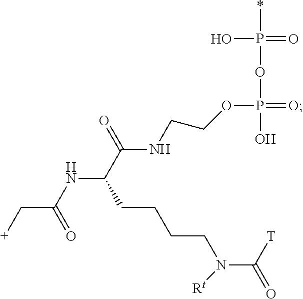

wherein: T is the water-soluble polymer; R^{t} is H or methyl; * denotes the connection to the —O— atom of Formula (II); and + denotes the connection to E.

A111. The ADC of any one of embodiments A85 to A110, wherein the water-soluble polymer is a (polyethylene)glycol (PEG) moiety.

A112. The ADC of embodiment A111, wherein the PEG moiety has a molecular weight within a range of about 100 Da to about 100,000 Da.

A113. The ADC of embodiment A112, wherein the PEG moiety has a molecular weight within a range of about 100 Da to about 10,000 Da, about 100 Da to about 5,000 Da, or about 100 Da to about 1,000 Da.

A114. The ADC of any one of embodiments A61 to A113, wherein E comprises an amide, an ester, a thioester, a pyrrolidine-2,5-dione, an oxime, a 1,2,3-triazole or a 1,4-dihydropyridazine, wherein the 1,2,3-triazole and the 1,4-dihydropyridazine are each optionally fused to an 8-membered ring.

A115. The ADC of embodiment A114, wherein E is selected from the group consisting of:

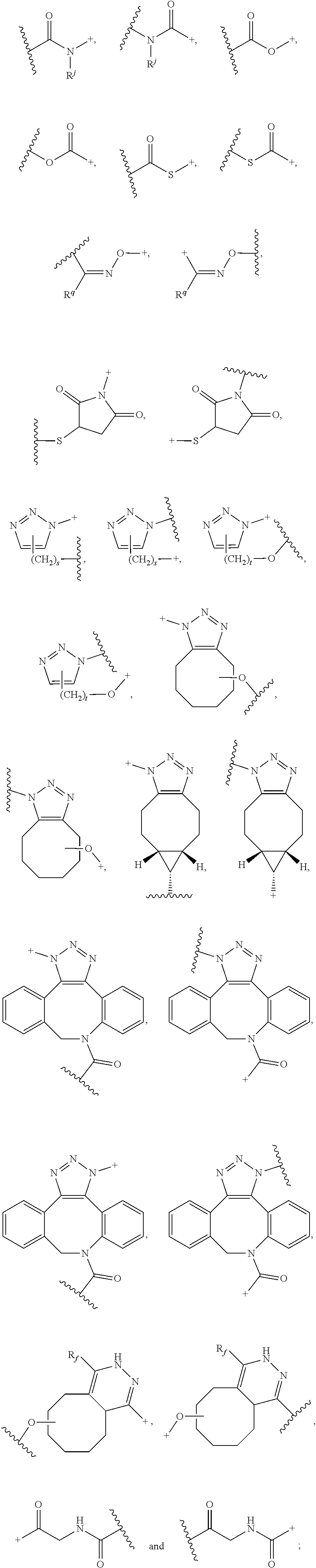

wherein:
   each R^{j} is independently H or unsubstituted C₁-C₆ alkyl;
   each R^{q} is independently unsubstituted C₁-C₆ alkyl;
   each R^{f} is independently H or unsubstituted C₁-C₆ alkyl;
   each s is independently 0, 1, 2, 3, 4, 5 or 6;
   each t is independently 0, 1, 2, 3, 4, 5 or 6;
   each + denotes connection to L; and
   each wavy line denotes connection to Ab.

A116. The ADC of any one of embodiments A61 to A115, wherein E is:

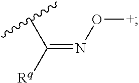

wherein R^{q} is unsubstituted C₁-C₆ alkyl.

A117. The ADC of embodiment A116, wherein R^{q} is methyl.

A118. The ADC of any one of embodiments A1 to A117, wherein E joins L to a non-natural amino acid of Ab.

A119. The ADC of any one of embodiments A61 to A118, wherein Ab is configured to bind to an antigen.

A120. The ADC of embodiment A119, wherein Ab is configured to bind to a tumor-associated antigen (TAA) or cancer antigen.

A121. The ADC of embodiment A119 or A120, wherein the antigen selected from the group consisting of PD-1, PD-L1, PSMA, CD70, CD3, HER2, HER3, TROP2, GPC3, VEGFR, EGFR, c-Met (HGFR), CD19, CD22, CD25 (IL-2R alpha), CD30, CD33, CD37, CD46, CD48, CD56 (NCAM-1), CD71 (Transferrin R), CD74, CD79b, CD123 (IL-3R alpha), CD138 (syndecan-1), CD142, CD166 (ALCAM), CD203c (ENPP3), CD205 (LY75), CD221 (IGF-1R), CD262 (TRAIL R2), CD276 (B7-H3), mesothelin, EpCAM, CEACAM5, CEACAM6, DLL3, ROR1, ROR2, GPNMB, GCC, GUCY2c, NaPi2b, Flt-1, Flt-3, folate receptor alpha, Tissue Factor (TF), CA6, MUC1, MUC16 (CA-125), BCMA, SLAMF7 (CS1), TIM1, CanAg, Ckit (CD117), EphA2, Nectin4, SLTRK6, FGFR2, LYPD3 (C4.4a), Cadherin 3, 5T4 (TPBG), STEAP1, PTK7, Ephrin-A4, LIV-1 (SLC39A6 or ZIP6), SLC1A5, TENB2, ETBR, integrin v3, Cripto, AGS-5 (SLC44A4), LY6E, AXL, LAMP1, LRRC15, TNF-alpha and MN/CA IX.

A122. The ADC of embodiment A121, wherein the antigen selected from the group consisting of TROP2, CD70, HER2, CD3, PSMA and GPC3.

A123. The ADC of embodiment A122, wherein the antigen is GPC3.

A124. The ADC of embodiment A122, wherein the antigen is CD70.

A125. The ADC of embodiment A124, wherein Ab is an anti-CD70 antibody comprising at least one sequence listed in Table 2.

A126. The ADC of embodiment A125, wherein the anti-CD70 Ab comprises a heavy chain sequence and a light chain sequence, wherein the heavy chain sequence comprises an amino acid sequence selected from the group consisting of the heavy chain sequences listed in Table 2, and the light chain sequence comprises an amino acid sequence selected from the group consisting of the light chain sequences listed in Table 2, wherein the heavy chain sequences comprises the one or more non-natural amino acids.

A127. The ADC of embodiment A126, wherein the anti-CD70 Ab comprises two heavy chain sequences selected from the group consisting of the heavy chain sequences listed in Table 2, wherein each heavy chain sequence comprises one non-natural amino acid.

A128. The ADC of any one of embodiments A61 to A127, wherein the one or more non-natural amino acids is selected from the group consisting of para-acetyl phenylalanine, 4-acetyl-L-phenylalanine (para-acetyl-L-phenylalanine (pAF)), 3-O—(N-acetyl-beta-D-glucosaminyl)-L-threonine, N4-(β-N-Acetyl-D-glucosaminyl)-L-asparagine, 0-allyl-L-tyrosine, alpha-N-acetylgalactosamine-O-L-serine, alpha-N-acetylgalactosamine-O-L-threonine, 2-aminooctanoic acid, 2-amino-L-phenylalanine, 3-amino-L-phenylalanine, 4-amino-L-phenylalanine, 2-amino-L-tyrosine, 3-amino-L-tyrosine, 4-azido-L-phenylalanine, 4-benzoyl-L-phenylalanine, (2,2-bipyridin-5yl)-L-alanine, 3-borono-L-phenylalanine, 4-borono-L-phenylalanine, 4-bromo-L-phenylalanine, p-carboxymethyl-L-phenylalanine, 4-carboxy-L-phenylalanine, p-cyano-L-phenylalanine, 3,4-dihydroxy-L-phenylalanine (L-DOPA), 4-ethynyl-L-phenylalanine, 2-fluoro-L-phenylalanine, 3-fluoro-L-phenylalanine, 4-fluoro-L-phenylalanine, 0-(3-O-D-galactosyl-N-acetyl-beta-D-galactosaminyl)-L-serine, L-homoglutamine, (8-hydroxyquinolin-3-yl)-L-alanine, 4-iodo-L-phenylalanine, 4-isopropyl-L-phenylalanine, 0-i-propyl-L-tyrosine, 3-isopropyl-L-tyrosine, 0-mannopyranosyl-L-serine, 2-methoxy-L-phenylalanine, 3-methoxy-L-phenylalanine, 4-methoxy-L-phenylalanine, 3-methyl-L-phenylalanine, O-methyl-L-tyrosine, 3-(2-naphthyl)-L-alanine, 5-nitro-L-histidine, 4-nitro-L-histidine, 4-nitro-L-leucine, 2-nitro-L-phenylalanine, 3-nitro-L-phenylalanine, 4-nitro-L-phenylalanine, 4-nitro-L-tryptophan, 5-nitro-L-tryptophan, 6-nitro-L-tryptophan, 7-nitro-L-tryptophan, 2-nitro-L-tyrosine, 3-nitro-L-tyrosine, 0-phospho-L-serine, 0-phospho-L-tyrosine, 4-propargyloxy-L-phenylalanine, O-2-propyn-1-yl-L-tyrosine, 4-sulfo-L-phenylalanine and O-sulfo-L-tyrosine.

A129. The ADC of any one of embodiments A61 to A128, wherein each of the one or more non-natural amino acids is para-acetyl phenylalanine.

A130. The ADC of any one of embodiments A61 to A129, wherein Ab comprises 1, 2, 3, 4, 5, 6, 7, 8, 9 or 10 non-natural amino acids.

A131. A pharmaceutical composition comprising a compound of any one of embodiments A1 to A60, or an ADC of any one of embodiments A61 to A130, and at least one pharmaceutically acceptable adjuvant, binder, buffer, carrier, diluent or excipient.

A132. The pharmaceutical composition of embodiment A131, further comprising a chemotherapeutic agent, hormonal agent, antitumor agent, immunostimulatory agent, immunomodulator, corticosteroid, or combination thereof.

A133. A method of treating a disease or condition in a subject, the method comprising administering to the subject a therapeutically effect amount of a compound of any one of embodiments A1 to A60, an ADC of any one of embodiments A61 to A130, or a pharmaceutical composition of embodiment A131 or A132.

A134. The method of embodiment A133, wherein the disease or condition is cancer.

A135. The method of embodiment A134, wherein the cancer is a blood cancer.

A136. The method of embodiment A135, wherein the blood cancer is a leukemia, a lymphoma or a myeloma.

A137. The method of any one of embodiments A133 to A136, wherein the treatment comprises administering an ADC of any one of embodiments A61 to A130, or a pharmaceutical composition comprising an ADC of any one of embodiments A61 to A130.

A138. The method of embodiment A137, wherein Ab is an anti-CD70 antibody.

A139. The method of embodiment A134, wherein the cancer is a liver cancer.

A140. The method of embodiment A139, wherein the liver cancer is hepatocellular carcinoma.

A141. The method of embodiment A139 or A140, wherein the treatment comprises administering an ADC of any one of embodiments A61 to A130, or a pharmaceutical composition comprising an ADC of any one of embodiments A61 to A130, wherein Ab an anti-GPC3 antibody.

Additional non-limiting embodiments of the invention are listed below.

B1. A compound of Formula (I) having the following structure:

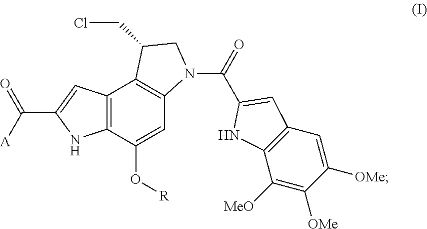

wherein:
R is H or L-W, wherein L is a linker and W is a reactive moiety; and
A is selected from the group consisting of formula (a), (b), (c) and (d), having the following structures:

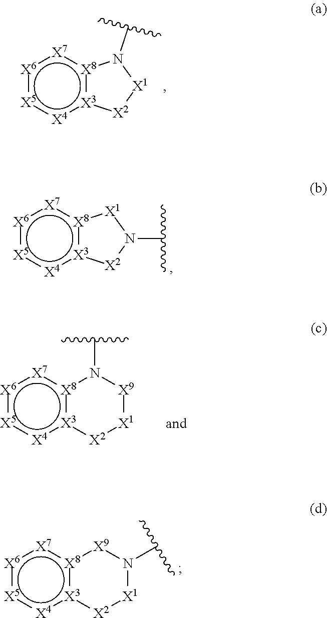

wherein:
   each X¹ is C(R^{1a})(R^{1b}); wherein each R^{1a} and R^{1b} is independently H, halogen, alkyl, alkenyl or alkynyl;
   each X² is C(R^{2a})(R^{2b}); wherein each R^{2a} and R^{2b} is independently H, halogen, alkyl, alkenyl or alkynyl;
   each X³ is C;
   each X⁴ is C(R⁴) or N, wherein R⁴ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
   each X⁵ is C(R⁵) or N, wherein R⁵ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
   each X⁶ is C(R⁶) or N, wherein R⁶ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
   each X⁷ is C(R⁷) or N, wherein R⁷ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{S});
   each X⁸ is C; and
   each X⁹, when present, is C(R^{9a})(R^{9b}); wherein each R^{9a} and R^{9b} is independently H, halogen, alkyl, alkenyl or alkynyl;
      wherein:
each R^{a} and R^{b} is independently H, alkyl, alkenyl or alkynyl;
each R^{c} is independently H, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl or heteroarylalkyl;
each R^{s} is independently H, halogen, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl or heteroarylalkyl; and
each m is independently 0, 1, 2 or 3;
   or a salt thereof.

B2. The compound of embodiment B1, wherein A has the structure of formula (a), and the compound is a compound of Formula (Ia) having the following structure:

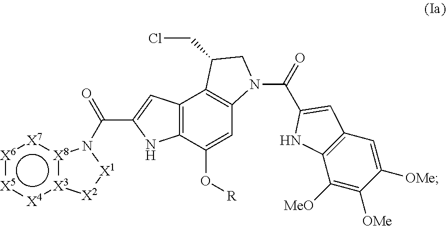

wherein:
R is H or L-W, wherein L is a linker and W is a reactive moiety;
X¹ is C(R^{1a})(R^{1b}); wherein each R^{1a} and R^{1b} is independently H, halogen, alkyl, alkenyl or alkynyl;
X² is C(R^{2a})(R^{2b}); wherein each R^{2a} and R^{2b} is independently H, halogen, alkyl, alkenyl or alkynyl;
X³ is C;
X⁴ is C(R⁴) or N, wherein R⁴ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
X⁵ is C(R⁵) or N, wherein R⁵ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
X⁶ is C(R⁶) or N, wherein R⁶ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
X⁷ is C(R⁷) or N, wherein R⁷ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{S}); and
X⁸ is C;
   wherein:
each R^{a} and R^{b} is independently H, alkyl, alkenyl or alkynyl;
each R^{c} is independently H, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl or heteroarylalkyl;
each R^{s} is independently H, halogen, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl or heteroarylalkyl; and
each m is independently 0, 1, 2 or 3.

B3. The compound of embodiment B1 or B2, wherein:
X¹ is C(R^{1a})(R^{1b}); wherein each R^{1a} and R^{1b} is independently H, halogen or unsubstituted alkyl;
X² is C(R^{2a})(R^{2b}); wherein each R^{2a} and R^{2b} is independently H, halogen or unsubstituted alkyl;
X³ is C;
X⁴ is C(R⁴) or N, wherein R⁴ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl;
X⁵ is C(R⁵) or N, wherein R⁵ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl;
X⁶ is C(R⁶) or N, wherein R⁶ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl;
X⁷ is C(R⁷) or N, wherein R⁷ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl;
X⁸ is C; and
X⁹, when present, is C(R^{9a})(R^{9b}); wherein each R^{9a} and R^{9b} is independently H, halogen or unsubstituted alkyl.

B4. The compound of embodiment B1, B2 or B3, wherein:
X¹ is C(R^{1a})(R^{1b}); wherein each R^{1a} and R^{1b} is H;
X² is C(R^{2a})(R^{2b}); wherein each R^{2a} and R^{2b} is H;
X³ is C;
X⁴ is C(R⁴) or N, wherein R⁴ is H;
X⁵ is C(R⁵) or N, wherein R⁵ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl;
X⁶ is C(R⁶) or N, wherein R⁶ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl;
X⁷ is C(R⁷) or N, wherein R⁷ is H;
X⁸ is C; and
X⁹, when present, is CH₂.

B5. The compound of any one of embodiments B1 to B4, wherein each said heteroalkyl is alkoxy.

B6. The compound of any one of embodiments B1 to B5, wherein:
X¹ is C(R^{1a})(R^{1b}); wherein each R^{1a} and R^{1b} is H;
X² is C(R^{2a})(R^{2b}); wherein each R^{2a} and R^{2b} is H;
X³ is C;
X⁴ is C(R⁴) or N, wherein R⁴ is H;
X⁵ is C(R⁵) or N, wherein R⁵ is H, halogen or alkoxy;
X⁶ is C(R⁶) or N, wherein R⁶ is H, halogen or alkoxy;
X⁷ is C(R⁷) or N, wherein R⁷ is H;
X⁸ is C; and
X⁹, when present, is CH₂.

B7. The compound of embodiment B6, wherein X⁵ is C(R⁵) or N, wherein R⁵ is H or alkoxy; and X⁶ is C(R⁶) or N, wherein R⁶ is H or alkoxy.

B8. The compound of any one of embodiments B1 to B7, wherein X⁴ is N, X⁵ is C(R⁵), X⁶ is C(R⁶) and X⁷ is C(R⁷).

B9. The compound of any one of embodiments B1 to B7, wherein X⁴ is C(R⁴), X⁵ is N, X⁶ is C(R⁶) and X⁷ is C(R⁷).

B10. The compound of any one of embodiments B1 to B7, wherein X⁴ is C(R⁴), X⁵ is C(R⁵), X⁶ is N and X⁷ is C(R⁷).

B11. The compound of any one of embodiments B1 to B7, wherein X⁴ is C(R⁴), X⁵ is C(R⁵), X⁶ is C(R⁶) and X⁷ is C(R⁷).

B12. The compound of any one of embodiments B1 to B7 and B9 to B11, wherein at least one of X⁴ and X⁷ is CH.

B13. The compound of any one of embodiments B1 to B12, wherein each of X⁴ and X⁷ is CH.

B14. The compound of any one of embodiments B1 to B13, wherein at least one of R⁵ and R⁶ is alkoxy.

B15. The compound of any one of embodiments B5 to B14, wherein each said alkoxy is independently —OR^{k}, wherein each R^{k} is independently alkyl optionally substituted with heterocyclyl or —N(R^{d})(R^{e}); wherein said heterocyclyl contains at least one nitrogen atom, and each R^{d} and R^{e} is independently H, alkyl, alkenyl or alkynyl.

B16. The compound of embodiment B15, wherein each said alkoxy is selected from the group consisting of —OCH₃, —OCH₂CH₃, —OCH₂CH₂CH₃, —OCH(CH₃)₂, —OCH₂CH₂N(CH₃)₂,

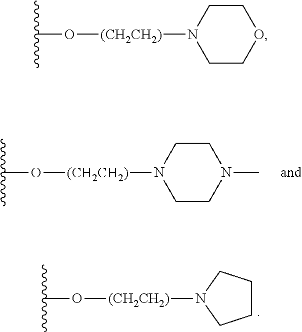

B17. The compound of any one of embodiments B1 to B16, wherein R is L-W.

B18. The compound of embodiment B17, wherein L is a phosphate-based linker.

B19. The compound of embodiment B18, wherein the phosphate-based linker comprises a phosphate-based moiety having the following structure:

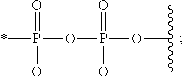 [SMILES: *OP(=O)(O)OP(*)(=O)O]

wherein * denotes the connection to the —O— atom at position R of Formula (I) or Formula (Ia); wherein L further comprises at least one additional moiety, and the wavy line of the phosphate-based moiety denotes the connection to one of the at least one additional moiety; wherein the at least one additional moiety is selected from the group consisting of unsubstituted alkylene, substituted alkylene, -(alkylene-O)—, optionally substituted arylene, —O—, —C(O)—, —N(R_{w})—, —S(O)₀₋₂—, a water-soluble polymer and an amino acid; wherein each R_{w} is independently H or C₁-C₈ alkyl; and combinations thereof.

B20. The compound of embodiment B19, wherein each at least one additional moiety is independently selected from the group consisting of unsubstituted alkylene, -(alkylene-O)—, —C(O)—, —N(R_{w})—, a water-soluble polymer and an amino acid; wherein each R_{w} is independently H or C₁-C₈ alkyl; and combinations thereof.

B21. The compound of any one of embodiments B1 to B17, wherein R is L-W, and L is selected from the group of linkers of Table 6.

B22. The compound of any one of embodiments B1 to B17, wherein R is L-W, and L is selected from the group of linkers of Table 7.

B23. The compound of any one of embodiments B1 to B17, wherein R is L-W, and L is selected from the group of linkers of Table 8.

B24. The compound of any one of embodiment B1 to B17, wherein R is L-W, and L has the following structure:

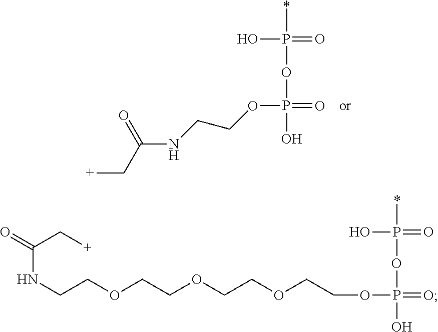

wherein * denotes the connection to the —O— atom at position R of Formula (I) or Formula (Ia); and + denotes the connection to W.

B25. The compound of any one of embodiments B1 to B17, wherein R is L-W, and L has the following structure:

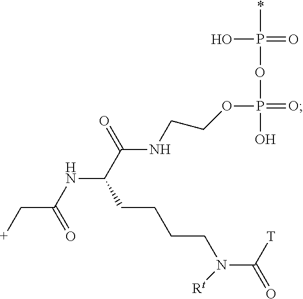

wherein T is a water-soluble polymer; R^{t} is H or methyl; * denotes the connection to the —O— atom at position R of Formula (I) or Formula (Ia); and + denotes the connection to W.

B26. The compound of embodiment B25, wherein the water-soluble polymer is a (polyethylene)glycol (PEG) moiety.

B27. The compound of embodiment B26, wherein the PEG moiety has a molecular weight within a range of about 100 Da to about 100,000 Da, about 100 Da to about 10,000 Da, about 100 Da to about 5,000 Da, or about 100 Da to about 1,000 Da.

B28. The compound of embodiment B26, wherein the PEG moiety is —(CH₂CH₂O)ₙCH₃, wherein n is an integer from 1 to 24.

B29. The compound of embodiment B26, wherein the PEG moiety is —(CH₂CH₂O)ₙCH₃, wherein n is 8, 9, 10, 11 or 12.

B30. The compound of any one of embodiments B1 to B29, wherein R is L-W, and the reactive moiety W comprises —N₃, —OH, —SH, —NH(R^{j}), —C(O)R^{q}, —C(O)OR^{x}, —C(O)CH₂NH₂, an activated ester, —O—NH₂, a maleimide, a tetrazine, an alkyne, a cyclooctyne or an (E)-cyclooctene; wherein R^{j} is H or unsubstituted alkyl, R^{q} is unsubstituted alkyl, and R^{x} is H, unsubstituted alkyl or a carboxylic acid protecting group.

B31. The compound of embodiment B30, wherein reactive moiety W is selected from the group consisting of:

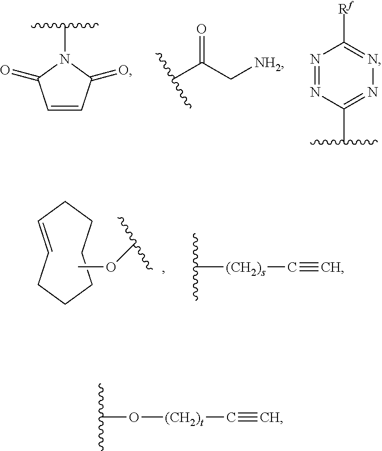

—N₃, —OH, —SH, —NH(R^{j}), —C(O)R^{q}, —C(O)OR^{x}, an activated ester, —O—NH₂ and an optionally substituted monocyclic or polycyclic group comprising the cyclooctyne;
wherein:
   R^{j} is H or unsubstituted C₁-C₆ alkyl,
   R^{q} is unsubstituted C₁-C₆ alkyl,
   R^{x} is H, unsubstituted C₁-C₆ alkyl or a carboxylic acid protecting group,
   R^{f} is H or unsubstituted C₁-C₆ alkyl,
   s is 0, 1, 2, 3, 4, 5 or 6, and
   t is 0, 1, 2, 3, 4, 5 or 6.

B32. The compound of anyone of embodiments B1 to B31, wherein W is —ONH₂.

B33. The compound of embodiment B1 or B2, wherein the compound is selected from the group consisting of:

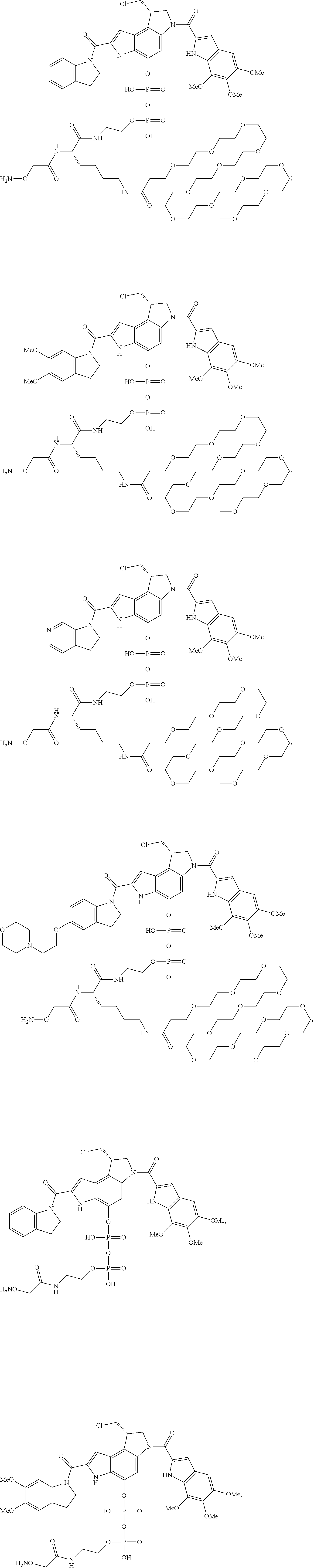

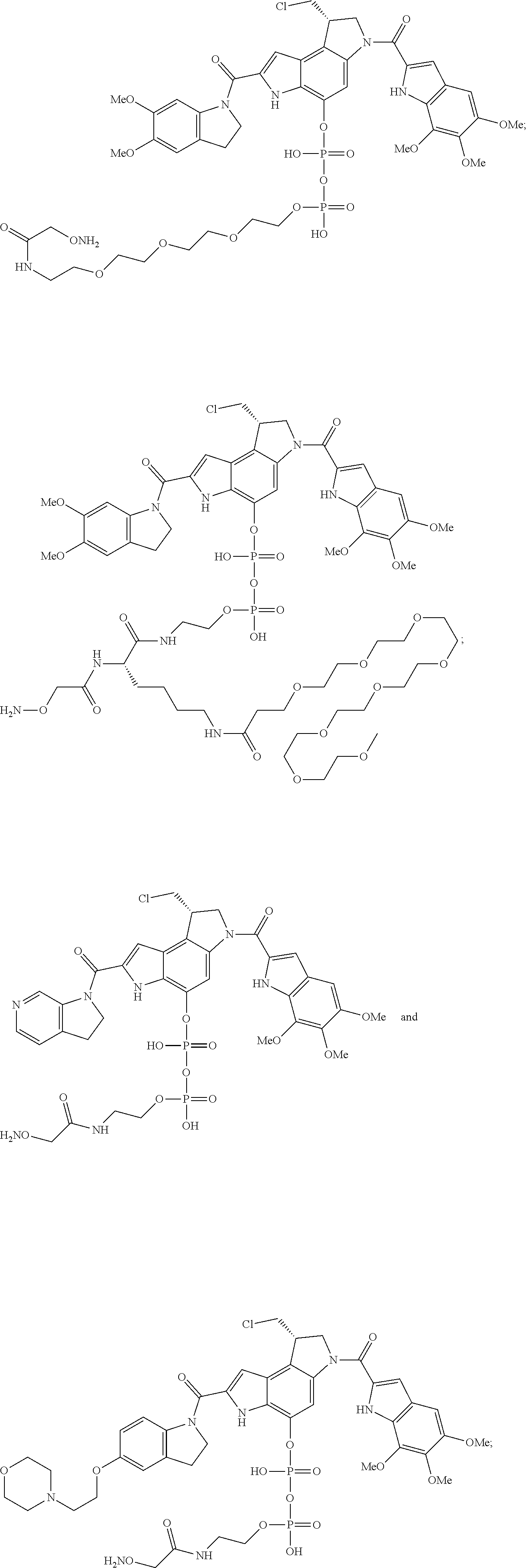

and salts thereof.

B34. The compound of embodiment B1 or B2, having the following structure:

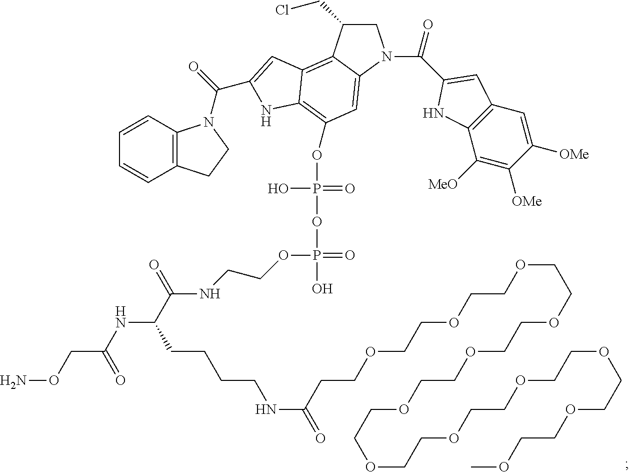 [SMILES: COCCOCCOCCOCCOCCOCCOCCOCCOCCOCCOCCOCCC(=O)NCCCC[C@H](NC(=O)CON)C(=O)NCCOP(=O)(O)OP(=O)(O)Oc1cc2c(c3cc(C(=O)N4CCc5ccccc54)[nH]c13)[C@H](CCl)CN2C(=O)c1cc2cc(OC)c(OC)c(OC)c2[nH]1]

or a salt thereof.

B35. The compound of embodiment B1 or B2, having the following structure:

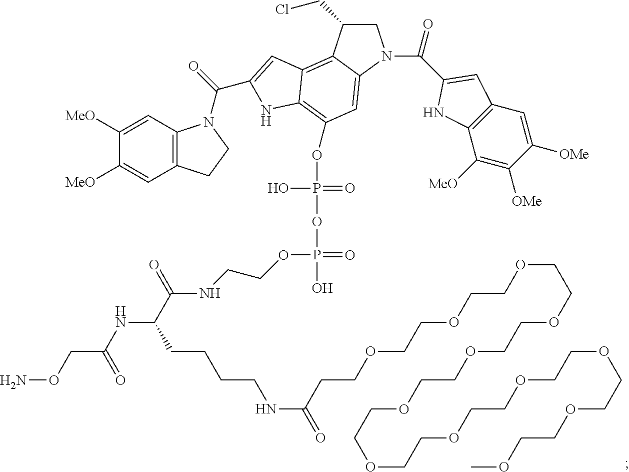 [SMILES: COCCOCCOCCOCCOCCOCCOCCOCCOCCOCCOCCOCCC(=O)NCCCC[C@H](NC(=O)CON)C(=O)NCCOP(=O)(O)OP(=O)(O)Oc1cc2c(c3cc(C(=O)N4CCc5cc(OC)c(OC)cc54)[nH]c13)[C@H](CCl)CN2C(=O)c1cc2cc(OC)c(OC)c(OC)c2[nH]1]

or a salt thereof.

B36. The compound of embodiment B1 or B2, having the following structure:

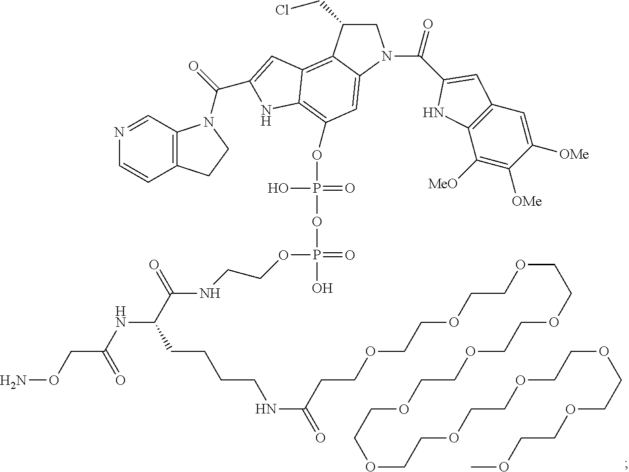 [SMILES: COCCOCCOCCOCCOCCOCCOCCOCCOCCOCCOCCOCCC(=O)NCCCC[C@H](NC(=O)CON)C(=O)NCCOP(=O)(O)OP(=O)(O)Oc1cc2c(c3cc(C(=O)N4CCc5ccncc54)[nH]c13)[C@H](CCl)CN2C(=O)c1cc2cc(OC)c(OC)c(OC)c2[nH]1]

or a salt thereof.

B37. The compound of embodiment B1 or B2, having the following structure:

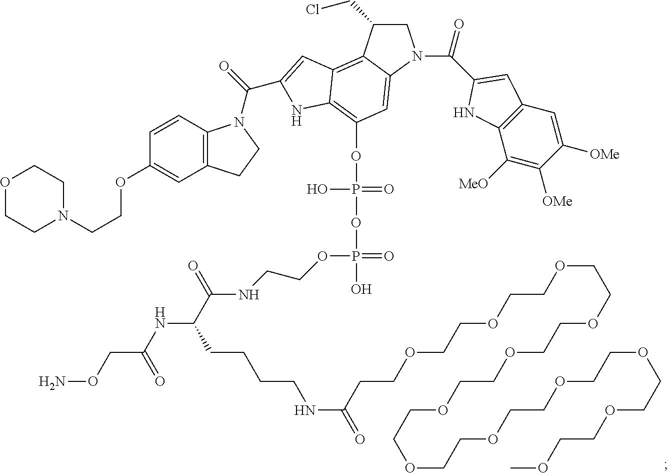 [SMILES: COCCOCCOCCOCCOCCOCCOCCOCCOCCOCCOCCOCCC(=O)NCCCC[C@H](NC(=O)CON)C(=O)NCCOP(=O)(O)OP(=O)(O)Oc1cc2c(c3cc(C(=O)N4CCc5cc(OCCN6CCOCC6)ccc54)[nH]c13)[C@H](CCl)CN2C(=O)c1cc2cc(OC)c(OC)c(OC)c2[nH]1]

or a salt thereof.

B38. The compound of any one of embodiments B1 to B16 wherein R is H.

B39. The compound of embodiment B1 or B2, wherein the compound is selected from the group consisting of:

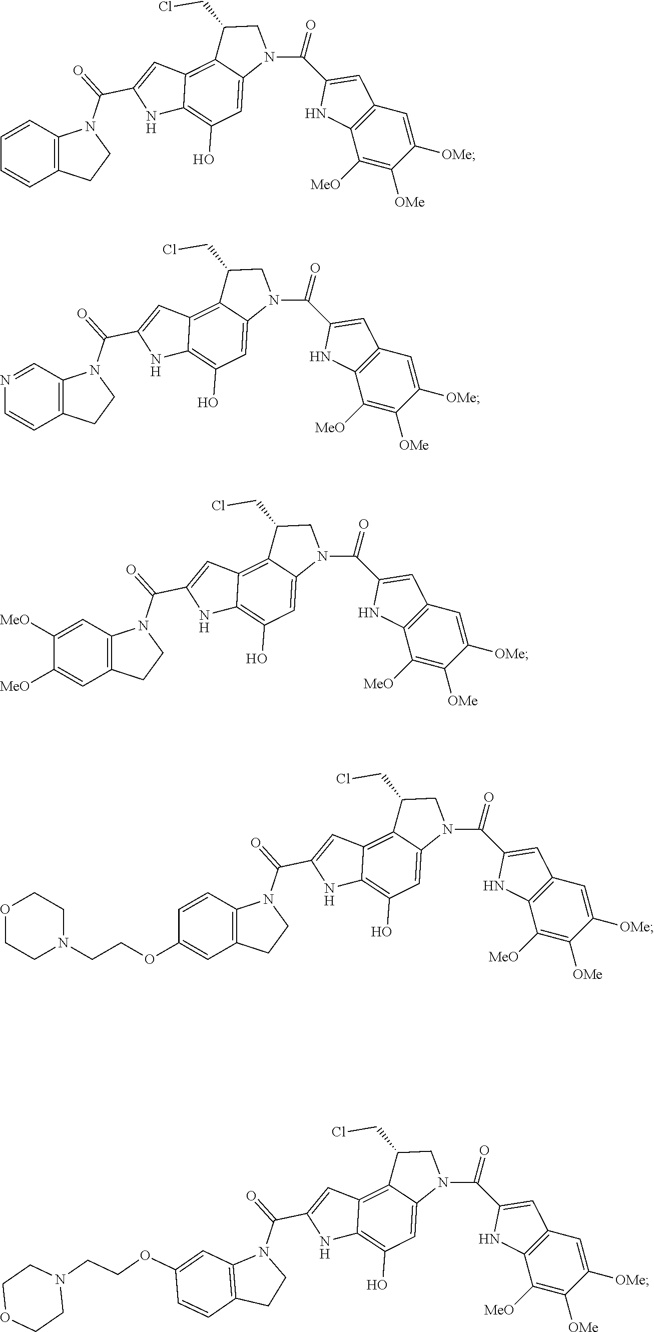 [SMILES: COc1cc2cc(C(=O)N3C[C@@H](CCl)c4c3cc(O)c3[nH]c(C(=O)N5CCc6ccccc65)cc43)[nH]c2c(OC)c1OC ; COc1cc2cc(C(=O)N3C[C@@H](CCl)c4c3cc(O)c3[nH]c(C(=O)N5CCc6ccncc65)cc43)[nH]c2c(OC)c1OC ; COc1cc2c(cc1OC)N(C(=O)c1cc3c4c(cc(O)c3[nH]1)N(C(=O)c1cc3cc(OC)c(OC)c(OC)c3[nH]1)C[C@H]4CCl)CC2 ; COc1cc2cc(C(=O)N3C[C@@H](CCl)c4c3cc(O)c3[nH]c(C(=O)N5CCc6cc(OCCN7CCOCC7)ccc65)cc43)[nH]c2c(OC)c1OC ; COc1cc2cc(C(=O)N3C[C@@H](CCl)c4c3cc(O)c3[nH]c(C(=O)N5CCc6ccc(OCCN7CCOCC7)cc65)cc43)[nH]c2c(OC)c1OC]

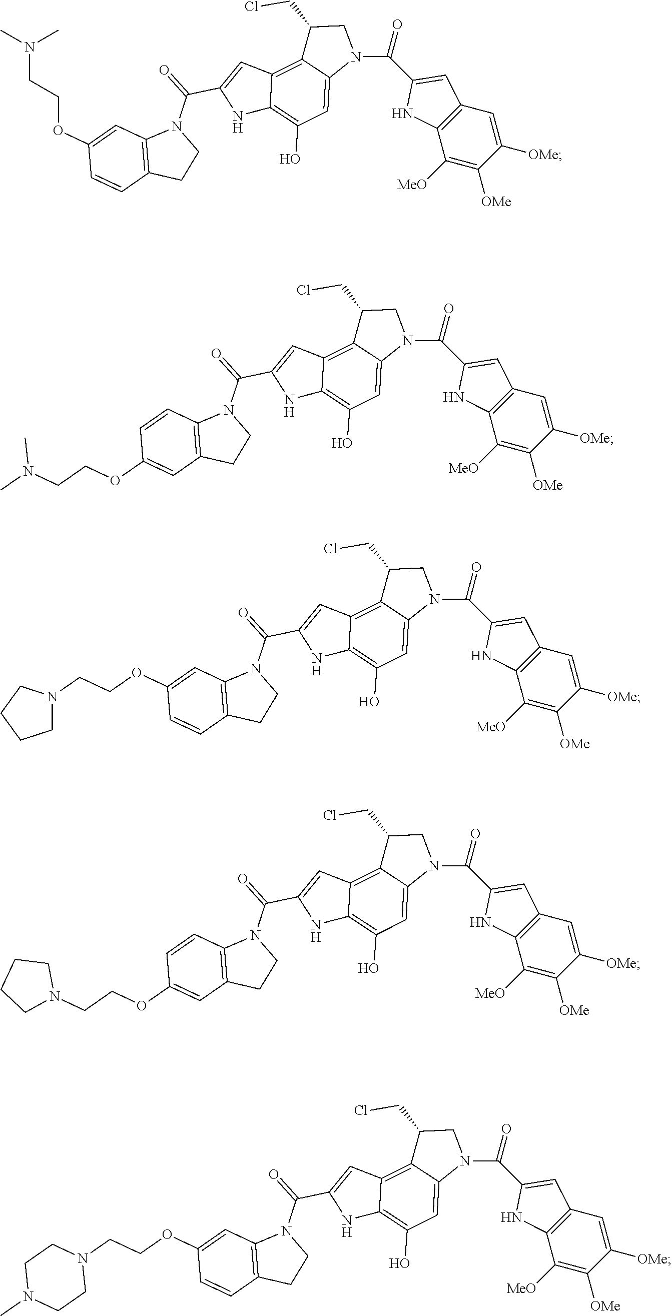 [SMILES: COc1cc2cc(C(=O)N3C[C@@H](CCl)c4c3cc(O)c3[nH]c(C(=O)N5CCc6ccc(OCCN(C)C)cc65)cc43)[nH]c2c(OC)c1OC ; COc1cc2cc(C(=O)N3C[C@@H](CCl)c4c3cc(O)c3[nH]c(C(=O)N5CCc6cc(OCCN(C)C)ccc65)cc43)[nH]c2c(OC)c1OC ; COc1cc2cc(C(=O)N3C[C@@H](CCl)c4c3cc(O)c3[nH]c(C(=O)N5CCc6ccc(OCCN7CCCC7)cc65)cc43)[nH]c2c(OC)c1OC ; COc1cc2cc(C(=O)N3C[C@@H](CCl)c4c3cc(O)c3[nH]c(C(=O)N5CCc6cc(OCCN7CCCC7)ccc65)cc43)[nH]c2c(OC)c1OC ; COc1cc2cc(C(=O)N3C[C@@H](CCl)c4c3cc(O)c3[nH]c(C(=O)N5CCc6ccc(OCCN7CCN(C)CC7)cc65)cc43)[nH]c2c(OC)c1OC]

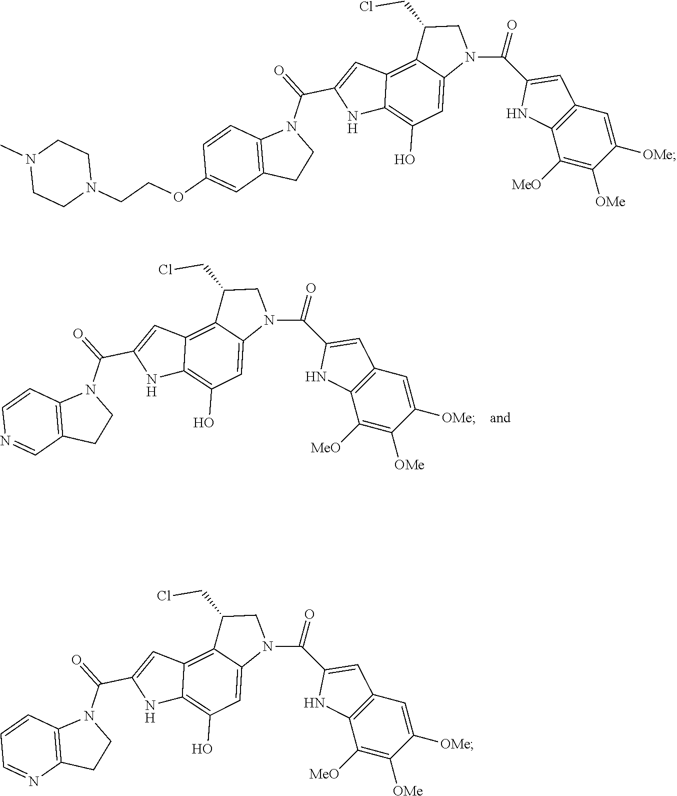 [SMILES: COc1cc2cc(C(=O)N3C[C@@H](CCl)c4c3cc(O)c3[nH]c(C(=O)N5CCc6cc(OCCN7CCN(C)CC7)ccc65)cc43)[nH]c2c(OC)c1OC ; COc1cc2cc(C(=O)N3C[C@@H](CCl)c4c3cc(O)c3[nH]c(C(=O)N5CCc6cnccc65)cc43)[nH]c2c(OC)c1OC ; COc1cc2cc(C(=O)N3C[C@@H](CCl)c4c3cc(O)c3[nH]c(C(=O)N5CCc6ncccc65)cc43)[nH]c2c(OC)c1OC]

and salts thereof.

B40. The compound of embodiment B1 or B2, wherein the compound is selected from the group consisting of:

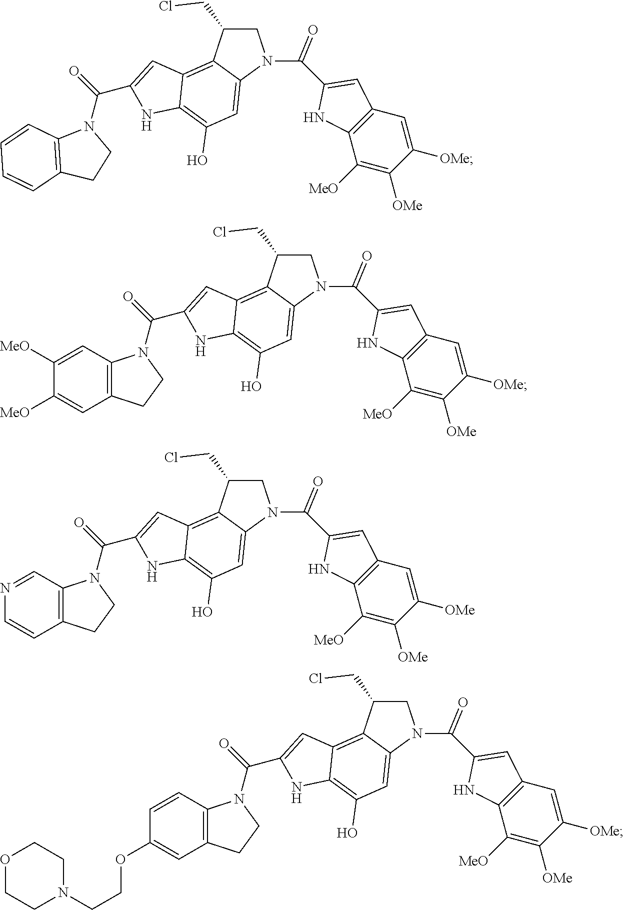

and salts thereof.

B41. An antibody-drug conjugate (ADC) of Formula (II):

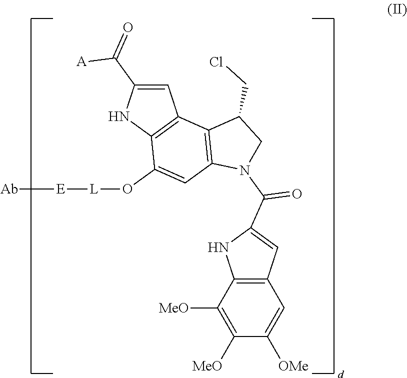

wherein:
Ab is an antibody, wherein Ab comprises one or more non-natural amino acids;
L is a linker;
E is a moiety joining Ab and L;
d is an integer from 1 to 10; and
A is selected from the group consisting of formula (a), (b), (c) and (d), having the following structures:

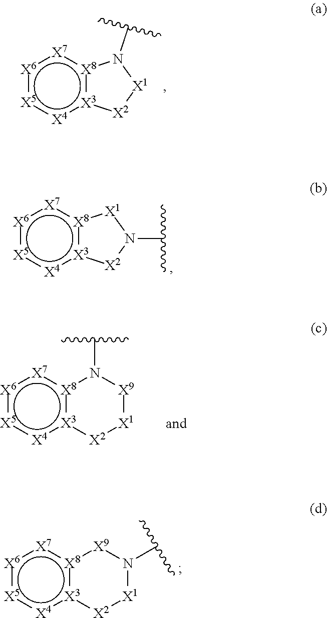

wherein:
   each X¹ is C(R^{1a})(R^{1b}); wherein each R^{1a} and R^{1b} is independently H, halogen, alkyl, alkenyl or alkynyl;
   each X² is C(R^{2a})(R^{2b}); wherein each R^{2a} and R^{2b} is independently H, halogen, alkyl, alkenyl or alkynyl;
   each X³ is C;
   each X⁴ is C(R⁴) or N, wherein R⁴ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
   each X⁵ is C(R⁵) or N, wherein R⁵ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
   each X⁶ is C(R⁶) or N, wherein R⁶ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
   each X⁷ is C(R⁷) or N, wherein R⁷ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
   each X⁸ is C; and
   each X⁹, when present, is C(R^{9a})(R^{9b}); wherein each R^{9a} and R^{9b} is independently H, halogen, alkyl, alkenyl or alkynyl;
      wherein:
each R^{a} and R^{b} is independently H, alkyl, alkenyl or alkynyl;
each R^{c} is independently H, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl or heteroarylalkyl;
each R^{s} is independently H, halogen, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl or heteroarylalkyl; and
each m is independently 0, 1, 2 or 3;
   or a pharmaceutically acceptable salt thereof.

B42. The ADC of embodiment B41, wherein A has the following structure:

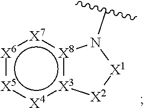

and wherein the remaining variables are as defined in embodiment 41.

B43. The ADC of embodiment B41 or B42, wherein:
X¹ is C(R^{1a})(R^{1b}); wherein each R^{1a} and R^{1b} is independently H, halogen or unsubstituted alkyl;
X² is C(R^{2a})(R^{2b}); wherein each R^{2a} and R^{2b} is independently H, halogen or unsubstituted alkyl;
X³ is C;
X⁴ is C(R⁴) or N, wherein R⁴ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl;
X⁵ is C(R⁵) or N, wherein R⁵ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl;
X⁶ is C(R⁶) or N, wherein R⁶ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl;
X⁷ is C(R⁷) or N, wherein R⁷ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl;
X⁸ is C; and
X⁹, when present, is C(R^{9a})(R^{9b}); wherein each R^{9a} and R^{9b} is independently H, halogen or unsubstituted alkyl.

B44. The ADC of embodiment B41, B42 or B43,

wherein:
X¹ is C(R^{1a})(R^{1b}); wherein each R^{1a} and R^{1b} is H;
X² is C(R^{2a})(R^{2b}); wherein each R^{2a} and R^{2b} is H;
X³ is C;
X⁴ is C(R⁴) or N, wherein R⁴ is H;
X⁵ is C(R⁵) or N, wherein R⁵ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl;
X⁶ is C(R⁶) or N, wherein R⁶ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl;
X⁷ is C(R⁷) or N, wherein R⁷ is H;
X⁸ is C; and
X⁹, when present, is CH₂.

B45. The ADC of any one of embodiments B41 to B44, wherein each said heteroalkyl is alkoxy.

B46. The ADC of any one of embodiments B41 to B45, wherein:
X¹ is C(R^{1a})(R^{1b}); wherein each R^{1a} and R^{1b} is H;
X² is C(R^{2a})(R^{2b}); wherein each R^{2a} and R^{2b} is H;
X³ is C;
X⁴ is C(R⁴) or N, wherein R⁴ is H;
X⁵ is C(R⁵) or N, wherein R⁵ is H, halogen or alkoxy;
X⁶ is C(R⁶) or N, wherein R⁶ is H, halogen or alkoxy;
X⁷ is C(R⁷) or N, wherein R⁷ is H;
X⁸ is C; and
X⁹, when present, is CH₂.

B47. The ADC of embodiment B46, wherein X⁵ is C(R⁵) or N, wherein R⁵ is H or alkoxy; and X⁶ is C(R⁶) or N, wherein R⁶ is H or alkoxy.

B48. The ADC of any one of embodiments B41 to B47, wherein X⁴ is N, X⁵ is C(R⁵), X⁶ is C(R⁶) and X⁷ is C(R⁷).

B49. The ADC of any one of embodiments B41 to B47, wherein X⁴ is C(R⁴), X⁵ is N, X⁶ is C(R⁶) and X⁷ is C(R⁷).

B50. The ADC of any one of embodiments B41 to B47, wherein X⁴ is C(R⁴), X⁵ is C(R⁵), X⁶ is N and X⁷ is C(R⁷).

B51. The ADC of any one of embodiments B41 to B47, wherein X⁴ is C(R⁴), X⁵ is C(R⁵), X⁶ is C(R⁶) and X⁷ is C(R⁷).

B52. The ADC of any one of embodiments B41 to B51, wherein at least one of X⁴ and X⁷ is CH.

B53. The ADC of any one of embodiments B41 to B47 and B49 to B52, wherein each of X⁴ and X⁷ is CH.

B54. The ADC of any one of embodiments B41 to B53, wherein at least one of R⁵ and R⁶ is alkoxy.

B55. The ADC of any one of embodiments B45 to B54, wherein each said alkoxy is independently —OR^{k}, wherein each R^{k} is independently alkyl optionally substituted with heterocyclyl or —N(R^{d})(R^{e}); wherein said heterocyclyl contains at least one nitrogen atom, and each R^{d} and R^{e} is independently H, alkyl, alkenyl or alkynyl.

B56. The ADC of embodiment B55, wherein each said alkoxy is selected from the group consisting of —OCH₃, —OCH₂CH₃, —OCH₂CH₂CH₃, —OCH(CH₃)₂, —OCH₂CH₂N(CH₃)₂,

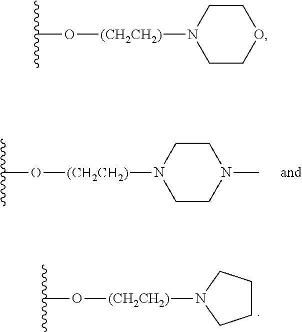

B57. The ADC of any one of embodiments B41 to B56, wherein d is 1, 2, 3 or 4.

B58. The ADC of any one of embodiments B41 to B57, wherein L is a phosphate-based linker.

B59. The ADC of embodiment B58, wherein the phosphate-based linker comprises a phosphate-based moiety having the following structure:

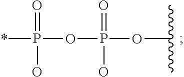 [SMILES: *OP(=O)(O)OP(*)(=O)O]

wherein * denotes the connection to the —O— atom at position L of Formula (II); wherein L further comprises at least one additional moiety, and the wavy line of the phosphate-based moiety denotes the connection to one of the at least one additional moiety; wherein each at least one additional moiety is independently selected from the group consisting of unsubstituted alkylene, -(alkylene-O)—, —C(O)—, —N(R_{w})—, a water-soluble polymer and an amino acid; wherein each R_{w} is independently H or C₁-C₈ alkyl; and combinations thereof.

B60. The ADC of any one of embodiments B41 to B57, wherein L is selected from the group of linkers of Table 6.

B61. The ADC of any one of embodiments B41 to B57, wherein L is selected from the group of linkers of Table 7.

B62. The ADC of any one of embodiments B41 to B57, wherein L is selected from the group of linkers of Table 8.

B63. The ADC of any one of embodiments B41 to B57, wherein L has the following structure:

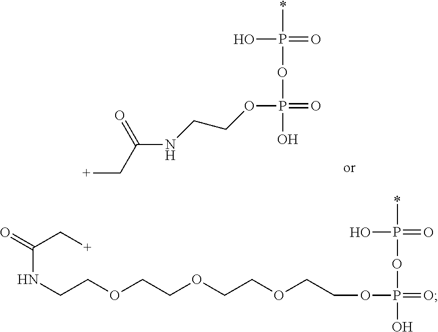

wherein * denotes the connection to the —O— atom at position L of Formula (II); and + denotes the connection to E.

B64. The ADC of any one of embodiments B41 to B57, wherein L has the following structure:

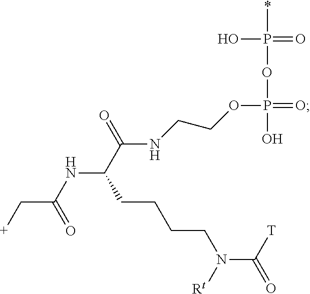

wherein T is a water-soluble polymer; R^{t} is H or methyl; * denotes the connection to the —O— atom at position L of Formula (II); and + denotes the connection to E.

B65. The ADC of embodiment B64, wherein the water-soluble polymer is a (polyethylene)glycol (PEG) moiety.

B66. The ADC of embodiment B65, wherein the PEG moiety has a molecular weight within a range of about 100 Da to about 100,000 Da, about 100 Da to about 10,000 Da, about 100 Da to about 5,000 Da, or about 100 Da to about 1,000 Da.

B67. The ADC of embodiment B65, wherein the PEG moiety is —(CH₂CH₂O)ₙCH₃, wherein n is an integer from 1 to 24.

B68. The ADC of embodiment B65, wherein the PEG moiety is —(CH₂CH₂O)ₙCH₃, wherein n is 8, 9, 10, 11 or 12.

B69. The ADC of any one of embodiments B41 to B68, wherein E comprises an amide, an ester, a thioester, a pyrrolidine-2,5-dione, an oxime, a 1,2,3-triazole or a 1,4-dihydropyridazine, wherein the 1,2,3-triazole and the 1,4-dihydropyridazine are each optionally fused to an 8-membered ring.

B70. The ADC of embodiment B69, wherein E is selected from the group consisting of:

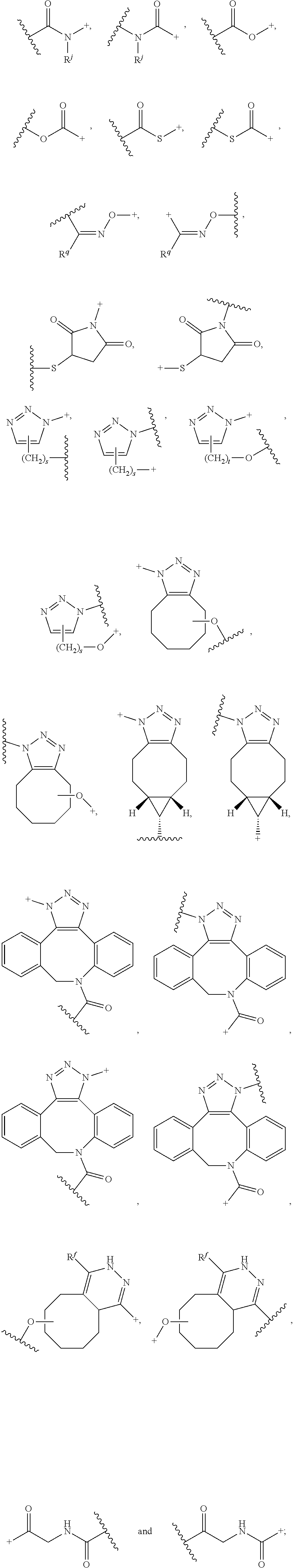

wherein each R^{j} is independently H or unsubstituted C₁-C₆ alkyl; each R^{q} is independently unsubstituted C₁-C₆ alkyl; each R^{f} is independently H or unsubstituted C₁-C₆ alkyl; each s is independently 0, 1, 2, 3, 4, 5 or 6; each t is independently 0, 1, 2, 3, 4, 5 or 6; each + denotes connection to L; and each wavy line denotes connection to Ab.

B71. The ADC of any one of embodiments B41 to B70, wherein E is:

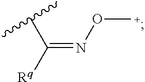

wherein R^{q} is unsubstituted C₁-C₆ alkyl.

B72. The ADC of embodiment B71, wherein R^{q} is methyl.

B73. The ADC of any one of embodiments B41 to B72, wherein E joins L to a non-natural amino acid of Ab.

B74. The ADC of any one of embodiments B41 to B73, wherein Ab is configured to bind to an antigen.

B75. The ADC of embodiment B74, wherein the antigen selected from the group consisting of PD-1, PD-Li, PSMA, CD70, CD3, HER2, HER3, TROP2, GPC3, VEGFR, EGFR, c-Met (HGFR), CD19, CD22, CD25 (IL-2R alpha), CD30, CD33, CD37, CD46, CD48, CD56 (NCAM-1), CD71 (Transferrin R), CD74, CD79b, CD123 (IL-3R alpha), CD138 (syndecan-1), CD142, CD166 (ALCAM), CD203c (ENPP3), CD205 (LY75), CD221 (IGF-1R), CD262 (TRAIL R2), CD276 (B7-H3), mesothelin, EpCAM, CEACAM5, CEACAM6, DLL3, ROR1, ROR2, GPNMB, GCC, GUCY2c, NaPi2b, Flt-1, Flt-3, folate receptor alpha, Tissue Factor (TF), CA6, MUC1, MUC16 (CA-125), BCMA, SLAMF7 (CS1), TIM1, CanAg, Ckit (CD117), EphA2, Nectin4, SLTRK6, FGFR2, LYPD3 (C4.4a), Cadherin 3, 5T4 (TPBG), STEAP1, PTK7, Ephrin-A4, LIV-1 (SLC39A6 or ZIP6), SLC1A5, TENB2, ETBR, integrin v3, Cripto, AGS-5 (SLC44A4), LY6E, AXL, LAMP1, LRRC15, TNF-alpha and MN/CA IX.

B76. The ADC of embodiment B75, wherein the antigen is TROP2, CD70, HER2, PSMA, HER3 or GPC3.

B77. The ADC of any one of embodiments B41 to B76, wherein Ab is an anti-CD70 antibody comprising a sequence listed in Table 2.

B78. The ADC of embodiment B77, wherein the anti-CD70 antibody comprises a heavy chain variable region having the amino acid sequence of SEQ ID NO: 26.

B79. The ADC of embodiment B77 or B78, wherein the anti-CD70 antibody comprises a light chain variable region having the amino acid sequence of SEQ ID NO: 27.

B80. The ADC of embodiment B77, B78 or B79, wherein the anti-CD70 antibody comprises a heavy chain having the amino acid sequence of SEQ ID NO: 25.

B81. The ADC of embodiment B77, B78 or B79, wherein the anti-CD70 antibody comprises a heavy chain having the amino acid sequence of SEQ ID NO: 20.

B82. The ADC of any one of embodiments B77 to B81, wherein the anti-CD70 antibody comprises a light chain having the amino acid sequence of SEQ ID NO: 19.

B83. The ADC of embodiment B77, wherein the anti-CD70 antibody comprises two heavy chains, each having the amino acid sequence of SEQ ID NO: 20, and two light chains, each having the amino acid sequence of SEQ ID NO: 19.

B84. The ADC of any one of embodiments A61 to A122 and B41 to B76, wherein Ab is an anti-TROP2 antibody comprising a sequence listed in Table 1.

B85. The ADC of embodiment B84, wherein the anti-TROP2 antibody comprises a heavy chain having the amino acid sequence of SEQ ID NO: 5.

B86. The ADC of embodiment B84 or B85, wherein the anti-TROP2 antibody comprises a light chain having the amino acid sequence of SEQ ID NO: 4.

B87. The ADC of any one of embodiments A61 to A122 and B41 to B76, wherein Ab is an anti-HER2 antibody comprising a sequence listed in Table 3.

B88. The ADC of embodiment B87, wherein the anti-HER2 antibody comprises a heavy chain having the amino acid sequence of SEQ ID NO: 29.

B89. The ADC of embodiment B87 or B88, wherein the anti-HER2 antibody comprises a light chain having the amino acid sequence of SEQ ID NO: 30.

B90. The ADC of any one of embodiments A61 to A122 and B41 to B76, wherein Ab is an anti-PSMA antibody comprising a sequence listed in Table 4.

B91. The ADC of embodiment B90, wherein the anti-PSMA antibody comprises a heavy chain having the amino acid sequence of SEQ ID NO: 39.

B92. The ADC of embodiment B90 or B91, wherein the anti-PSMA antibody comprises a light chain having the amino acid sequence of SEQ ID NO: 40.

B93. The ADC of any one of embodiments A61 to A122 and B41 to B76, wherein Ab is an anti-HER3 antibody comprising a sequence listed in Table 5.

B94. The ADC of embodiment B93, wherein the anti-HER3 antibody comprises a heavy chain having the amino acid sequence of SEQ ID NO: 58.

B95. The ADC of embodiment B93 or B94, wherein the anti-HER3 antibody comprises a light chain having the amino acid sequence of SEQ ID NO: 47.

B96. The ADC of any one of embodiments B41 to B95, wherein the antibody comprises two heavy chains, and one non-natural amino acid is incorporated into each said heavy chain.

B97. The ADC of any one of embodiments B41 to B96, wherein the non-natural amino acid is para-acetyl-L-phenylalanine.

B98. The compound of any one of embodiments B1 to B4, or the ADC of any one of embodiments B41 to B97, wherein the corresponding amine of moiety A has a C log P value of at least about 1.

B99. A pharmaceutical composition comprising a compound of any one of embodiments B1 to B40 and B98, or an ADC of any one of embodiments B41 to B98, and at least one pharmaceutically acceptable adjuvant, binder, buffer, carrier, diluent or excipient.

B100. A method of treating a disease or condition in a subject, the method comprising administering to the subject a therapeutically effect amount of a compound of any one of embodiments A1 to A60, Bi to B40 and B98, an ADC of any one of embodiments A61 to A130, B41 to B98, or a pharmaceutical composition of embodiment A131, A132 or B99.

B101. The method of embodiment B100, wherein the disease or condition is cancer.

B102. The method of embodiment B101, wherein the cancer is a CD70-expression cancer.

B103. The method of embodiment B101 or B102, wherein the cancer is renal cell carcinoma.

B104. The method of embodiment B101 or B102, wherein the cancer is a blood cancer.

B105. The method of embodiment B104, wherein the blood cancer is a leukemia, lymphoma or myeloma.

While certain embodiments of the inventions have been described, these embodiments have been presented by way of example only and are not intended to limit the scope of the disclosure. Indeed, the novel compositions, methods and systems described herein may be embodied in a variety of other forms. Furthermore, various omissions, substitutions and changes in the compositions, systems and methods described herein may be made without departing from the spirit of the disclosure. The accompanying claims and their equivalents are intended to cover such forms or modifications as would fall within the scope and spirit of the disclosure. Accordingly, the scope of the present inventions is defined only by reference to the appended claims.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the compositions, steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the compositions, steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Although features may be described above as acting in certain combinations, one or more features from a claimed combination can, in some cases, be excised from the combination, and the combination may be claimed as a subcombination or variation of a subcombination.

The features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

As used herein and in the appended claims, the singular forms “a,” “an,” and “the” include plural reference unless the context clearly indicates otherwise.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” “substantial” and “substantially” as used herein represent a value, amount, quantity or characteristic close to the stated value, amount, quantity or characteristic that still performs a desired function or achieves a desired result. For example, the terms “approximately”, “about”, “generally,” “substantial” and “substantially” may refer to an amount that is within less than 10% of, within less than 5% of, within less than 1% of: within less than 0.1% of, and within less than 0.01% of the stated amount.

As used herein and in the appended claims, the term “comprising” is open ended, and the broadest reasonable interpretation of the term applies. The present disclosure contemplates alternative embodiments wherein the term “consisting of” can be used in place of each recitation of the term “comprising” herein.

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

1. A compound of Formula (I) having the following structure: 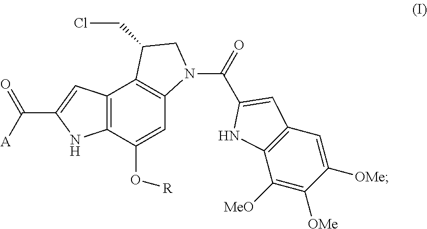
wherein:
R is H or L-W, wherein L is a phosphate-based linker and W is a reactive moiety; and
A is selected from the group consisting of formula (a), (b), (c) and (d), having the following structures: 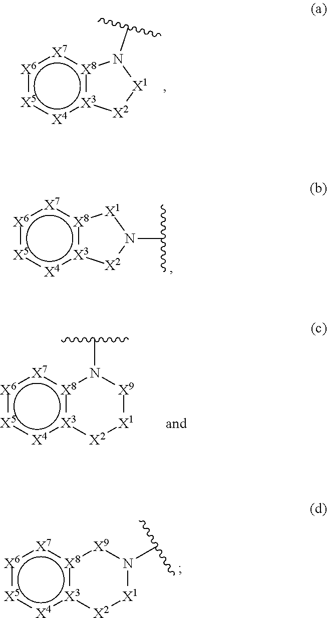
wherein:
each X¹ is C(R^{1a})(R^{1b}); wherein each R^{1a} and R^{1b} is independently H, halogen, alkyl, alkenyl or alkynyl;
each X² is C(R^{2a})(R^{2b}); wherein each R^{2a} and R^{2b} is independently H, halogen, alkyl, alkenyl or alkynyl;
each X³ is C;
each X⁴ is C(R⁴) or N, wherein R⁴ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
each X⁵ is C(R⁵) or N, wherein R⁵ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
each X⁶ is C(R⁶) or N, wherein R⁶ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
each X⁷ is C(R⁷) or N, wherein R⁷ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
each X⁸ is C; and
each X⁹, when present, is C(R^{9a})(R^{9b}); wherein each R^{9a} and R^{9b} is independently H, halogen, alkyl, alkenyl or alkynyl;
wherein:
each R^{a} and R^{b} is independently H, alkyl, alkenyl or alkynyl;
each R^{c} is independently H, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl or heteroarylalkyl;
each R^{s} is independently H, halogen, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl or heteroarylalkyl; and
each m is independently 0, 1, 2 or 3;
or a salt thereof.

2. The compound of claim 1, wherein A has the structure of formula (a), and the compound is a compound of Formula (Ia) having the following structure: 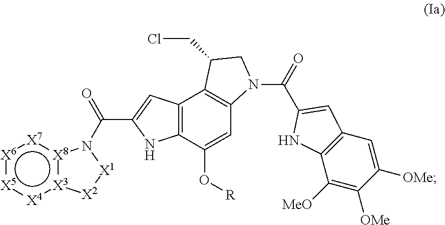
wherein:
R is H or L-W, wherein L is a phosphate-based linker and W is a reactive moiety;
X¹ is C(R^{1a})(R^{1b}); wherein each R^{1a} and R^{1b} is independently H, halogen, alkyl, alkenyl or alkynyl;
X² is C(R^{2a})(R^{2b}); wherein each R^{2a} and R^{2b} is independently H, halogen, alkyl, alkenyl or alkynyl;
X³ is C;
X⁴ is C(R⁴) or N, wherein R⁴ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
X⁵ is C(R⁵) or N, wherein R⁵ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
X⁶ is C(R⁶) or N, wherein R⁶ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s});
X⁷ is C(R⁷) or N, wherein R⁷ is H, halogen, —OH, —SH, —NO₂, —CN, —N₃, —N(R^{a})(R^{b}), acyl, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl, heteroarylalkyl, —C(O)R^{c}, —C(O)OR^{c}, —C(O)N(R^{a})(R^{b}), —C(S)R^{c}, —C(S)OR^{c}, —C(S)N(R^{a})(R^{b}), —C(O)SR^{c} or —S(O)ₘ(R^{s}); and
X⁸ is C;
wherein:
each R^{a} and R^{b} is independently H, alkyl, alkenyl or alkynyl;
each R^{c} is independently H, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl or heteroarylalkyl;
each R^{s} is independently H, halogen, alkyl, alkenyl, alkynyl, carbocyclyl, carbocyclylalkyl, heteroalkyl, heteroalkenyl, heteroalkynyl, heterocyclyl, heterocyclylalkyl, aryl, arylalkyl, heteroaryl or heteroarylalkyl; and
each m is independently 0, 1, 2 or 3.

3. The compound of claim 1, wherein:
X¹ is C(R^{1a})(R^{1b}); wherein each R^{1a} and R^{1b} is independently H, halogen or unsubstituted alkyl;
X² is C(R^{2a})(R^{2b}); wherein each R^{2a} and R^{2b} is independently H, halogen or unsubstituted alkyl;
X³ is C;
X⁴ is C(R⁴) or N, wherein R⁴ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl;
X⁵ is C(R⁵) or N, wherein R⁵ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl;
X⁶ is C(R⁶) or N, wherein R⁶ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl;
X⁷ is C(R⁷) or N, wherein R⁷ is H, halogen, alkyl, alkenyl, alkynyl, carbocyclyl, carbocyclylalkyl, heteroalkyl, heteroalkenyl, heteroalkynyl, heterocyclyl, heterocyclylalkyl or heteroarylalkyl;
X⁸ is C; and
X⁹, when present, is C(R^{9a})(R^{9b}); wherein each R^{9a} and R^{9b} is independently H, halogen or unsubstituted alkyl.

4. (canceled)

5. The compound of claim 1, wherein each said heteroalkyl is alkoxy.

6.-14. (canceled)

15. The compound of claim 5, wherein each said alkoxy is independently —OR^{k}, wherein each R^{k} is independently alkyl optionally substituted with heterocyclyl or —N(R^{d})(R^{e}); wherein said heterocyclyl contains at least one nitrogen atom, and each R^{d} and R^{e} is independently H, alkyl, alkenyl or alkynyl.

16. The compound of claim 15, wherein each said alkoxy is selected from the group consisting of —OCH₃, —OCH₂CH₃, —OCH₂CH₂CH₃, —OCH(CH₃)₂, —OCH₂CH₂N(CH₃)₂,

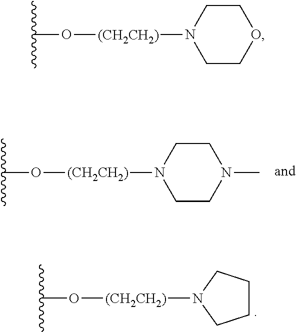

17. (canceled)

18. (canceled)

19. The compound of claim 1, wherein the phosphate-based linker comprises a phosphate-based moiety having the following structure: 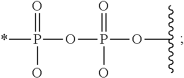 [SMILES: *OP(=O)(O)OP(*)(=O)O]
wherein * denotes the connection to the —O— atom at position R of Formula (I) or Formula (Ia); wherein L further comprises at least one additional moiety, and the wavy line of the phosphate-based moiety denotes the connection to one of the at least one additional moiety; wherein the at least one additional moiety is selected from the group consisting of unsubstituted alkylene, substituted alkylene, -(alkylene-O)—, optionally substituted arylene, —O—, —C(O)—, —N(R_{w})—, —S(O)₀₋₂—, a water-soluble polymer and an amino acid; wherein each R_{w} is independently H or C₁-C₈ alkyl; and combinations thereof.

20. The compound of claim 19, wherein each at least one additional moiety is independently selected from the group consisting of unsubstituted alkylene, -(alkylene-O)—, —C(O)—, —N(R_{w})—, a water-soluble polymer and an amino acid; wherein each R_{w} is independently H or C₁-C₈ alkyl; and combinations thereof.

21.-23. (canceled)

24. The compound of claim 1, wherein R is L-W, and L has the following structure: 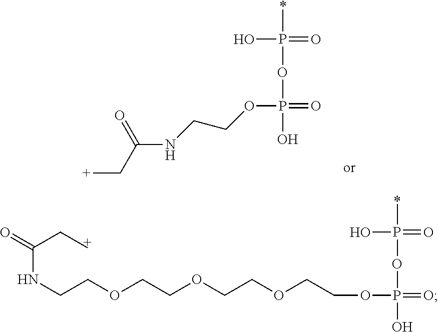
wherein * denotes the connection to the —O— atom at position R of Formula (I) or Formula (Ia); and + denotes the connection to W.

25. The compound of claim 1, wherein R is L-W, and L has the following structure: 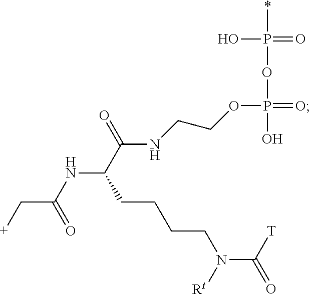
wherein T is a water-soluble polymer; R is H or methyl; * denotes the connection to the —O— atom at position R of Formula (I) or Formula (Ia); and + denotes the connection to W.

26. The compound of claim 25, wherein the water-soluble polymer is a (polyethylene)glycol (PEG) moiety.

27. The compound of claim 26, wherein the PEG moiety has a molecular weight within a range of about 100 Da to about 100,000 Da, about 100 Da to about 10,000 Da, about 100 Da to about 5,000 Da, or about 100 Da to about 1,000 Da.

28. The compound of claim 26, wherein the PEG moiety is —(CH2CH2O)nCH3, wherein n is an integer from 1 to 24.

29. (canceled)

30. The compound of claim 1, wherein R is L-W, and the reactive moiety W comprises —N₃, —OH, —SH, —NH(R^{j}), —C(O)R^{q}, —C(O)OR^{x}, —C(O)CH₂NH₂, an activated ester, —O—NH₂, a maleimide, a tetrazine, an alkyne, a cyclooctyne or an (E)-cyclooctene; wherein R^{j} is H or unsubstituted alkyl, R^{q} is unsubstituted alkyl, and R^{x} is H, unsubstituted alkyl or a carboxylic acid protecting group.

31. The compound of claim 30, wherein reactive moiety W is selected from the group consisting of: 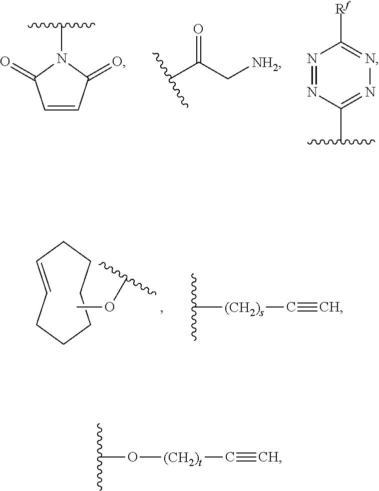
—N₃, —OH, —SH, —NH(R^{j}), —C(O)R^{q}, —C(O)OR^{x}, an activated ester, —O—NH₂ and an optionally substituted monocyclic or polycyclic group comprising the cyclooctyne;
wherein:
R^{j} is H or unsubstituted C₁-C₆ alkyl,
R^{q} is unsubstituted C₁-C₆ alkyl,
R^{x} is H, unsubstituted C₁-C₆ alkyl or a carboxylic acid protecting group,
R^{f} is H or unsubstituted C₁-C₆ alkyl,
s is 0, 1, 2, 3, 4, 5 or 6, and
t is 0, 1, 2, 3, 4, 5 or 6.

32. The compound of claim 1, wherein W is —ONH₂.

33. The compound of claim 1, wherein the compound is selected from the group consisting of: 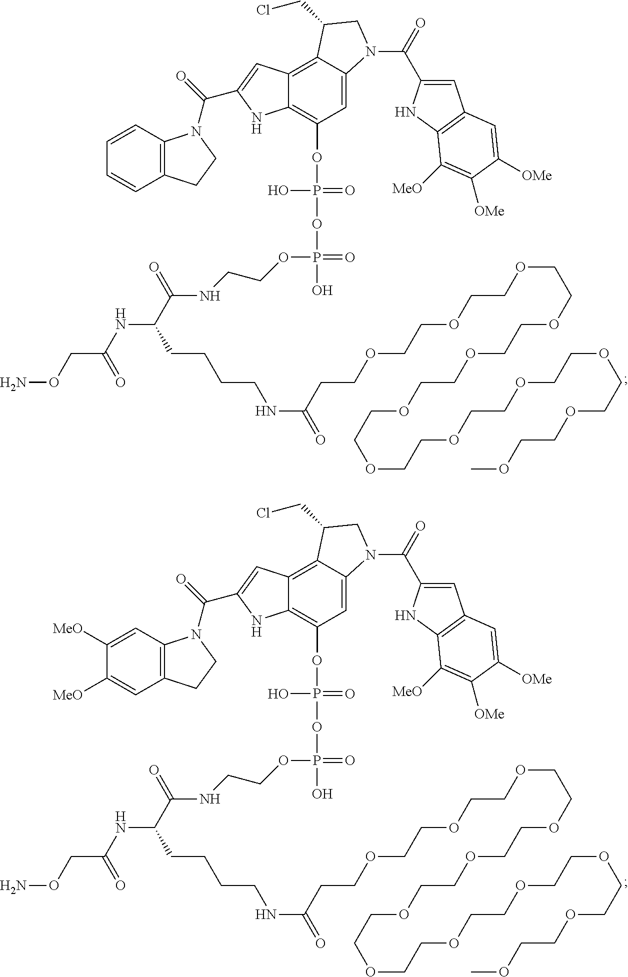 [SMILES: COCCOCCOCCOCCOCCOCCOCCOCCOCCOCCOCCOCCC(=O)NCCCC[C@H](NC(=O)CON)C(=O)NCCOP(=O)(O)OP(=O)(O)Oc1cc2c(c3cc(C(=O)N4CCc5ccccc54)[nH]c13)[C@H](CCl)CN2C(=O)c1cc2cc(OC)c(OC)c(OC)c2[nH]1 ; COCCOCCOCCOCCOCCOCCOCCOCCOCCOCCOCCOCCC(=O)NCCCC[C@H](NC(=O)CON)C(=O)NCCOP(=O)(O)OP(=O)(O)Oc1cc2c(c3cc(C(=O)N4CCc5cc(OC)c(OC)cc54)[nH]c13)[C@H](CCl)CN2C(=O)c1cc2cc(OC)c(OC)c(OC)c2[nH]1] 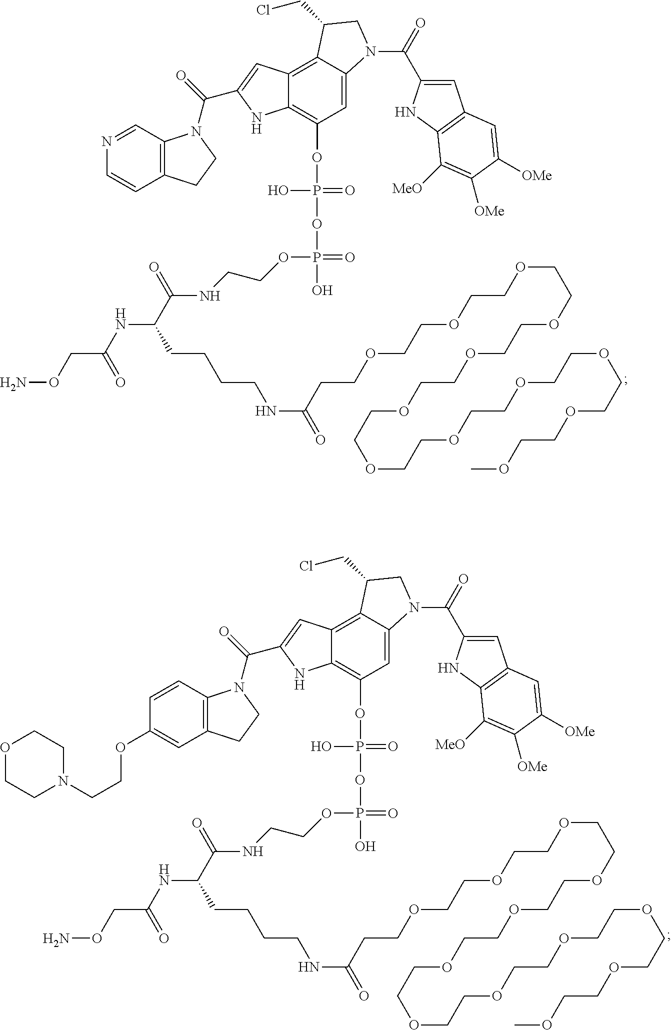 [SMILES: COCCOCCOCCOCCOCCOCCOCCOCCOCCOCCOCCOCCC(=O)NCCCC[C@H](NC(=O)CON)C(=O)NCCOP(=O)(O)OP(=O)(O)Oc1cc2c(c3cc(C(=O)N4CCc5ccncc54)[nH]c13)[C@H](CCl)CN2C(=O)c1cc2cc(OC)c(OC)c(OC)c2[nH]1 ; COCCOCCOCCOCCOCCOCCOCCOCCOCCOCCOCCOCCC(=O)NCCCC[C@H](NC(=O)CON)C(=O)NCCOP(=O)(O)OP(=O)(O)Oc1cc2c(c3cc(C(=O)N4CCc5cc(OCCN6CCOCC6)ccc54)[nH]c13)[C@H](CCl)CN2C(=O)c1cc2cc(OC)c(OC)c(OC)c2[nH]1] 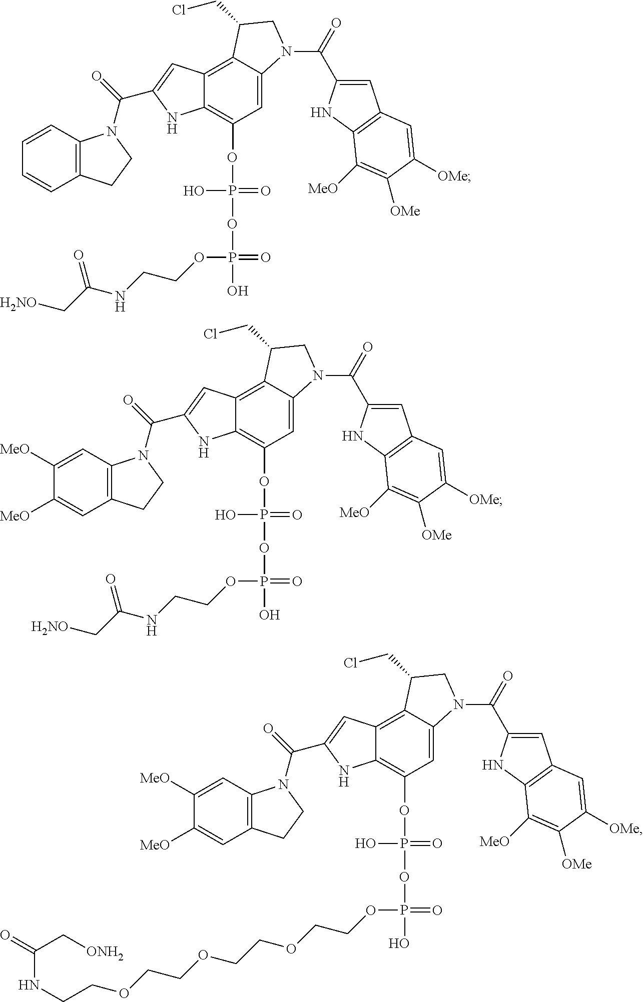 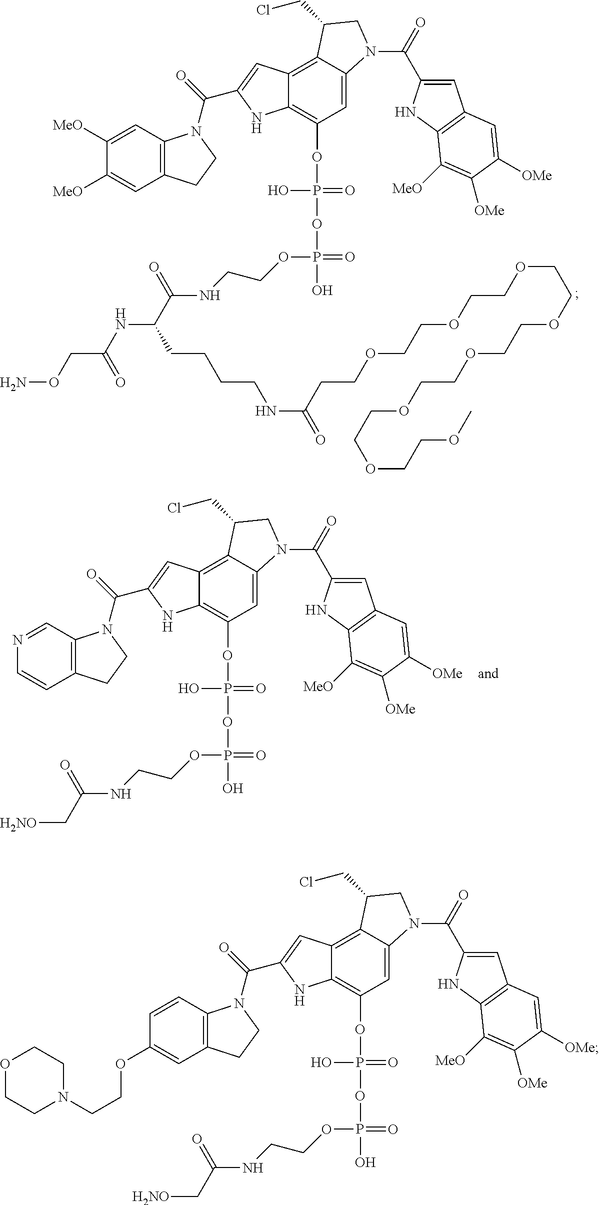
and salts thereof.

34. The compound of claim 1, having the following structure: 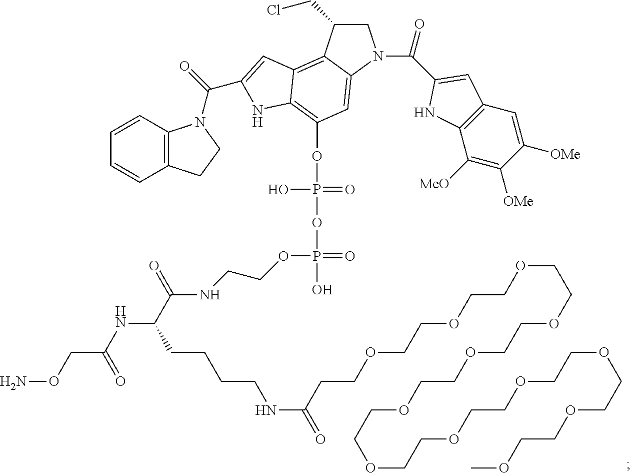 [SMILES: COCCOCCOCCOCCOCCOCCOCCOCCOCCOCCOCCOCCC(=O)NCCCC[C@H](NC(=O)CON)C(=O)NCCOP(=O)(O)OP(=O)(O)Oc1cc2c(c3cc(C(=O)N4CCc5ccccc54)[nH]c13)[C@H](CCl)CN2C(=O)c1cc2cc(OC)c(OC)c(OC)c2[nH]1]
or a salt thereof.

35. The compound of claim 1, having the following structure: 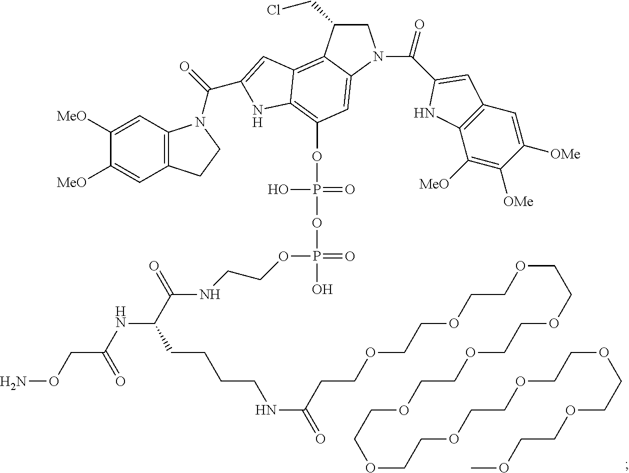 [SMILES: COCCOCCOCCOCCOCCOCCOCCOCCOCCOCCOCCOCCC(=O)NCCCC[C@H](NC(=O)CON)C(=O)NCCOP(=O)(O)OP(=O)(O)Oc1cc2c(c3cc(C(=O)N4CCc5cc(OC)c(OC)cc54)[nH]c13)[C@H](CCl)CN2C(=O)c1cc2cc(OC)c(OC)c(OC)c2[nH]1]
or a salt thereof.

36. The compound of claim 1, having the following structure: 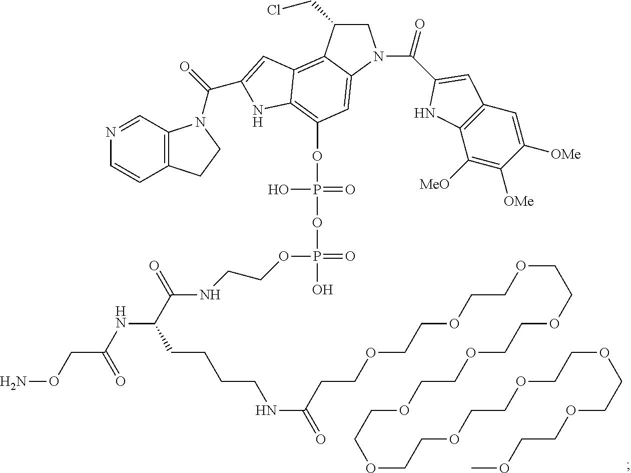 [SMILES: COCCOCCOCCOCCOCCOCCOCCOCCOCCOCCOCCOCCC(=O)NCCCC[C@H](NC(=O)CON)C(=O)NCCOP(=O)(O)OP(=O)(O)Oc1cc2c(c3cc(C(=O)N4CCc5ccncc54)[nH]c13)[C@H](CCl)CN2C(=O)c1cc2cc(OC)c(OC)c(OC)c2[nH]1]
or a salt thereof.

37. The compound of claim 1, having the following structure: 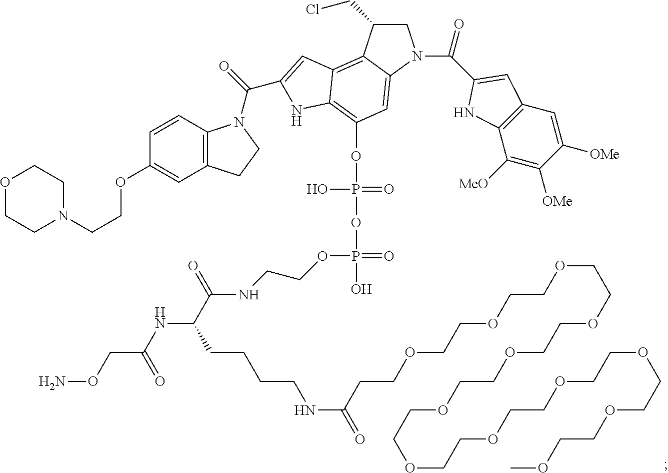 [SMILES: COCCOCCOCCOCCOCCOCCOCCOCCOCCOCCOCCOCCC(=O)NCCCC[C@H](NC(=O)CON)C(=O)NCCOP(=O)(O)OP(=O)(O)Oc1cc2c(c3cc(C(=O)N4CCc5cc(OCCN6CCOCC6)ccc54)[nH]c13)[C@H](CCl)CN2C(=O)c1cc2cc(OC)c(OC)c(OC)c2[nH]1]
or a salt thereof.

38. (canceled)

39. The compound of claim 1, wherein the compound is selected from the group consisting of: 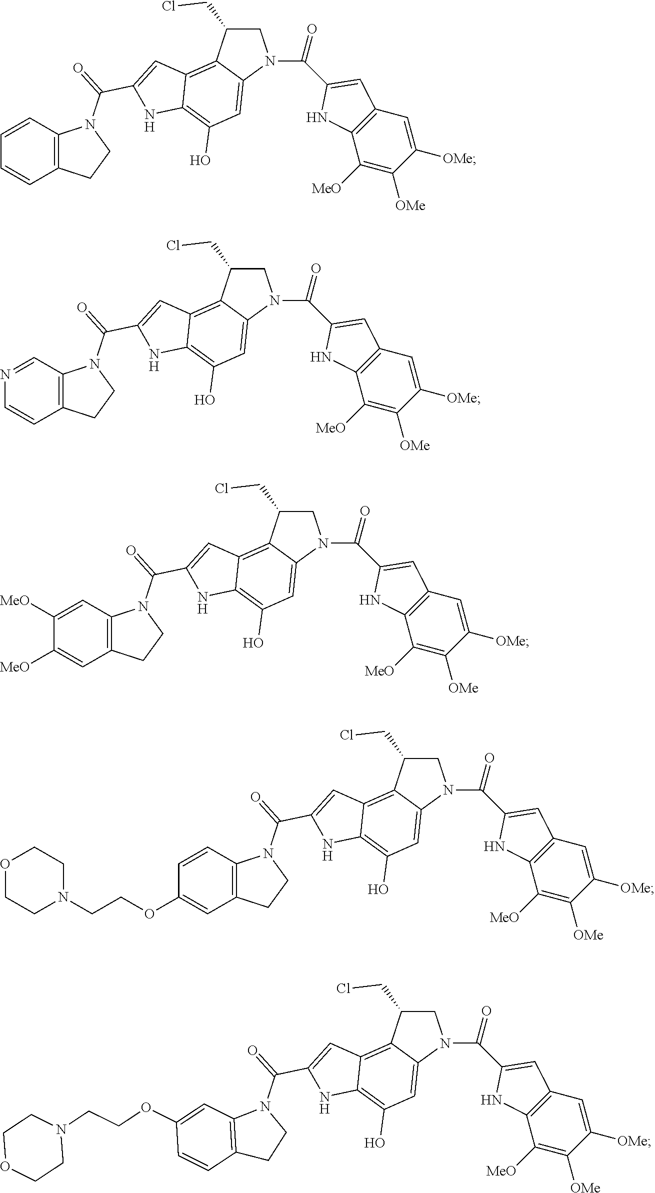 [SMILES: COc1cc2cc(C(=O)N3C[C@@H](CCl)c4c3cc(O)c3[nH]c(C(=O)N5CCc6ccccc65)cc43)[nH]c2c(OC)c1OC ; COc1cc2cc(C(=O)N3C[C@@H](CCl)c4c3cc(O)c3[nH]c(C(=O)N5CCc6ccncc65)cc43)[nH]c2c(OC)c1OC ; COc1cc2c(cc1OC)N(C(=O)c1cc3c4c(cc(O)c3[nH]1)N(C(=O)c1cc3cc(OC)c(OC)c(OC)c3[nH]1)C[C@H]4CCl)CC2 ; COc1cc2cc(C(=O)N3C[C@@H](CCl)c4c3cc(O)c3[nH]c(C(=O)N5CCc6cc(OCCN7CCOCC7)ccc65)cc43)[nH]c2c(OC)c1OC ; COc1cc2cc(C(=O)N3C[C@@H](CCl)c4c3cc(O)c3[nH]c(C(=O)N5CCc6ccc(OCCN7CCOCC7)cc65)cc43)[nH]c2c(OC)c1OC] 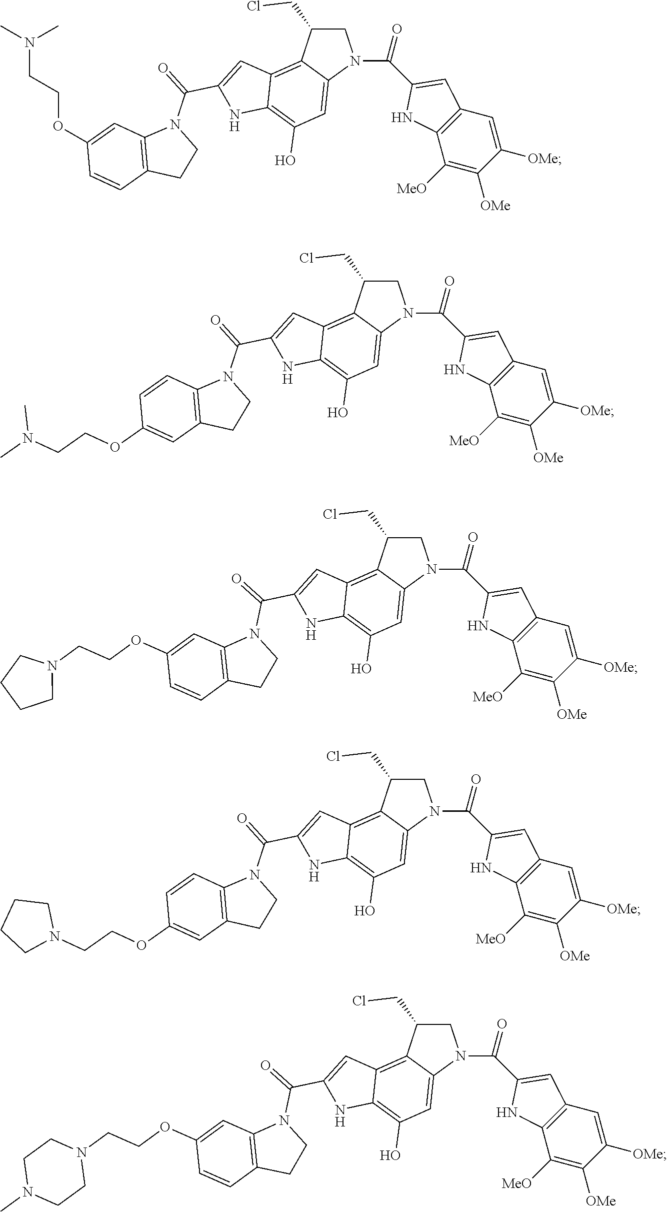 [SMILES: COc1cc2cc(C(=O)N3C[C@@H](CCl)c4c3cc(O)c3[nH]c(C(=O)N5CCc6ccc(OCCN(C)C)cc65)cc43)[nH]c2c(OC)c1OC ; COc1cc2cc(C(=O)N3C[C@@H](CCl)c4c3cc(O)c3[nH]c(C(=O)N5CCc6cc(OCCN(C)C)ccc65)cc43)[nH]c2c(OC)c1OC ; COc1cc2cc(C(=O)N3C[C@@H](CCl)c4c3cc(O)c3[nH]c(C(=O)N5CCc6ccc(OCCN7CCCC7)cc65)cc43)[nH]c2c(OC)c1OC ; COc1cc2cc(C(=O)N3C[C@@H](CCl)c4c3cc(O)c3[nH]c(C(=O)N5CCc6cc(OCCN7CCCC7)ccc65)cc43)[nH]c2c(OC)c1OC ; COc1cc2cc(C(=O)N3C[C@@H](CCl)c4c3cc(O)c3[nH]c(C(=O)N5CCc6ccc(OCCN7CCN(C)CC7)cc65)cc43)[nH]c2c(OC)c1OC] 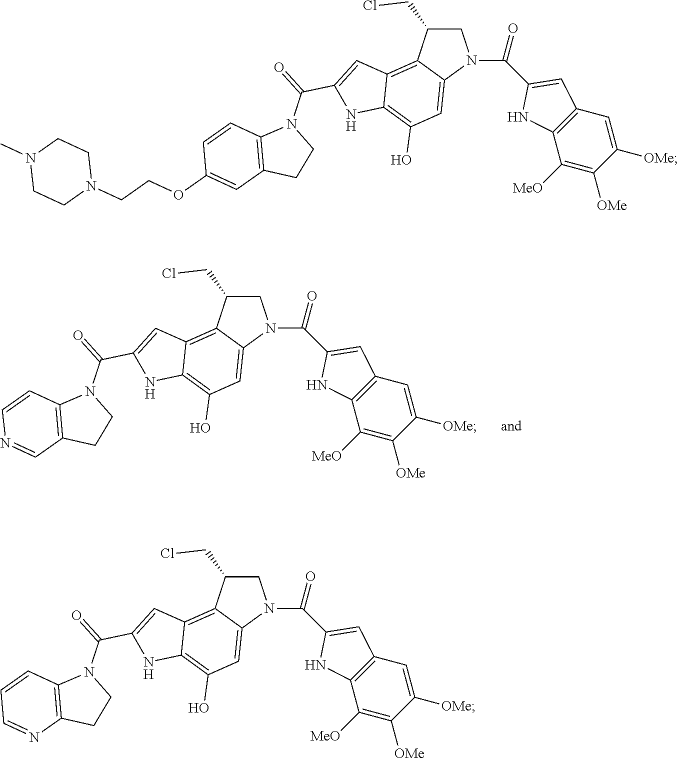 [SMILES: COc1cc2cc(C(=O)N3C[C@@H](CCl)c4c3cc(O)c3[nH]c(C(=O)N5CCc6cc(OCCN7CCN(C)CC7)ccc65)cc43)[nH]c2c(OC)c1OC ; COc1cc2cc(C(=O)N3C[C@@H](CCl)c4c3cc(O)c3[nH]c(C(=O)N5CCc6cnccc65)cc43)[nH]c2c(OC)c1OC ; "and" ; COc1cc2cc(C(=O)N3C[C@@H](CCl)c4c3cc(O)c3[nH]c(C(=O)N5CCc6ncccc65)cc43)[nH]c2c(OC)c1OC]
and salts thereof.

40. The compound of claim 1, wherein the compound is selected from the group consisting of: 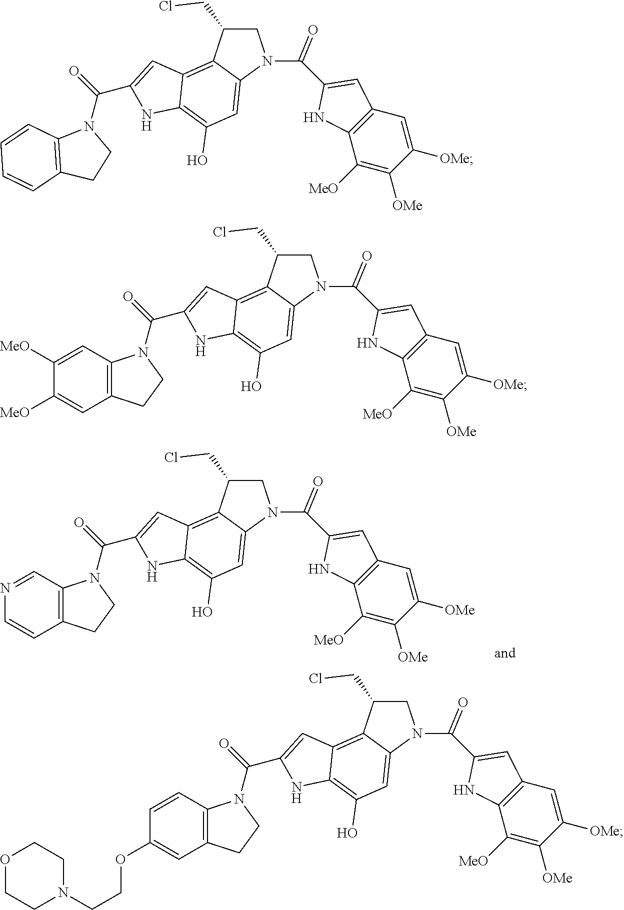
and salts thereof.

41.-104. (canceled)
